(19) United States
(12) Patent Application Publication
(10) Pub. No.: US 2021/0115476 A1
(43) Pub. Date: Apr. 22, 2021

(54) ENZYMES OF LUCIFERIN BIOSYNTHESIS AND USE THEREOF

(71) Applicant: LIGHT BIO, INC, Ketchum, ID (US)

(72) Inventor: Il'ya Viktorovich YAMPOL'SKIY, Moscow (RU)

(21) Appl. No.: 17/135,163

(22) Filed: Dec. 28, 2020

Related U.S. Application Data
(63) Continuation of application No. PCT/RU2019/050097, filed on Jun. 27, 2019.

(30) Foreign Application Priority Data
Jun. 28, 2018 (RU) 2018123601

(51) Int. Cl. C12P 1/02; C07K 14/37

(52) U.S. Cl. CPC C12P 1/02; C12Y 113/12007; C07K 14/37

(57) ABSTRACT

Present invention is aimed at identification of new fungal luciferin biosynthesis enzymes, nucleic acids able to encode these enzymes, and proteins able to catalyze certain stages of the fungal luciferin biosynthesis. The invention also provides for application of nucleic acids for producing said enzymes in a cell or organism. Methods for in vitro or in vivo preparation of chemical compounds identical to fungal luciferins and preluciferins are also provided. Vectors comprising nucleic acid described in the present invention are also provided. In addition, the present invention provides expression cassettes comprising the nucleic acid of the present invention and regulatory elements necessary for nucleic acid expression in a selected host cell. Besides, cells, stable cell lines, transgenic organisms (e.g. plants, animals, fungi, or microorganisms) including nucleic acids, vectors, or expression cassettes of the present invention are also provided. Present invention also provides combinations of nucleic acids to obtain autonomously luminous cells, cell lines, or transgenic organisms. In preferred embodiments, cells or transgenic organisms are capable to produce fungal luciferin from precursors. In some embodiments, cells or transgenic organisms are capable to produce fungal preluciferin from precursors. In some embodiments, cells or transgenic organisms are capable of bioluminescence in the presence of a fungal luciferin precursor. In some embodiments, cells or transgenic organisms are capable of autonomous bioluminescence. Combinations of proteins for producing luciferin or its precursors from more simple chemical compounds are also provided. A kit containing nucleic acids, vectors, or expression cassettes of the present invention for producing luminous cells, cell lines, or transgenic organisms is also provided.

### FIELD OF INVENTION

The group of inventions relates to the field of biotechnology and genetic engineering. In particular, the invention relates to enzymes of bioluminescent system of fungi.

### BACKGROUND OF THE INVENTION

Enzymes that can catalyze oxidation of low molecular compounds of luciferins, which is accompanied by light emission or bioluminescence, are referred to as the “luciferases”. Luciferin oxidation results in release of oxyluciferin from a complex with the luciferase enzyme.

Luciferases are widely used as the reporter genes in a number of biomedical applications and biotechnologies. For example, luciferases are used to determine viability of cells and activity of promoters or other components of living systems, in studies of carcinogenesis in animal models, in methods for detecting microorganisms or toxic agents in media, as indicators for determining concentrations of various substances, to visualize passage of signaling cascades, etc. [Scott et al., Annu Rev Anal Chem, 2011, 4: 297-319; Badr and Tannous, Trends Biotechnol. 2011, 29: 624-33; Andreu et al., FEMS Microbiol Rev. 2011, 35: 360-94]. Many applications of luciferases are described in reviews [Kaskova et al., Chem Soc Rev., 2016, 45: 6048-6077; Scott et al., Annu Rev Anal Chem, 2011, 4: 297-319; Widder and Falls, IEEE Journal of Selected Topics in Quantum Electronics, 2014, 20: 232-241]. All main applications of luciferases are based on detection of light emitted depending on the phenomenon or signal being studied. Such detection, as a rule, is performed using a luminometer or modified optical microscope.

Thousands of species capable of bioluminescence are known, for which about a dozen of luciferins with various structures and several dozens of corresponding luciferase enzymes have been described. It has been shown that the bioluminescence systems arose independently in various organisms in course of evolution more than forty times [Herring, Journal of Bioluminescence and Chemiluminescence, 1987, 1: 147-63; Haddock et al., Annual Review of Marine Science, 2010; 2: 443-93].

A group of insect luciferases catalyzing oxidation of D-luciferin has been described [de Wet et al., Proc. Natl. Acad. Sci. USA, 1985, 82: 7870-3; de Wet et al., Proc. Natl. Acad. Sci. USA, 1987, 7: 725-37]. A group of luciferases catalyzing oxidation of coelenterazine has been described [O. Shimomura, Bioluminescence: Chemical Principles and Methods, World Scientific Publishing Co. Pte. Ltd, Singapore, 2006, 470 p.]. Bioluminescent systems of ostracods of Cypridina genus are known, which are characterized by highly chemically active luciferin and highly stable luciferase [Shimomura et al., Science, 1969, 164: 1299-300]. Bioluminescent systems of dinoflagellates and euphausiids are also known. At present, genes encoding three luciferases from this group are cloned [O. Shimomura, Bioluminescence: Chemical Principles and Methods, World Scientific Publishing Co. Pte. Ltd, Singapore, 2006]. However, this system is still poorly studied, in particular, complete luciferase sequences have not been established yet.

In the last years, a group of luciferases and luciferin of fungi bioluminescent system have been described. Fungi bioluminescence was known over hundreds of years, but the fungal luciferin had been identified only in 2015: it turned out to be 3-hydroxyhispidin, a metabolite capable to penetrate through cell membranes [Purtov et al., Angewandte Chemie, 2015, 54: 8124-28]. The same publication confirms the presence of an enzyme able to hydroxylate hispidin to form luciferin in the fungi lysates, but the said enzyme was not identified. The patent application 2017102986 of Jan. 30, 2017 describes luciferase genes from several fungi that contain luciferin in the form of 3-hydroxyhispidin with the following structure:

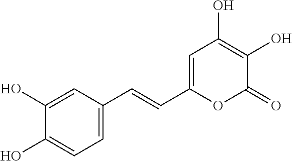 [SMILES: O=c1oc(/C=C/c2ccc(O)c(O)c2)cc(O)c1O]

It was shown that fungal luciferases can also catalyze light-emitting oxidation of other chemical compounds with structures shown in Table 1 [Kaskova et al., Sci. Adv. 2017; 3: e1602847]. All these compounds, which are fungal luciferins, including 3-hydroxyhispidin, belong to the group of 6-(2-arylvinyl)-3,4-dihydroxy-2H-pyran-2-ones and have the general formula:

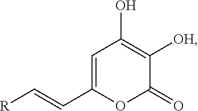

where R is aryl or heteroaryl.

TABLE 1
Examples of fungal luciferins

| IUPAC name of compound | | IUPAC name of |
|---|---|---|
| (other names of | | substitution group |
| compound) | Compound formula | (“R”) |
| (E)-6-(3,4-dihydroxystyryl)- 3,4-dihydroxy-2H-pyran-2- one (3-hydroxyhispidin) | 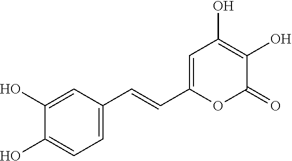 [SMILES: O=c1oc(/C=C/c2ccc(O)c(O)c2)cc(O)c1O] | 3,4-dihydroxyphenyl |
| (E)-3,4-dihydroxy-6-styryl-2H- pyran-2-one | 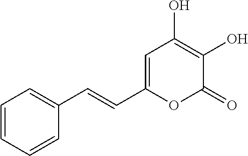 [SMILES: O=c1oc(/C=C/c2ccccc2)cc(O)c1O] | phenyl |
| (E)-3,4-dihydroxy-6-(4- hydroxystyryl)-2H-pyran-2- one | 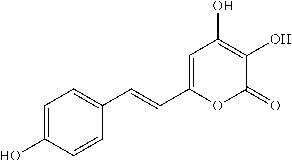 [SMILES: O=c1oc(/C=C/c2ccc(O)cc2)cc(O)c1O] | 4-hydroxyphenyl |
| (E)-3,4-dihydroxy-6-(2- hydroxystyryl)-2H-pyran-2- one | 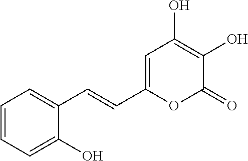 [SMILES: O=c1oc(/C=C/c2ccccc2O)cc(O)c1O] | 2-hydroxyphenyl |
| (E)-3,4-dihydroxy-6-(2,4- dihydroxystyryl)-2H-pyran-2- one | 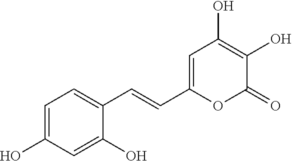 [SMILES: O=c1oc(/C=C/c2ccc(O)cc2O)cc(O)c1O] | 2,4-dihydroxyphenyl |
| (E)-3,4-dihydroxy-6-(4- hydroxy-3,5-dimethoxystyryl)- 2H-pyran-2-one | 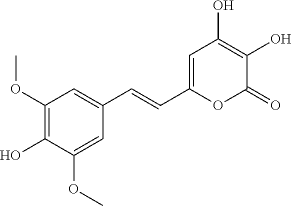 [SMILES: COc1cc(/C=C/c2cc(O)c(O)c(=O)o2)cc(OC)c1O] | 4-hydroxy-3,5- dimethoxyphenyl |
| (E)-3,4-dihydroxy-6-(4- hydroxy-3-methoxystyryl)-2H- pyran-2-one | 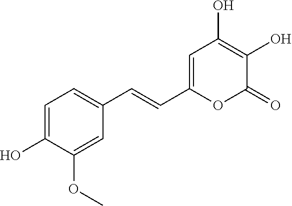 [SMILES: COc1cc(/C=C/c2cc(O)c(O)c(=O)o2)ccc1O] | 4-hydroxy-3- methoxyphenyl |
| (E)-3,4-dihydroxy-6-(2-(6- hydroxynaphthalen-2- yl)vinyl)-2H-pyran-2-one | 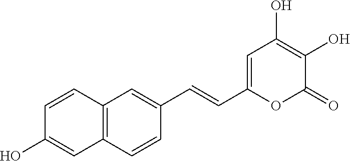 [SMILES: O=c1oc(/C=C/c2ccc3cc(O)ccc3c2)cc(O)c1O] | 6-hydroxynaphthalen- 2-yl |
| (E)-6-(4-aminostyryl)-3,4- dihydroxy-2H-pyran-2-one | 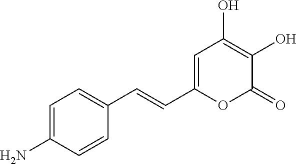 [SMILES: Nc1ccc(/C=C/c2cc(O)c(O)c(=O)o2)cc1] | 4-aminophenyl |
| (E)-6-(4-(diethylamino)styryl)- 3,4-hydroxy-2H-pyran-2-one | 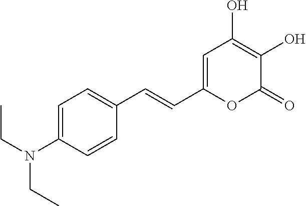 [SMILES: CCN(CC)c1ccc(/C=C/c2cc(O)c(O)c(=O)o2)cc1] | 4-diethylaminophenyl |
| (E)-6-(2-(1H-indol-3-yl)vinyl)- 3,4-dihydroxy-2H-pyran-2-one | 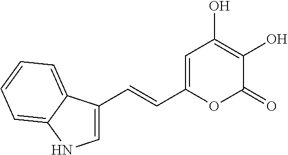 [SMILES: O=c1oc(/C=C/c2c[nH]c3ccccc23)cc(O)c1O] | 1H-indol-3-yl |
| (E)-3,4-dihydroxy-6-(2,3,6,7- tetrahydro-1H,5H- pyrido[3,2,1-ij]quinolin-9- yl)vinyl)-2H-pyran-2-one | 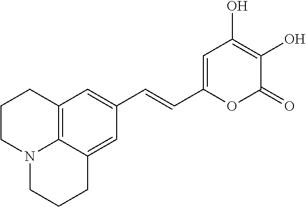 [SMILES: O=c1oc(/C=C/c2cc3c4c(c2)CCCN4CCC3)cc(O)c1O] | 2,3,6,7-tetrahydro- 1H,5H-pyrido[3,2,1- ij]quinolin-9-yl |

Enzymes that promote either synthesis of luciferins in a living organism or reduction of oxyluciferins back to luciferins are unknown in the overwhelming majority of cases. Therefore, most bioluminescent applications of luciferins involve introducing exogenous luciferase-containing luciferins (e.g. cell culture or organisms) to a system. As a consequence, use of bioluminescent systems stays limited due to a number of reasons comprising, in particular, poor penetrating ability of many luciferins through a cell membrane, chemical instability of luciferins, and complex, multistage, and expensive process of luciferins synthesis.

Enzymes that promote synthesis of luciferin are identified for the only bioluminescent system described in marine bacteria. However, this system is significantly different from other bioluminescent systems. The bacterial luciferin (myristic aldehyde) is oxidized during the reaction, but emits no light [O. Shimomura, Bioluminescence: Chemical Principles and Methods, World Scientific Publishing Co. Pte. Ltd, Singapore, 2006, 470 p.]. Besides the luciferin, key components of the luminescent reaction also include NAD (nicotinamide adenine dinucleotide) and FMN-H₂ (flavin mononucleotide). It is the oxidized derivative of FMN-H₂ that acts as a true light source. The bioluminescent system of marine bacteria is the only one to date that can be fully encoded in a heterologous expression system and considered as the closest prior art of the present invention. However, this system is generally applicable only to prokaryotic organisms. To obtain autonomous bioluminescence, the luxCDABE operon is used, which encodes luciferases (luxA and luxB heterodimers) and luxCDE luciferin biosynthesis proteins acting as the bioluminescence substrate (Meighen 1991). In 2010, this system was used to achieve autonomous luminescence in human cells. However, low bioluminescence intensity level, only 12 times higher than the signal emanating from non-bioluminescent cells, did not allow to apply the developed system for solving the most of applied problems [Close et al. PloS One, 2010, 5 (8):e12441]. Attempts to increase intensity of emitted light were unsuccessful due to toxicity of the bacterial system components for eukaryotic cells [Hollis et al. FEBS Letters, 2001, 506 (2):140-42].

In this view, identification of enzymes that promote synthesis of luciferin from stable and/or abundant in cells precursor compounds as well as reduction of oxyluciferin back to luciferin is an urgent problem. Identification of such enzymes would enable a simpler and cheaper method for synthesis of luciferin and open the way to creation of autonomous bioluminescent systems. Among them, the bioluminescent systems non-toxic for eukaryotic cells are of particular interest.

### SUMMARY OF INVENTION

Applicants have decoded stages of luciferin biosynthesis in the bioluminescent system of fungi and identified the enzymes involved in cyclic circulation of fungal luciferin and the nucleic acid sequences encoding them.

Flowchart below shows stages of fungal luciferin turnover:

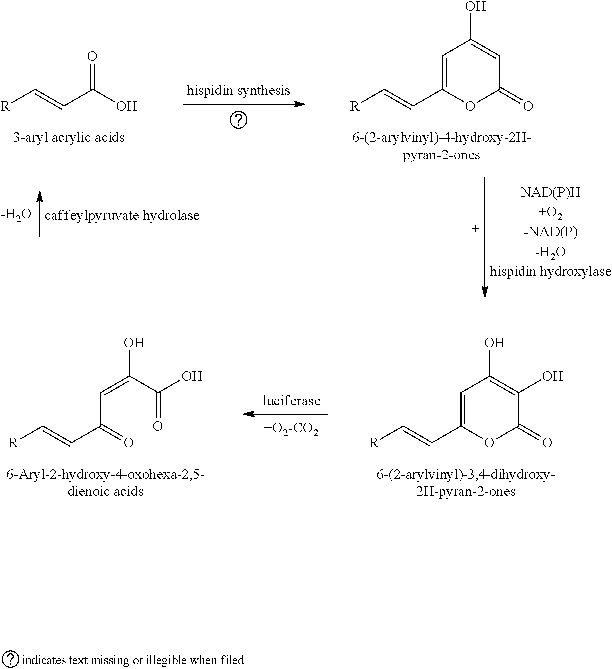

Thus, the present invention first provides isolated fungal luciferin biosynthetic proteins as well as nucleic acids encoding them.

In preferred embodiments, the present invention provides hispidin hydroxylases characterized by the amino acid sequence selected from the following SEQ ID NOs group: 2, 4, 6, 8, 10, 12, 14, 16, 18, 20, 22, 24, 26, 28, as well as essentially similar proteins, homologues, mutants, and derivatives of these hispidin hydroxylases.

In some embodiments, the hispidin hydroxylases of the present invention are characterized by an amino acid sequence that within at least 350 amino acids has at least 60% identity, or at least 65% identity, or at least 70% identity, or at least 75% identity, for example, at least 80% identity, at least 85% identity, at least 90% identity (for example, at least 90%, 91%, 92%, 93%, 94%, 95%, 96%, 97%, 98%, 98% or 99% identity) with the amino acid sequence selected from the following SEQ ID NOs group: 2, 4, 6, 8, 10, 12, 14, 16, 18, 20, 22, 24, 26, 28.

In some embodiments, the amino acid sequence of the hispidin hydroxylase of the present invention is characterized by presence of several consensus sequences separated by non-conservative amino acid insertion segments characterized by the following SEQ ID NOs: 29-33.

The hispidin hydroxylases of the present invention catalyze the reaction of 6-(2-arylvinyl)-4-hydroxy-2H-pyran-2-one with the structural formula

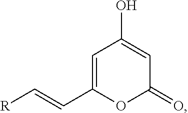

conversion into 6-(2-arylvinyl)-3,4-dihydroxy-2H-pyran-2-one with the structural formula

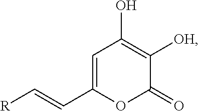

ere R is aryl or heteroaryl.

The present invention also provides hispidin synthases characterized by the amino acid sequence selected from the following SEQ ID NOs group: 35, 37, 39, 41, 43, 45, 47, 49, 51, 53, 55, as well as essentially similar proteins, homologues, mutants, and derivatives of these hispidin synthases.

In some embodiments, the amino acid sequence of the hispidin synthase of the present invention is characterized by presence of several consensus sequences separated by non-conservative amino acid insertion segments characterized by the following SEQ ID NOs: 56-63.

In some embodiments, hispidin synthases of the present invention are characterized by an amino acid sequence that has at least 40% identity, for example, at least 45% identity, or at least 50% identity, or at least 55% identity, or at least 60% identity, or at least 65% identity, or at least 70% identity, or at least 75% identity, for example, at least 80% identity, at least 85% identity, at least 90% identity (for example, at least 90%, 91%, 92%, 93%, 94%, 95%, 96%, 97%, 98%, 98% or 99% identity) with the amino acid sequence selected from the following SEQ ID NOs group: 35, 37, 39, 41, 43, 45, 47, 49, 51, 53, 55.

The hispidin synthases of the present invention catalyze the reaction of 3-aryl acrylic acid with the structural formula

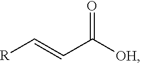

where R is selected from the group aryl or heteroaryl, conversion into 6-(2-arylvinyl)-4-hydroxy-2H-pyran-2-one with the structural formula

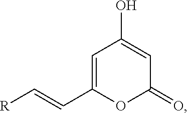

where R is aryl or heteroaryl.

In addition, the present invention provides caffeylpyruvate hydrolases characterized by the amino acid sequence selected from the following SEQ ID NOs group: 65, 67, 69, 71, 73, 75, as well as essentially similar proteins, homologues, mutants, and derivatives of these caffeylpyruvate hydrolases.

In some embodiments, the amino acid sequence of the caffeylpyruvate hydrolase of the present invention is characterized by presence of several consensus sequences separated by non-conservative amino acid insertion segments characterized by the following SEQ ID NOs: 76-78.

In some embodiments, a caffeylpyruvate hydrolase of the present invention is characterized by an amino acid sequence that has at least 60% identity, or at least 65% identity, or at least 70% identity, or at least 75% identity, for example, at least 80% identity, at least 85% identity, at least 90% identity (for example, at least 90%, 91%, 92%, 93%, 94%, 95%, 96%, 97%, 98%, 98% or 99% identity) with the amino acid sequence selected from the following SEQ ID NOs group: 65, 67, 69, 71, 73, 75.

The caffeylpyruvate hydrolases of the present invention catalyze the reaction of 6-aryl-2-hydroxy-4-oxohexa-2,5-dienoic acids with the structural formula

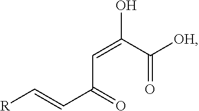

where R is aryl or heteroaryl, conversion into 3-arylacrylic acid with the structural formula

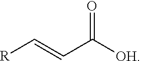

In preferred embodiments, hispidin hydroxylases of the present invention catalyze reaction of preluciferin conversion into fungal luciferin, for example, hispidin conversion into the 3-hydroxyhispidin.

In preferred embodiments, hispidin synthases of the present invention catalyze the conversion of a precursor of preluciferin into the preluciferin, for example, conversion of caffeic acid to hispidin.

In preferred embodiments, caffeylpyruvate hydrolases of the present invention catalyze conversion of fungal oxyluciferin to a precursor of preluciferin, for example, conversion of caffeylpyruvate to caffeic acid.

The present invention also provides application of a protein having the amino acid sequence that within at least 350 amino acids has at least 60% identity, or at least 65% identity, or at least 70% identity, or at least 75% identity, for example, at least 80% identity, at least 85% identity, at least 90% identity (for example, at least 90%, 91%, 92%, 93%, 94%, 95%, 96%, 97%, 98%, 98% or 99% identity) with the amino acid sequence selected from the following SEQ ID NOs group: 2, 4, 6, 8, 10, 12, 14, 16, 18, 20, 22, 24, 26, 28, and/or containing consensus sequences with the SEQ ID NOs 29-33 separated by non-conservative amino acid insertion segments, as hispidin hydroxylase to catalyze in vitro or in vivo reaction of 6-(2-arylvinyl)-4-hydroxy-2H-pyran-2-one with the structural formula

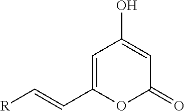

conversion into 6-(2-arylvinyl)-3,4-dihydroxy-2H-pyran-2-one with the structural formula

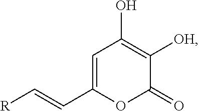

where R is aryl or heteroaryl.

The present invention also provides application of a protein having the amino acid sequence that has at least 45% identity, or at least 50% identity, or at least 55% identity, or at least 60% identity, or at least 65% identity, or at least 70% identity, or at least 75% identity, for example, at least 80% identity, at least 85% identity, at least 90% identity (for example, at least 90%, 91%, 92%, 93%, 94%, 95%, 96%, 97%, 98%, 98% or 99% identity) with the amino acid sequence selected from the following SEQ ID NOs group: 35, 37, 39, 41, 43, 45, 47, 49, 51, 53, 55, and/or containing consensus sequences with the SEQ ID NOs 56-63 separated by non-conservative amino acid insertion segments, as hispidin synthase to catalyze in vitro or in vivo reaction of 3-aryl acrylic acid with the structural formula

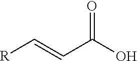

conversion into 6-(2-arylvinyl)-4-hydroxy-2H-pyran-2-one with the structural formula

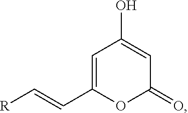

where R is aryl or heteroaryl.

The present invention also provides application of a protein having the amino acid sequence that has at least 60% identity, or at least 65% identity, or at least 70% identity, or at least 75% identity, for example, at least 80% identity, at least 85% identity, at least 90% identity (for example, at least 90%, 91%, 92%, 93%, 94%, 95%, 96%, 97%, 98%, 98% or 99% identity) with the amino acid sequence selected from the following SEQ ID NOs group: 65, 67, 69, 71, 73, 75, and/or containing consensus sequences with the SEQ ID NOs 76-78 separated by non-conservative amino acid insertion segments, as caffeylpyruvate hydrolase to catalyze in vitro or in vivo reaction of 6-aryl-2-hydroxy-4-oxohexa-2,5-dienoic acids with the structural formula

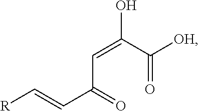

where R is aryl or heteroaryl, conversion into 3-arylacrylic acid with the structural formula

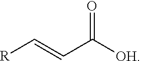

The present invention also provides nucleic acids encoding said hispidin hydroxylases, hispidin synthases, and caffeylpyruvate hydrolases.

In some embodiments, hispidin hydroxylase encoding nucleic acids are provided with amino acid sequence selected from the group:

(a) amino acid sequence presented as the following SEQ ID NOs: 2, 4, 6, 8, 10, 12, 14, 16, 18, 20, 22, 24, 26, 28;
(b) amino acid sequence that within at least 350 amino acids has at least 60% identity, or at least 65% identity, or at least 70% identity, or at least 75% identity, for example, at least 80% identity, at least 85% identity, at least 90% identity (for example, at least 90%, 91%, 92%, 93%, 94%, 95%, 96%, 97%, 98%, 98% or 99% identity) with the amino acid sequence selected from the following SEQ ID NOs group: 2, 4, 6, 8, 10, 12, 14, 16, 18, 20, 22, 24, 26, 28;
(c) amino acid sequence containing consensus sequences presented as the following SEQ ID NOs: 29-33.

In some embodiments, hispidin synthase encoding nucleic acids are provided with amino acid sequence selected from the group:

(a) amino acid sequence presented as the following SEQ ID NOs: 35, 37, 39, 41, 43, 45, 47, 49, 51, 53, 55;
(b) amino acid sequence that has at least 40% identity, for example, at least 45% identity, or at least 50% identity, or at least 55% identity, or at least 60% identity, or at least 65% identity, or at least 70% identity, or at least 75% identity, for example, at least 80% identity, at least 85% identity, at least 90% identity (for example, at least 90%, 91%, 92%, 93%, 94%, 95%, 96%, 97%, 98%, 98% or 99% identity) with the amino acid sequence selected from the following SEQ ID NOs group: 35, 37, 39, 41, 43, 45, 47, 49, 51, 53, 55;
(c) amino acid sequence containing consensus sequences presented as the following SEQ ID NOs: 56-63.

In some embodiments, caffeylpyruvate hydrolases encoding nucleic acids are provided with amino acid sequence selected from the group:

(a) amino acid sequence presented as the following SEQ ID NOs: 65, 67, 69, 71, 73, 75;
(b) amino acid sequence that has at least 60% identity, or at least 65% identity, or at least 70% identity, or at least 75% identity, for example, at least 80% identity, at least 85% identity, at least 90% identity (for example, at least 90%, 91%, 92%, 93%, 94%, 95%, 96%, 97%, 98%, 98% or 99% identity) with the amino acid sequence selected from the following SEQ ID NOs group: 65, 67, 69, 71, 73, 75;
(c) amino acid sequence containing consensus sequences with the SEQ ID NOs 76-78 separated by non-conservative amino acid insertion segments.

The present invention also provides application of the nucleic acid encoding a protein with amino acid sequence that within at least 350 amino acids has at least 60% identity, or at least 65% identity, or at least 70% identity, or at least 75% identity, for example, at least 80% identity, at least 85% identity, at least 90% identity (for example, at least 90%, example, 93%, 94%, 95%, 96%, 97%, 98%, 98% or 99% identity) with the amino acid sequence selected from the following SEQ ID NOs group: 2, 4, 6, 8, 10, 12, 14, 16, 18, 20, 22, 24, 26, 28, and/or containing consensus sequences with the SEQ ID NOs 29-33 separated by non-conservative amino acid insertion segments, to produce in in vitro or in vivo systems the hispidin hydroxylase catalyzing the reaction of 6-(2-arylvinyl)-4-hydroxy-2H-pyran-2-one with the structural formula

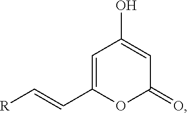

conversion into 6-(2-arylvinyl)-3,4-dihydroxy-2H-pyran-2-one with the structural formula

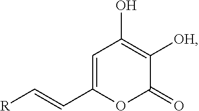

where R is aryl or heteroaryl.

The present invention also provides application of the nucleic acid encoding a protein with amino acid sequence that has at least 45% identity, or at least 50% identity, or at least 55% identity, or at least 60% identity, or at least 65% identity, or at least 70% identity, or at least 75% identity, for example, at least 80% identity, at least 85% identity, at least 90% identity (for example, at least 96%, 97%, 98%, 98% or 99% identity) with the amino acid sequence selected from the following SEQ ID NOs group: 35, 37, 39, 41, 43, 45, 47, 49, 51, 53, 55, and/or containing consensus sequences with the SEQ ID NOs 56-63 separated by non-conservative amino acid insertion segments, to produce in in vitro or in vivo systems the hispidin synthase catalyzing the reaction of 3-aryl acrylic acid with the structural formula

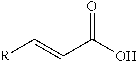

conversion into 6-(2-arylvinyl)-4-hydroxy-2H-pyran-2-one with the structural formula

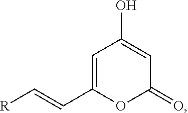

where R is aryl or heteroaryl.

The present invention also provides application of nucleic acid encoding a protein with amino acid sequence that has at least 60% identity, or at least 65% identity, or at least 70% identity, or at least 75% identity, for example, at least 80% identity, at least 85% identity, at least 90% identity (for example, at least 96%, 97%, 98%, 98% or 99% identity) with the amino acid sequence selected from the following SEQ ID NOs group: 65, 67, 69, 71, 73, 75, and/or containing consensus sequences with the SEQ ID NOs 76-78 separated by non-conservative amino acid insertion segments, to produce in in vitro or in vivo systems the caffeylpyruvate hydrolase catalyzing the reaction of 6-aryl-2-hydroxy-4-oxohexa-2,5-dienoic acids with the structural formula

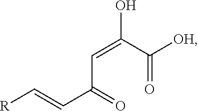

where R is aryl or heteroaryl, conversion into 3-arylacrylic acid with the structural formula

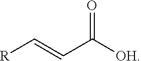

The present invention also provides a fusion protein comprising operatively, directly or via amino acid linkers, cross-linked at least one hispidin hydroxylase of the invention, and/or at least one hispidin synthase of the invention, and/or at least one caffeylpyruvate hydrolase of the invention, and intracellular localization signal, and/or signal peptide, and/or luciferase capable to oxidize the fungal luciferin with light emission.

The luciferase capable to oxidize the fungal luciferin with light emission is known in the art. In preferred embodiments, it has an amino acid sequence substantially similar or identical to an amino acid sequence selected from the following SEQ ID NOs group: 80, 82, 84, 86, 88, 90, 92, 94, 96, 98. For example, it may have an amino acid sequence that is at least 40% identical, for example, at least 45% identical, or at least 50% identical, or at least 55% identical, or at least 60% identical, or at least 70% identical, or at least 75% identical, or at least 80% identical, or at least 85% identical to an amino acid sequence selected from the following SEQ ID NOs group: 80, 82, 84, 86, 88, 90, 92, 94, 96, 98. In many embodiments, the amino acid sequence of said luciferase has at least 90% identity, or at least 95% identity, (e.g. at least 96%, 97%, 98%, 98%, or 99% identity) with an amino acid sequence selected from the following SEQ ID NOs group: 80, 82, 84, 86, 88, 90, 92, 94, 96, 98.

In some embodiments, the fusion protein has the amino acid sequence with SEQ ID NO 101.

The present invention also provides a nucleic acid encoding said fusion protein.

The present invention also provides an expression cassette comprising (a) a domain of transcription initiation, which is functional in a host cell; (b) a nucleic acid encoding a fungal luciferin biosynthesizing enzyme, i.e. hispidin synthase, hispidin hydroxylase or caffeylpyruvate hydrolase, or a fusion protein according to the invention; (c) a domain of transcription termination, which is functional in the host cell.

The present invention also provides a vector for transferring a nucleic acid into a host cell comprising a nucleic acid encoding a fungal luciferin biosynthesizing enzyme of the invention, i.e. hispidin synthase, hispidin hydroxylase, or caffeylpyruvate hydrolase, or a fusion protein of the invention.

The present invention also provides a host cell comprising, as a part of an extrachromosomal element or integrated into genome of the cell as a result of introducing said cassette into said cell, an expression cassette that contains a nucleic acid encoding hispidin synthase and/or hispidin hydroxylase and/or caffeylpyruvate hydrolase of the present invention. Such cell produces at least one of said fungal luciferin biosynthesizing enzymes due to expression of said introduced nucleic acid.

The present invention also provides an antibody obtained using a protein of the invention.

The present invention also provides a method for producing fungal luciferin with the chemical formula 6-2-arylvinyl)-3,4-dihydroxy-2H-pyran-2-one and the structural formula

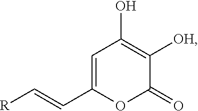

where R is aryl or heteroaryl, in either in vitro or in vivo system, which comprises combining at least one molecule of hispidin hydroxylase according to the invention with at least one molecule of 6-(2-arylvinyl)-4-hydroxy-2H-pyran-2-one having the structural formula

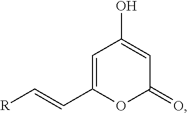

at least one NAD(P)H molecule, and at least one molecular oxygen molecule under physiological conditions.

The present invention also provides a method for producing fungal preluciferin with the chemical formula 6-(2-arylvinyl)-4-hydroxy-2H-pyran-2-one and the structural formula

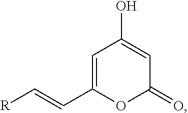

where R is aryl or heteroaryl, in either in vitro or in vivo system, which comprises combining at least one molecule of 3-arylacrylic acid with the structural formula

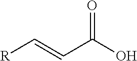

with at least one molecule of hispidin synthase according to the invention, at least one molecule of coenzyme A (CoA), at least one ATP molecule, and at least two malonyl-CoA molecules under physiological conditions.

The present invention also provides a method for in vitro or in vivo producing fungal luciferin, which comprises combining at least one hispidin hydroxylase molecule according to the invention with at least one 3-aryl acrylic acid molecule, at least one molecule of hispidin synthase according to the invention, at least one molecule of coenzyme A, at least one ATP molecule, at least two molecules of malonyl-CoA, at least one NAD(P)H molecule, and at least one molecule of molecular oxygen under physiological conditions.

Methods for producing fungal luciferin and preluciferin can be implemented in a cell or an organism. In this case, said methods comprise introducing into the cell nucleic acids encoding the corresponding luciferin biosynthesizing enzymes (hispidin synthase and/or hispidin hydroxylase) capable of expressing said enzymes in the cell or organism. In preferred embodiments, the nucleic acids are introduced into a cell or organism as a part of an expression cassette or vector of the invention.

In some embodiments, a nucleic acid encoding a 4′-phosphopantotheinyl transferase capable to transfer the 4-phosphopantetheinyl from coenzyme A to serine in the acyl transfer domain of polyketide synthases is additionally introduced into the cell or organism. In some embodiments, the 4′-phosphopantotheinyl transferase has an amino acid sequence substantially similar or identical to SEQ ID NO 105.

The present invention also provides application of the polyketide synthase (PKS) with amino acid sequence that is at least 40%, or at least 45%, or at least 50%, or at least 55%, or at least 60%, at least 65%, or at least 70%, or at least 80%, or at least 85%, or at least 90%, or at least 91%, or at least 92%, or at least 93%, or at least 94%, or at least 95%, or at least 96%, or at least at least 97%, or at least 98%, or at least 99% identical to a sequence selected from the following SEQ ID NOs group: 119, 121, 123, 125, 127, 129, 131, 133, 135, 137, 139 to produce hispidin in an in vitro or in vivo system.

In some embodiments, the method for preparing hispidin comprises combining at least one PKS molecule with at least two malonyl-CoA molecules and at least one caffeyl-CoA molecule under physiological conditions. In some embodiments, said method comprises combining at least one PKS molecule with at least two malonyl-CoA molecules, at least one caffeic acid molecule, at least one coenzyme A molecule, at least one coumarate-CoA ligase molecule, and at least one ATP molecule under physiological conditions.

For the purposes of present invention, any coumarate-CoA ligase can be used that catalyzes conversion of caffeic acid into caffeyl-CoA. For example, coumarate-CoA ligase may have an amino acid sequence that is at least 40%, or at least 45%, or at least 50%, or at least 55%, or at least 60%, or at least 65%, or at least 70%, or at least 80%, or at least 85%, or at least 90%, or at least 91%, or at least 92%, or at least 93%, or at least 94%, or at least 95%, or at least 96%, or at least 97%, or at least 98%, or at least 99% identical to the sequence with SEQ ID NO 141.

Said reaction can be used in any of said methods instead of reaction for producing fungal preluciferin from precursors of preluciferin using the hispidin synthase of the present invention. For example, the reaction can be performed in a cell or organism by introducing an expression cassette with a PKS encoding nucleic acid into the cell or organism. If necessary, a coumarate-CoA ligase encoding nucleic acid can be additionally introduced into the cell or organism.

In some embodiments, 3-aryl acrylic acid biosynthesizing enzymes encoding nucleic acids are further introduced into the same cell or organism. For example, these can be nucleic acids encoding tyrosine ammonia-lyase with an amino acid sequence substantially similar or identical to the amino acid sequence of tyrosine ammonia-lyase of Rhodobacter capsulatus having SEQ ID NO 107 or nucleic acids encoding the HpaB and HpaC components of 4-hydroxyphenylacetate 3-monooxygenase reductase with the amino acid sequences substantially similar to the sequences of HpaB and HpaC components of 4-hydroxyphenylacetate 3-monooxygenase reductase of E. coli having SEQ ID NOs 109 and 111. In some embodiments, the phenylalanine ammonia-lyase encoding nucleic acid with amino acid sequence substantially similar to the amino acid sequence having SEQ ID NO 117 is used.

The present invention also provides methods for producing transgenic bioluminescent cells or organisms, comprising cells or organisms of plants, animals, bacteria, or fungi.

In preferred embodiments, methods for producing transgenic bioluminescent cells or organisms comprise introducing at least one nucleic acid of the invention together with a nucleic acid encoding the luciferase capable to oxidize fungal luciferin with light emission into a cell or organism. Nucleic acids are introduced into a cell or organism in a form such as to enable their expression and production of functional protein products. For example, nucleic acids may be contained in an expression cassette. Nucleic acids can occur in cells as parts of either extrachromosomal elements or integrated into genome of the cell due to insertion of an expression cassette into said cell.

In preferred embodiments, methods for producing transgenic bioluminescent cells or organisms comprise introducing a nucleic acid encoding a hispidin hydroxylase of the invention and a nucleic acid encoding a luciferase capable to oxidize fungal luciferin with light emission into the cell or organism. As a result, said cell or organism acquires the ability to bioluminescence in the presence of fungal preluciferin with the chemical formula 6-(2-arylvinyl)-4-hydroxy-2H-pyran-2-one and structural formula

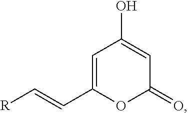

where R is aryl or heteroaryl.

In some embodiments, a hispidin hydroxylase and luciferase s ion protein encoding nucleic acid is introduced into the cell instead of nucleic acids encoding hispidin synthase and luciferase.

In some embodiments, methods for producing transgenic bioluminescent cells or organisms also comprise introducing a hispidin synthase encoding nucleic acid of the invention into the cell or organism. Said cell or organism acquires the ability to bioluminescence in the presence of the precursor of fungal preluciferin in the form of 3-arylacrylic acid with the structural formula

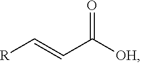

where R is aryl or heteroaryl.

In some embodiments, a PKS encoding nucleic acid is introduced into a cell instead of hispidin synthase encoding nucleic acid.

In some embodiments, methods for producing transgenic bioluminescent cells or organisms also comprise introducing a caffeylpyruvate hydrolase encoding nucleic acid of the invention into the cell or organism to increase intensity of the bioluminescence.

In some embodiments, methods for producing transgenic bioluminescent cells or organisms also include introducing a 4′-phosphopantotheinyl transferase encoding nucleic acid into the cell or organism.

In some embodiments, methods for producing transgenic bioluminescent cells or organisms also comprise introducing a coumarate-CoA ligase encoding nucleic acid into the cell or organism.

In some embodiments, methods for producing transgenic bioluminescent cells or organisms also include introducing 3-aryl acrylic acid biosynthesizing enzymes encoding nucleic acids into the cell or organism.

The present invention also provides transgenic bioluminescent cells and organisms obtained by the said methods and containing one or more nucleic acids of the invention as part of an extrachromosomal element or integrated into the genome of the cell.

In some embodiments, transgenic bioluminescent cells and organisms of the invention are capable of autonomous bioluminescence without exogenous addition of luciferin, preluciferin, and precursor of preluciferin.

The present invention also provides combinations of proteins and nucleic acids of the invention as well as products and kits containing the proteins and nucleic acids of the invention. For example, combinations of nucleic acids are provided for producing autonomously luminous cells, cell lines, or transgenic organisms; assaying the activity of promoters, or labeling cells.

In some embodiments, kits for producing fungal luciferin and/or fungal preluciferin are provided comprising said hispidin hydroxylase, and/or hispidin synthase, and/or PKS, or encoding them nucleic acids.

In some embodiments, kits are provided for producing a bioluminescent cell or bioluminescent transgenic organism comprising a hispidin hydroxylase encoding nucleic acid and a luciferase encoding nucleic acid, said luciferase being capable to oxidize fungal luciferin with light emission. The kit may also contain a caffeylpyruvate hydrolase encoding nucleic acid. The kit may also contain a hispidin synthase or PKS encoding nucleic acid. The kit may also contain 4′-phosphopantotheinyl transferase encoding nucleic acid and/or coumarate-CoA ligase encoding nucleic acid and/or 3-aryl acrylic acid biosynthesizing enzymes encoding nucleic acids. The kit may also contain additional components such as buffer solutions, antibodies, fungal luciferin, fungal preluciferin, precursor of fungal preluciferin, etc. The kit may also contain the kit application guide. In some embodiments, the nucleic acids are provided in expression cassettes or vectors for introduction into cells or organisms.

In preferred embodiments, cells or transgenic organisms of the invention are capable to produce fungal luciferin from precursors. In some embodiments, cells and transgenic organisms of the invention are capable of bioluminescence in presence of precursor of fungal luciferin. In some embodiments, cells or transgenic organisms of the invention are capable of autonomous bioluminescence.

In preferred embodiments of above disclosed methods and application, the preluciferin with chemical formula 6-(2-arylvinyl)-4-hydroxy-2H-pyran-2-one selected from the following group:
(E)-6-(3,4-dihydroxystyryl)-4-hydroxy-2H-pyran-2-one (hispidin),
(E)-4-dihydroxy-6-styryl-2H-pyran-2-one,
(E)-4-hydroxy-6-(4-hydroxystyryl)-2H-pyran-2-one (bisnoryangonin),
(E)-4-hydroxy-6-(2-hydroxystyryl)-2H-pyran-2-one,
(E)-4-hydroxy-6-(2,4-dihydroxystyryl)-2H-pyran-2-one,
(E)-4-hydroxy-6-(4-hydroxy-3,5-dimethoxystyryl)-2H-pyran-2-one,
(E)-4-hydroxy-6-(4-hydroxy-3-methoxystyryl)-2H-pyran-2-one,
(E)-4-hydroxy-6-(2-(6-hydroxynaphthalen-2-yl)vinyl)-2H-pyran-2-one,
(E)-6-(4-aminostyryl)-4-hydroxy-2H-pyran-2-one,
(E)-6-(4-(diethylamino)styryl)-4-hydroxy-2H-pyran-2-one,
(E)-6-(2-(1H-indol-3-yl)vinyl)-4-hydroxy-2H-pyran-2-one,
(E)-4-hydroxy-6-(2,3,6,7-tetrahydro-1H,5H-pyrido[3,2,1-ij]quinolin-9-yl)vinyl)-2H-pyran-2-one is used.

In preferred embodiments, a 3-aryl acrylic acid selected from the group comprising caffeic acid, cinnamic acid, paracoumaric acid, coumaric acid, umbellic acid, sinapic acid, and ferulic acid is suitable for the purposes of the present invention.

In preferred embodiments, 3-hydroxyhispidine is used as the luciferin, hispidin as the preluciferin, and caffeic acid as the precursor of preluciferin.

One object of the present invention is to provide an effective method for producing autonomous bioluminescent systems with visible luminescence, including those based on eukaryotic non-luminous cells and organisms.

Another object of the present invention is to provide a new effective method for synthesizing hispidin or functional analogues thereof.

Another object of the present invention is to provide a new effective method for synthesizing fungal luciferins or functional analogues thereof.

Another object of the present invention is to provide autonomously luminous cells or organisms.

The object of the present invention is achieved by identifying stages of luciferin conversion in bioluminescent fungi and identifying amino acid and nucleotide sequences of proteins involved in luciferin biosynthesis. The function of all proteins has been demonstrated for the first time.

### BRIEF DESCRIPTION OF DRAWINGS

FIG. 1 shows a multiple amino acid sequence alignment of hispidin hydroxylases. FAD/NAD(P)-binding domain is underlined. Consensus sequences are shown below alignment.

FIG. 2 shows a multiple amino acid sequence alignment of hispidin synthases. Consensus sequences are shown below alignment.

FIG. 3 shows a multiple amino acid sequence alignment of caffeylpyruvate hydrolases. Consensus sequences are shown below alignment.

FIG. 4 shows luminescence intensities of Pichia pastoris cells expressing hispidin hydroxylase and luciferase (A) or only luciferase (B), and luminescence intensities of wild type yeast (C), when the colonies are sprayed with 3-hydroxyhispidin (luciferin, left plot) or hispidin (preluciferin, right plot).

FIG. 5 presents luminescence intensity of HEK293NT cells expressing hispidin hydroxylase and luciferase compared with that of HEK293NT cells expressing luciferase only when adding hispidin.

FIG. 6 shows luminescence curves of HEK293T cells expressing: (1) hispidin hydroxylase and luciferase genes separately when adding hispidin; (2) hispidin hydroxylase and luciferase chimeric protein gene when adding hispidin; (3) hispidin hydroxylase and luciferase chimeric protein gene when adding 3-hydroxyhispidine.

FIG. 7 illustrates ability of transfected Pichia pastoris cells to autonomous bioluminescence in contrast to wild-type cells. On the left: cells on Petri dish under daylight, on the right: cells in the dark.

FIG. 8 shows luminescence of a culture of transfected Pichia pastoris cells in the dark.

FIG. 9 shows autonomously bioluminescent transgenic plants Nicotiana benthamiana. Photo on the left was taken in ambient light, photo on the right was taken in the dark.

### EMBODIMENTS OF INVENTION

### Definitions

Various terms related with objects of the present invention are used above as well as in the description and in claims below. The terms “comprises” and “comprising” in the description of this invention are interpreted as “comprises, but not limited to”. The said terms are not intended to be interpreted as “consists only of”.

Terms “luminescence” and “bioluminescence” are interchangeable for the purposes of present invention and refer to the phenomenon of light emission in course of a chemical reaction catalyzed by the enzyme luciferase.

Terms “capable to react”, “promote a reaction” and the like in relation to the activity of a protein mean that said protein is an enzyme that catalyzes the indicated reaction.

For the purposes of present invention, term “luciferase” means a protein that has ability to catalyze oxidation of a chemical compound (luciferin) by molecular oxygen such that the oxidation reaction is accompanied by light emission (luminescence or bioluminescence) and formation of oxidized luciferin.

For the purposes of present invention, term “fungal luciferin” means a chemical compound selected from the group of 6-(2-arylvinyl)-3,4-dihydroxy-2H-pyran-2-ones with the structural formula

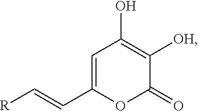

where R is aryl or heteroaryl.

Fungal luciferin is oxidized by a group of luciferases, hereinafter referred to as “luciferases capable to oxidize fungal luciferin with light emission” or the like. Such luciferases were found in bioluminescent fungi, for example, they are described in application RU2017102986/10 (005203) dated 30 Jan. 2017. Amino acid sequences of the luciferases useful for methods and combinations of the present invention are substantially similar or identical to amino acid sequences selected from the following SEQ ID NOs group: 80, 82, 84, 86, 88, 90, 92, 94, 96, 98. In many embodiments of the present invention, luciferases useful for purposes of the present invention are characterized by amino acid sequences that are at least 40% identical, for example, at least 45% identical, or at least 50% identical, or at least 55% identical, or at least 60% identical, or at least 70% identical, or at least 75% identical, or at least 80% identical, or at least 85% identical to an amino acid sequence selected from the following SEQ ID NOs group: 80, 82, 84, 86, 88, 90, 92, 94, 96, 98. In many cases luciferases are characterized by amino acid sequences that have at least 90% identity (for example, at least 91%, at least 92%, at least 93%, at least 94%, at least 95%, at least 96%, at least 97%, at least 98%, at least 99% identity or 100% identity) with an amino acid sequence selected from the following SEQ ID NOs group: 80, 82, 84, 86, 88, 90, 92, 94, 96, 98.

Oxidation of fungal luciferin produces a “fungal oxyluciferin”, a product with the chemical formula 6-aryl-2-hydroxy-4-oxohexa-2,5-dienoic acid and the structural formula

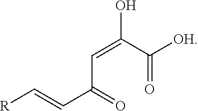

Term “fungal preluciferin” or simply “preluciferin” is used herein to refer to compounds from the group of 6-(2-arylvinyl)-4-hydroxy-2H-pyran-2-ones with the structural formula

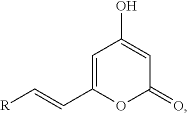

where R is aryl or heteroaryl. The preluciferin is converted to fungal luciferin in a chemical reaction catalyzed by an enzyme of the present invention.

Term “precursor of preluciferin” is used herein to refer to compounds belonging to a group of 3-aryl acrylic acids with the structural formula

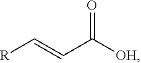

where R is aryl or heteroaryl. Preluciferins are formed from 3-aryl acrylic acids in course of a chemical reaction catalyzed by an enzyme of the present invention.

Examples of fungal luciferins are presented in Table 1. Examples of fungal luciferin related preluciferins, oxyluciferins, and of preluciferins are shown in Table 2.

TABLE 2
Examples of fungal luciferin related preluciferins, oxyluciferins, and of preluciferins
(names of compounds are presented in accordance with IUPAC nomenclature; traditional
names are shown in bold under structural formulas).

| | Precursor of |
|---|---|
| Luciferin | preluciferin |
| (E)-6-(3,4- dihydroxy- styryl)-3,4- dihydroxy- 2H-pyran-2- one(3- hydroxy- hispidin) | 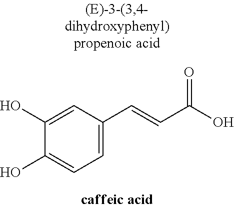 [SMILES: O=C(O)/C=C/c1ccc(O)c(O)c1] |
| (E)-3,4- dihydroxy-6- styryl-2H- pyran-2-one | 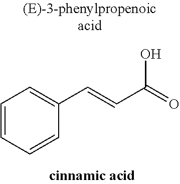 [SMILES: O=C(O)/C=C/c1ccccc1] |
| (E)-3,4- dihydroxy-6- (4- hydroxystyryl)- 2H-pyran-2- one | 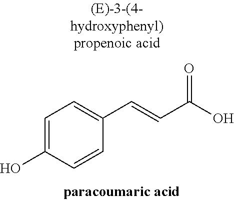 [SMILES: O=C(O)/C=C/c1ccc(O)cc1] |
| (E)-3,4- dihydroxy-6- (2- hydroxystyryl)- 2H-pyran- 2-one | 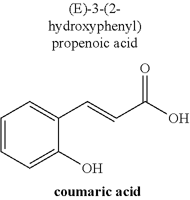 [SMILES: O=C(O)/C=C/c1ccccc1O] |
| (E)-3,4- dihydroxy-6- (2,4- dihydroxy- styryl)-2H- pyran-2-one | 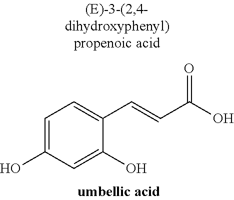 [SMILES: O=C(O)/C=C/c1ccc(O)cc1O] |
| (E)-3,4- dihydroxy-6- (4-hydroxy- 3,5- dimethoxy- styryl)-2H- pyran-2-one | 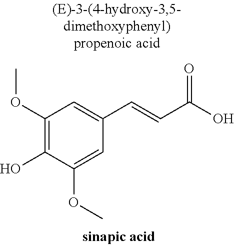 [SMILES: COc1cc(/C=C/C(=O)O)cc(OC)c1O] |
| (E)-3,4- dihydroxy-6- (4-hydroxy- 3- methoxy- styryl)-2H- pyran-2-one | 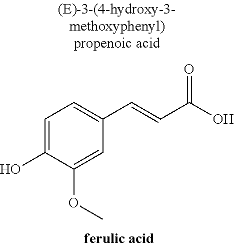 |
| (E)-3,4- dihydroxy-6- (2-(6- hydroxy- naphthalen-2- yl)vinyl)-2H- pyran-2-one | 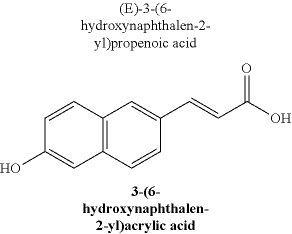 [SMILES: O=C(O)/C=C/c1ccc2cc(O)ccc2c1] |
| (E)-6-(4- aminostyryl)- 3,4- dihydroxy- 2H-pyran-2- one | 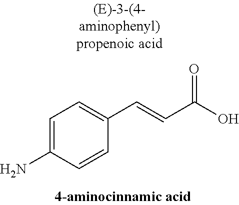 [SMILES: Nc1ccc(/C=C/C(=O)O)cc1] |
| (E)-6-(4- (diethylamino) styryl)-3,4- hydroxy-2H- pyran-2-one | 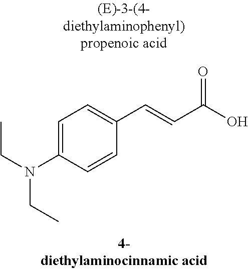 [SMILES: CCN(CC)c1ccc(/C=C/C(=O)O)cc1] |
| (E)-6-(2- (1H-indol-3- yl)vinyl)-3,4- dihydroxy- 2H-pyran-2- one | 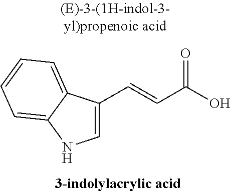 [SMILES: O=C(O)/C=C/c1c[nH]c2ccccc12] |
| (E)-3,4- dihydroxy-6- (2,3,6,7- tetrahydro- 1H,5H- pyrido[3,2,1- ij]quinolin-9- yl)vinyl)-2H- pyran-2-one | 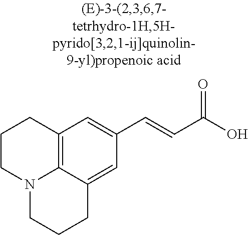 [SMILES: O=C(O)/C=C/c1cc2c3c(c1)CCCN3CCC2] |
| Preluciferin | Oxyluciferin |
| 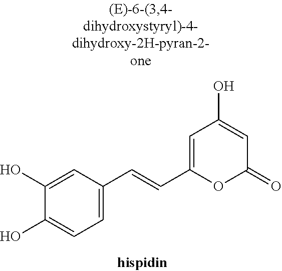 [SMILES: O=c1cc(O)cc(/C=C/c2ccc(O)c(O)c2)o1] | 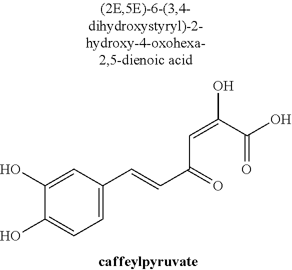 [SMILES: O=C(/C=C/c1ccc(O)c(O)c1)/C=C(/O)C(=O)O] |
| 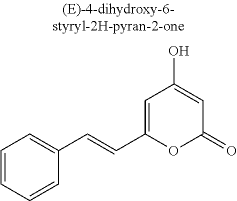 [SMILES: O=c1cc(O)cc(/C=C/c2ccccc2)o1] | 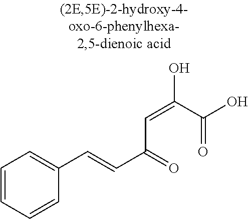 [SMILES: O=C(/C=C/c1ccccc1)/C=C(/O)C(=O)O] |
| 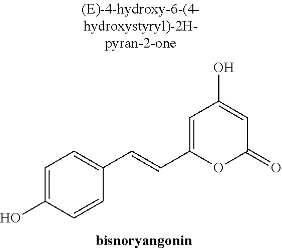 [SMILES: O=c1cc(O)cc(/C=C/c2ccc(O)cc2)o1] | 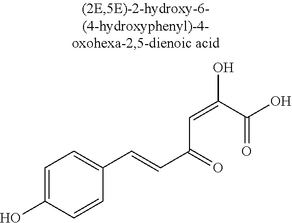 [SMILES: O=C(/C=C/c1ccc(O)cc1)/C=C(/O)C(=O)O] |
| 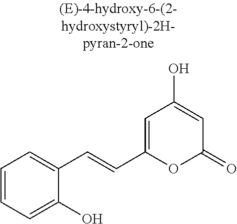 [SMILES: O=c1cc(O)cc(/C=C/c2ccccc2O)o1] | 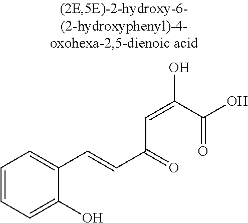 [SMILES: O=C(/C=C/c1ccccc1O)/C=C(/O)C(=O)O] |
| 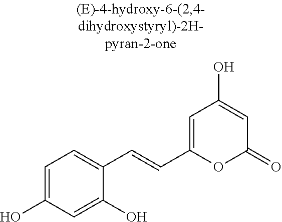 [SMILES: O=c1cc(O)cc(/C=C/c2ccc(O)cc2O)o1] | 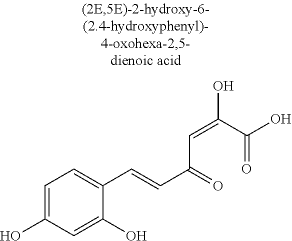 [SMILES: O=C(/C=C/c1ccc(O)cc1O)/C=C(/O)C(=O)O] |
| 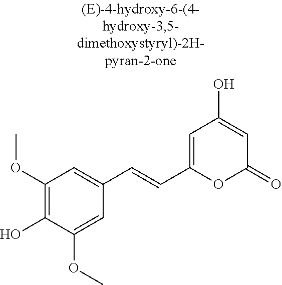 [SMILES: COc1cc(/C=C/c2cc(O)cc(=O)o2)cc(OC)c1O] | 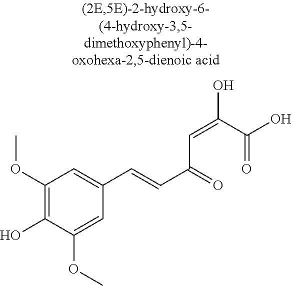 [SMILES: COc1cc(/C=C/C(=O)/C=C(/O)C(=O)O)cc(OC)c1O] |
| 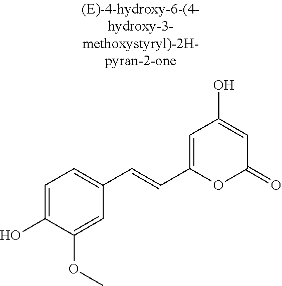 [SMILES: COc1cc(/C=C/c2cc(O)cc(=O)o2)ccc1O] | 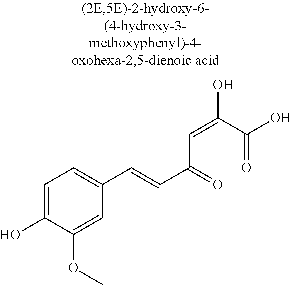 [SMILES: COc1cc(/C=C/C(=O)/C=C(/O)C(=O)O)ccc1O] |
| 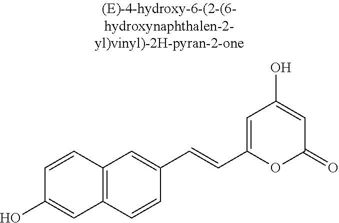 [SMILES: O=c1cc(O)cc(/C=C/c2ccc3cc(O)ccc3c2)o1] | 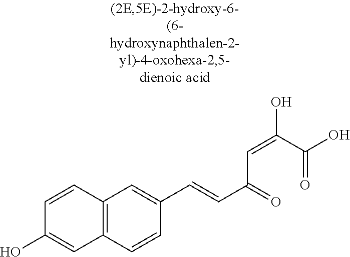 [SMILES: O=C(/C=C/c1ccc2cc(O)ccc2c1)/C=C(/O)C(=O)O] |
| 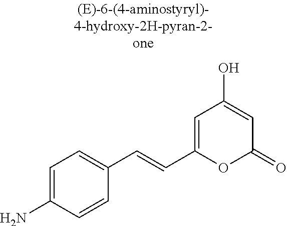 [SMILES: Nc1ccc(/C=C/c2cc(O)cc(=O)o2)cc1] | 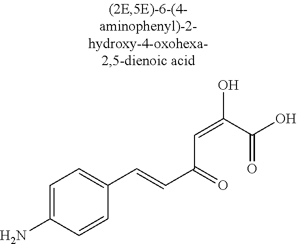 [SMILES: Nc1ccc(/C=C/C(=O)/C=C(/O)C(=O)O)cc1] |
| 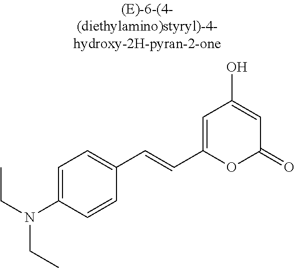 [SMILES: CCN(CC)c1ccc(/C=C/c2cc(O)cc(=O)o2)cc1] | 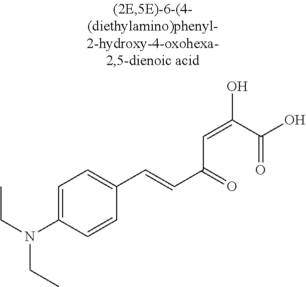 [SMILES: CCN(CC)c1ccc(/C=C/C(=O)/C=C(/O)C(=O)O)cc1] |
| 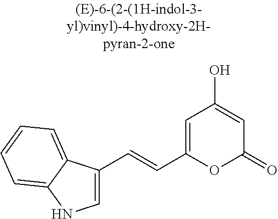 [SMILES: O=c1cc(O)cc(/C=C/c2c[nH]c3ccccc23)o1] | 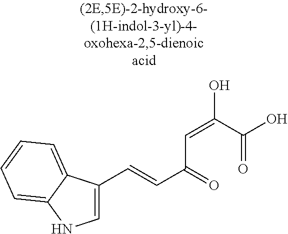 [SMILES: O=C(/C=C/c1c[nH]c2ccccc12)/C=C(/O)C(=O)O] |
| 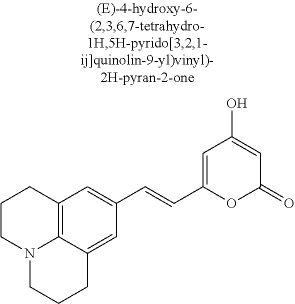 [SMILES: O=c1cc(O)cc(/C=C/c2cc3c4c(c2)CCCN4CCC3)o1] | 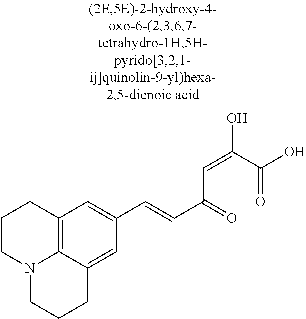 [SMILES: O=C(/C=C/c1cc2c3c(c1)CCCN3CCC2)/C=C(/O)C(=O)O] |

Term “aryl” or “aryl substituent” refers to an aromatic radical in a single or fused carbocyclic ring system containing from five to fourteen ring members. In a preferred embodiment, the ring system contains from six to ten ring members. In addition, one or more hydrogen atoms can be replaced with a substituent selected from acyl, acylamino, acyloxy, alkenyl, alkoxy, alkyl, alkynyl, amino, aryl, aryloxy, azido, carbamoyl, carboalkoxy, carboxy, carboxyamido, carboxyamino, cyano, disubstituted amino, formyl, guanidino, halogen, heteroaryl, heterocyclyl, hydroxy, iminoamino, monosubstituted amino, nitro, oxo, phosphonamino, sulfinyl, sulfonamino, sulfonyl, thio, thioacylamino, thioureido or ureido group. Examples of aryl groups include, but are not limited to, phenyl, naphthyl, biphenyl, and terphenyl. Besides, term “aryl”, as used herein, refers to groups with the aromatic ring linked to one or more non-aromatic rings.

Term “heterocyclic aromatic substituent”, “heteroaryl substituent” or “heteroaryl” refers to an aromatic radical that contains from one to four heteroatoms or hetero groups selected from O, N, S, or SO, in a single or fused heterocyclic ring system containing from five up to fifteen ring members. In a preferred embodiment, the heteroaryl ring system contains from six to ten ring members. In addition, one or more hydrogen atoms can be replaced with a substituent selected from acyl, acylamino, acyloxy, alkenyl, alkoxy, alkyl, alkynyl, amino, aryl, aryloxy, carbamoyl, carboalkoxy, carboxy, carboxyamido, carboxyamino, cyano, disubstituted amino, formyl, guanidino, halogen, heteroaryl, heterocyclyl, hydroxy, iminoamino, monosubstituted amino, nitro, oxo, phosphonamino, sulfinyl, sulfonamino, sulfonyl, thio, thioacylamino, thioureido or ureido group. Examples of heteroaryl groups include, but are not limited to, pyridinyl, thiazolyl, thiadiazolyl, isoquinolinyl, pyrazolyl, oxazolyl, oxadiazoyl, triazolyl, and pyrrolyl groups. Besides, term “heteroaryl”, as used herein, refers to groups with the heteroaromatic ring linked to one or more non-aromatic rings.

Names of chemical compounds are used in the present invention in accordance with the international IUPAC nomenclature. Traditional names are presented as well (if any).

Term “luciferin biosynthesizing enzyme”, or “enzyme involved in cyclic turnover of luciferin conversions”, or the like is used to mean an enzyme that catalyzes the conversion of a preluciferin precursor to preluciferin, and/or preluciferin to fungal luciferin, and/or oxyluciferin to a preluciferin precursor, in in vitro and/or in vivo systems. The term “fungal luciferin biosynthesizing enzyme” does not cover luciferases, unless otherwise specified.

Term “hispidin hydroxylase” is used herein to describe the enzyme that catalyzes reaction of converting preluciferin to fungal luciferin, for example, synthesizing 3-hydroxyhispidin from hispidin.

Term “hispidin synthase” is used herein to describe an enzyme capable to catalyze synthesis of fungal preluciferin from a precursor of preluciferin, for example, synthesis of hispidin from caffeic acid.

Term “PKS” is used herein to describe an enzyme belonging to the group of type III polyketide synthases capable to catalyze synthesis of hispidin from caffeyl-CoA.

Term “caffeylpyruvate hydrolase” is used herein to describe an enzyme capable to catalyze decomposition of fungal oxyluciferin into simpler compounds, for example, to form a precursor of preluciferin. For example, it can catalyze conversion of caffeylpyruvate to caffeic acid.

Term “functional analogue” is used in the present invention to describe chemical compounds or proteins that perform the same function and/or can be used for the same purpose. For example, all fungal luciferins listed in Table 1 are functional analogs of each other.

Term “ATP” refers to adenosine triphosphate, which is the main carrier of energy in the cell and has the structural formula:

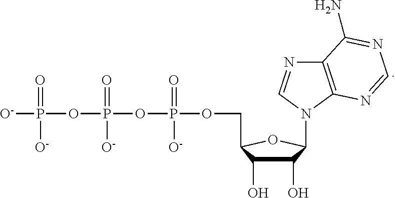

Term “NAD(P)H” is used herein to refer to the reduced nicotinamide adenine dinucleotide phosphate (NADPH) moiety or nicotinamide adenine dinucleotide (NADH) moiety. Term “NAD(P)” is used to refer to the oxidized form of nicotinamide adenine dinucleotide phosphate (NADP) or nicotinamide adenine dinucleotide (NAD). Nicotinamide adenine dinucleotide:

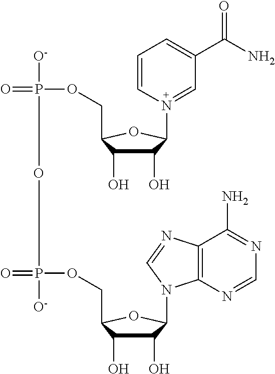

and nicotinamide adenine dinucleotide phosphate:

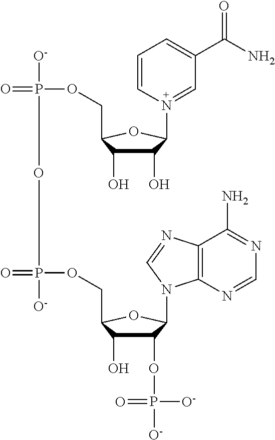

are dinucleotides built from nicotinic acid amide and adenine linked by a chain consisting of two D-ribose residues and two phosphoric acid residues. NADP differs from NAD by presence of additional phosphoric acid residue attached to hydroxyl of a D-ribose residue. Both compounds are widespread in nature and participate in many redox reactions, performing function of carriers of electrons and hydrogen, which it receives from oxidized substances. The reduced forms transfer the received electrons and hydrogen to other substances.

Terms “coenzyme A” or “CoA” refers to a coenzyme well known from the prior art, which is involved in oxidation or synthesis of fatty acids, biosynthesis of fats, oxidative transformations of carbohydrate decomposition products and has the structural formula:

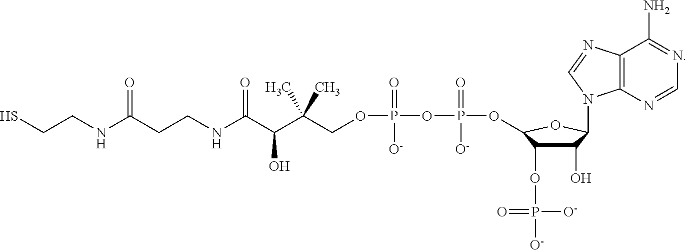

Term “malonyl-CoA” refers to a derivative of coenzyme A formed during synthesis of fatty acids and containing a malonic acid residue:

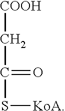

Term “coumaroyl-CoA” refers to the thioester of coenzyme A and coumaric acid:

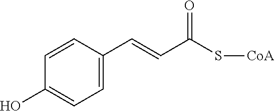

Term “caffeyl-CoA” refers to the thioester of coenzyme A and caffeic acid:

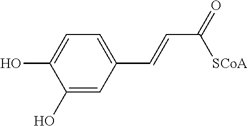

Term “mutant” or “derivative”, as used herein, refers to a protein disclosed in the present invention, wherein one or more amino acids are added to, and/or substituted at, and/or removed (deleted) from, and/or incorporated (inserted) into N-terminus, and/or C-terminus, and/or a native amino acid sequence within a protein of the present invention. As used here, the term “mutant” refers to a nucleic acid moiety that encodes a mutant protein. Besides, the term “mutant”, as used herein, refers to any variant that is shorter or longer than the protein or nucleic acid disclosed in the present invention.

Term “homology” is used to describe the relationship between nucleotide or amino acid sequences, which is determined by the degree of identity and/or similarity between said sequences under comparison.

As used herein, an amino acid or nucleotide sequence is “substantially identical” or “substantially the same” as a reference sequence, if the amino acid or nucleotide sequence has at least 40% identity with the sequence selected within the reference domain. Hence, the substantially similar sequences include those having, for example, at least 40% identity, or at least 50% identity, or at least 55% identity, or at least 60% identity, or at least 62% identity, or at least 65% identity, or at least 70% identity, or at least 75% identity, for example, at least 80% identity, at least 85% identity, at least 90% identity (for example, 90%, 91%, 92%, 93%, 94%, 95%, 96%, 97%, 98%, 98% or 99% identity). Two sequences that are identical to one another are also substantially similar. For the purposes of the present invention, length of sequences to be compared must be at least 100 or more amino acids, preferably at least 200 amino acids, for example, 300 amino acids or more. In particular, it is possible to compare full length amino acid sequences of proteins. For nucleic acids, length of sequences to be compared must be at least at least 300 or more nucleotides; preferably at least 600 nucleotides, including 900 or more nucleotides.

One example of the algorithm suitable for determining sequence identity percentage and sequence similarity is the BLAST algorithm described by Altschul et al., J. Mol. Biol. 215: 403-410 (1990). Software for performing BLAST analyzes is available through the National Biotechnology Information Center (http://www.ncbi.nlm.nih.gov/). This algorithm comprises, first of all, search of high-scoring segment pairs (HSP) by identifying short words of length W in the test sequence, which either completely coincide or satisfy a certain positive-valued threshold score T when aligned with a word of the same length in a database sequence. T is referred to as the neighborhood word score threshold (Altschul et al., 1990). These initial neighborhood word hits act as seeds for initiating searches of longer HSPs containing them. Then these word hits are extended in both directions along each sequence for as far as the cumulative alignment score can be increased. For nucleotide sequences, cumulative scores are calculated using parameters M (reward score set for a pair of matching residues; it is always >0) and N (penalty score set for mismatching residues; it is always <0). To calculate the cumulative value for amino acid sequences, a scoring matrix is used. Extension of the word hits in each direction is halted when the cumulative alignment score falls off by the quantity X from its maximum achieved value; the cumulative score goes to zero or below due to accumulation of one or more negative-scoring residue alignments; or the end of either sequence is reached. BLAST algorithm parameters W, T, and X determine the sensitivity and speed of alignment. In the BLASTN program (for nucleotide sequences), the default word length (W) is 11, the expected value (E) is 10, the drop-off (cutoff) is 100, M=5, N=−4, and comparison is performed on both strands. In the BLASTP program (for amino acid sequences), the default word length (W) is 3, the expected value (E) is 10, and a BLOSUM62 scoring matrix is used (see Henikoff and Henikoff, Proc. Natl. Acad. Sci. USA 89:10915 (1989)).

In addition to calculating the sequence identity percentage, BLAST algorithm also performs statistical similarity analysis between two sequences (see, for example, Karin and Altschul, Proc. Nat'l. Acad. Sci. USA 90: 5873-5787 (1993)). One of parameters provided by the BLAST algorithm to determine the similarity is the lowest cumulative probability (P(N)), which indicates the probability of random coincidence between two nucleotide or amino acid sequences. For example, a test nucleic acid sequence is considered to be similar to a reference sequence if the lowest cumulative probability in comparing the test nucleic acid sequence with the reference nucleic acid sequence is less than 0.1, more preferably less than 0.01, and most preferably less than 0.001.

Term “consensus sequence” refers to an archetypal amino acid sequence used as a reference for comparison of all variants of a particular protein or sequence of interest. Consensus sequences and methods for determining them are well known to those skilled in the art. For example, a consensus sequence can be determined from multiple comparisons of known homologous proteins by identifying the amino acids most frequently occurring at a given position in the entire set of related sequences.

Term “conserved sequence” is used to designate a nucleotide sequence in a nucleic acid or a sequence of amino acids in a polypeptide chain that stays completely or virtually unchanged in the course of evolution in different organisms. Accordingly, a “non-conserved sequence” is a sequence that varies considerably among the compared organisms.

Term “amino acid insertion segment” means one or more amino acids within a polypeptide chain that are between protein fragments (protein domains, linkers, consensus sequences) under consideration. It should be obvious to those skilled in the art that the amino acid insertion segments and fragments under consideration are operatively linked and form a single polypeptide chain.

Domain structure of a protein can be determined using any suitable software known in the art. For example, a Simple Modular Architecture Research Tool (SMART) software available in Internet at http://smart.embl-heidelberg.de can be used for this purpose [Schultz et al., PNAS 1998; 95: 5857-5864; Letunic I, Doerks T, Bork P, Nucleic Acids Res 2014; doi:10.1093/nar/gku949].

Term “operatively linked” or the like in description of fusion proteins refers to polypeptide sequences that occur in a physical and functional relationship with one another. In most preferred embodiments, functions of polypeptide components of the chimeric molecule are not altered as compared with functional properties of the isolated polypeptide components. For example, the hispidin hydroxylase of the present invention can be operatively linked to a fusion partner of interest, e.g. luciferase. In this case, the fusion protein retains the properties of hispidin hydroxylase while the polypeptide of interest retains its original biological activity, for example, the ability to oxidize luciferin with light emission. In some embodiments of the present invention, activities of the fusion partners may be reduced compared with activities of the isolated proteins. Such fusion proteins also find application within the scope of the present invention.

Term “operatively linked” or the like in description of nucleic acids means that the nucleic acids are covalently linked in such a way that there are no reading frame malfunctions or stop signs at their junctions. As it is obvious to any person skilled in the art, nucleotide sequences encoding a fusion protein with the “operatively linked” components (proteins, polypeptides, linker sequences, amino acid insertion segments, protein domains, etc.) are composed of fragments encoding said components, these fragments being covalently linked in such a way that a full-length fusion protein is produced during transcription and translation of the nucleotide sequence.

Term “operatively linked” in description of a nucleic acid relationship with regulatory coding sequences (promoters, enhancers, transcription terminators) means that the sequences are located and linked in such a way that the regulatory sequence will affect the expression level of the coding nucleic acid or nucleic acid sequence.

In the context of the present invention, “linking” of nucleic acids means that two or more nucleic acids are linked together using any means known in the art. As a non-limiting example, nucleic acids can be linked together using DNA ligase or polymerase chain reaction (PCR) during annealing. Nucleic acids can also be linked by chemical synthesis of a nucleic acid using a sequence of two or more separate nucleic acids.

Terms “regulatory elements” or “regulatory sequences” refer to the sequences involved in a coding nucleic acid expression regulation. Regulatory elements include promoters, termination signals, and other sequences that affect the expression of a nucleic acid. They typically also comprise the sequences required for proper translation of the nucleotide sequence.

Term “promoter” is used to describe an untranslated and non-transcribed DNA sequence upstream of the coding region that contains a RNA polymerase binding site as well as transcription initiating DNA binding site. Promoter region can also comprise another gene expression regulating elements.

Term “functional”, as used here, refers to a nucleotide or amino acid sequence that can play a role in a particular test or task. Term “functional”, if used to describe luciferases, means that the protein has the ability to produce the reaction of luciferin oxidation accompanied by luminescence. The same term “functional”, if used to describe hispidin hydroxylases, means that the protein has the ability to catalyze reaction of converting at least one of the preluciferins shown in Table 2 to the corresponding luciferin. The same term “functional”, if used to describe hispidin synthases, means that the protein has the ability to catalyze reaction of converting at least one of precursors of preluciferin to preluciferin, for example, converting caffeic acid to hispidin. The same term “functional”, if used to describe caffeylpyruvate hydrolases, means that the protein has the ability to catalyze reaction of converting at least one of oxyluciferins to precursor of preluciferin (for example, converting caffeylpyruvate to caffeic acid).

Term “enzymatic properties”, as used here, refers to the ability of a protein to catalyze a given chemical reaction.

Term “biochemical properties”, as used here, refers to protein folding and comprises maturation rate, half-life, catalysis rate, pH and temperature stability, and other similar properties.

Term “spectral properties”, as used here, refers to spectra, quantum yield, luminescence intensity, and other similar properties.

Reference to a nucleotide sequence “encoding” a polypeptide means that the polypeptide is produced during mRNA transcription and translation in accordance with this nucleotide sequence. At that, both the coding strand, identical to the mRNA and generally used in the sequence listing, and the complementary strand, which is used as a template for transcription, can be indicated. As it is obvious to any person skilled in the art, this term also covers any degenerate nucleotide sequences encoding the same amino acid sequence. Nucleotide sequences encoding a polypeptide comprise sequences containing introns.

Terms “expression cassette” or “cassette of expression” are used herein in sense of a nucleic acid sequence capable to regulate expression of a particular nucleotide sequence in an appropriate host cell. As a rule, the “expression cassette” contains a heterologous nucleic acid encoding a protein or a functional fragment thereof operatively linked to a promoter and termination signals. Typically, it also contains sequences required for proper translation of a significant nucleotide sequence. The expression cassette may be one that occurs in nature (including host cells), but has been produced in a recombinant form useful for expression of the heterologous nucleic acid. However, in many cases, the “expression cassette” is heterologous with respect to the host, i.e. particular nucleic acid sequence of this expression cassette does not occur naturally in the host cell and must be introduced into the host cell or into progenitor of the host cell by means of transformation. Expression of the nucleotide sequence can be regulated by a constitutive promoter or an inducible promoter that initiates transcription only when the host cell is open to a specific external stimulus. In the case of a multicellular organism, the promoter may also have specificity to a particular tissue, or organ, or developmental stage.

“Heterologous” or “exogenous” nucleic acid means a nucleic acid never occurring in a wild-type host cell.

Term “endogenous” refers to a native protein or nucleic acid in its natural position within genome of the organism.

Term “specifically hybridizes”, as used herein, refers to an association between two single-stranded nucleic acid molecules or sufficiently complementary sequences such as to permit the hybridization under predetermined conditions commonly used in the art (sometimes the term “substantially complementary” is used).

An “isolated” nucleic acid moiety or isolated protein is a nucleic acid moiety or protein occurring separately from its natural environment due to human activities and therefore is not a product of nature. An isolated nucleic acid molecule or an isolated protein can occur in a purified form or in an unnatural environment such as, for example (which is not meant to be limited), a recombinant prokaryotic cell, plant cell, animal cell, non-bioluminescent fungus cell, transgenic organism (fungus, plant, animal), etc.

“Transformation” is the process for introducing a heterologous nucleic acid into a host cell or organism. In particular, “transformation” means a stable integration of DNA moiety into genome of a target organism of interest.

Term “transformed/transgenic/recombinant” refers to a host organism such as bacterium, plant, fungus, or animal, which was modified by introducing a heterologous nucleic acid moiety. This nucleic acid moiety may be either stably integrated into the host genome, or occur as an extrachromosomal moiety. Such an extrachromosomal moiety may be capable of self-replication. It should be understood that transgenic or stably transformed cells, tissues or organisms include both end products of the transformation process, but also transgenic progeny. Terms “non-transformed,” “non-transgenic,” “non-recombinant,” or “wild-type” refer to a natural host organism or host cell, for example, a bacterium or plant, that contain no heterologous nucleic acid moieties.

Term “autonomously luminous” or “autonomously bioluminescent” refers to transgenic organisms or host cells that are capable of bioluminescence without exogenous addition of luciferins, preluciferins, or precursors of preluciferins.

Term “4′-phosphopantotheinyl transferase” is used herein to mean an enzyme that transfers 4-phosphopantotheinyl from coenzyme A to serine in the acyl transfer domain of polyketide synthase. 4′-phosphopantotheinyl transferases are naturally expressed by many plants and fungi and are known in the art [Gao Menghao et al., Microbial Cell Factories 2013, 12:77]. It will be obvious to those skilled in the art that any functional variant of 4′-phosphopantotheinyl transferase can be used for purposes of the present invention. For example, the NpgA 4′-phosphopantotheinyl transferase of Aspergillus nidulans (SEQ ID NOs 104, 105) described in [Gao Menghao et al., Microbial Cell Factories 2013, 12:77], or a homologue or mutant thereof, i.e. a protein with amino acid sequence substantially similar or identical to the sequence having SEQ ID NO 105. Another example is a 4′-phosphopantotheinyl transferase having at least 40% identity, including at least 50% identity, or at least 55% identity, or at least 60% identity, or at least 62% identity, or at least 65% identity, or at least 70% identity, or at least 75% identity, for example, at least 80% identity, or at least 85% identity, or at least 90% identity (for example, 90%, 91%, 92%, 93%, 94%, 95%, 96%, 97%, 98%, 98% or 99% identity) with the sequence characterized by SEQ ID NO 105.

Nucleotides are designated according to their bases using the following standard abbreviations: adenine (A), cytosine (C), thymine (T) and guanine (G). Similarly, amino acids are designated by the following standard abbreviations: alanine (Ala; A), arginine (Arg; R), asparagine (Asn; N), aspartic acid (Asp; D), cysteine (Cys; C), glutamine (Gln; Q), glutamic acid (Glu; E), glycine (Gly; G), histidine (His; H), isoleucine (He; 1), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

Present invention is aimed to identification of new fungal luciferin biosynthesis enzymes, nucleic acids able to encode these enzymes, and proteins able to catalyze certain stages of the fungal luciferin biosynthesis. The invention also provides for application of nucleic acids for producing said enzymes in a cell or organism. Methods for in vitro or in vivo preparation of chemical compounds identical to fungal luciferins and preluciferins are also provided. Vectors comprising nucleic acid described in the present invention are also provided. In addition, the present invention provides expression cassettes comprising the nucleic acid of the present invention and regulatory elements necessary for nucleic acid expression in a selected host cell. Besides, cells, stable cell lines, transgenic organisms (e.g. plants, animals, fungi, or microorganisms) including nucleic acids, vectors, or expression cassettes of the present invention are also provided. Present invention also provides combinations of nucleic acids to obtain autonomously luminous cells, cell lines, or transgenic organisms. In preferred embodiments, cells or transgenic organisms are capable to produce fungal luciferin from precursors. In some embodiments, cells or transgenic organisms are capable to produce fungal preluciferin from precursors. In some embodiments, cells or transgenic organisms are capable of bioluminescence in the presence of a fungal luciferin precursor. In some embodiments, cells or transgenic organisms are capable of autonomous bioluminescence. Combinations of proteins for producing luciferin or its precursors from more simple chemical compounds are also provided. The present invention also provides a kit containing nucleic acids, vectors, or expression cassettes of the present invention for producing luminous cells, cell lines, or transgenic organisms.

### Proteins

As previously stated, this invention provides for proteins involved in fungal luciferin biosynthesis (cyclic system of transformations) as enzymes.

Proteins of this invention could be obtained from natural sources or by means of recombinant technologies. For example, wild-type proteins could be isolated from bioluminescent fungi, e.g. fungi of Basidiomycota type, predominantly of Basidiomycetes class, in particular, Agaricales order. For example, wild-type proteins could be isolated from such fungi as Neonothopanus nambi, Armillaria fuscipes, Armillaria mellea, Guyanagaster necrorhiza, Mycena citricolor, Neonothopanus gardneri, Omphalotus olearius, Panellus stipticus, Armillaria gallica, Armillaria ostoyae, Mycena chlorophos, etc. Proteins of this invention could also be obtained by expression of recombinant nucleic acid, coding protein sequence in respective host or in cell-free expression system, as described in the “Nucleic Acids” section. In some embodiments proteins are used inside host cells, in which nucleic acids capable of expression are introduced to code the said proteins.

In preferred embodiments the claimed proteins are quickly folded after expression in a host cell. “Quick folding” is understood to be the fact that proteins reach their tertiary structure which ensures their enzymic property over a short period of time. In these embodiments, proteins are folded within the period of time which generally does not exceed approximately 3 days, normally does not exceed approximately 2 days and prevalently does not exceed approximately 12-24 hours.

In some embodiments, proteins are used in isolated form. Any common techniques, where suitable methods of protein purification are described in the Guide to Protein Purification (Deuthser ed., Academic Press, 1990), could be used for protein purification. For example, lysate could be prepared from the initial source and purified using HPLC, displacement chromatography, gel electrophoresis, affinity chromatography, etc.

If proteins of the invention are in isolated form, it means that this protein is substantially free from other proteins or other natural biological molecules, such as oligosaccharides, nucleic acids and their fragments, etc., where the term “substantially free from” in this case means that less than 70%, normally less than 60% and prevalently less than 50% of the said composition, comprising the isolated protein, is the other natural biological molecule. In some embodiments the said proteins are substantially in purified form, where the term “substantially purified form” means purity equal at least 95%, normally equal at least 97% and prevalently equal at least 99%.

Proteins of the invention retain activity at temperatures below 50° C., prevalently at temperatures maximum 45° C., i.e. they retain activity at temperatures 20-42° C. and could be used in heterologous expression systems in vitro and in vivo.

The claimed proteins have pH stability within the range from 4 to 10, prevalently within the range from 6.5 to 9.5. Optimum pH stability of the claimed proteins is within the range from 6.8 to 8.5, e.g. between 7.3-8.3.

The claimed proteins are active in physiological conditions. The term “physiological conditions” in this invention is intended to refer to a medium having the temperature within the range from 20 to 42° C., pH within the range from 6.8 to 8.5, saline and osmolarity of 300-400 mOsm/l. In particular, the term “physiological conditions” includes intracellular medium, cell-free preparation and liquids extracted from living organisms, such as blood plasma. “Physiological conditions” could be created artificially. For example, reaction mixtures, ensuring “physiological conditions”, could be created by combining known chemical compounds. Methods of such media creation are well known from the prior art. Non-limiting examples include:

1) Ringer's Solution Isotonic to Mammal Blood Plasma.

Ringer's solution consists of 6.5 g of NaCl, 0.42 g of KCl and 0.25 g of CaCl₂, dissolved in 1 litre of double-distilled water. When preparing the solution, the salts are added sequentially, each subsequent salt is added only after dissolving the previous one. In order to prevent from calcium carbonate sedimentation, it is recommended to pass carbon dioxide through sodium bicarbonate solution. The solution is prepared with fresh distilled water.

2) Versene Solution

The Versene solution is a mixture of EDTA and inorganic salts dissolved in distilled water or in water for injection sterilized by membrane filtration using filters with final pore size of 0.22 μm. 1 l of Versene solution comprises 8.0 g of NaCl, 0.2 g of KCl, 1.45 g of disodium phosphate dodecahydrate, 0.2 g of potassium dihydrogen phosphate, 0.2 g of palkelate, double-distilled water—up to 1 l. Versene solution buffer capacity should be minimum 1.4 ml. Chloride ion content—from 4.4 to 5.4 g/l, EDTA—minimum 0.6 mmol/l.

3) Phosphate-Buffered Saline (PBS, Na-Phosphate Buffer)

Na-phosphate buffer consists of 137 mM of NaCl, 10 mM of Na₂HPO₄, 1.76 mM of KH₂PO₄. The buffer could also contain KCl at concentration of up to 2.7 mM. The following is used to prepare 1 litre of normal strength Na-phosphate buffer: 8.00 g of NaCl, 1.44 g of Na₂HP₄, 0.24 g of KH₂PO₄, 0.20 g of KCl (optionally). Dissolving in 800 ml of distilled water. The required pH is adjusted using hydrochloric acid or sodium hydroxide. Then distilled water is added to a total volume of 1 liter.

Specific proteins of interest are enzymes involved in cyclic fungal luciferin biosynthesis, their mutants, homologs and derivatives. Each of these specific types of polypeptide structures of interest will be further individually analyzed in more details.

Hispidin-Hydroxylases

Hispidin-hydroxylases of this invention are proteins able to catalyze luciferin synthesis from preluciferin. In other words, these are enzymes catalyzing reaction of transformation of 6-(2-arylvinyl)-4-hydroxy-2H-pyran-2-one, having the structural formula

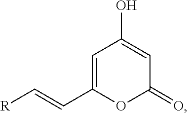

into 6-(2-arylvinyl)-3,4-dihydroxy-2H-pyran-2-one, having the structural formula

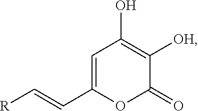

where R-aryl or heteroaryl.

The reaction is carried out in physiological conditions in vitro and in vivo in the presence of at least one molecule of NAD(P)H and at least of one molecule of molecular oxygen (O₂) per one molecule of 6-(2-arylvinyl)-4-hydroxy-2H-pyran-2-one:

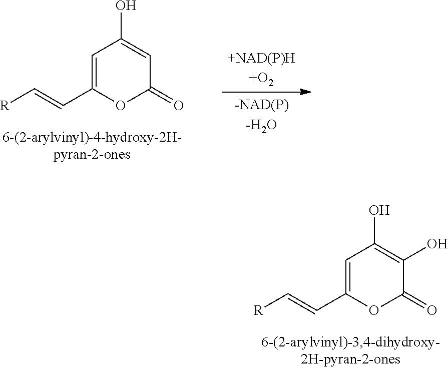

Hispidin-hydroxylases of interest include proteins from bioluminescent fungi Neonothopanus nambi, Armillaria fuscipes, Armillaria mellea, Guyanagaster necrorhiza, Mycena citricolor, Neonothopanus gardneri, Omphalotus olearius, Panellus stipticus, Armillaria gallica, Armillaria ostoyae, Mycena chlorophos, which amino acid sequences are shown in SEQ ID NOs: 2, 4, 6, 8 10, 12, 14, 16, 18, 20, 22, 24, 26, 28, and also their functional mutants, homologs and derivatives.

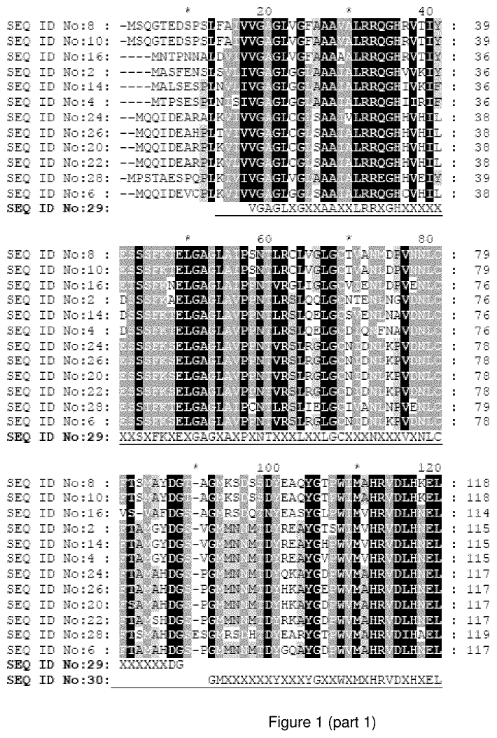

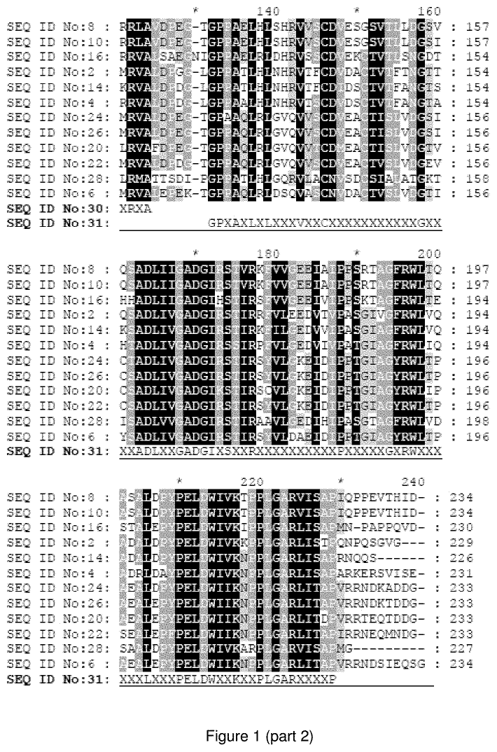

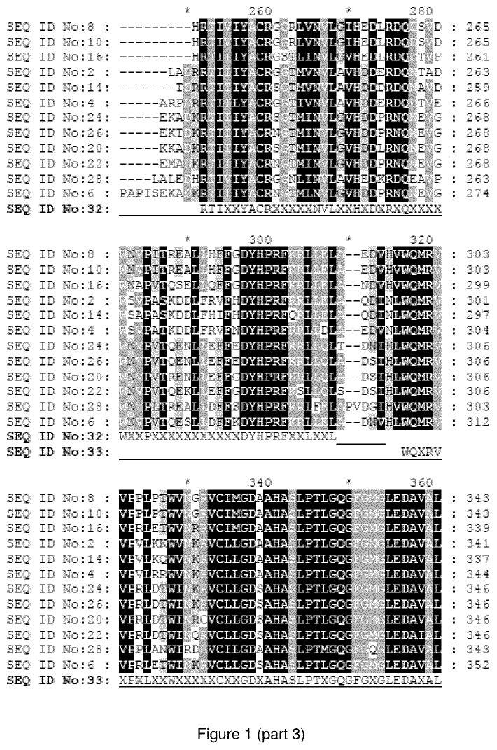

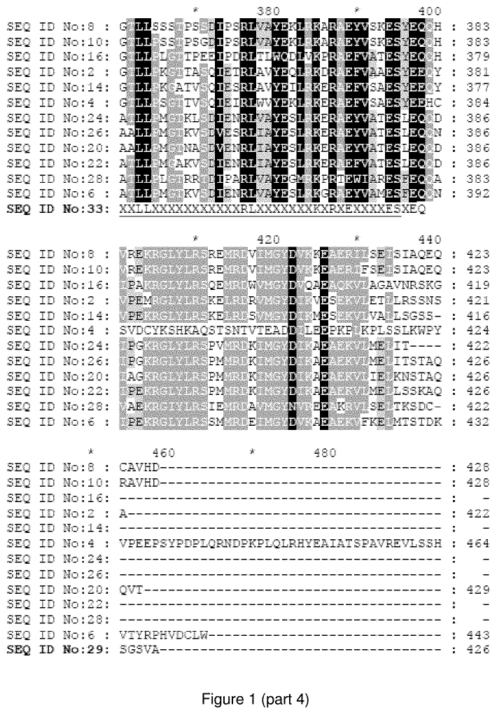

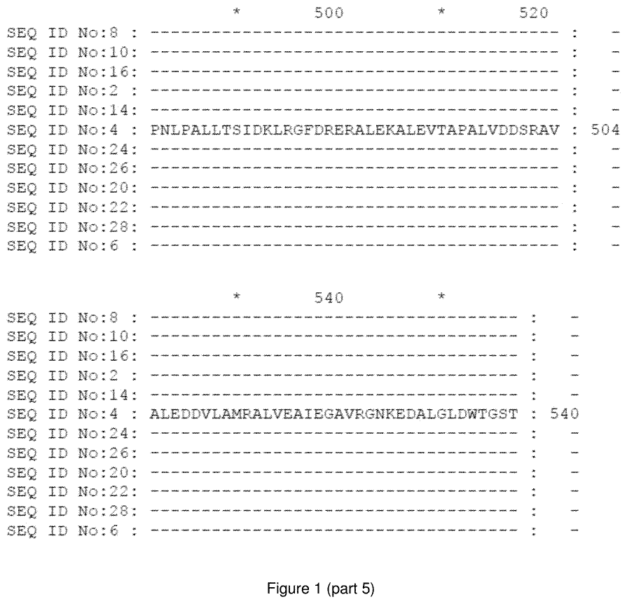

In preferred embodiments hispidin hydroxylases of this invention are characterized by presence of FAD/NAD(P) binding domain, IPR002938—code of InterPro public database available on the Internet at the website http://www.ebi.ac.uk/interpro). The said domain is involved in binding flavine adenine dinucleotide (FAD) and nicotinamide adenine dinucleotide (NAD) in multiple enzymes, adding the hydroxyl group to substrate, and multiple organisms found in metabolic pathways. Hispidin-hydroxylases of this invention comprise the said domain with the length of 350-385 amino acids, prevalently 360-380 amino acids, e.g. 364-377 amino acids, floxed N- and C-terminal non-conservative amino acid sequences having lower percentage of identity with each other. Position of FAD/NAD binding domain in the claimed hispidin hydroxylases is illustrated at multiple alignment of individual protein amino acid sequences in FIG. 1.

Hispidin-hydroxylase homologs or mutants are also provided, which sequence differs from the above mentioned specific amino acid sequences claimed in the invention, i.e. SEQ ID NO: 2, 4, 6, 8 10, 12, 14, 16, 18, 20, 22, 24, 26, 28. Homologs or mutants of interest have at least minimum 40% of identity, e.g. minimum 45% of identity, or minimum 50% of identity, or minimum 55% of identity, or minimum 60% of identity, or minimum 65% of identity, or minimum 70% of identity, or minimum 75% of identity, e.g. minimum 80% of identity, minimum 85% of identity, minimum 90% of identity (e.g. at least 90%, 91%, 92%, 93%, 94%, 95%, 96%, 97%, 98%, 98% or 99% of identity) with protein, which amino acid sequence is selected from the group of SEQ ID NOs: 2, 4, 6, 8 10, 12, 14, 16, 18, 20, 22, 24, 26, 28, for at least 350 amino acids. Particularly it relates to amino acid sequences which provide for protein functional sites, i.e. to the sequence of FAD/NAD binding domain being the part of hispidin hydroxylases.

In preferred embodiments hispidin hydroxylase amino acid sequence of this invention is characterized by presence of several conservative amino acid motifs (consensus sequences) typical of this enzyme group only. These consensus sequences are shown in SEQ ID NOs: 29-33. Consensus sites inside hispidin hydroxylase amino acid sequences are operatively bound via amino acid inserts with lower insertions.

Hispidin-Synthases

Hispidin-synthases of this invention are proteins able to catalyze preluciferin synthesis from its precursors. In other words, these are enzymes catalyzing reaction of transformation of 3-arylacrylic acid with the structural formula

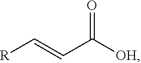

where R-aryl or heteroaryl in 6-2-arylvinyl)-4-hydroxy-2H-pyran-2-one, having the structural formula

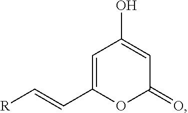

where R-aryl or heteroaryl.

Examples of 3-arylacrylic acids being the precursors of preluciferins are given in Table 2.

The reaction is carried out in physiological conditions in vitro and in vivo in the presence of at least one molecule of coenzyme A, at least one molecule of ATP and at least two molecules of malonyl-CoA:

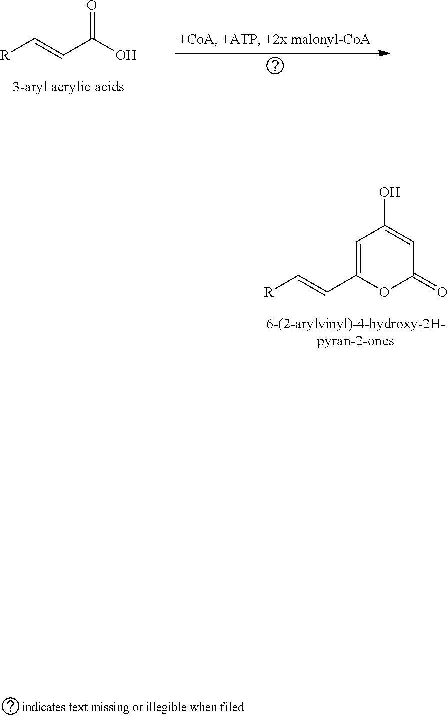

Hispidin-synthases of interest include proteins from bioluminescent fungi Neonothopanus nambi, Armillaria fuscipes, Armillaria mellea, Guyanagaster necrorhiza, Mycena citricolor, Neonothopanus gardneri, Omphalotus olearius, Panellus stipticus, Armillaria gallica, Armillaria ostoyae, Mycena chlorophos, which amino acid sequences are shown in SEQ ID NOs: 35, 37, 39, 41, 43, 45, 47, 49, 51, 53, 55, and also their functional mutants, homologs and derivatives.

In preferred embodiments hispidin-synthase amino acid sequence of this invention is characterized by presence of several conservative amino acid motifs (consensus sequences) typical of this enzyme group only. These consensus sequences are shown in SEQ ID NOs: 56-63. Consensus sites inside hispidin-synthase amino acid sequences are operatively bound via amino acid inserts with lower insertions.

In many embodiments of this invention the relevant amino acid sequences of homologs and mutants of specific hispidin-synthases are characterized by substantial identity with sequences shown in SEQ ID NOs: 35, 37, 39, 41, 43, 45, 47, 49, 51, 53, 55, which is, for example, at least minimum 40% of identity, e.g. minimum 45% of identity, or minimum 50% of identity, or minimum 55% of identity, or minimum 60% of identity, or minimum 65% of identity, or minimum 70% of identity, or minimum 75% of identity, e.g. minimum 80% of identity, minimum 85% of identity, minimum 90% of identity (e.g. at least 90%, 91%, 92%, 93%, 94%, 95%, 96%, 97%, 98%, 98% or 99% of identity) for all protein amino acid sequence.

In preferred embodiments hispidin-synthases of this invention are polydomain proteins related to polyketide synthase superfamily. In preferred embodiments hispidin-synthases of this invention are subjected to post-translation modification, namely, transfer of 4-phosphopantetheinyl from coenzyme A to serine in acyl carrier domain of polyketide synthase is required for their maturation. Enzymes—4′-phosphopantetheinyl transferases performing such modification are known from the prior art [Gao Menghao et al., Microbial Cell Factories 2013, 12:77]. 4′-phosphopantetheinyl transferases are expressed in nature by many plants and fungi, in which cells the functional hispidin-synthase of this invention maturates without introduction of additional enzymes or nucleic acids coding them. At the same time introduction of 4′-phosphopantetheinyl transferase coding sequence into host cells is required for maturation of hispidin-synthase in cells of some lower fungi (e.g. yeast) and animals. It is obvious to those skilled in the art that any functional variant of 4′-phosphopantetheinyl transferase, known from the prior art, could be used for the purposes of this invention. For example, there could be used 4′-phosphopantetheinyl transferase NpgA from Aspergillus nidulans (SEQ ID NO 104, 105), described in [Gao Menghao et al., Microbial Cell Factories 2013, 12:77], any it's homolog or mutant with confirmed activity.

Caffeoyl Pyruvate Hydrolases

Caffeoyl pyruvate hydrolases of this invention are proteins able to catalyze transformation of oxyluciferin, which is 6-aryl-2-hydroxy-4-oxohexa-2,5-diene acid having the structural formula

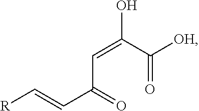

where R is aryl or heteroaryl, conversion into 3-arylacrylic acid with the structural formula

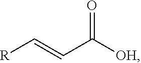

where R-aryl or heteroaryl.

Examples of oxyluciferins are given in Table 2.

The reaction is carried out in physiological conditions in vitro and in vivo:

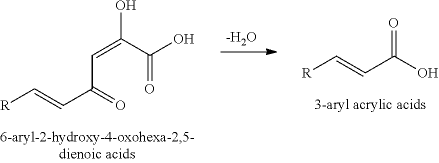

In preferred embodiments caffeylpyruvate hydrolases of this invention transform caffeylpyruvate into caffeic acid. In preferred embodiments they transform oxyluciferin shown in Table 2 into preluciferin precursor.

Caffeoyl pyruvate hydrolases of interest include proteins from bioluminescent fungi Neonothopanus nambi, Armillaria fuscipes, Armillaria mellea, Guyanagaster necrorhiza, Mycena citricolor, Neonothopanus gardneri, Omphalotus olearius, Panellus stipticus, Armillaria gallica, Armillaria ostoyae, Mycena chlorophos, which amino acid sequences are shown in SEQ ID NOs: 65, 67, 69, 71, 73, 75, and also their functional mutants, homologs and derivatives.

In preferred embodiments caffeylpyruvate hydrolase amino acid sequence of this invention (including homologs and mutants of interest) is characterized by presence of several conservative amino acid motifs (consensus sequences) typical of this enzyme group only. These consensus sequences are shown in SEQ ID NOs: 76-78. Consensus sites inside caffeylpyruvate hydrolase amino acid sequences are operatively bound via amino acid inserts with lower insertions.

In many embodiments of this invention the relevant amino acid sequences of caffeylpyruvate hydrolase are characterized by substantial identity with sequences shown in SEQ ID NOs: 65, 67, 69, 71, 73, 75, which is, for example, at least minimum 40% of identity, e.g. minimum 45% of identity, or minimum 50% of identity, or minimum 55% of identity, or minimum 60% of identity, or minimum 65% of identity, or minimum 70% of identity, or minimum 75% of identity, e.g. minimum 80% of identity, minimum 85% of identity, minimum 90% of identity (e.g. at least 90%, 91%, 92%, 93%, 94%, 95%, 96%, 97%, 98%, 98% or 99% of identity) for all protein amino acid sequence.

Homologs of the above-described specific proteins (i.e proteins with amino acid sequences SEQ ID NO: 2, 4, 6, 8 10, 12, 14, 16, 18, 20, 22, 24, 26, 28, 35, 37, 39, 41, 43, 45, 47, 49, 51, 53, 55, 65, 67, 69, 71, 73, 75) could be isolated from natural sources. Homologs could be found in many organisms (fungi, plants, microorganisms, animals). In particular, homologs could be found in different kinds of bioluminescent fungi, e.g. fungi of Basidiomycota type, predominantly of Basidiomycetes class, in particular, Agaricales order. Also, non-bioluminescent fungi and plants producing hispidin, such as Pteris ensiformis, are of special interest as a source of protein homologs of this invention [Yung-Husan Chen et al., «Identification of phenolic antioxidants from Sword Brake fem (Pteris ensiformis Burm.)», Food Chemistry, Volume 105, Issue 1, 2007, pp. 48-56], Inonotus xeranticus [In-Kyoung Lee et al., «Hispidin Derivatives from the Mushroom Inonotus xeranticus and Their Antioxidant Activity», J. Nat. Prod., 2006, 69 (2), pp. 299-301], Phellinus sp. [In-Kyoung Lee et al., «Highly oxygenated and unsaturated metabolites providing a diversity of hispidin class antioxidants in the medicinal mushrooms Inonotus and Phellinus». Bioorganic & Medicinal Chemistry. 15 (10): 3309-14.], Equisetum arvense [Markus Herderich et al., «Establishing styrylpyrone synthase activity in cell free extracts obtained from gametophytes of Equisetum arvense L. by high performance liquid chromatography-tandem mass spectrometry». Phytochem. Anal., 8:194-197.].

Proteins which are derivatives or mutants of the above-described proteins naturally occurring are also provided. Mutants and derivatives can retain biological properties of wild-type proteins (e.g. naturally occurring) or can have biological properties different from wild-type proteins. Mutations include replacements of one or more amino acids, deletion or insertion of one or more amino acids, N-terminal replacements or truncations, or extensions, C-terminal replacements or truncations, or extensions, etc. Mutants and derivatives can be obtained using standard methods of molecular biology, as described in details in the “Nucleic Acids” section. Mutants are substantially identical to wild-type proteins, i.e. have at least 40% of identity with them inside the region selected for comparison. Therefore, substantially similar sequences include those which have, for example, at least 40% of identity, or at least 50% of identity, or at least 55% of identity, or at least 60% of identity, or at least 62% of identity, or at least 65% of identity, or at least 70% of identity, or at least 75% of identity, for example, at least 80% of identity, or at least 85% of identity, or at least 90% of identity (e.g. 90%, 91%, 92%, 93%, 94%, 95%, 96%, 97%, 98%, 98% or 99% of identity) inside the region selected for comparison. In many embodiments homologs of interest have far higher identity of sequence, for example, 70%, 75%, 80%, 85%, 90% (e.g. 92%, 93%, 94%) or higher, e.g. 95%, 96%, 97%, 98%, 99%, 99.5%, especially for a sequence of amino acids, which provide protein functional regions.

Derivatives can also be obtained using standard methods and include changing by means of RNA, chemical modifications, modifications after translation and after transcription, etc. For example, derivatives could be obtained by such methods as modified phosphorylation or glycosylation, or acetylation, or lipidation, or by different types of segregation at maturation, etc.

Methods well known to those skilled in the art are used for searching functional mutants, homologs and derivatives. For example, functional screening of the expression library comprising variants (e.g. protein mutant forms or homologous proteins, or protein derivatives). Expression library is obtained by cloning of nucleic acids coding the tested variants of proteins into expression vector and their entry into appropriate host cells. Methods of operation with nucleic acids are described in detail in the “Nucleic Acids” section. In order to identify functional enzymes of this invention an appropriate substrate is added to the cells expressing the nucleic acids being tested. Formation of the expected product of reaction catalyzed by functional enzyme could be detected by HPLC methods using synthetic variants of the expected reaction products as standards. For example, hispidin or other preluciferin, shown in Table 2, can be used as a substrate to identify functional hispidin hydroxylases. The expected reaction product is fungal luciferin. Preluciferin precursor (e.g. caffeic acid) can be used as a substrate to identify hispidin-synthases, and the corresponding fungi preluciferin is the reaction product. It should be noted that host cells shall express 4′-phosphopantetheinyl transferase, promoting protein post-translational modification, for screening functional hispidin-synthases.

Oxyluciferin (Table 2) is used as a substrate for searching functional caffeylpyruvate hydrolases, and the tested reaction product is a preluciferin precursor—3-arylacrylic acid.

In many embodiments of this invention the bioluminescent reaction can be used for searching functional enzymes of this invention. In this case, for the purpose of expression library preparation the cells producing luciferase able to oxidize fungal luciferin with luminescence emission, and functional enzymes promoting production of fungal luciferin from a product of enzymatic reaction performed by test protein.

Thus, host cells producing functional luciferase, which substrate is fungal luciferin, are used for screening functional hispidin hydroxylases. When adding preluciferin to the cells comprising functional variant of hispidin hydroxylase, fungal luciferin is formed, and luminescence appears due to fungal luciferin oxidation with luciferase.

Host cells additionally producing functional luciferase, which substrate is fungal luciferin, and functional hispidin hydroxylase, are used for screening functional hispidin synthases. When adding preluciferin precursor to such cells, fungal luciferin is formed, and luminescence appears due to fungal luciferin oxidation with luciferase.

Host cells producing functional luciferase, which substrate is fungal luciferin, functional hispidin hydroxylase and functional hispidin synthase, are used for screening functional caffeylpyruvate hydrolases. When adding oxyluciferin to such cells, fungal luciferin is formed, and luminescence appears due to fungal luciferin oxidation with luciferase.

Any luciferases able to oxidize luciferin with luminescence emission, selected from the group of 6-(2-arylvinyl)-3,4-dihydroxy-2H-pyran-2-ones having the common formula

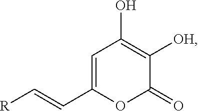

where R-aryl or heteroaryl, can be used for screening. Non-limiting examples of luciferins are given in Table 1. Non-limiting examples of suitable luciferases are described in the section “Application, combinations and methods of use” below.

Luciferin oxidation with luciferase is accompanied with emission of luminescence detected. Light emitted during the oxidation can be detected by standard methods (for example, visual observation, observation by means of night vision devices, spectrophotometry, spectrofluorimetry, using of image photographic recording, using of special equipment for detection of luminescence and fluorescence, such as, e.g. IVIS Spectrum In Vivo Imaging System (Perkin Elmer), etc.). Recorded luminescence could be emitted within intensity range from one photon to luminescence easily perceptible to the eye, e.g. with intensity of 1 cd and bright luminescence with intensity, e.g. 100 cd and more. Light emitted at oxidation of 3-hydroxy hispidin is within the range from 400 to 700 nm, prevalently within the range from 450 to 650 nm, with emission maximum at 520-590 nm. Light emitted at oxidation of other 6-(2-arylvinyl)-3,4-dihydroxy-2H-pyran-2-ones could have emission maximum shift (Table 3).

TABLE 3
Emission maximums for a series of fungi

| | Emission |
|---|---|
| Substance | maximum, nm |
| 3-hydroxy hispidin | 538 |
| (E)-3,4-dihydroxy-6-(4-hydroxystyryl)-2H-pyran- | 520 |
| 2-one | |
| (E)-6-(2-(1H-indol-3-yl)vinyl)-3,4-dihydroxy-2H- | 480 |
| pyran-2-one, | |
| (E)-6-(4-(diethylamino)styryl)-3,4-dihydroxy-2H- | 504 |
| pyran-2-one, | |
| (E)-3,4-dihydroxy-6-(2-(2,3,6,7-tetrahydro-1H,5H- | 534 |
| pyrido[3,2,1-ij]quinolin-9-yl)vinyl)-2H-pyran-2-one, | |
| (E)-3,4-dihydroxy-6-(2-(6-hydroxynaphthalen-2- | 564 |
| yl)vinyl)-2H-pyran-2-one | |

Examples of functional screening using bioluminescence are described in the experimental part below.

The invention also covers fusion proteins, including protein of this invention. Its homolog. mutant, including shortened or elongated form. The protein of the invention could be operatively fused with intracellular localization signal (e.g. nuclear localization signal, localization signal in mitochondria, or in peroxisomes, or in lysosomes, or in Goldgi apparatus, or in other cell organelles), signal peptide promoting protein isolation into intercellular space, transmembrane domain or with any protein or polypeptide (fusion partner) of interest. Fusion proteins could include operatively cross-linked, e.g. hispidin hydroxylase and/or hispidin synthase, and/or caffeylpyruvate hydrolase, claimed in the invention, with fusion partner linked to C- or N-terminal. Non-limiting examples of fusion partners could include proteins of this invention having other enzymic function, antibodies or their linking fragments, ligands or receptors, luciferases able to us fungi luciferins as substrates in bioluminescent reaction. In some embodiments a fusion partner and protein of the invention are operatively cross-linked via linking sequence (peptide linker) promoting independent fusion protein folding and functioning. Methods of fusion proteins production are well known to those skilled in the art.

In some embodiments fusion proteins include hispidin hydroxylase of the invention and luciferase able to oxidize fungal luciferin with luminescence emission, which are operatively cross-linked via short peptide linker. Such fusion protein can be used for obtaining bioluminescence in vitro and in vivo in the presence of a preluciferin (e.g. in the presence of hispidin). It is obvious to those skilled in the art that any functional hispidin hydroxylase described above could be used with any functional luciferase to produce a fusion protein. Specific examples of fusion proteins are described in the Experimental Part below. Examples of luciferases which could be used at producing fusion proteins are described in the section “Application, combinations and methods of use” below.

### Nucleic Acids

This invention provides for nucleic acids coding enzymes of fungal luciferin biosynthesis, mutants and homologs of these proteins, including shortened and elongated forms.

Nucleic acid, as herein used, is an isolated DNA molecule, such as genomic DNA molecule or cDNA molecule, or RNA molecule, such as mRNA molecule. In particular, the said nucleic acids are cDNA molecules having open reading frame, which codes luciferin biosynthesis enzyme of the invention, and capable, under appropriate conditions, to ensure enzyme expression of the invention.

The term “cDNA” is for description of nucleic acids, which reflect arrangement of sequence elements located in native, mature mRNA, where sequence elements are exons and 5-′ and 3′-noncoding regions. Immature mRNA could have exons separated by intervening introns, which, if present, are removed during post-translational RNA spicing to form mature mRNA having open reading frame.

Genomic sequence of interest could include nucleic acid present between initiating codon and terminating codon, as determined in the said sequences, including all introns, which are normally present in a native chromosome. Genomic sequence of interest could additionally include 5′- and 3′-untranslated regions in the mature mRNA, as well as specific transcrpitional and translational regulatory sequences, such as promoters, enhancers, etc., including flanking genomic DNA approximately 1 kbp in size, but possibly even more, at 5′- or 3′-terminal of the transcribed region.

The invention also covers nucleic acids, which are homologous, substantially similar, identical, derivatives or mimetics of nucleic acids coding proteins of this invention.

The claimed nucleic acids are present in the environment different from their natural medium, e.g. they are isolated, present in enriched quantities, or present or expressed in vitro or in a cell, or in an organism, other than their naturally occurring environment.

Specific nucleic acids of interest include nucleic acids, which code hispidin hydroxylase or hispidin synthase, or caffeylpyruvate hydrolase described in “Proteins” section above. Each of these specific nucleic acids of interest is individually disclosed in more details.

Nucleic Acids Coding Hispidin Hydroxylases.

In preferred embodiments nucleic acids of the invention code proteins able to catalyze reaction of transformation of 6-(2-arylvinyl)-4-hydroxy-2H-pyran-2-one (preluciferin) having the structural formula

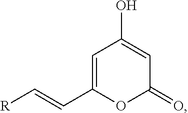

into 6-(2-arylvinyl)-3,4-dihydroxy-2H-pyran-2-one (fungal luciferin), having the structural formula

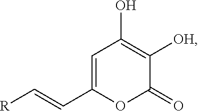

where R-aryl or heteroaryl.

In preferred embodiments nucleic acids code hispidin hydroxylases, which amino acid sequences are characterized by presence of several conservative amino acid motifs (consensus sequences) shown in SEQ ID NO: 29-33.

Specific examples of nucleic acids include nucleic acids coding hispidin hydroxylases, which amino acid sequences are shown in SEQ ID NOs: 2, 4, 6, 8 10, 12, 14, 16, 18, 20, 22, 24, 26, 28. Examples of nucleic acids, coding the said proteins, are given in SEQ ID NOs: 1, 3, 5, 7, 9, 11, 13, 15, 17, 19, 21, 23, 25, 27. Also, functional mutants, homologs and derivatives of the above mentioned specific nucleic acids are of interest.

In preferred embodiments nucleic acids of the invention code proteins, which amino acid sequences are at least 60%, or at least 65%, or at least 70%, or at least 80%, or at least 85%, or at least 90%, or at least 91%, or at least 92%, or at least 93%, or at least 94%, or at least 95%, or at least 96%, or at least 97%, or at least 98%, or at least 99% identical to the sequences shown in SEQ ID NOs: 2, 4, 6, 8 10, 12, 14, 16, 18, 20, 22, 24, 26, 28, for at least 350 amino acids.

Nucleic Acids Coding Hispidin-Synthases

In preferred embodiments nucleic acids of the invention code proteins able to catalyze reaction of transformation of 3-arylacrylic acid with the structural formula

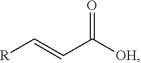

where R-aryl or heteroaryl in 6-2-arylvinyl)-4-hydroxy-2H-pyran-2-one, having the structural formula

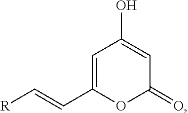

where R-aryl or heteroaryl.

In preferred embodiments nucleic acids code hispidin-synthases, which amino acid sequences are characterized by presence of several conservative amino acid motifs (consensus sequences) shown in SEQ ID NOs: 56-63.

Specific examples of nucleic acids include nucleic acids coding hispidin-synthases of the invention, which amino acid sequences are shown in SEQ ID NOs: 35, 37, 39, 41, 43, 45, 47, 49, 51, 53, 55. Examples of nucleic acids, coding the said proteins, are given in SEQ ID NOs: 34, 36, 38, 40, 42, 44, 46, 48, 50, 52, 54.

Also, functional mutants, homologs and derivatives of the above mentioned specific nucleic acids are of interest.

In preferred embodiments nucleic acids of the invention code proteins, which amino acid sequences are at least 45%, normally at least 50%, e.g. at least 55%, or at least 60%, or at least 65%, or at least 70%, or at least 80%, or at least 85%, or at least 90%, or at least 91%, or at least 92%, or at least 93%, or at least 94%, or at least 95%, or at least 96%, or at least 97%, or at least 98%, or at least 99% identical to the sequences shown in SEQ ID NOs: 35, 37, 39, 41, 43, 45, 47, 49, 51, 53, 55, for all protein polypeptide chain.

Nucleic Acids Coding Caffeylpyruvate Hydrolases

In preferred embodiments nucleic acids of the invention code proteins able to catalyze reaction of transformation of oxyluciferin with the structural formula

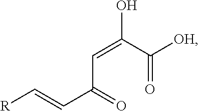

where R is aryl or heteroaryl, conversion into 3-arylacrylic acid with the structural formula

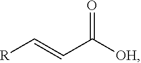

where R is selected from aryl, heteroaryl group.

In preferred embodiments nucleic acids code caffeylpyruvate hydrolases, which amino acid sequences are characterized by presence of several conservative amino acid motifs (consensus sequences) shown in SEQ ID NOs: 76-78. Specific examples of nucleic acids include nucleic acids coding caffeylpyruvate hydrolases, which amino acid sequences are shown in SEQ ID NOs: 65, 67, 69, 71, 73, 75. Examples of nucleic acids, coding the said proteins, are given in SEQ ID NOs: 64, 66, 68, 70, 72, 74.

Also, nucleic acids, coding functional mutants, homologs and derivatives of the above-mentioned proteins, are of interest.

In preferred embodiments nucleic acids of the invention code proteins, which amino acid sequences are at least 60%, or at least 65%, or at least 70%, or at least 80%, or at least 85%, or at least 90%, or at least 91%, or at least 92%, or at least 93%, or at least 94%, or at least 95%, or at least 96%, or at least 97%, or at least 98%, or at least 99% identical to the sequences shown in SEQ ID NOs: 65, 67, 69, 71, 73, 75, for all protein polypeptide chain.

Nucleic acids of interest (for example, nucleic acids coding homologs of proteins characterized by amino acid sequences shown in SEQ ID NOs: 2, 4, 6, 8 10, 12, 14, 16, 18, 20, 22, 24, 26, 28, 35, 37, 39, 41, 43, 45, 47, 49, 51, 53, 55, 65, 67, 69, 71, 73, 75), could be isolated from any organisms (fungi, plants, microorganisms, animals), in particular, from different kinds of bioluminescent fungi, e.g. fungi of Basidiomycota type, predominantly of Basidiomycetes class, in particular, Agaricales order, e.g. from bioluminescent fungi Neonothopanus nambi, Armillaria fuscipes, Armillaria mellea, Guyanagaster necrorhiza, Mycena citricolor, Neonothopanus gardneri, Omphalotus olearius, Panellus stipticus, Armillaria gallica, Armillaria ostoyae, Mycena chlorophos, etc. TAlso, non-bioluminescent fungi and plants producing hispidin, such as Pteris ensiformis, are of special interest as a source of nucleic acids coding homologs of proteins of this invention [Yung-Husan Chen et al., «Identification of phenolic antioxidants from Sword Brake fem (Pteris ensiformis Burm.)», Food Chemistry, Volume 105, Issue 1, 2007, pp. 48-56], Inonotus xeranticus [In-Kyoung Lee et al., «Hispidin Derivatives from the Mushroom Inonotus xeranticus and Their Antioxidant Activity», J. Nat. Prod., 2006, 69 (2), pp. 299-301], Phellinus sp. [In-Kyoung Lee et al., «Highly oxygenated and unsaturated metabolites providing a diversity of hispidin class antioxidants in the medicinal mushrooms Inonotus and Phellinus». Bioorganic & Medicinal Chemistry. 15 (10): 3309-14.], Equisetum arvense [Markus Herderich et al., «Establishing styrylpyrone synthase activity in cell free extracts obtained from gametophytes of Equisetum arvense L. by high performance liquid chromatography-tandem mass spectrometry». Phytochem. Anal., 8: 194-197.].

Homologs are identified by any of the variety of methods. cDNA fragment of this invention could be used as a hybridization probe versus cDNA library from the target organism, using low stringency conditions. The probe could be a large fragment or one or shorter degenerate primer. Nucleic acids, having sequence similarity, are detected by hybridization in low stringency conditions, for example, at 50° C. and 6×SSC (0.9 M of sodium chloride/0.09 M of sodium citrate) followed by washing at 55° C. in 1×SSC (01.15 M of sodium chloride/0.015 M of sodium citrate). Sequence identity could be determined by hybridization in high stringency conditions, for example, at 50° C. or higher and 0.1×SSC (15 mM of sodium chloride/1.5 mM of sodium citrate). Nucleic acids having the region substantially identical to the presented sequences, e.g. allelic variants, genetically modified variants of nucleic acid, etc., are bound with the presented sequences in high stringency conditions of hybridization. Using probes, in particular, labeled probes of DNA sequences, enables to recover homologous or similar genes.

Homologs could be identified by means of polymerase chain reaction from genomic or cDNA library. Oligonucleotide primers, representing the fragments of known sequences of specific nucleic acids, could be used as primers for PCR. In preferable aspect oligonucleotide primers have degenerate structure and correspond to nucleic acid fragments coding conservative regions of protein amino acid sequence, e.g. consensus sequences are shown in SEQ ID NOs: 29-33, 56-63, 76-78. Full-length coding sequences then could be detected by means of 3′- and 5′-RACE methods, well known from the prior art.

Homologs could also be identified in the results of whole-genome sequencing of organisms by comparison of amino acid sequences deduced on the basis of sequencing and amino acid sequences SEQ ID NOs: 2, 4, 6, 8 10, 12, 14, 16, 18, 20, 22, 24, 26, 28, 35, 37, 39, 41, 43, 45, 47, 49, 51, 53, 55, 65, 67, 69, 71, 73, 75. Sequence identity is determined based on reference sequence. Algorithms for sequence analysis are known in the art, e.g. BLAST, described in Altschul et al., J. Mol. Biol., 215, pp. 403-10 (1990). For the purposes of this invention, in order to determine the level of identity and similarity between nucleotide sequences and amino acid sequences there could be used a comparison of nucleotide and amino acid sequences performed by means of Blast software package provided by National Center for Biotechnology Information (http://www.ncbi.nlm.nih.gov/blast) using nicked alignment with standard parameters.

Nucleic acids which are hybridized with the above nucleic acids in stringent conditions, preferably in high stringency conditions (i.e. complementary to nucleic acids described before) are also provided. Example of hybridization in high stringency conditions is hybridization at 50° C. or higher and 0.1×SSC (15 mM of sodium chloride/1.5 mM of sodium citrate). Other example of hybridization in high stringency conditions is overnight incubation at 42° C. in 50% formamide, 5×SSC (150 mM NaCl, 15 mM trisodium citrate), 50 mM sodium phosphate (pH 7.6), 5×Denhardt's solution, 10% dextran sulphate and 20 μg/ml of salmon sperm denatured cut DNA, with preliminarily washing in 0.1×SSC at approximately 65° C. Other high stringency conditions of hybridization are known in the art and also can be used for identification of nucleic acids of the invention.

Nucleic acids coding variants, mutants or derivatives of proteins of the invention are also provided. Mutants or derivatives could be obtained from nucleic acid template, selected from the above described nucleic acids, by modification, deletion or adding of one or more nucleotides in template sequence or their combination to obtain a variant of nucleic acid template. Modifications, additions or deletions could be performed by any method known in the art (see, for example, Gustin et al., Biotechniques (1993) 14: 22; Barany, Gene (1985) 37: 111-123; and Colicelli et al., Mol. Gen. Genet. (1985) 199:537-539, Sambrook et al., Molecular Cloning: A Laboratory Manual, (1989), CSH Press, pp. 15.3-15.108), including error-prone PCR, shuffling, oligonucleotide-directed mutagenesis, assembly PCR, paired PCR mutagenesis, mutagenesis in vivo, cassette mutagenesis, recursive ensemble mutagenesis, exponential ensemble mutagenesis, oligonucleotide-directed mutagenesis, random mutagenesis, genetic reassembly, gene site saturation mutagenesis (GSSM), synthetic ligation reassembly (SLR) or their combinations. Modifications, additions or deletions could also be performed by method including recombination, recursive sequence recombination, phosphorothioate-modified DNA mutagenesis, uracil template mutagenesis, double-skip mutagenesis, point reducing mismatch mutagenesis, recovery deficient strain mutagenesis, chemical mutagenesis, radiation mutagenesis, deleted mutagenesis, restriction selective mutagenesis, restriction mutagenesis with purification, artificial gene synthesis, multiple mutagenesis, creation of chimeric multiple nucleic acids and their combinations. Nucleic acids coding shortened and elongated variants of the said luciferases are also under the scope of this invention. As used herein, these protein variants comprise amino acid sequences with modified C-, N-, or both terminals of polypeptide chain.

In preferred embodiments the homologs and mutants under discussion are functional enzymes able to perform fungal luciferin biosynthesis, e.g. fungi luciferin. Homologs and mutants of the interest could have altered properties, such as rate of maturation in a host cell, aggregability or dimerizability, half-life period or other biochemical properties, including substrate binding constant, thermal stability, pH stability, activity temperature optimum, activity pH optimum, Michaelis-Menten constant, substrate specificity, side issue range. In some embodiments homologs and mutants have the same properties as the claimed proteins.

Nucleic acids, coding functional homologs and mutants of this invention, could be identified during functional tests, for example, at expression library functional screening, described in “Proteins” section.

Besides, degenerate variants of nucleic acids, which code proteins of this invention, are also provided. Degenerate variants of nucleic acids include replacements of nucleic acid codons by other codons coding the same amino acids. In particular, the degenerate variants of nucleic acids are created to increase expression in a host cell. In this embodiment nucleic acid codons, which are not preferable or are less preferable in host cell genes, are replaced by codons which are excessively presented in the coding sequences in the host cell genes, where the said replaced codons code the same amino acid. In particular, humanized versions of nucleic acids of this invention are of the interest. As used herein, the term “humanized” refers to the replacements done in nucleic acid sequence to optimize codons for protein expression in mammal cells (Yang et al., Nucleic Acids Research (1996) 24: 4592-4593). See also U.S. Pat. No. 5,795,737, describing protein humanization, which disclosure is incorporated herein by reference. Variants of nucleic acids optimized for expression in plant cells are of particular interest. Examples of such nucleic acids, coding proteins of this invention, are given in SEQ ID NOs: 103, 113 and 114.

The claimed nucleic acids could be isolated and obtained substantially in purified form. Principally, the purified form means that nucleic acids are at least approximately 50% pure, normally at least approximately 90% pure and normally are “recombinant”, i.e. floxed by one or more nucleotides, which it is normally not bound with in a chromosome naturally occurring in its natural host organism.

The claimed nucleic acids could be artificially synthesized. Methods for producing nucleic acids are well known from the prior art. For example, accessibility of information about amino acid sequence or information about nucleotide sequence enables to produce isolated molecules of nucleic acids of this invention by means of oligonucleotide synthesis. In case of availability of information about amino acid sequence there could be synthesized several nucleic acids different from each other due to degeneracy of genetic code. Methods for selection of codon variants for the required host are well known in the art.

Synthetic oligonucleotides could be produced by phosphoramidite method and obtained constructs could be purified by such methods well known in the art as high performance liquid chromatography (HPLC) or other methods as described, for example, in Sambrook et al., Molecular Cloning: A Laboratory Manual, 2nd Ed., (1989) Cold Spring Harbor Press, Cold Spring Harbor, N.Y., and according to the instruction described, for example, in United States Dept. of HHS, National Institute of Health (NIH) Guidelines for Recombinant DNA Research. Long two-stranded DNA molecules of this invention could be synthesized as follows: several smaller fragments, which contain suitable terminals capable of cohesion with adjacent fragment, could be synthesized with the required complementarity. Adjacent fragments could be cross-linked by means of DNA lygase, recombination-based methods, or PCR-based method.

Nucleic acids coding fusion proteins, including proteins of this invention, are also provided. Examples of such proteins are given in “Proteins” section above. Nucleic acids coding fusion proteins could be artificially synthesized as described above.

Expression cassettes or systems, used inter alia for obtaining the claimed proteins (i.e. hispidin hydroxylases, hispidin synthases and caffeylpyruvate hydrolases) or fusion proteins on their basis or for replication of the claimed nucleic acid molecules, are also provided. Expression cassette could exist as extrachromosomal element or could be included into cell genome resulting from introduction of the said expression cassette into the cell. When introducing expression cassette into the cell a protein product is formed coded by nucleic acid of the invention; in this case it is said that protein is “produced” or “expresses” by the cell. Any expression system, including, for example, bacterial systems, yeast, plants, insects, amphibians or mammal cells, is applicable. Target nucleic acid in the expression cassette is operatively bound with regulatory sequences, which could include promoters, enhancers, terminator sequences, operators, repressors and inductors. Generally, the expression cassette comprises at least (a) transcription initiation region, functional in the host cell; (b) nucleic acid of the invention and (c) transcription termination region, functional in the host cell. Methods for obtaining expression cassettes or systems able to express the desired product are known to those skilled in the art.

Vector and other nucleic acid structures, comprising the claimed nucleic acids, are also provided. Suitable vectors include viral and nonviral vectors, plasmids, cosmids, phages, etc., and are used for cloning, amplification, expression, transfer, etc. of the nucleic acid sequence of this invention into suitable host. Selection of suitable vector is obvious to those skilled in the art. Full-length nucleic acid or its part is generally introduced to the vector by DNA lygase linking to the site split by restriction enzymes in the vector. Alternatively, the desired nucleotide sequence could be inserted by homologous recombination in vivo, normally, by linking homologous regions to the vector at flanks of the desired nucleotide sequence. Homologous regions are added by oligonucleotide ligation or by polymerase chain reaction, using primers, including, for example, as homologous regions, as a part of the desired nucleotide sequence. The vector, as a rule, has an origin of replication, promoting its reproduction in host cells as a result of its introduction into the cell as an extrachromosomal element. The vector could also comprise regulatory elements promoting expression of nucleic acid in the host cell and obtaining recombinant functional protein. In the expression vector the said nucleic acid is functionally bound to a regulatory sequence, which could include promoters, enhancers, terminators, operators, repressors, silencers, insulators, and inductors. For the purpose of expression of functional proteins or their shortened forms the coding nucleic acids are operatively cross-linked to the nucleic acids comprising at least regulatory sequences and transcription start site. Also, these nucleic acids could comprise sequences coding histidine tag (6 His tag), signal peptide or functional protein domains. In many embodiments the vectors promote integration of nucleic acid, operatively bound with regulatory elements, into the host cell genome. A vector could comprise expression cassette for a selectable marker, such as fluorescent protein (e.g. gfp), antibiotic resistance gene (e.g. ampicillin, or kanamycin, or neomycin, or hygromycin, etc. resistance gene), genes conditioning resistance to herbicides, such as genes conditioning resistance to phosphinothricin and sulphonamide herbicides, or other selectable marker known from the prior art.

A vector could comprise additional expression cassettes, including nucleic acids coding 4′-phosphopantetheinyl transferase, 3-arylacrylic acid synthesis proteins (for example, described in the section “Application, combinations and methods of use”), luciferases, etc.

The above expression systems could be used in prokaryotic or eukaryotic hosts. To obtain protein, there could be used such host cells as E. coli, B. subtilis, S. cerevisiae, insect cells or higher organism cells, which are not human embryonic cells, such as yeast, plants (e.g. Arabidopsis thaliana, Nicotiana benthamiana, Physcomitrella patens), vertebrata, e.g. COS 7 cells, HEK 293, CHO, Xenopus oocytes, etc.

Cell lines, which steadily produce proteins of the invention, could be selected by methods known in the art (for example, co-transfection with selectable marker, such as dhfr, gpt, antibiotic resistance genes (ampicillin, or kanamycin, or neomycin, or hygromycin, etc.), that enables to identify and isolate transfected cells, which comprise a gene included into the genome or incorporated into the extrachromosomal element.

If any above said host cell or other host cells or organisms suitable for replication and/or expression of nucleic acids of the invention are used, the obtained replicated nucleic acid, expressed protein or polypeptide are within the scope of the invention as a product of the host cell or organism. A product could be isolated by suitable method known in the art.

In many embodiments of this invention the cell is co-transfected with several expression cassettes comprising nucleic acids of the invention coding different enzymes of fungal luciferin biosynthesis. In some embodiments the expression cassette comprising nucleic acid coding luciferase, able to oxidize fungal luciferin with luminescence emission, is additionally introduced to the cell. In some cases, the expression cassettes are combined in one vector, which is used for cell transformation. In some embodiments the nucleic acids coding 4′-phosphopantetheinyl transferase and/or 3-arylacrylic acid synthesis proteins are additionally introduced to the cell.

Short DNA fragments of the claimed nucleic acids, which are used as PCR primers, rolling circle amplifications, hybridization screening probes, etc. are also provided. Long DNA fragments are used to obtain encoded polypeptides, as described above. However, for geometric amplification reactions, such as PCR, a pair of short DNA fragments, i.e. primers, is used. Exact primer sequence is not critical for the invention, however, for the most of applications the primers will be hybridized with the claimed sequence in stringent conditions, as known in the art. It is preferable to select a pair of primers, which give an amplification product from at least approximately 50 nucleotides, preferably from at least approximately 100 nucleotides, and could extend for the entire sequence of nucleic acid. Algorithms of primer sequences selection are normally known and available in commercial software packages. Amplification primers are hybridized with complementary DNA chains and will seed amplification counter reactions.

Nucleic acid molecules of this inventions can also be used to determine gene expression in biological specimen. The method where cells are examined for presence of specific nucleotide sequences, such as genomic DNA or RNA, is well known in the art. In brief, DNA or mRNA is isolated from a cell specimen. mRNA could be amplified by means of RT-PCR, using reverse transcriptase to form complementary DNA chain followed by amplification by means of polymerase chain reaction, using specific primers for the claimed DNA sequences. Alternatively, mRNA specimen is isolated by means of gel electrophoresis, transferred to a suitable carrier, e.g. nitrocellulose, nylone, etc., and then it is tested by a fragment of the claimed DNA as a probe. There also could be used other methods, such as oligonucleotide ligation analyses, hybridization in situ and hybridization by DNA-probes, immobilized on a hard array. Detection of mRNA hybridizing with the claimed sequence indicates gene expression in the specimen.

### Transgenic Organisms

Transgenic organisms, transgenic cells and transgenic cell lines expressing nucleic acids of this invention are also provided. Transgenic cells of this invention include one or several nucleic acids under examination in this invention, which are present as transgene. For the purposes of this invention there could be used any suitable host cell, including prokaryotic (e.g. Escherichia coli, Streptomyces sp., Bacillus subtilis, Lactobacillus acidophilus, etc.) or eukaryotic host cells, which are not human embryonic cells. Transgenic organisms of this invention could be prokaryotic or eukaryotic organisms, including bacteria, cyanobacteria, fungi, plants and animals, where one or more organism cells comprising heterologous nucleic acid of the invention are introduced to by incorporating it due to human manipulation, for example, in line with transgenic techniques known in the art.

In one embodiment of this invention the transgenic organism could be a prokaryotic organism. Methods for transformation of prokaryotic host cells are well known in the art (see, for example, Sambrook et al. (Molecular Cloning: A Laboratory Manual, 2nd Ed., (1989) Cold Spring Harbor Laboratory Press and Ausubel et al., Current Protocols in Molecular Biology (1995) John Wiley & Sons, Inc).

In the other embodiment of this invention the said transgenic organism could be a fungus, e.g. yeast. Yeasts are widely used as a carrier for heterologous gene expression (see, for example, Goodey et al., Yeast biotechnology, D R Berry et al., eds, (1987) Allen and Unwin, London, pp. 401-429, and Kong et al., Molecular and Cell Biology of Yeasts, E. F. Walton and G. T. Yarronton, eds, Blackie, Glasgow (1989) pp. 107-133). There are several yeast vectors available, including integrating vectors, which require recombination with host genome for its maintenance, and also autonomously replicating plasmid vectors.

The other host organism is an animal organism. Transgenic animals could be obtained using transgenic techniques known in the art and described in standard manuals (such as: Pinkert, Transgenic Animal Technology: A Laboratory Handbook, 2nd edition (2003) San Diego: Academic Press; Gersenstein and Vinterstein, Manipulating the Mouse Embryo: A Laboratory Manual, 3rd ed, (2002) Nagy A. (Ed), Cold Spring Harbor Laboratory; Blau et al., Laboratory Animal Medicine, 2nd Ed., (2002) Fox J. G., Anderson L. C., Loew F. M., Quimby F. W. (Eds), American Medical Association, American Psychological Association; Gene Targeting: A Ptactical Approach by Alexandra L. Joyner (Ed.) Oxford University Press; 2nd edition (2000)). For example, transgenic animals could be obtained by homologous recombination within a framework of which an endogenous locus is changed. Alternatively, nucleic acid structure is integrated into a genome in random mode. Vectors for stable integration include plasmids, retroviruses and other animal viruses, YAC, etc.

Nucleic acid could be introduced int a cell directly or indirectly due to introduction to the cell precursor by means of cautious genetic manipulation, such as microinjection, or by recombinant virus infection or using recombinant virus vector, transfection, transformation, gene gun delivery or transconjugation. Techniques of nucleic acid (e.g. DNA) molecules transfer into such organisms are well known and described in standard manuals, such as Sambrook et al. (Molecular Cloning: A Laboratory Manual, 3nd Ed., (2001) Cold Spring Harbor Press, Cold Spring Harbor, N.Y.).

The term “genetic manipulation” does not include classic crossbreeding or in vitro fertilization but rather refers to introduction of nucleic acid recombinant molecule. The said nucleic acid molecule could be integrated into a chromosome or could be extrachromosomal replicating DNA.

DNA structures for homologous recombination include at least a part of nucleic acid of the invention, where nucleic acid of the invention is operatively linked to homology regions, to target locus. For random integration it is not necessary to include homology regions into DNA structures to facilitate recombination. Positive and negative selection markers could also be included. Methods for obtaining the cells comprising target gene modifications by homologous recombination are known in the art. Different techniques of mammal cells transfection are described, for example, in the paper Keown et al., Meth. Enzymol. (1990) 185:527-537).

In case of embryonic stem cells (ES) there could be used ES cell line, or embryonic cells could be obtained fresh from a host organism, such as a mouse, rat, guineapig, etc. Such cells are grown on a corresponding fibroblast nurse layer or are grown in the presence of leukemia inhibitory factor (LIF). Transformed ES or embryonic cells could be used for creation of transgenic animals using the relevant technique known in the art.

Transgenic animals could be any animals different from a human, including mammal, different from a human (e.g. mouse or rat), bird or amphibia, etc., and they are used in functional tests, at drug screening, etc.

Transgenic plants could also be obtained. Methods for obtaining transgenic plant cells are described in the U.S. Pat. Nos. 5,767,367, 5,750,870, 5,739,409, 5,689,049, 5,689,045, 5,674,731, 5,656,466, 5,633,155, 5,629,470, 5,595,896, 5,576,198, 5,538,879 and 5,484,956, which descriptions are referenced to in this invention. Methods for obtaining transgenic plants are summarized in the following reviews: Plant Biochemistry and Molecular Biology (eds. Lea and Leegood, John Wiley & Sons (1993) pp. 275-295 and Plant Biotechnology and Transgenic Plants (eds. Oksman-Caldentey and Barz) (2002) 719 p.

For obtaining transgenic host organism there could be used, for example, embryogenic explants comprising somatic cells. After cells or tissues harvesting the exogenous DNA of interest is introduced into plant cells, and there are many different techniques available for such introduction. Availability of isolated protoplasts enables the introduction using DNA-mediated gene transfer protocols, including incubation of protoplasts with deproteinized DNA, such as plasmid, including exogenous coding sequence of interest, in the presence of multivalent cations (e.g. PEG or poly-L-ornithine); or according to the protoplast electroporation method in the presence of naked DNA, including exogenous sequence of interest. Then, there is selecting the protoplasts, which succeeded in exogenous DNA uptake, growing them up to callus formation and finally obtaining the transgenic plants by contacting the enhancing factors, such as auxins and cytokinins, taken in relevant quantities and ratio.

Plants could be obtained by other suitable methods, such as “gene gun”-based method or Agrobacterium-mediated transformation, known to those skilled in the art.

### Antibodies

The term “antibody” herein refers to a polypeptide or a group of polypeptides, including at least one antibody active site (antigen-binding site). The term “antigen-binding site” refers to a space structure, which surface parameters and charge distribution are complementary to antigen epitope: it promotes antibody binding with the relevant antigen. The term “antibody” covers, for example, antibodies of vertebrate animals, chimeric antibodies, hybrid antibodies, humanized antibodies, modified antibodies, monovalent antibodies, Fab fragments, and single-domain antibodies.

Antibodies specific for proteins of this invention are applicable in affinity chromatography, immunological screening, in detection and identification of proteins of the invention (hispidin hydroxylases, hispidin synthases and caffeylpyruvate hydrolases). Antibodies of interest are bound with antigen polypeptides or proteins, or protein fragments, which are described in “Protein” section. Antibodies of the invention could be immobilized to a carrier and used in immunological screening or affinity chromatographic column to detect and/or separate polypeptides, proteins or protein fragments, or cells including such polypeptides, proteins, or protein fragments. Alternatively, such polypeptides, proteins or protein fragments could be immobilized in such a way as to detect antibodies capable of linking with them specifically.

Antibodies specific for proteins of this invention, as polyclonal as monoclonal, could be obtained using standard methods. Generally, first of all, a protein is used to immunize suitable mammal, preferably, a mouse, rat, rabbit or goat. Rabbits and goats are preferable objects for obtaining polyclonal sera due to obtaining considerable volume of blood serum, and also availability of marked antirabbit and antigoat antibodies. Normally, immunization is carried out by mixing or emulsifying the specific protein in physiological saline, preferably with an adjuvant, such as Freund adjuvant, followed by introduction of the obtained mixture or emulsion parenterally (normally, by hypodermic or intramuscular injection). Normally, sufficient doses are 50-200 μg per one injection.

In different embodiments of the invention recombinant or natural proteins are used for immunization in native or denatured form. Protein fragments or synthetic polypeptides, comprising part of protein amino acid sequence of the invention, could also be used for immunization.

Immunization is normally boosted in 2-6 weeks by one or several additional protein injections in physiological saline, preferably with incomplete Freund adjuvant. Alternatively, antibodies could also be obtained by in vitro immunization using methods known in the art, which are equivalent to in vitro immunization from the perspective of this invention purposes. Polyclonal antisera are obtained by blood sampling from immunized animals into glass or plastic vessel followed by blood incubation at 25° C. within 1 hour and then by incubation at 4° C. within 2-18 hours. Serum is extracted by centrifugation (for example, at 1000 g within 10 minutes). 20-50 ml of blood could be obtained from rabbits at a time.

Monoclonal antibodies are obtained using standard Kohler-Milstein technique (Kohler & Milstein, 1975, Nature, 256, 495-496) or its modifications. Normally, a mouse or rat is immunized in accordance with the above information. However, in contrast to blood sampling from animals to obtain serum, this technique involves splenectomy (and, what is not necessary, extraction of some large lymph nodes) and tissue maceration to separate individual cells. If desired, spleen cells could be screened (after extraction of non-specifically adherent cells) by application of cell suspension on a plate or in a separate plate well coated by protein-antigen. B-lymphocytes, expressing membrane-bound immunoglobulin specific for the tested antigen, are bound on the plate in such a way that they are not washed from it with suspension residue. Then, there is fusing the resulting B-lymphocytes or all macerated splenocytes with myeloma cells resulting in formation of hybridomas: then, they are incubated in a selective medium (e.g. in HAT medium, comprising hypoxanthine, aminopterin and thymidine). The resulting hybridomas are plated in limiting incubation and tested for response of antibodies, which are specifically bound with antigen used for immunization (and which are not bound with extraneous agents). Then, the selected hybridomas secreting monoclonal antibodies (mAb) are incubated either in vitro (e.g. in fermentors in the form of a hollow fibre bundle or in glass vessels for tissue cultures), or in vivo (in ascites fluid in mice).

Antibodies (as polyclonal as monoclonal) could be tagged using standard methods. The suitable tags are fluorophores, chromophores, radionuclides (in particular, 32P and 1251), electron-dense reagents, enzymes, and ligands, for which specific binding partners are known). Enzymes are normally detected by their catalytic activity. For example, horseradish peroxidase is generally detected by its ability to convert 3,3′,5,5′-tetramethylbenzidine (TMB) into blue pigment, quantitatively assessed at spectrophotometer. The term “specific binding partner” refers to a protein able to bind molecule-ligand at high specificity level, as for example, in case with antigen and monoclonal antibody specific for it. The other examples of specific binding partners are biotin and avidin (or streptavidin), immunoglobulin-G and protein-A, and also multiple pairs of receptors and their ligands, known in the art. Other variants and capabilities are obvious to those skilled in the art and are considered as equivalent in the scope of this invention.

Antigens, immunogens, polypeptides, proteins, or protein fragments of this invention cause formation of specific binding partners—antibodies. The said antigens, immunogens, polypeptides, proteins, or protein fragments of this invention include immunogenic compositions of this invention. Such immunogenic compositions could additionally comprise or include adjuvants, carriers, or other compositions, which stimulate or enhance, or stabilize antigens, polypeptides, proteins or protein fragments of this invention. Such adjuvants and carriers are obvious to those skilled in the art.

### Application, Combinations, and Methods of Use

This invention provides for application of fungal luciferin biosynthesis proteins as enzymes catalyzing reactions (1) of luciferin synthesis (namely, 6-(2-arylvinyl)-3,4-dihydroxy-2H-pyran-2-one (fungal luciferin), having the structural formula

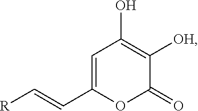

where R-aryl or heteroaryl, from preluciferin (namely, 6-2-arylvinyl)-4-hydroxy-2H-pyran-2-one), having the structural formula

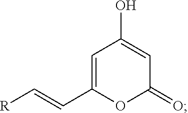

or preluciferin synthesis from 3-arylacrylic acid (preluciferin precursor) with the structural formula

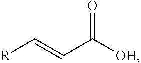

where R is selected from aryl or heteroaryl group; or 3-arylacrylic acid synthesis from 6-aryl-2-hydroxy-4-oxohexa-2,5-diene acid (oxyluciferin) with the structural formula

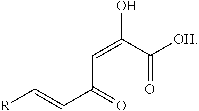

Fungal luciferin biosynthesis proteins are applied in many embodiments of this invention, and their non-limiting examples are given in this chapter below.

Fungal luciferin biosynthesis proteins, which application is ensured by this invention, could be obtained from different natural sources or by recombinant technologies. For example, wild-type proteins could be isolated from bioluminescent fungi, e.g. fungi of Basidiomycota type, predominantly of Basidiomycetes class, in particular, Agaricales order. For example, wild-type proteins could be isolated from bioluminescent fungi Neonothopanus nambi, Armillaria fuscipes, Armillaria mellea, Guyanagaster necrorhiza, Mycena citricolor, Neonothopanus gardneri, Omphalotus olearius, Panellus stipticus, Armillaria gallica, Armillaria ostoyae, Mycena chlorophos, etc. They could also be obtained by expression of recombinant nucleic acid, coding protein sequence in respective host or in cell-free expression system.

In some embodiment's proteins are applied inside host cells, where they are expressed and perform fungal luciferin cyclic transformations. In other embodiments isolated recombinant or natural proteins or extracts comprising proteins of the application are used.

Fungal luciferin biosynthesis proteins are active in physiological conditions.

In some embodiment's proteins—hispidin hydroxylases are applied in vitro and in vivo to obtain luciferin, which is oxidized by bioluminescent fungi luciferases, their homologs and mutants with luminescence emission. Therefore, this invention provided for application of hispidin hydroxylases of this invention to catalyze the transformation of 6-(2-arylvinyl)-4-hydroxy-2H-pyran-2-one (preluciferin) having the structural formula

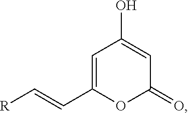

into 6-(2-arylvinyl)-3,4-dihydroxy-2H-pyran-2-one (fungal luciferin), having the structural formula

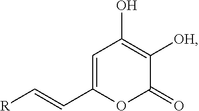

where R-aryl or heteroaryl.

Method for obtaining fungal luciferin from preluciferin includes combination of at least one molecule of hispidin hydroxylase with at least one molecule of 6-(2-arylvinyl)-4-hydroxy-2H-pyran-2-one, with at least one molecule of NAD(P)H and with at least one molecule of molecular oxygen (02). The reaction is carried out in physiological conditions in vitro and in vivo at the temperature from 20 to 42° C., and also the reaction could be carried out in cells, tissues and host organisms expressing hispidin hydroxylase. In preferred embodiments the said cells, tissues and organisms comprise sufficient amount of NAD(P)H and molecular oxygen to carry out the reaction. Exogenously delivered 6-(2-arylvinyl)-4-hydroxy-2H-pyran-2-one or endogenous 6-(2-arylvinyl)-4-hydroxy-2H-pyran-2-one produced in cells, tissues and organisms could be used in the reaction.

In preferred embodiments hispidin hydroxylases of this invention synthesize 3-hydroxyhispidin from hispidin. In preferred embodiments they synthesize at least one functional analogue of 3-hydroxyhispidin from the corresponding preluciferin shown in Table 2. In some embodiments hispidin hydroxylases of this invention synthesize 6-(2-arylvinyl)-3,4-dihydroxy-2H-pyran-2-one from any corresponding 6-(2-arylvinyl)-4-hydroxy-2H-pyran-2-one, having the structural formula

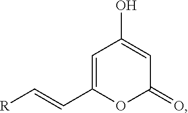

where R-aryl or heteroaryl.

The obtained 6-(2-arylvinyl)-3,4-dihydroxy-2H-pyran-2-one is applied for luminescence emitting in vitro and in vivo systems comprising functional luciferase, identifying fungal luciferin as a substrate.

For this invention purposes the proteins, which amino acid sequences are shown in SEQ ID NOs: 2, 4, 6, 8 10, 12, 14, 16, 18, 20, 22, 24, 26, 28, and also their mutants, homologs and derivatives are applicable as hispidin-hydroxylases. For example, there could be used functional hispidin hydroxylases 2, 4, 6, 8 10, 12, 14, 16, 18, 20, 22, 24, 26, 28, or at least 60%, or at least 65%, or at least 70%, or at least 80%, or at least 85%, or at least 90%, or at least 91%, or at least 92%, or at least 93%, or at least 94%, or at least 95%, or at least 96%, or at least 97%, or at least 98%, or at least 99% identical for at least 350 amino acids. For example, they could be at least 60%, or at least 65%, or at least 70%, or at least 80%, or at least 85%, or at least 90%, or at least 91%, or at least 92%, or at least 93%, or at least 94%, or at least 95%, or at least 96%, or at least 97%, or at least 98%, or at least 99% identical for all protein polypeptide chain.

In preferred embodiments for this invention purposes the proteins, which amino acid sequences are characterized by presence of several conservative amino acid motifs (consensus sequences) shown in SEQ ID NOs: 29-33, are applicable as hispidin-hydroxylases. Consensus sites inside hispidin hydroxylase amino acid sequences are operatively linked via amino acid inserts with lower insertions (FIG. 1).

In some embodiments hispidin-synthase proteins are applied in vitro and in vivo to produce fungal luciferin from its precursor, i.e applied to catalyze the transformation of 3-arylacrylic acids with the structural formula

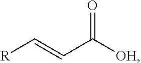

where R is selected from aryl, heteroaryl group, into 6-(2-arylvinyl)-4-hydroxy-2H-pyran-2-one, having the structural formula

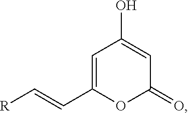

where R-aryl or heteroaryl.

Method for obtaining preluciferin includes combination of at least one molecule of hispidin-synthase with at least one molecule of 3-arylacrylic acid, with at least one molecule of coenzyme A, at least one molecule of AMP and at least two molecules of malonyl-CoA.

The reaction is carried out in physiological conditions at the temperature from 20 to 42° C., and also the reaction could be carried out in cells, tissues and host organisms expressing hispidin-synthase. In preferred embodiments the said cells, tissues and organisms comprise sufficient amount of coenzyme A, malonyl-CoA and AMP to carry out the reaction.

Exogenously delivered 3-arylacrylic acid or 3-arylacrylic acid produced in cells, tissues and organisms could be used in the reaction.

For example, hispidin-synthases of this invention could be used for producing hispidin from caffeic acid. In preferred embodiments they synthesize functional analogue of hispidin (6-(2-arylvinyl)-4-hydroxy-2H-pyran-2-one) from 3-arylacrylic acid shown in Table 2.

The obtained 6-(2-arylvinyl)-4-hydroxy-2H-pyran-2-one is applied for producing fungal luciferin in the presence of hispidin hydroxylase of this invention. Hispidin and its functional analogues are also applied in the medical field, since they exhibit antioxidant and antitumor properties; there is some evidence that hispidin is able to prevent obesity [Be Tu et al., Drug Discov Ther. 2015 June; 9 (3): 197-204; Nguyen et al., Drug Discov Ther. 2014 December; 8 (6): 238-44; Yousfi et al., Phytother Res. 2009 September; 23 (9):1237-42].

For this invention purposes the proteins, which amino acid sequences are shown in SEQ ID NOs: 35, 37, 39, 41, 43, 45, 47, 49, 51, 53, 55, and also their mutants, homologs and derivatives are applicable as hispidin-synthases. For example, there could be used functional hispidin-synthases with amino acid sequence identical to the sequence selected from the group of SEQ ID NOs: 35, 37, 39, 41, 43, 45, 47, 49, 51, 53, 55, at least 40%, prevalently at least 45%, normally at least 50%, e.g. at least 55%, or at least 60%, or at least 65%, or at least 70%, or at least 80%, or at least 85%, or at least 90%, or at least 91%, or at least 92%, or at least 93%, or at least 94%, or at least 95%, or at least 96%, or at least 97%, or at least 98%, or at least 99% identical.

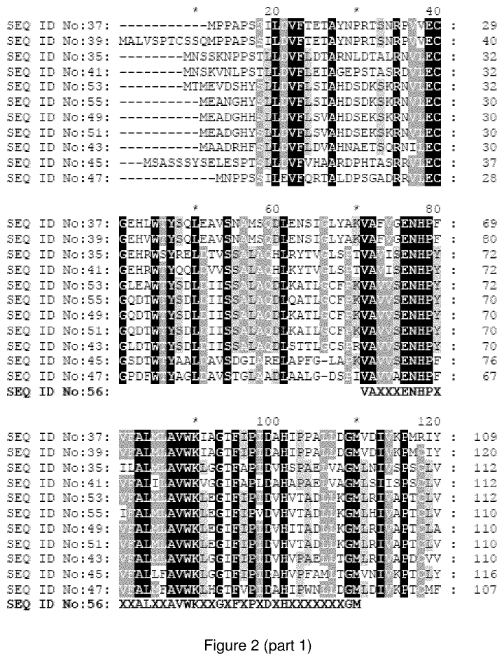

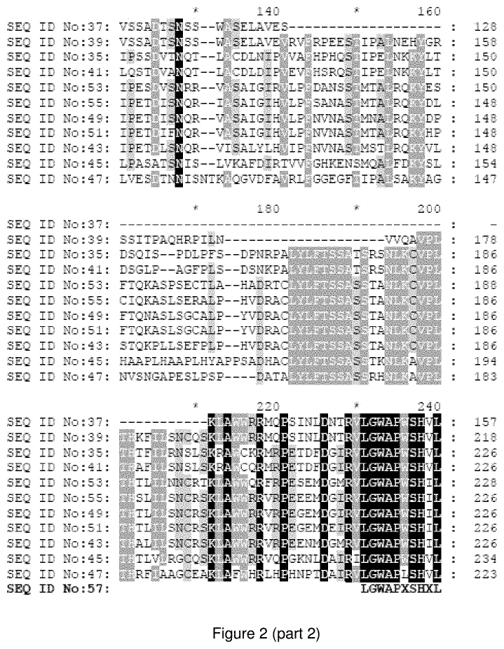

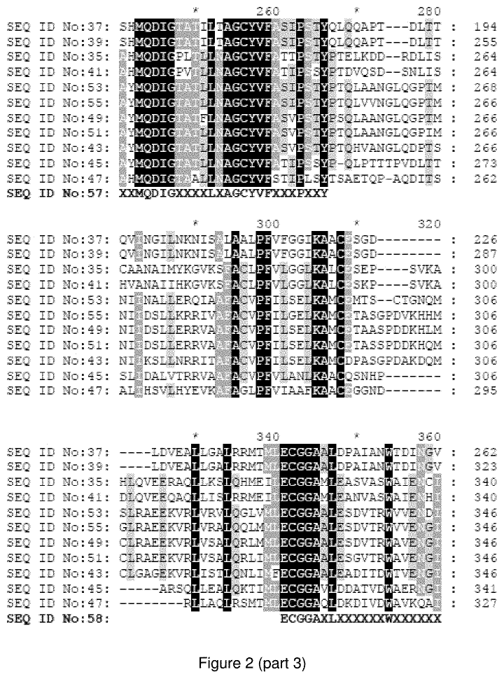

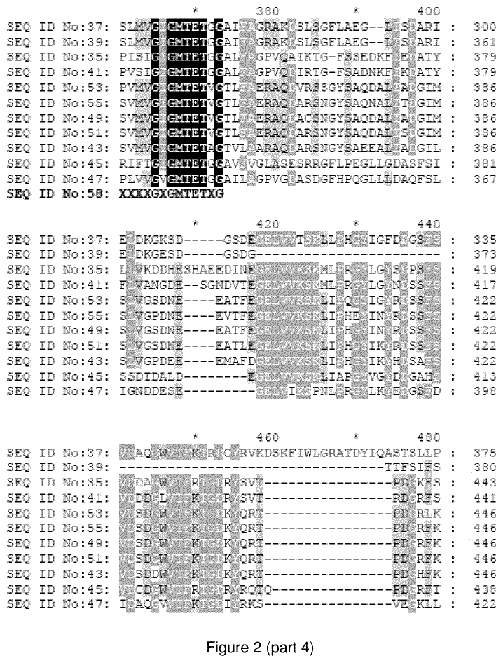

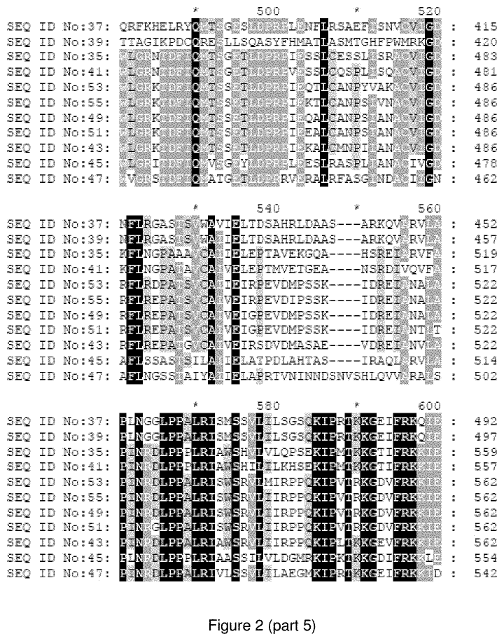

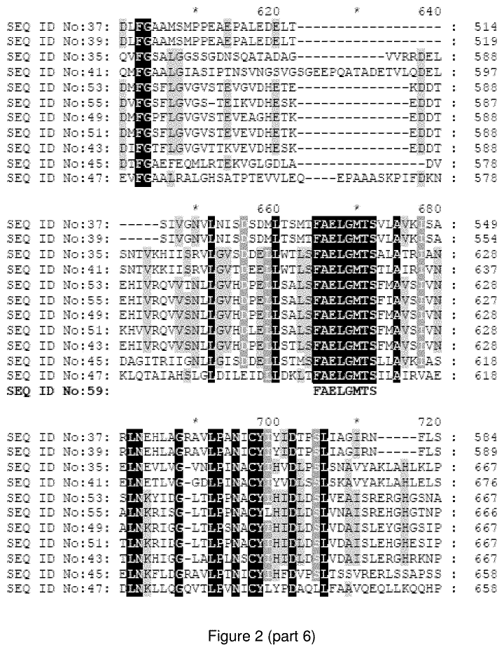

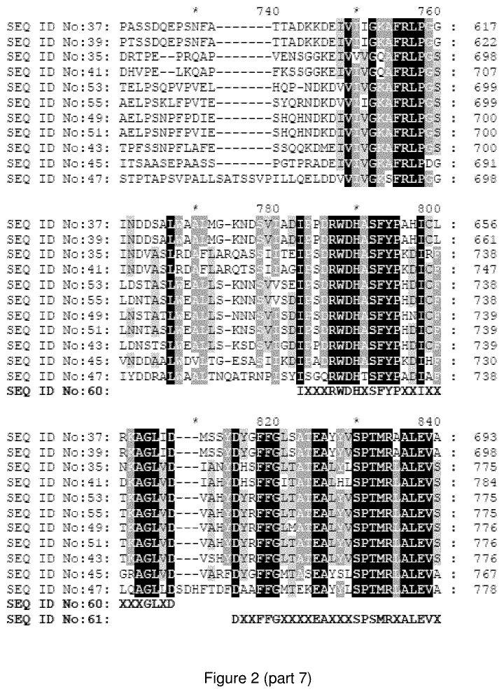

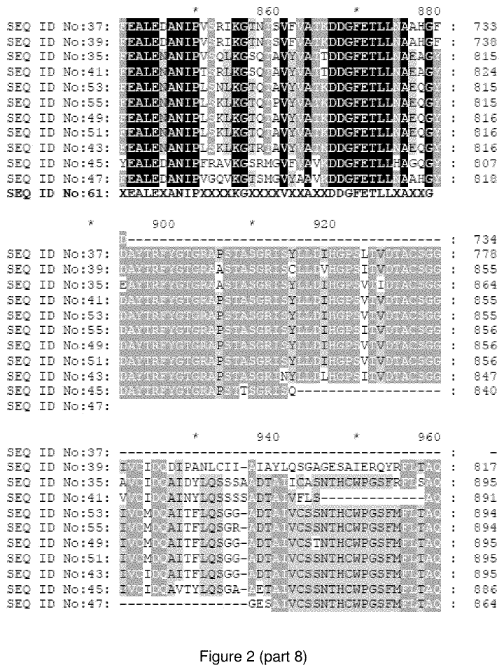

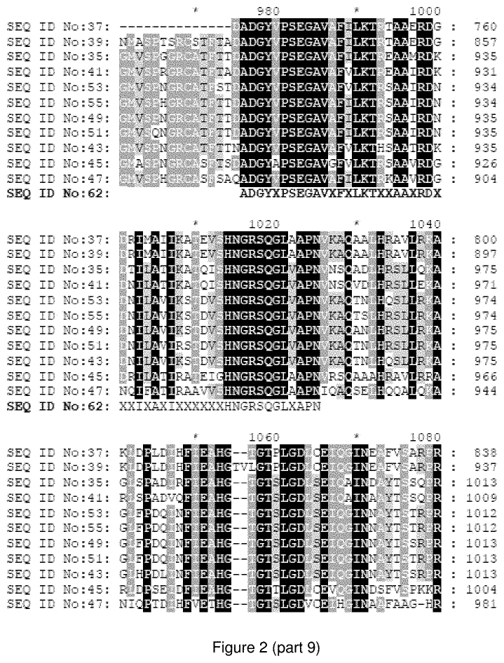

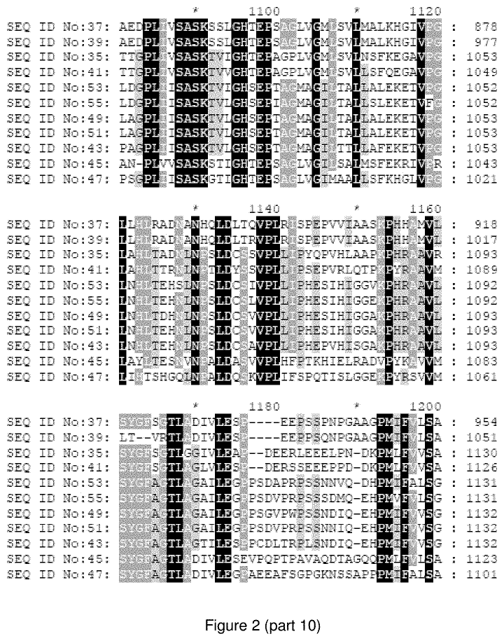

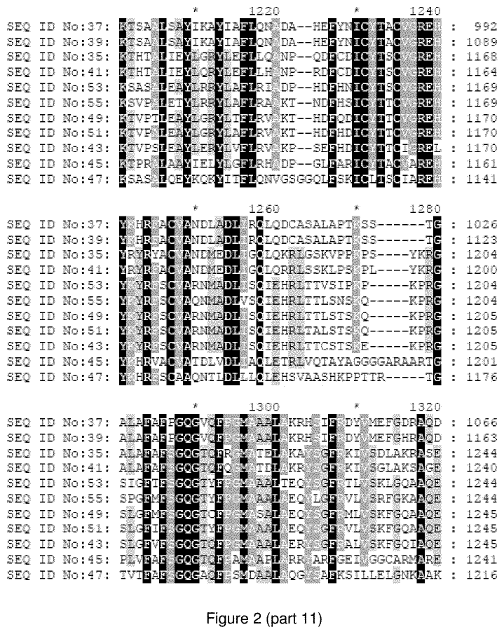

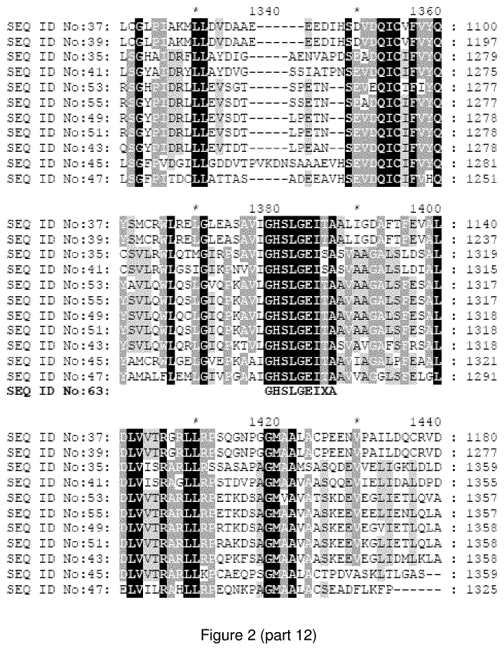

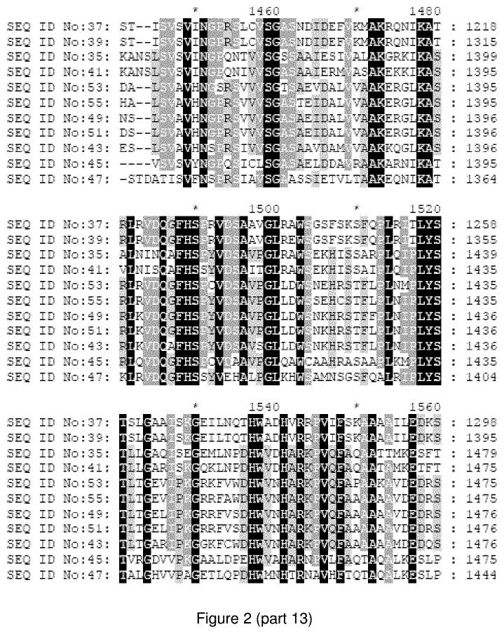

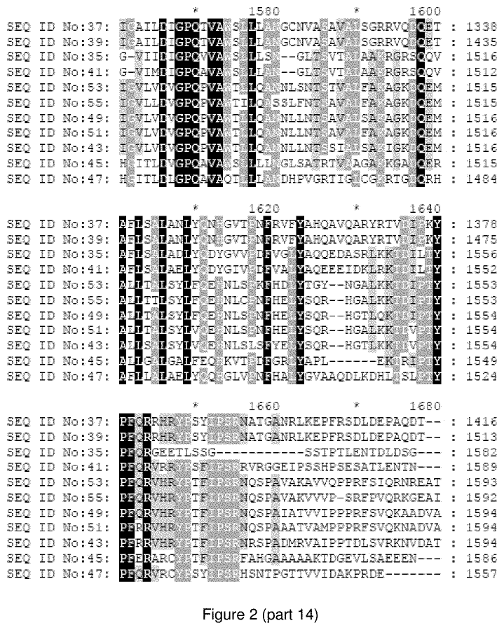

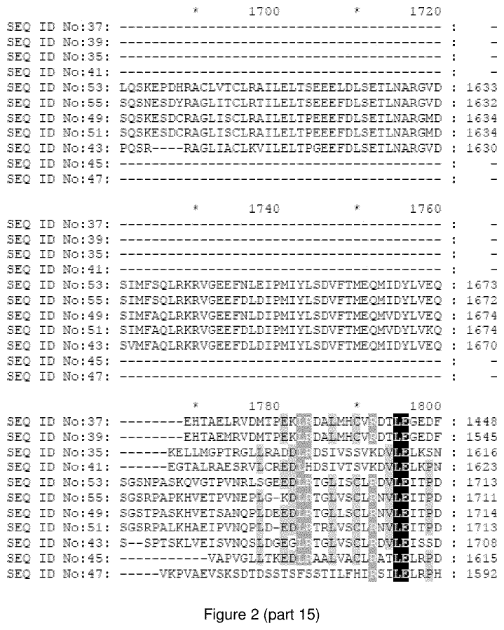

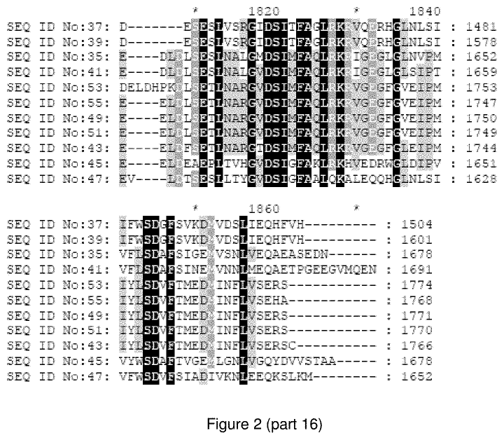

In preferred embodiments for this invention purposes the proteins, which amino acid sequences are characterized by presence of several conservative amino acid motifs (consensus sequences) shown in SEQ ID NOs: 56-63, are applicable as hispidin-synthases. Consensus sites inside hispidin-synthase amino acid sequences are operatively linked via amino acid inserts with lower insertions (FIG. 2).

In some embodiments caffeylpyruvate hydrolase proteins are applied in vitro and in vivo for producing 3-arylacrylic acids with the structural formula

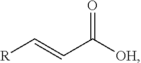

where R is selected from aryl, heteroaryl group, from 6-aryl-2-hydroxy-4-oxohexa-2,5-diene acid with the structural formula

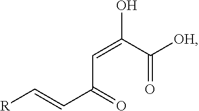

where R-aryl or heteroaryl. The reaction is carried out in physiological conditions in vitro and in vivo. Caffeoyl pyruvate hydrolases of this invention are applied in autonomous bioluminescence systems described in detail below.

For this invention purposes the proteins, which amino acid sequences are shown in SEQ ID NOs: 65, 67, 69, 71, 73, 75, and also their functional mutants, homologs and derivatives are applicable as caffeoyl pyruvate hydrolases. For example, there could be used functional caffeylpyruvate hydrolases with amino acid sequence identical to the sequence selected from the group of SEQ ID NOs: 65, 67, 69, 71, 73, 75, at least 60%, at least 65%, at least 70%, at least 80%, at least 85%, at least 90%, or at least 91%, or at least 92%, or at least 93%, or at least 94%, or at least 95%, or at least 96%, or at least 97%, or at least 98%, or at least 99% identical.

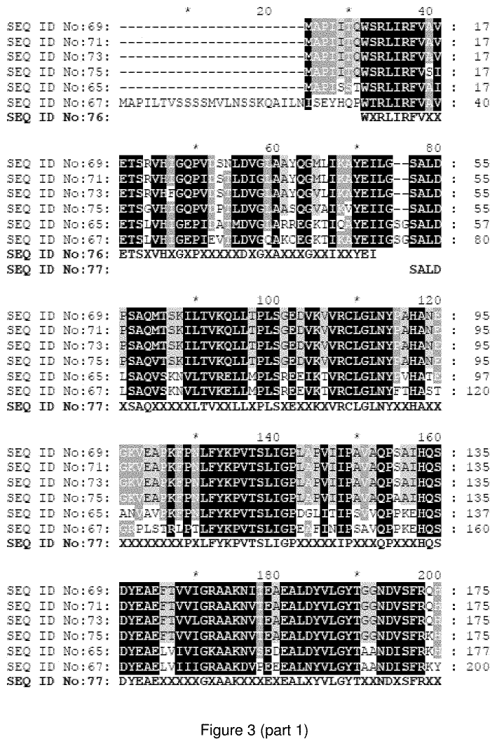

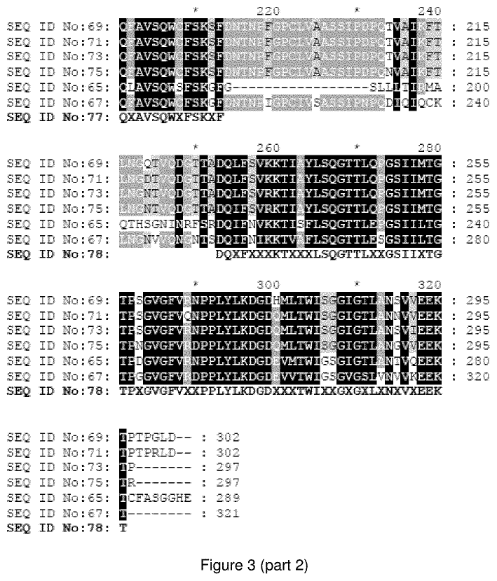

In preferred embodiments for this invention purposes the proteins, which amino acid sequences are characterized by presence of several conservative amino acid motifs (consensus sequences) shown in SEQ ID NOs: 76-78, are applicable as caffeoyl pyruvate hydrolases. Consensus sites inside caffeylpyruvate hydrolase amino acid sequences are operatively linked via amino acid inserts with lower insertions (FIG. 3).

Protein combinations applicable in the methods of this invention are also provided. In preferred embodiments the combinations include functional hispidin hydroxylase and functional hispidin synthase. This combination is applied for producing 6-(2-arylvinyl)-3,4-dihydroxy-2H-pyran-2-one from 3-arylacrylic acid with the structural formula

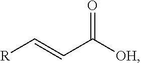

where R is aryl or heteroaryl. For example, the combination could be used for producing caffeic acid hydroxyhispidin. The reaction is carried out in physiological conditions in the presence of at least one molecule of hispidin hydroxylase, at least one molecule of hispidin synthase, at least one molecule of 3-arylacrylic acid, at least one molecule of coenzyme A, at least one molecule of AMP, at least two molecules of malonyl-CoA, at least one molecule of NAD(P)H and at least of one molecule of molecular oxygen (O2).

In some embodiments the combination also includes luciferase able to use 6-(2-arylvinyl)-3,4-dihydroxy-2H-pyran-2-one with the structural formula

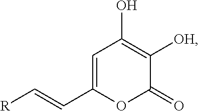

where R-aryl or heteroaryl, as luciferin. Oxidation of the said 6-(2-arylvinyl)-3,4-dihydroxy-2H-pyran-2-one by such luciferase is accompanied with bioluminescence and formation of oxyluciferin (6-aryl-2-hydroxy-4-oxohexa-2,5-diene acid).

Any protein characterized by the above activity could be used as a luciferase. For example, known luciferases from bioluminescent fungi, including those described in the application RU NQ 2017102986/10(005203) dd 30 Jan. 2017, and also their homologs, mutants and fused proteins having luciferase activity.

In many embodiments of this invention the luciferases, applicable for this invention purposes, are characterized by amino acid sequences, which are at least 40% identical, e.g. at least 45% identical, or at least 50% identical, or at least 55% identical, or at least 60% identical, or at least 70% identical, or at least 75% identical, or at least 80% identical, or at least 85% identical to the amino acid sequence selected from the group of SEQ ID NOs: 80, 82, 84, 86, 88, 90, 92, 94, 96, 98. Luciferases are often characterized by amino acid sequences, which have the following identity to the amino acid sequence selected from the group of SEQ ID NOs: 80, 82, 84, 86, 88, 90, 92, 94, 96, 98, minimum 90% of identity (e.g. at least 91%, minimum 92%, minimum 93%, minimum 94%, minimum 95%, minimum 96%, minimum 97%, minimum 98%, minimum 99% of identity or 100% of identity).

Mutants can retain biological properties of wild-type luciferase, from which they have been obtained, or can have biological properties different from wild-type proteins. The term “biological properties” of luciferases according to this invention refers, without limitation, to capability to oxidize different luciferins; biochemical properties, such as in vivo and/or in vitro stability (e.g. half-life); rate of maturation; tendency to aggregation or oligomerization, and also other similar properties. Mutations include changes of one or more amino acids, deletion, or insertion of one or more amino acids, replacements or truncations, or N-terminal truncations or extensions, C-terminal truncations or extensions, etc.

In some embodiments of the invention the luciferases are used in isolated form, i.e. they are substantially free from other proteins or other natural biological molecules, such as oligosaccharides, nucleic acids and their fragments, etc., where the term “substantially free from” in this case means that less than 70%, normally less than 60% and prevalently less than 50% of the said composition, comprising the isolated protein, is the other natural biological molecule. In some embodiments the said proteins are substantially in purified form, where the term “substantially purified form” means purity equal at least 95%, normally equal at least 97% and prevalently equal at least 99%.

In some embodiments the luciferases are used as part of extracts obtained from bioluminescent fungi or host cells comprising nucleic acids coding recombinant luciferases.

In many embodiments the luciferases are in heterologous expression systems (in cells or organisms of this invention), which comprise nucleic acids coding recombinant luciferases.

Methods for producing recombinant proteins, in particular, luciferases, as in isolated form, or as part of extracts, or in heterologous expression systems, are well known in the art and described in “Nucleic Acids” section. Protein purification methods are described in “Proteins” section.

In preferred embodiments the luciferases retain activity at temperatures below 50° C., prevalently at temperatures maximum 45° C., i.e. they retain activity at temperatures 20-42° C. and could be used in heterologous expression systems in vitro and in vivo. Normally, the described luciferases have pH stability within the range from 4 to 10, prevalently within the range from 6.5 to 9.5. Optimum pH stability of the claimed proteins is within the range from 7.0 to 8.5, e.g. between 7.3-8.0. In preferred embodiments the said luciferases are active in physiological conditions.

Combination of hispidin hydroxylase and luciferase oxidizing fungal luciferin with luminescence emission is applied in methods of hispidin and its functional analogues identification in biological objects: cells, tissues or organisms. The method includes contact of the test biological object or extract, obtained from it, with combination of isolated hispidin hydroxylase and said luciferase in suitable reaction buffer creating physiological conditions and comprising the required components to carry out reactions. A person skilled in the art could make a variety of reaction buffers satisfying this condition. Non-limiting example of the reaction buffer could be 0.2 M sodium phosphate buffer (pH 7.0-8.0) laced with 0.5 M of Na₂SO₄, 0.1% of dodecylmaltoside (DDM), 1 mM of NADPH.

Presence of hispidin or its functional analogue is determined by occurrence of the detectable luminescence—bioluminescence. Methods for detecting the detectable luminescence are described above in “Proteins” section when describing the functional screening. methods.

Combination of hispidin hydroxylase, hispidin synthase and luciferase oxidizing fungal luciferin with luminescence emission is applied in methods for identifying 3-arylacrylic acid with the structural formula

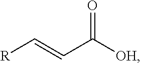

where R is aryl or heteroaryl, in biological objects. The method includes contact of the test biological object or extract, obtained from it, with combination of isolated hispidin hydroxylase, hispidin synthase and luciferase creating physiological conditions and comprising the required components to carry out reactions. A person skilled in the art could make a variety of reaction buffers satisfying this condition. Non-limiting example of the reaction buffer could be 0.2 M sodium phosphate buffer (pH 7.0-8.0) laced with 0.5 M of Na₂SO₄, 0.1% of dodecylmaltoside (DDM), 1 mM of NADPH, 10 mM of ATP, 1 mM of CoA, 1 mM of malonyl-CoA.

Presence of 3-arylacrylic acid is determined by occurrence of the detectable luminescence—bioluminescence. Methods for detecting the detectable luminescence are described above in “Proteins” section when describing the functional screening. methods.

In some embodiments instead of the combination of hispidin hydroxylase and luciferase oxidizing fungal luciferin with luminescence emission there could be used a fusion protein described in “Protein” section above. A fusion protein simultaneously exhibits hispidin hydroxylase activity and luciferase activity and it could be used in any methods instead of the combination of the said enzymes.

In some embodiments instead of the above hispidin-synthase there is used a type III polyketide synthase characterized by amino acid sequence identical to the amino acid sequence selected from the group of SEQ ID NOs: 119, 121, 123, 125, 127, 129, 131, 133, 135, 137, 139. For the purposes of this invention there are applicable type II polyketide synthases having the amino acid sequence identical to the sequence selected from the group of SEQ ID NOs: 119, 121, 123, 125, 127, 129, 131, 133, 135, 137, 139 at least 40%, prevalently at least 45%, normally at least 50%, e.g. at least 55%, or at least 60%, or at least 65%, or at least 70%, or at least 80%, or at least 85%, or at least 90%, or at least 91%, or at least 92%, or at least 93%, or at least 94%, or at least 95%, or at least 96%, or at least 97%, or at least 98%, or at least 99% identical.

The representatives of the said polyketide synthases (PKS) are identified in many plant organisms; their and/or their mutant capability to catalyze bisnoryangonin synthesis from coumaryl-CoA is known in the art [Lim et al., Molecules, 2016 Jun. 22; 21(6)]. The Applicants have demonstrated that the said enzymes are also able to catalyze hispidin synthesis from caffeyl-CoA in vitro and in vivo:

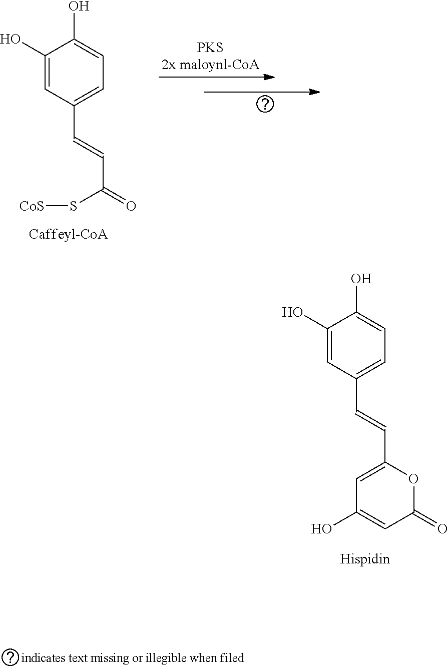

Therefore, application of the said proteins for hispidin synthesis is also within the scope of this invention.

In some embodiments of the invention PKS are used in isolated form, i.e. they are substantially free from other proteins or other natural biological molecules, such as oligosaccharides, nucleic acids and their fragments, etc., where the term “substantially free from” in this case means that less than 70%, normally less than 60% and prevalently less than 50% of the said composition, comprising the isolated protein, is the other natural biological molecule. In some embodiments the said proteins are substantially in purified form, where the term “substantially purified form” means purity equal at least 95%, normally equal at least 97% and prevalently equal at least 99%.

In many embodiments PKS are in heterologous expression systems (in cells or organisms of this invention), which comprise nucleic acids coding recombinant enzymes.

Methods for producing recombinant proteins, as in isolated form, or as part of extracts, or in heterologous expression systems, are well known in the art and described in “Nucleic Acids” section. Protein purification methods are described in “Proteins” section.

In preferred embodiments PKS retain activity at temperatures below 50° C., prevalently at temperatures maximum 45° C., i.e. they retain activity at temperatures 20-42° C. and could be used in heterologous expression systems in vitro and in vivo. Normally, the described PKS have pH stability within the range from 4 to 10, prevalently within the range from 6.0 to 9.0. Optimum pH stability of the claimed proteins is within the range from 6.5 to 8.5, e.g. between 7.0-7.5. In preferred embodiments the said PKS are active in physiological conditions.

Method for obtaining hispidin includes combination of at least one molecule of type III polyketide synthases, described above, with at least two molecules of malonyl-CoA and at least one molecule of caffeyl-CoA.

In some embodiments the method includes producing caffeyl-CoA from caffeic acid during enzymatic reaction catalyzed by coumarate-CoA ligase. In this case the method includes combination of type III polyketide synthases, described above, with at least one molecule of caffeic acid, with at least one molecule of coenzyme A, at least one molecule of coumarate-CoA ligase, at least one molecule of ATP and at least two molecules of malonyl-CoA.

For the purposes of this invention there could be used any coumarate-CoA ligase enzymes, known in the art, which perform reaction of coenzyme A addition to caffeic acid with caffeyl-CoA formation:

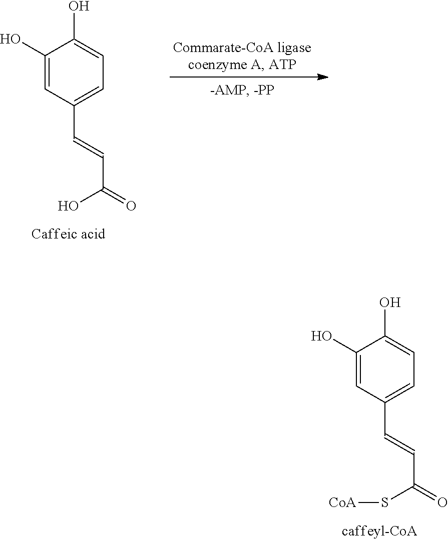

In particular, there could be used coumarate-CoA ligase 1 from Arabidopsis thaliana, having amino acid and nucleic sequences shown in SEQ ID NO: 141, and also its functional mutants and homologs. For example, for the purposes of this invention it is applicable the functional coumarate-CoA ligase, which amino acid sequence has minimum 40% of identity, e.g. minimum 45% of identity, or minimum 50% of identity, or minimum 55% of identity, or minimum 60% of identity, or minimum 65% of identity, or minimum 70% of identity, or minimum 75% of identity, e.g. minimum 80% of identity, minimum 85% of identity, minimum 90% of identity (e.g. at least 90%, 91%, 92%, 93%, 94%, 95%, 96%, 97%, 98%, 98% or 99% of identity) with amino acid sequence shown in SEQ ID NO: 141.

All the said reactions are carried out in physiological conditions at the temperature from 20 to 50° C., and also the reaction could be carried out in cells, tissues and host organisms expressing functional enzymes.

PKS and coumarate-CoA ligase combined with hispidin hydroxylase of this invention could be used for producing 3-hydroxyhispidin from caffeic acid. The reaction is carried out in physiological conditions in the presence of at least one molecule of hispidin hydroxylase, at least one molecule of PKS, at least one molecule of coumarate-CoA ligase, at least one molecule of caffeic acid or caffeyl-CoA, at least one molecule of coenzyme A, with at least one molecule of ATP, with at least one molecule of NAD(P)H, with at least of one molecule of oxygen, and at least two molecules of malonyl-CoA.

Also, this invention provides for application of nucleic acids coding enzymes of fungal luciferin biosynthesis, mutants and homologs of these proteins, including shortened and elongated forms, and fusion proteins to obtain enzymes involved in fungal luciferin biosynthesis in vitro and\or in vivo.

In preferred embodiments there is provided application of nucleic acids coding hispidin hydroxylases of the invention, namely proteins characterized by amino acid sequences shown in SEQ ID NOs: 2, 4, 6, 8 10, 12, 14, 16, 18, 20, 22, 24, 26, 28, and also their functional homologs, mutants and derivatives. In preferred embodiments nucleic acids code proteins, which amino acid sequences are at least 40%, prevalently at least 45%, normally at least 50%, e.g. at least 55%, at least 60%, at least 65%, at least 70%, at least 80%, at least 85%, at least 90%, or at least 91%, or at least 92%, or at least 93%, or at least 94%, or at least 95%, or at least 96%, or at least 97%, or at least 98%, or at least 99% identical to the sequences shown in SEQ ID NOs: 2, 4, 6, 8 10, 12, 14, 16, 18, 20, 22, 24, 26, 28, for at least 350 amino acids. In preferred embodiments nucleic acids code proteins, which amino acid sequences are characterized by presence of several conservative amino acid motifs (consensus sequences) shown in SEQ ID NO: 29-33.

There is also provided application of nucleic acids coding hispidin-synthases, namely proteins characterized by amino acid sequences shown in SEQ ID NOs: 35, 37, 39, 41, 43, 45, 47, 49, 51, 53, 55, and also their functional homologs, mutants and derivatives. In preferred embodiments nucleic acids of the invention code proteins, which amino acid sequences are at least 40%, prevalently at least 45%, normally at least 50%, e.g. at least 55%, or at least 60%, or at least 65%, or at least 70%, or at least 80%, or at least 85%, or at least 90%, or at least 91%, or at least 92%, or at least 93%, or at least 94%, or at least 95%, or at least 96%, or at least 97%, or at least 98%, or at least 99% identical to the sequences shown in SEQ ID NOs: 35, 37, 39, 41, 43, 45, 47, 49, 51, 53, 55, for all protein polypeptide chain. In preferred embodiments nucleic acids code proteins, which amino acid sequences are characterized by presence of several conservative amino acid motifs (consensus sequences) shown in SEQ ID NOs: 56-63.

There is also provided application of nucleic acids coding caffeylpyruvate hydrolases, namely proteins characterized by amino acid sequences shown in SEQ ID NOs: 65, 67, 69, 71, 73, 75, and also their functional homologs, mutants and derivatives. In preferred embodiments nucleic acids of the invention code proteins, which amino acid sequences are at least 40%, prevalently at least 45%, normally at least 50%, e.g. at least 55%, or at least 60%, or at least 65%, or at least 70%, or at least 80%, or at least 85%, or at least 90%, or at least 91%, or at least 92%, or at least 93%, or at least 94%, or at least 95%, or at least 96%, or at least 97%, or at least 98%, or at least 99% identical to the sequences shown in SEQ ID NOs: 65, 67, 69, 71, 73, 75, for all protein polypeptide chain. In preferred embodiments nucleic acids code proteins, which amino acid sequences are characterized by presence of several conservative amino acid motifs (consensus sequences) shown in SEQ ID NOs: 76-78.

The above groups of nucleic acids are applied for producing recombinant proteins of hispidin hydroxylases, hispidin synthases and caffeylpyruvate hydrolases, and also for expression of these proteins in heterologous expression systems.

In particular, nucleic acids coding hispidin hydroxylases are applied for obtaining producer cells of 6-(2-arylvinyl)-3,4-dihydroxy-2H-pyran-2-one, having the structural formula

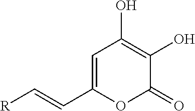

from exogenous or endogenous 6-(2-arylvinyl)-4-hydroxy-2H-pyran-2-one, having the structural formula

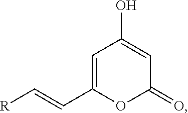

where R-aryl or heteroaryl.

Nucleic acids coding caffeylpyruvate hydrolases are applied for obtaining cells and organisms able to transform oxyluciferin into preluciferin precursor.

Nucleic acids coding hispidin-synthases are applied for obtaining producer cells of the above 6-(2-arylvinyl)-4-hydroxy-2H-pyran-2-one from the corresponding 3-arylacrylic acid. For example, cells expressing hispidin-synthase are applied for producing hispidin from caffeic acid.

In some embodiment's nucleic acids coding hispidin-synthases are applied for producing hispidin from tyrosine. In the said embodiments, nucleic acids, coding enzymes promoting synthesis of caffeic acid from tyrosine, are additionally introduced into the cells. Such enzymes are known in the art. For example, there could be used a combination of nucleic acids coding tyrosine-ammonia-lyase Rhodobacter capsulatus, and the components HpaB and HpaC of E. coli 4-hydroxyphenyl acetate 3-monooxygenase-reductase as described in [Lin and Yan. Microb Cell Fact. 2012, 4; 11:42]. It is obvious to those skilled in the art that alternatively there could be used any other known in the art enzymes transforming tyrosine into caffeic acid, for example, enzymes, which amino acid sequences are substantially identical to the amino acid sequences of tyrosine-ammonia-lyase Rhodobacter capsulatus, and the components HpaB and HpaC of E. coli 4-hydroxyphenyl acetate 3-monooxygenase-reductase, shown in SEQ ID NOs: 107, 109 and 111. For example, the said enzymes could have amino acid sequences which have minimum 40% of identity, e.g. minimum 45% of identity, or minimum 50% of identity, or minimum 55% of identity, or minimum 60% of identity, or minimum 65% of identity, or minimum 70% of identity, or minimum 75% of identity, e.g. or minimum 80% of identity, or minimum 85% of identity, or minimum 90% of identity (e.g. at least 90%, 91%, 92%, 93%, 94%, 95%, 96%, 97%, 98%, 98% or 99% of identity) with amino acid sequence shown in SEQ ID NO: 107, 109 and 111 respectively.

In some embodiments, nucleic acids coding hispidin-synthases are applied for obtaining producer cells of hispidin functional analogues from aromatic compounds, including aromatic amino acids and their derivatives. In the said embodiments, nucleic acids, coding enzymes promoting synthesis of 3-arylacrylic acids, from which hispidin functional analogues are biosynthesized, are additionally introduced into the cells. Such enzymes are known in the art. For example, for cinnamic acid biosynthesis there could be used nucleic acid coding phenylalanine-ammonia-lyase Streptomyces maritimus, as described in [Bang, H. B., Lee, Y. H., Kim, S. C. et al. Microb Cell Fact (2016) 15: 16. https://doi.org/10.1186/s12934-016-0415-9]. It is obvious to those skilled in the art that alternatively there could be used any other known in the art enzymes transforming aromatic amino acids and other aromatic compounds into 3-aryl acrylic acids. For example, for cinnamic acid biosynthesis there could be any functional phenylalanine-ammonia-lyase, e.g. phenylalanine-ammonia-lyase, which amino acid sequence is substantially similar to the sequence shown in SEQ ID NOs: 117, for example, which sequence is identical to the sequence of SEQ ID NO: 117 at least 40%, including minimum 45% of identity, or minimum 50% of identity, or minimum 55% of identity, or minimum 60% of identity, or minimum 65% of identity, or minimum 70% of identity, or minimum 75% of identity, e.g. minimum 80% of identity, minimum 85% of identity, minimum 90% of identity (e.g. at least 90%, 91%, 92%, 93%, 94%, 95%, 96%, 97%, 98%, 98% or 99% of identity).

In some embodiments for obtaining host cells expressing functional hispidin-synthase it is required to co-transfect them by nucleic acid coding hispidin-synthases of the invention and by nucleic acid coding 4′-phosphopantetheinyl transferase able to transfer 4′-phosphopantetheinyl from coenzyme A to serine in acyl carrier domain of polyketide synthases. In other embodiments the selected host cells, for example, plant cells or cells of some lower fungi (e.g. Aspergillus), comprise endogenous 4′-phosphopantetheinyl transferase and co-transfection is not required.

Application of nucleic acid combinations of the invention is also provided. Thus, a combination of the nucleic acids, coding hispidin hydroxylase and hispidin synthase, is applied for obtaining producer cells of 6-(2-arylvinyl)-3,4-dihydroxy-2H-pyran-2-one from 3-arylacrylic acid, for example, for producing 3-hydroxyhispidin from caffeic acid and/or tyrosine. In other embodiments a combination of the nucleic acids includes a nucleic acid coding 4′-phosphopantetheinyl transferase. In some embodiments a combination of the nucleic acids includes the nucleic acids coding enzymes promoting 3-arylacrylic acid synthesis from the cell metabolites, e.g. enzymes promoting caffeic acid synthesis from tyrosine or cinnamic acid synthesis from phenylalanine.

In some embodiments a combination of the nucleic acids, coding PKS and coumarate-CoA ligase, is used for obtaining hispidin producer cells from caffeic acid. For the purposes of this invention it is applicable a nucleic acid coding functional PKS, which amino acid sequence is substantially similar or identical to the sequence selected from the group SEQ ID NOs: 119, 121, 123, 125, 127, 129, 131, 133, 135, 137, 139; e.g. PKS, which amino acid sequence is identical to the sequence selected from the group SEQ ID NOs: 119, 121, 123, 125, 127, 129, 131, 133, 135, 137, 139 at least 40%, prevalently at least 45%, normally at least 50%, e.g. at least 55%, or at least 60%, or at least 65%, or at least 70%, or at least 80%, or at least 85%, or at least 90%, or at least 91%, or at least 92%, or at least 93%, or at least 94%, or at least 95%, or at least 96%, or at least 97%, or at least 98%, or at least 99% identical. The nucleic acid coding functional coumarate-CoA ligase, catalyzing reaction of coenzyme A addition to caffeic acid with caffeyl-CoA formation, is also applicable for the purposes of this invention. For example, there could be used a nucleic acid coding functional coumarate-CoA ligase, which amino acid sequence is identical to the sequence shown SEQ ID NO: 141, or has minimum 40% of identity, e.g. minimum 45% of identity, or minimum 50% of identity, or minimum 55% of identity, or minimum 60% of identity, or minimum 65% of identity, or minimum 70% of identity, or minimum 75% of identity, e.g. minimum 80% of identity, minimum 85% of identity, minimum 90% of identity (e.g. at least 90%, 91%, 92%, 93%, 94%, 95%, 96%, 97%, 98%, 98% or 99% of identity).

In some embodiments a combination of the nucleic acid coding hispidin hydroxylases of the invention and the nucleic acid coding PKS is used. In preferred embodiments the combination also includes a nucleic acid coding coumarate-CoA ligase. The combination is applied for obtaining 3-hydroxyhispidin producer cells from caffeic acid and/or caffeyl-CoA.

In some embodiments a combination of nucleic acids includes nucleic acids coding enzymes promoting synthesis of caffeic acid from tyrosine.

Combinations of the nucleic acids of the inventions used together with the nucleic acid coding luciferase, able to oxidize fungal luciferin with luminescence emission, are of special interest. Nucleic acid molecules, coding luciferases for the purposes of this invention, could be cloned from biological sources, for example, from fungi of Basidiomycota type, predominantly of Basidiomycetes class, in particular, Agaricales order, or obtained by techniques of genetic modification. Luciferase mutants having luciferase activity could be obtained using standard techniques of molecular biology, such as described above in details in “Nucleic Acids” section. Mutations include changes of one or more amino acids, deletion, or insertion of one or more amino acids, replacements or truncations, or N-terminal truncations or extensions, C-terminal truncations or extensions, etc. In preferred embodiments these nucleic acids code luciferases, which amino acid sequences are at least 40% identical, e.g. at least 45% identical, or at least 50% identical, or at least 55% identical, or at least 60% identical, or at least 70% identical, or at least 75% identical, or at least 80% identical, or at least 85% identical to the amino acid sequence selected from the group of SEQ ID NOs: 80, 82, 84, 86, 88, 90, 92, 94, 96, 98. For example, they could have amino acid sequences which have minimum 90% of identity (e.g. minimum 91%, minimum 92%, minimum 93%, minimum 94%, minimum 95%, minimum 96%, minimum 97%, minimum 98%, minimum 99% or 100% of identity) with amino acid sequence selected from the group SEQ ID NOs: 80, 82, 84, 86, 88, 90, 92, 94, 96, 98. Non-limiting examples of nucleic acids, coding luciferases, are given in SEQ ID NOs: 79, 81, 83, 85, 87, 89, 91, 93 and 95.

In some embodiments a combination of the nucleic acid coding hispidin hydroxylase of the invention and the nucleic acid coding the above luciferase is used. The combination is widely applicable when labeling organisms, tissues, cells, cell organelles or proteins by bioluminescence. Methods for labeling organisms, tissues, cells, cell organelles or proteins by luciferase are well known in the art and presuppose introduction of a nucleic acid, coding luciferase, into a host cell, for example being a part of an expression cassette promoting luciferase expression in the said cell, tissue or organism. When adding the suitable luciferin to cells, tissue or organism, expressing a luciferase, detectable luminescence occurs. When labeling cell organelles or proteins, the nucleic acid, coding luciferase, is operatively bound with the nucleic acid coding respectively the localization signal in the test cell organelle or test protein. At co-expression in the cells of luciferase and hispidin-synthase of this invention the biological objects (cells, tissues, organisms, cell organelles or proteins) acquire the ability to emit luminescence in the presence of not only fungal luciferin, but of preluciferin as well (the latter in most cases is more stable in the presence of ambient oxygen).

Also, the combination of the nucleic acids is applicable in study of two promoters activity dependency in heterologous expression systems. In this case a nucleic acid operatively bound with promoter A, coding luciferase, and a nucleic acid operatively bound with promoter B, coding hispidin hydroxylase, are introduced into a host cell. Adding luciferin or preluciferin, or preluciferin and luciferase mixture to cell (or cell extracts) aliquots, it is possible to detect by occurrence of luminescence emission the activity of one promoter A (luminescence emission is detected in the presence of luciferin only), of one promoter B (luminescence emission is detected in the presence of preluciferin and luciferase mixture) or of both promoters (luminescence emission is detected in all cases).

In some embodiments the combination also comprises a nucleic acid coding hispidin-synthase. In some embodiments the combination additionally comprises a nucleic acid coding 4′-phosphopantetheinyl transferase.

In some embodiments the combination comprises a nucleic acid coding hispidin-synthase, a nucleic acid coding luciferase, a nucleic acid coding PKS, a nucleic acid coding coumarate-CoA ligase.

The combinations are widely applicable when labeling organisms, tissues, cells, cell organelles or proteins by bioluminescence. In this embodiment in order to obtain luminescence emission, a suitable preluciferin precursor, e.g. caffeic acid or coumaric acid, is added to biological objects expressing hispidin hydroxylase, luciferase and hispidin synthase or hispidin hydroxylase, luciferase, PKS and coumarate-CoA ligase.

The combinations are also applicable in methods of study of three promoters activity dependency in heterologous expression systems. The methods presuppose introduction of nucleic acid coding luciferase under promoter A control, of nucleic acids, coding hispidin hydroxylase under promoter B control and nucleic acid coding hispidin synthase (or PKS), under promoter B control, into the host cell. If co-expression of 4′-phosphopantetheinyl transferase is required for maturation of functional hispidin-synthase, it is also introduced into the cell under control of any suitable constitutive or inducible promoter. When adding a suitable preluciferin precursor to the cells (or their extracts) the detectable luminescence appears, it indicates a simultaneous activation of all three promoters.

The combinations are also applicable at producing transgenic luminous organisms. In preferred embodiments the transgenic organisms are obtained from the organisms, which wild type is not capable of bioluminescence. Nucleic acids, coding target proteins, are introduced into a transgenic organism as a part of expression cassette or vector, which exist in the organism as extrachromosomal elements, or are integrated into the organism genome, as described above in “Transgenic Organisms” section, and promote expression of target proteins. Transgenic organisms of the invention are different in that they express at least hispidin hydroxylase, except for luciferase, which substrate is fungal luciferin. In preferred embodiments they also express hispidin-synthase. In other preferred embodiments they also express PKS. In other preferred embodiments they also express PKS. In some embodiments they also express coumarate-CoA ligase. It is known that endogenous coumarate-CoA ligase is present in many plant organisms, therefore, its additional introduction is carried out in cases, when endogenous coumarate-CoA ligase is absent.

In some embodiments they also express caffeylpyruvate hydrolase. In contrast to the organisms expressing only luciferase, the transgenic organisms, obtained by using nucleic acids of the invention, acquire the ability to emit luminescence in the presence of preluciferins and/or preluciferin precursors—3-arylacrylic acids (prevalently, caffeic acid)—which are the cheapest and the most stable substrate for obtaining bioluminescence, which could be added to water for plant watering, or to microorganism culture medium, or to feed or to animal (e.g. fish) habitat. Bioluminescent transgenic organisms (plants, or animals, or fungi) are applicable as luminescence sources and also they are used for ornamental purposes. Bioluminescent transgenic organisms, cells and cell cultures could also be used in different screenings, where bioluminescence intensity is changed depending on external influence. For example, they could be used at analysis of different factors effect on activity of promoters controlling expression of exogenous nucleic acids.

Autonomously bioluminescent transgenic organisms, which are also provided by this invention, are of special interest.

In some embodiments the said organisms have at least one 3-arylacrylic acid, as a metabolite, with the structural formula

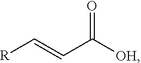

where R is aryl or heteroaryl.

higher and lower plants, including flowering plants and mosses could be mentioned as non-limiting examples. In order to obtain autonomously luminescence-producing transgenic plants, the nucleic acids, coding hispidin hydroxylase, hispidin synthase and luciferase able to oxidize fungal luciferin with luminescence emission, and able to express the corresponding enzymes, are introduced into these plants. Since plants normally comprise endogenous 4′-phosphopantetheinyl transferase, additional introduction of nucleic acid, coding this enzyme to obtain autonomously luminescence-producing plants, is generally not required.

In some embodiments the organisms, which do not naturally produce 3-arylacrylic acids, are used to obtain autonomously bioluminescent transgenic organisms. The examples of such organisms are animals and a variety of microorganisms, e.g. yeasts and bacteria. In this case, nucleic acids, capable of expression, coding enzymes promoting 3-arylacrylic acid biosynthesis from the cell metabolites, for example, caffeic acid from tyrosine, are additionally introduced into organisms to obtain autonomous bioluminescence. If necessary, nucleic acid coding 4′-phosphopantetheinyl transferase is also introduced into organisms.

In some embodiments to obtain autonomously luminescence-producing organisms, the nucleic acids, able to express corresponding enzymes, coding PKS, hispidin hydroxylase and luciferase able to oxidize fungal luciferin with luminescence emission, are introduced into these organisms. In preferred embodiments the said cells, tissues and organisms comprise sufficient amount of caffeyl-CoA and malonyl-CoA to carry out hispidin synthesis.

In cases, when transgenic organism does not produce sufficient amount of caffeyl-CoA during normal metabolic processes, the nucleic acid coding coumarate-CoA ligase, and also, if necessary, enzymes of caffeic acid biosynthesis from tyrosine, is also introduced into the said cells or organisms.

In preferred embodiments the combination of nucleic acids for obtaining autonomously bioluminescent cells or transgenic organisms also comprises a nucleic acid coding caffeylpyruvate hydrolase. As demonstrated in the experimental part below, caffeylpyruvate hydrolase expression results in increasing bioluminescence intensity of autonomously bioluminescent cells or transgenic organisms. In preferred embodiments bioluminescence intensity increases at least 1.5 times, prevalently at least 2 times, normally at least 5 times, e.g. 7-9 times, e.g. 8 or more times.

Autonomously bioluminescent transgenic organisms (plants, or animals, or fungi) and also cells and cell structures are different from transgenic organisms, cells and cell cultures expressing luciferase only and known in the art, in that no exogenous adding of luciferin or its precursor is required for their luminescence.

In some embodiments instead of combination of nucleic acids coding hispidin hydroxylase and luciferase the nucleic acid coding fusion protein of these two enzymes is used. It is obvious to those skilled in the art that the said fusion protein and the combination of nucleic acids coding hispidin hydroxylase and luciferase are interchangeable objects in all methods of use. It is also obvious that on the basis of the nucleic acids of the invention there could be produced other fusion proteins, which will retain properties of fusion partners; such fusion proteins and nucleic acids coding them could be used without limitation instead of combinations of individual proteins and nucleic acids.

In all applications and methods described above the nucleic acids could be in the form of expression cassettes, which could be used to promote the coding sequence expression in a host cell. Nucleic acid could be introduced into a host cell as a part of the vector for expression in suitable host cell or not including it into the vector, for example, it could be integrated into a liposome or viral particle. Alternatively, the purified molecule of nucleic acid could be integrated directly into the host cell using suitable means, e.g. by direct endocytic uptake. Gene construct could be introduced directly into the host organism cells (e.g. plant) by transfection, infection, microinjection, cell fusion, protoplast fusion, using microparticle bombardment or by means of “gene gun” (gun for shooting with microparticles carrying gene constructs).

Application of polyclonal and monoclonal antibodies of the invention is also provided. They are applied in staining tissues, cells, or organisms to localize expressed or natural hispidin hydroxylases, hispidin synthases and caffeylpyruvate hydrolases of the invention. Methods for staining by means of specific antibodies are well known in the art and described, for example, in [V. L. Bykov Cytology and general histology]. Direct immunohistochemical technique is based on the reaction of specific binding labeled antibodies directly with detectable substance, indirect immunohistochemical technique is based on that unlabeled primary antibodies are bound with detectable substance and then they are detected by means of secondary labeled antibodies, provided that, the primary antibodies are antigens for secondary antibodies. Antibodies are also applicable for stopping enzymatic reaction. Contact of antibody with specific binding partner results in inhibiting the enzymatic reaction. Antibodies are also applicable in methods for purification of recombinant and natural proteins of the invention by affinity chromatography. Affinity chromatography techniques are known in the art and described, for example, in Ninfa et al (2009). Fundamental Laboratory Approaches for Biochemistry and Biotechnology (2 ed.). Wiley. p. 133.; Cuatrecasas (1970). JBC. Retrieved Nov. 22, 2017].

### Sets and Products

The next embodiment of the invention is a product, which includes the above described hispidin hydroxylase, or hispidin synthases, or caffeylpyruvate hydrolases, or nucleic acid coding the above enzyme, preferably with the elements for promoting target protein expression in host cell, e.g. expression vector or cassette, comprising nucleic acid coding the target protein. Alternatively, nucleic acids could comprise flanking sequences for its incorporation into the target vector. Nucleic acids could be included in promoter-free vectors intended for easy cloning of target regulatory elements. Recombinant proteins could be lyophilized or dissolved in a buffer solution. Nucleic acids could be lyophilized or precipitated in an alcoholic solution or dissolved in water or buffer solution.

In some embodiment the product includes cells expressing one or several above nucleic acids.

In some embodiment the product includes a transgenic organism expressing one or several above nucleic acids.

In some embodiment the product includes antibodies for staining and/or inhibition and/or affinity chromatography of the above enzymes.

The product is a container with a label and instructions for use attached thereto. The acceptable containers are, for example, bottles, ampoules, glass tubes, syringes, cell plates, Petri dishes, etc. The container could be made of different materials, such as glass or polymer materials. Selection of suitable container is obvious to those skilled in thee art.

Besides, the product could include other products required commercially or from a consumer point of view, e.g.: reaction buffer or components for its preparation, buffer for dilution and/or solution and/or storage of proteins and nucleic acids, or components for its preparation, deionized water, secondary antibodies to specific antibodies of the invention, cell culture medium or components for its preparation, nutrition for transgenic organism.

The products also include instructions for implementation of the proposed methods. The instructions could be in different forms, provided that, one or several such forms could be attached to the product, e.g. the instruction could be a file in electronic format and/or on paper.

The invention also relates to the kits which could be applied for different purposes. The kit could include a combination of proteins of the invention or combination of nucleic acids of the invention, preferably with the elements for promoting target protein expression in host cell, e.g. expression vector or cassette, comprising nucleic acid coding the target protein. In some embodiments the kit could also comprise a nucleic acid coding luciferase, able to oxidize fungal luciferin with luminescence emission. In some embodiments the kit could also comprise nucleic acids coding enzymes involved in biosynthesis of caffeic acid from tyrosine. In some embodiments the kit could also comprise a nucleic acid coding 4′-phosphopantetheinyl transferase. In some embodiments the kit could also comprise a nucleic acid coding PKS. In some embodiments the kit could also comprise a nucleic acid coding coumarate-CoA ligase.

In some embodiments the kit could also comprise antibodies for purification of recombinant proteins or for staining the expressed proteins in host cells. In some embodiments the kit could also comprise primers, complementary to regions of the said nucleic acid, for amplification of nucleic acid or its fragment. In some embodiments the kit could also comprise one or several fungal luciferins and/or preluciferins and/or preluciferin precursors. The said compounds could be in the form of dry powder, in the form of organic solvent solution, in the form of water solution. In some embodiment the kit could include cells comprising one or several above nucleic acids. In some embodiments the kit could comprise a transgenic organism of the invention, e.g. producer strain or transgenic autonomously bioluminescent plant. All the kit components are placed into suitable containers. Generally, the kits also include instructions for use.

The following examples are given for better understanding the invention. These examples are given for illustration purposes only and shall not be interpreted as limiting the scope of invention in any way.

All publications, patents and patent applications mentioned in this specification are incorporated herein by reference. Though the above invention has been described in considerable details by illustration and example for purposes of clarity, it is obvious to those skilled in the art, based on the ideas disclosed in this invention, that some alterations and modifications could be introduced without departing from the spirit and scope of the proposed embodiments of the invention.

### EXPERIMENTAL PART (EXAMPLES)

### Example 1. Isolation of Hispidin Hydroxylase Sequences

Total RNA from Neonothopanus nambi mycelium was isolated according to the method described in [Chomczynski and Sacchi, Anal. Biochem., 1987, 162, 156-159]. cDNA was amplified by means of SMART PCR cDNA Synthesis Kit (Clontech, USA) according to the manufacturer's protocol. The obtained cDNA was used for amplification of coding sequence of luciferase, which nucleotide and amino acid sequence are shown in SEQ ID NOs: 79, 80. Coding sequence was cloned into pGAPZ vector (Invitrogen, USA) according to the manufacturer's protocol and transformed into E. coli competent cells of XL1 Blue strain. Bactria were cultivated on Petri dishes in the presence of antibiotic Zeocin. In 16 hours the colonies were rinsed from the dishes, intensively mixed, and plasmid DNA was isolated from them by means of plasmid DNA isolation kit (Evrogen, Russia). The isolated plasmid DNA was linearized at restriction site AvrII and used for transformation of Pichia pastoris GS115 cells. Electroporation was carried out according to the method, using lithium acetate and dithiothreitol, described in [Wu and Letchworth, Biotechniques, 2004, 36:152-4]. Electroporated cells were dispersed in Petri dishes with RDB medium, comprising 1 M of sorbitol, 2% (weight/volume) of glucose, 1.34% (weight/volume) of yeast nitrogen base (YNB), 0.005% (weight/volume) of amino acids mixture, 0.00004% (weight/volume) of biotin and 2% (weight/volume) of agar. The obtained colonies were sprayed with 3-hydroxyhispidin solution, detecting luciferase presence in cells by occurrence of luminescence. Luminescence emitted by colonies was detected by means of IVIS Spectrum CT (PerkinElmer, USA). Colonies, where luminescence was detected in response to addition of 3-hydroxyhispidin, were selected for further work.

Then, the amplified total cDNA from Neonothopanus nambi was cloned into pGAPZ vector and transformed into E. coli competent cells of XL1 Blue strain. Bactria were cultivated on Petri dishes in the presence of antibiotic Zeocin. In 16 hours, the colonies were rinsed from the dishes, intensively mixed, and plasmid DNA was isolated from them by means of plasmid DNA isolation kit (Evrogen, Russia). The isolated plasmid DNA was linearized at restriction site Avril and used for transformation of Pichia pastoris GS115 yeast cells, constitutively expressing Neonothopanus nambi luciferase. Transformation was carried out by electrocorporation technique, as described above. The cells were dispersed in Petri dishes with RDB medium, comprising 1 M of sorbitol, 2% (weight/volume) of glucose, 1.34% (weight/volume) of yeast nitrogen base (YNB), 0.005% (weight/volume) of amino acids mixture, 0.00004% (weight/volume) of biotin and 2% (weight/volume) of agar. Diversity in resulting library of Neonothopanus nambi cDNA in yeasts was about one million of clones. The obtained colonies were sprayed with hispidin solution, detecting hispidin hydroxylase presence in cells by occurrence of luminescence. Luminescence emitted by colonies was detected by means of IVIS Spectrum CT (PerkinElmer, USA). Cells expressing luciferase only and wild yeast cells were used as negative control. When screening the library, the colonies, where luminescence was detected, were selected and used for PCR as a matrix with standard plasmid primers. PCR products were sequenced by Sanger method to determine sequence of the expressed gene. The obtained sequence of hispidin hydroxylase nucleic acid is shown in SEQ ID NO: 1. The amino acid sequence coded by it is shown in SEQ ID NO: 2.

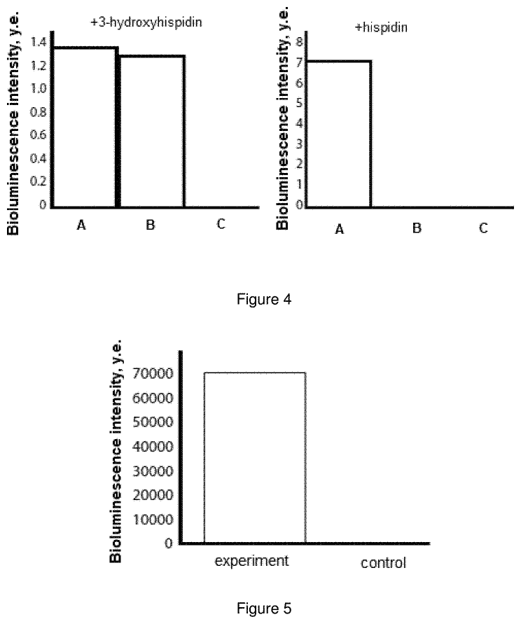

FIG. 4 illustrates luminescence of Pichia pastoris cells expressing hispidin hydroxylase and luciferase or luciferase only, or wild yeasts when spraying the colonies with 3-hydroxyhispidin (luciferin) and hispidin (preluciferin). The data demonstrate that luciferin is produced in cells in the presence of hispidin hydroxylase.

At the next step genomic DNA was isolated from the fungi Armillaria fuscipes, Armillaria gallica, Armillaria ostoyae, Armillaria mellea, Guyanagaster necrorhiza, Mycena citricolor, Mycena chlorophos, Neonothopanus nambi, Neonothopanus gardneri, Omphalotus olearius and Panellus stipticus, and whole-genome sequencing was performed by Illumina HiSeq technique (Illumina, USA) according to the manufacturer's recommendations. Sequencing results were used for prediction of hypothetical protein amino acid sequences and to search for hispidin hydroxylase homologs from Neonothopanus nambi. Homologs search was carried out by means of a software provided by National Center for Biotechnology Information. Search for amino acid sequences in the data of fungal genome sequencing in NCBI Genbank database. Standard search parameters blastp were used at search. As result, there were identified the sequences of hispidin hydroxylase homologs from Neonothopanus nambi—in Armillaria fuscipes, Armillaria mellea, Guyanagaster necrorhiza, Mycena citricolor, Neonothopanus gardneri, Omphalotus olearius, Panellus stipticus, Armillaria gallica, Armillaria ostoyae, Mycena chlorophos.

Nucleotide and amino acid sequences of hispidin-synthase homologs of Neonothopanus nambi are shown in SEQ ID NOs: 3-28.

All identified enzymes are substantially identical to each other. Degree of amino acid sequences identity is shown in Table 4.

TABLE 4
Percent identity of hispidin hydroxylase full-length natural protein amino acid sequences.

| | SEQ | SEQ | SEQ | SEQ | SEQ | SEQ | SEQ | SEQ | SEQ | SEQ | SEQ |
|---|---|---|---|---|---|---|---|---|---|---|---|
| | ID | ID | ID | ID | ID | ID | ID | ID | ID | ID | ID |
| | NO: 2 | NO: 4 | NO: 6 | NO: 16 | NO: 8 | NO: 14 | NO: 20 | NO: 22 | NO: 24 | NO: 26 | NO: 28 |
| SEQ ID | 100 | 61 | 71 | 72 | 70 | 89 | 72 | 73 | 73 | 73 | 69 |
| NO: 2 | | | | | | | | | | | |
| SEQ ID | 61 | 100 | 50 | 48 | 48 | 63 | 49 | 51 | 50 | 50 | 45 |
| NO: 4 | | | | | | | | | | | |
| SEQ ID | 71 | 50 | 100 | 69 | 69 | 71 | 85 | 86 | 87 | 88 | 67 |
| NO: 6 | | | | | | | | | | | |
| SEQ ID | 72 | 48 | 69 | 100 | 76 | 72 | 71 | 72 | 72 | 72 | 71 |
| NO: 6 | | | | | | | | | | | |
| SEQ ID | 72 | 48 | 70 | 99 | 76 | 72 | 71 | 73 | 73 | 73 | 71 |
| NO: 18 | | | | | | | | | | | |
| SEQ ID | 70 | 48 | 69 | 76 | 100 | 71 | 70 | 70 | 71 | 71 | 73 |
| NO: 8 | | | | | | | | | | | |
| SEQ ID | 70 | 48 | 69 | 76 | 99 | 71 | 70 | 70 | 71 | 71 | 73 |
| NO: 10 | | | | | | | | | | | |
| SEQ ID | 89 | 63 | 71 | 72 | 71 | 100 | 72 | 75 | 73 | 74 | 71 |
| NO: 14 | | | | | | | | | | | |
| SEQ ID | 72 | 49 | 85 | 71 | 70 | 72 | 100 | 91 | 92 | 93 | 68 |
| NO: 20 | | | | | | | | | | | |
| SEQ ID | 73 | 51 | 86 | 72 | 70 | 75 | 91 | 100 | 93 | 93 | 69 |
| NO: 22 | | | | | | | | | | | |
| SEQ ID | 73 | 50 | 87 | 72 | 71 | 73 | 92 | 93 | 100 | 95 | 69 |
| NO: 24 | | | | | | | | | | | |
| SEQ ID | 73 | 50 | 88 | 72 | 71 | 74 | 93 | 93 | 95 | 100 | 69 |
| NO: 26 | | | | | | | | | | | |
| SEQ ID | 69 | 45 | 67 | 71 | 73 | 71 | 68 | 69 | 69 | 69 | 100 |
| NO: 28 | | | | | | | | | | | |

From Panellus stipticus and Mycena citricolor there were isolated several highly homologous hispidin hydroxylase amino acid sequences characterized by single amino acid substitutions. Their nucleotide and amino acid sequences are shown in SEQ ID NOs 7-13 (Panellus stipticus) and SEQ ID NOs 15-18 (Mycena citricolor). Further study of the said proteins' properties had not detect influence of these substitutions on enzymatic properties.

Coding sequences of the detected homologs (SEQ ID NOs: 3-28) were cloned and transformed into Pichia pastoris GS115 cells, constitutively expressing Neonothopanus nambi luciferase according to the above protocol. The obtained colonies were sprayed with hispidin solution, detecting hispidin hydroxylase presence in cells by occurrence of luminescence. Luminescence emitted by colonies was detected by means of IVIS Spectrum CT (PerkinElmer, USA). All colonies, expressing the test genes (SEQ ID NOs: 3, 5, 7, 9, 11, 13, 15, 17, 19, 21, 23, 25, 27), produced luminescence 1000-100000000 times more at spraying with hispidin solution, than control cells, that confirms a capability of enzymes coded by tested genes to catalyze hispidin transformation into 3-hydroxyhispidin (fungal luciferin).

Structural analysis of detected enzymes amino acid sequences was done, Analysis performed by means of software SMART (Simple Modular Architecture Research Tool), available on the Internet at the website http://smart.embl-heidelberg.de [Schultz et al., PNAS 1998; 95: 5857-5864; Letunic, Doerks T, Bork P Nucleic Acids Res 2014; doi:10.1093/nar/gku949] have revealed that all detected proteins comprise FAD/NAD(P)-binding domain, IPR002938—code of InterPro public database available on the Internet at the website http://www.ebi.ac.uk/interpro). This domain is involved in binding FAD and NAD in some enzymes, in particular, monooxygenases—the representatives of a large enzyme family, adding the hydroxyl group to substrate, and multiple organisms found in metabolic pathways. The detected hispidin hydroxylases, except for FAD/NAD(P)-binding domain, comprise N- and C-terminal amino acid sequences operatively bound with it (FIG. 1). Using multiple alignment and comparison of amino acid sequences of the detected hispidin hydroxylases (FIG. 1) it was revealed that they comprise several conservative amino acid motifs (consensus sequences) typical of this enzyme group only (SEQ ID NOs: 29-33). Consensus sites inside amino acid sequences are operatively bound via amino acid inserts.

### Example 2. Expression of Hispidin Hydroxylase and Fungal Luciferase in Mammal Cells and their Combined Use for Cell Labeling

Coding sequences of hispidin hydroxylase and luciferase from Neonothopanus nambi, obtained according to Example 1, were optimized (humanized) for expression in mammal cells. Optimized nucleic acids (SEQ ID NOs: 99 and 100) were obtained synthetically. Coding sequence of hispidin hydroxylase was cloned into pmKate2-keratin vector (Evrogen, Russia), using restriction sites NheI and NotI instead of the sequence coding fusion protein mKate2-keratin. Luciferase sequence was amplified by PCR, treated by restriction endonucleases NheI and EcoRV (New England Biolabs, Ipswich, Mass.) and ligated into lentiviral vector pRRLSIN.cPPT.EF1. Plasmid DNA was purified by means of plasmid DNA purification kits (Evrogen). Plasmid DNA, comprising luciferase gene, was used for development of stably expressing lines HEK293NT. Vector particles were obtained by calcium-phosphate transfection (Invitrogen, Carlsbad, Calif.) of HELK293T cells according to the protocol provided at the manufacturer website. 1,500,000 cells were put in 60 mm cultural dish 24 hours before transfection. About 4 and 1.2 μg of packaging plasmids pR8.91 and pMD.G, and also 5 μg of transfer plasmid, comprising luciferase sequence, were used for transfection. Viral particles were harvested 24 hours after transfection, 10 times concentrated and used for transduction of HEK293NT cells. About 100% of HEK293NT cells stably expressed Neonothopanus nambi luciferase.

The obtained cells were subjected to re-transfection by the vector comprising coding sequence of hispidin hydroxylase using transfection reagent FuGENE HD (Promega, USA) according to the manufacturer's protocol. 24 hours after transfection hispidin at concentration of 800 μg/ml was added to the medium and cell luminescence was detected by means of IVIS Spectrum CT (PerkinElmer). The obtained cells emitted luminescence with intensity more than by two orders of magnitude exceeding the signal outgoing from untransfected control cells (FIG. 5).

The cells were visualized in transmitted light in green luminescence detection channel. Expression of Neonothopanus nambi hispidin hydroxylase in human cells resulted in occurrence of distinct luminous signal in green spectrum in the presence of hispidin enabling to distinguish transfected cells from untransfected ones.

### Example 3. Use of Hispidin Hydroxylase with Hispidin Analogues in Cell Lysate

HEK293NT cells, expressing luciferase and hispidin hydroxylase of Neonothopanus nambi, obtained according to Example 2, were rinsed from Petri dishes 24 hours after transfection with Versene solution laced with 0.025% of trypsin, the medium was replaced by phosphate-buffered saline with pH 8.0 by centrifugation, the cells were resuspended, lysed by ultrasound in Bioruptor (Diagenode, Belgium) within 7 minutes at 0° C. in conditions recommended by the manufacturer, and 1 mM of NADPH (Sigma-Aldrich, USA), and also hispidin or one of its analogues were added to the medium: (E)-4-hydroxy-6-(4-hydroxystyryl)-2H-pyran-2-one, (E)-6-(2-(1H-indol-3-yl)vinyl)-4-hydroxy-2H-pyran-2-one, (E)-6-(2-(1,2,3,5,6,7-hexahydropyrido[3,2,1-ij]quinolin-9-yl)vinyl)-4-hydroxy-2H-pyran-2-one, E)-6-(4-(diethylamino)styryl)-4-hydroxy-2H-pyran-2-one, or (E)-4-hydroxy-6-(2-(6-hydroxynaphthalene-2-yl)vinyl)-2H-pyran-2-one at concentration of 660 μg/ml. Bioluminescence spectra were detected by spectrofluorometer Varian Cary Eclipse. Luminescence in lysates was observed at addition of all said hispidin functional analogues. Depending on luciferin used the expected displacement of luminescence peak was observed.

### Example 4. Obtaining Recombinant Hispidin Hydroxylases

Polyhistidine sequence (His tag) was operatively attached to 5′-end of nucleic acids coding hispidin-3-hydroxylases and luciferase from Neonothopanus nambi, obtained according to the examples 1 and 2. The obtained structures were cloned into pET-23 vector by means of restriction endonucleases BamHI and HindIII. The vector was used for transformation of Escherichia coli cells of BL21-DE3 strain. The cells were dispersed in Petri dishes with LB medium, comprising 1.5% of agar, 100 μg/ml of ampicillin, and incubated overnight at 37° C. Then, Escherichia coli colonies were transferred into 4 ml of liquid LB medium laced with ampicillin, incubated overnight at 37° C. with fluctuation. 1 ml of overnight culture was transferred into 100 ml of Overnight Express Autoinduction medium (Novagen), where ampicillin was preliminarily added to. The culture was grown at 37° C. within 2.5 hours until reaching optical density of 0.6 OE at 600 nm, and then it was grown at room temperature within 16 hours. Then, the cells were pelleted at 4500 rpm within 20 minutes in centrifuge Eppendorf 5810R, resuspended in 35 ml of the buffer (50 mM of Tris HCl pH 8.0, 150 mM of NaCl). The cells were sonicated and pelleted again. TALON resin metal affinity chromatography (Clontech, USA) was used for purification of recombinant proteins. Presence of the expected recombinant product was confirmed by electrophoresis.

Aliquots of isolated recombinant hispidin hydroxylases were used for testing functionality and stability. For determination of functionality 15 μl of isolated recombinant protein solution were put into a glass tube, comprising 100 μl of the buffer (0.2 M of Na-phosphate buffer, 0.5 M of Na₂SO₄, 0.1% dodecylmaltoside (DDM) pH 8.0), 0.5 μl of purified recombinant luciferase of Neonothopanus nambi, 1 mM of NADPH and 0.2 μM of hispidin. The glass tube was placed into a luminometer. Activity of isolated recombinant proteins resulted in luminescence at combination with hispidin and its analogues described in Example 3, in the presence of Neonothopanus nambi luciferase. In all cases the luminescence intensity when using hispidin was the highest when using Neonothopanus nambi hispidin hydroxylase and the lowest when using Armillaria mellea hispidin hydroxylase.

### Example 5. Obtaining of 3-hydroxyhispidin, (E)-3,4-dihydroxy-6-styryl-2H-pyran-2-one and (E)-3,4-dihydroxy-6-(4-hydroxystyryl)-2H-pyran-2-one Using Recombinant Hispidin Hydroxylase

The isolated recombinant hispidin hydroxylase from Neonothopanus nambi, obtained according to Example 4, was added to reaction mixtures comprising 1 mM of NADPH and 0.2 μM of hispidin, (E)-4-hydroxy-6-styryl-2H-pyran-2-one or (E)-4-hydroxy-6-(4-hydroxystyryl)-2H-pyran-2-one in 100 μl of the buffer (0.2 M of Na-phosphate buffer, 0.5 M of Na₂SO₄, 0.1% dodecylmaltoside (DDM) pH 8.0). In 30 minutes, the reaction mixture was analyzed by HPLC using synthetic luciferins as standards. Chromatography demonstrated occurrence of peaks corresponding to 3d position hydroxylated derivatives: 3-hydroxyhispidin, (E)-3,4-dihydroxy-6-styryl-2H-pyran-2-one and (E)-3,4-dihydroxy-6-(4-hydroxystyryl)-2H-pyran-2-one.

### Example 6. Bioluminescence Detection by Hispidin Hydroxylase and Luciferase Fusion Protein

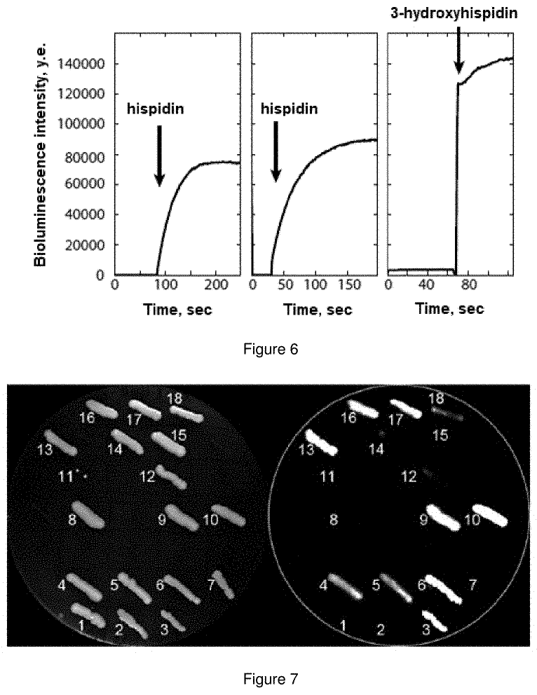

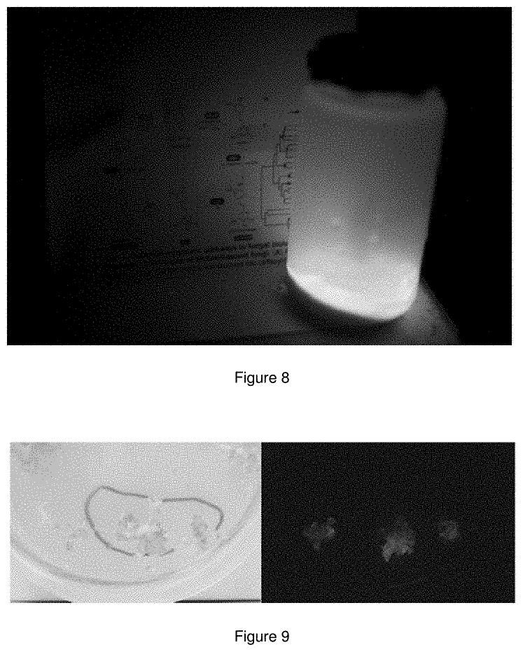

Humanized DNA sequences coding hispidin-hydroxylase and luciferase of Neonothopanus nambi, obtained according to Example 2, were operatively cross-linked to each other by flexible short peptide linker with amino acid sequence GGSGGSGGS (SEQ ID NOs:115). Nucleotide and amino acid sequences of the obtained fused protein are shown in SEQ ID NO 101 and 102. Nucleic acid coding fused protein was cloned into pEGFP-N1 vector (Clontech, USA) instead of EGFP gene under Cytomegaloviral promoter control. The obtained structure was transfected into HEK293T cells. Analogous vectors, comprising individual genes of hispidin hydroxylase and luciferase, were also co-transfected. Transfection was performed by transfection agent FuGENE HD (Promega, USA) according to the manufacturer's protocol. 24 hours after transfection 1 million of cells were resuspended in 0.5 ml of PBS, and luminescence without adding hispidin and with addition of hispidin (10 μg per 1 million of cells) was recorded by luminometer. Addition of hispidin caused cell luminescence in green spectrum (FIG. 6). Addition of 3-hydroxyhispidin also caused bioluminescent signal. Expression of hispidin hydroxylase and luciferase fusion proteins enables to use more stable luciferin precursors (hispidin, bisnoryangonin and others) instead of one luciferase for cell bioluminescent labeling and it does not require co-transfection of two nucleic acids into cells.

### Example 7. Preparation of Polyclonal Antibodies

Coding sequences of hispidin hydroxylases of Neonothopanus nambi (SEQ ID NO: 1) and Armillaria mellea (SEQ ID NO: 19) were synthetically obtained in the form of linear double-stranded DNA and cloned into expression vectors pQE-30 (Qiagen, Germany) in such a way, that recombinant proteins comprised histidine tag at N-terminal. After expression in E. coli, recombinant proteins were purified by metal affinity resin TALON (Clontech, USA) in denaturating conditions. Purified protein products emulsified in Freund adjuvant were used for four rabbit immunizations at month intervals. Rabbit blood was sampled the tenth or eleventh day after immunizations. Activity of the obtained polyclonal antisera was tested by ELISA and Western immunoblotting methods on the panel of purified recombinant hispidin hydroxylases obtained according to Example 4.

Antibodies, obtained at rabbit immunization with protein from Neonothopanus nambi, demonstrated activity against denatured and nondenaturated hispidin hydroxylase of Neonothopanus nambi and against denatured hispidin hydroxylase of Neonothopanus gardneri.

Antibodies, obtained at rabbit immunization with protein from Armillaria mellea were active against denatured and nondenaturated hispidin hydroxylase of Armillaria mellea, Armillaria gallica, Armillaria ostoyae and Armillaria fuscipes.

### Example 8. Obtaining of Transgenic Plants Expressing Neonothopanus nambi Hispidin Hydroxylase and Luciferase

Coding sequences of Neonothopanus nambi hispidin hydroxylase and luciferase were optimized for expression in Physcomitrella patens moss cells. Then, in silico there was created an expression cassette comprising promoter of rice aktl gene, human cytomegalovirus 5′-untranslated region coding hispidin hydroxylase sequence optimized for expression in plant cells (SEQ ID NO 103), terminating codon, Agrobacterium osc gene terminator sequence, rice ubiquitin promoter, coding sequence of Neonothopanus nambi luciferase (SEQ ID NO 112) optimized for expression in moss cells, Agrobacterium tumefaciens nos gene terminator.

The obtained sequence was synthesized in such a way, that all said fragments appeared to be operatively cross-linked to each other, and cloned by Gibson assembly technique [Gibson et al., Nat Methods, 2009, 6:343-5] into expression vector pLand #1 (Institut Jean-Pierre Bourgin, France), between DNA fragments coincident with locus of Physcomitrella patens moss genomic DNA between sequences of highly expressed moss genes Pp3c16_6440V3.1 and Pp3c16_6460V3.1. The vector pLand #1 also comprised a guide RNA (sgRNA) sequence for Cas9 nuclease, complementary to the region of the same DNA locus.

Plasmid DNA product was co-transformed together with the expression vector comprising Cas9 nuclease sequence under Arabidopsis thaliana ubiquitin promoter, into Physcomitrella patens moss protoplasts according to the polyethylenglycol transformation protocol described in [Cove et al., Cold Spring Harb Protoc., 2009, 2]. Then protoplasts were incubated in BCD medium within two days under darkroom conditions with fluctuation at 50 rpm to regenerate cell wall. Then protoplasts were transferred to Petri dishes comprising agar and BCD medium and grown at 16 hours lighting within a week. Transformed moss colonies were screened from external genomic primers by PCR to determine the progress of gene construct integration into genome, transferred to fresh Petri dishes and grown in the same lighting conditions within 30 days.

The obtained moss gametophytes were soaked in BCD medium comprising hispidin at concentration of 900 μg/ml, and analyzed by means of IVIS Spectrum In Vivo Imaging System (Perkin Elmer). All analyzed transgenic plant demonstrated bioluminescence with intensity minimum by two orders of magnitude exceeding the signal of control plants expressing luciferase only, incubated in the same solution with hispidin.

### Example 9. Identification of Hispidin-Synthases and Caffeylpyruvate Hydrolases

Fungal luciferin precursors, such as hispidin, relate to a large group of chemical compounds—polyketide derivatives. Such compounds could be theoretically obtained from 3-arylacrylic acids, in which aromatic substituent, including aryl or heteroaryl are 3d position substituents. It is known in the art that enzymes involved in synthesis of polyketides and their derivatives are multidomain complexes related to polyketide synthase protein superfamily. At the same time, no polyketide synthase, able to catalyze transformation of 3-arylacrylic acid into substituted 4-hydroxy-2H-pyran-2-one, has been known in the art. Screening of Neonothopanus nambi cDNA library was used to search for target polyketide synthase.

It is known, that to obtain functional polyketide synthases in heterologous expression yeast system, it is required to introduce additionally into the culture a gene expressing 4′-phosphopantetheinyl transferase—enzyme transferring 4-phosphopantetheinyl from coenzyme A to serine in acyl carrier domain of polyketide synthase [Gao Menghao et al., Microbial Cell Factories 2013, 12:77]. NpgA gene of 4′-phosphopantetheinyl transferase from Aspergillus nidulans (SEQ ID NOs 104, 105), known in the art, was obtained synthetically and cloned into pGAPZ vector. Plasmid was linearized at restriction site Avril and used for transformation of Pichia pastoris GS115 yeast line constitutively expressing Neonothopanus nambi, luciferase and hispidin hydroxylase, obtained according to Example 1. Diversity in resulting library of Neonothopanus nambi cDNA in yeasts was about one million of clones.

Neonothopanus nambi cDNA library expressed in the said Pichia pastoris yeast line was obtained according to the protocol given in Example 1 and was used for identification of hispidin-synthases and caffeylpyruvate hydrolases. The cells were dispersed in Petri dishes with RDB medium, comprising 1 M of sorbitol, 2% (weight/volume) of glucose, 1.34% (weight/volume) of yeast nitrogen base (YNB), 0.005% (weight/volume) of amino acids mixture, 0.00004% (weight/volume) of biotin and 2% (weight/volume) of agar.

The obtained colonies were sprayed with caffeic acid (potential hispidin precursor) solution, detecting hispidin-synthase presence in cells by occurrence of luminescence. Luminescence emitted by colonies was detected by means of IVIS Spectrum CT (PerkinElmer, USA). Cells expressing only luciferase and hispidin hydroxylase, and wild yeast cells were used as negative control. When screening the library, the colonies, where luminescence was detected, were selected and used for PCR as a matrix with standard plasmid primers. PCR products were sequenced by Sanger method to determine sequence of the expressed gene. The obtained sequence of hispidin-synthase nucleic acid is shown in SEQ ID NO: 34. The amino acid sequence coded by it is shown in SEQ ID NO: 35.

Then, the obtained Pichia pastoris yeast line, comprising Neonothopanus nambi luciferase, hispidin hydroxylase and hispidin synthase genes integrated into genome, and also NpgA gene of 4′-phosphopantetheinyl transferase from Aspergillus nidulans, was used for identification of enzyme catalyzing transformation of oxyluciferin ((2E,5E)-6-(3,4-dihydroxyphenyl)-2-hydroxy-4-oxohexa-2,5-diene acid) into caffeic acid. The cell line was again transformed by linearized plasmid library of Neonothopanus nambi genes, which was obtained at the first step of work. The colonies were sprayed with caffeoyl pyruvate solution, detecting target enzyme presence in cells by occurrence of luminescence. Luminescence emitted by colonies was detected by means of IVIS Spectrum CT (PerkinElmer, USA). Cells expressing only luciferase and hispidin hydroxylase, and wild yeast cells were used as negative control. When screening the library, the colonies, where luminescence was detected, were selected and used for PCR as a matrix with standard plasmid primers. PCR products were sequenced by Sanger method to determine sequence of the expressed gene. The obtained sequence of isolated enzyme nucleic acid is shown in SEQ ID NO: 64. The amino acid sequence coded by it is shown in SEQ ID NO: 65. The identified enzyme was called as caffeylpyruvate hydrolase.

### Example 10. Identification of Neonothopanus nambi Hispidin-Synthase and Neonothopanus nambi Caffeylpyruvate Hydrolase Homologs

Data of whole-genome sequencing from bioluminescent fungi, obtained according to Example 1, were used to search for homologs of Neonothopanus nambi hispidin-synthase and caffeylpyruvate hydrolase. Homologs search was carried out by means of a software provided by National Center for Biotechnology Information. Search for amino acid sequences in the data of fungal genome sequencing in NCBI Genbank database. Standard search parameters blastp were used at search.

There were identified the sequences of hispidin-synthase homologs from Neonothopanus nambi—in Armillaria fuscipes, Armillaria mellea, Guyanagaster necrorhiza, Mycena citricolor, Neonothopanus gardneri, Omphalotus olearius, Panellus stipticus, Armillaria gallica, Armillaria ostoyae, Mycena chlorophos. Their nucleotide and amino acid sequences are shown in SEQ ID NO 36-55. All identified enzymes were substantially identical to each other. Degree of amino acid sequences identity is shown in Table 5.

TABLE 5
Percent identity of hispidin-synthase full-length natural protein amino acid sequences.

| | SEQ ID | SEQ ID | SEQ ID | SEQ ID | SEQ ID | SEQ ID | SEQ ID | SEQ ID | SEQ ID | SEQ ID |
|---|---|---|---|---|---|---|---|---|---|---|
| | NO: 35 | NO: 53 | NO: 43 | NO: 45 | NO: 37 | NO: 41 | NO: 55 | NO: 47 | NO: 49 | NO: 51 |
| SEQ ID | 100 | 56 | 57 | 56 | 51 | 81 | 57 | 50 | 57 | 57 |
| NO: 35 | | | | | | | | | | |
| SEQ ID | 56 | 100 | 80 | 52 | 45 | 55 | 88 | 46 | 88 | 88 |
| NO: 53 | | | | | | | | | | |
| SEQ ID | 57 | 80 | 100 | 52 | 47 | 56 | 83 | 45 | 85 | 86 |
| NO: 43 | | | | | | | | | | |
| SEQ ID | 56 | 52 | 52 | 100 | 53 | 54 | 52 | 54 | 53 | 53 |
| NO: 45 | | | | | | | | | | |
| SEQ ID | 51 | 45 | 47 | 53 | 100 | 51 | 46 | 51 | 47 | 47 |
| NO: 37 | | | | | | | | | | |
| SEQ ID | 81 | 55 | 56 | 54 | 51 | 100 | 55 | 50 | 56 | 56 |
| NO: 41 | | | | | | | | | | |
| SEQ ID | 57 | 88 | 83 | 52 | 46 | 55 | 100 | 46 | 90 | 91 |
| NO: 55 | | | | | | | | | | |
| SEQ ID | 50 | 46 | 45 | 54 | 51 | 50 | 46 | 100 | 46 | 46 |
| NO: 47 | | | | | | | | | | |
| SEQ ID | 57 | 88 | 85 | 53 | 47 | 56 | 90 | 46 | 100 | 95 |
| NO: 49 | | | | | | | | | | |
| SEQ ID | 57 | 88 | 86 | 53 | 47 | 56 | 91 | 46 | 95 | 100 |
| NO: 51 | | | | | | | | | | |

From Panellus stipticus there were isolated two highly homologous hispidin-synthase amino acid sequences characterized by single amino acid substitution. Their nucleotide and amino acid sequences are shown in SEQ ID NO 36-39.

The identified enzymes were tested for capability to transform caffeic acid into hispidin using technique described in Example 9.

Multiple alignment of identified protein amino acid sequences enabled to identify several highly homologous fragments of amino acid sequence typical of this enzyme group. Consensus sequences for these fragments are shown in SEQ ID NOs: 70-77. The said sequences are separated by long amino acid sequences as shown in FIG. 2.

Neonothopanus nambi caffeylpyruvate hydrolase homolog sequences were identified in Neonothopanus gardneri, Armillaria mellea, Armillaria fuscipes, Armillaria gallica, Armillaria ostoyae. Nucleotide and amino acid sequences of the identified homologs are shown in SEQ ID NOs: 66-75. The identified enzymes were tested for capability to transform caffeoyl pyruvate into caffeic acid using technique described in Example 9.

All identified enzymes are substantially identical to each other and have a length of 280-320 amino acids. Degree of amino acid sequences identity is shown in Table 6.

TABLE 6
Percent identity of caffeoyl pyruvate hydrolase
full-length natural protein amino acid sequences.

| | SEQ ID | SEQ ID | SEQ ID | SEQ ID | SEQ ID | SEQ ID |
|---|---|---|---|---|---|---|
| | NO: 65 | NO: 73 | NO: 75 | NO: 67 | NO: 69 | NO: 71 |
| SEQ ID NO: 65 | 100 | 64 | 64 | 64 | 64 | 64 |
| SEQ ID NO: 73 | 64 | 100 | 92 | 62 | 96 | 95 |
| SEQ ID NO: 75 | 64 | 92 | 100 | 62 | 90 | 90 |
| SEQ ID NO: 67 | 64 | 62 | 62 | 100 | 60 | 61 |
| SEQ ID NO: 69 | 64 | 96 | 90 | 60 | 100 | 97 |
| SEQ ID NO: 71 | 64 | 95 | 90 | 61 | 97 | 100 |

Analysis performed by means of software SMART (Simple Modular Architecture Research Tool), available on the Internet at the website http://smart.embl-heidelberg.de [Schultz et al., PNAS 1998; 95: 5857-5864; Letunic I, Doerks T, Bork P Nucleic Acids Res 2014; doi:10.1093/nar/gku949] revealed that all detected proteins comprise a fumarylacetoacetase domain (EC 3.7.1.2) of about 200 amino acids long, located closer to C-terminal, however, conserved region starts approximately from 8 amino acid according to numbering of Neonothopanus nambi caffeylpyruvate hydrolase amino acids. Multiple alignment enabled to identify consensus sequences (SEQ ID NOs 76-78), typical of this protein group, separated by amino acid inserts with lower identity. Position of consensus sequences is shown in (FIG. 3).

### Example 11. Obtaining of Recombinant Hispidin-Synthases and Caffeylpyruvate Hydrolase and their Use for Obtaining Bioluminescence

Polyhistidine (His tag) coding sequence was operatively attached to 5′-ends of nucleic acids coding hispidin-synthase and caffeylpyruvate hydrolase of Neonothopanus nambi, obtained according to Example 9, and the obtained structures were cloned into pET-23 vector by means of restriction endonucleases NotI and SacI. The vectors were used for transformation of Escherichia coli cells of BL21-DE3-codon+ strain, performed by electroporation. The tmsformed cells were dispersed in Petri dishes with LB medium, comprising 1.5% of agar, 100 μg/ml of ampicillin, and incubated overnight at 37° C. Then, Escherichia coli colonies were transferred into 4 ml of liquid LB medium comprising 100 μg/ml of ampicillin, incubated overnight at 37° C. with fluctuation. 1 ml of overnight culture was transferred into 200 ml of Overnight Express Autoinduction medium (Novagen), where ampicillin was preliminarily added to. The culture was incubated at 37° C. within 3 hours until reaching optical density of 0.6 OE at 600 nm, and then it was incubated at room temperature within 16 hours. Then, the cells were pelleted at 4500 rpm within 20 minutes in centrifuge Eppendorf 5810R, resuspended in 20 ml of the buffer (50 mM of Tris HCl pH 8.0, 150 mM of NaCl), lysed by ultrasound in Bioruptor (Diagenode, Belgium) within 7 minutes at 0° C. in conditions recommended by the manufacturer and pelleted again. Protein was obtained from lysate by Talon resin affinity chromatography (Clontech, USA). Presence of the expected recombinant product was confirmed by electrophoresis as bands of the expected length were available.

Aliquots of isolated recombinant proteins were used for testing functionality and stability.

For determination of hispidin synthase functionality 30 μl of isolated recombinant protein solution were put into a glass tube, comprising 100 μl of the buffer (0.2 M of Na-phosphate buffer, 0.5 M of Na₂SO₄, 0.1% dodecylmaltoside (DDM) pH 8.0, all components—Sigma-Aldrich, USA), 0.5 μl of purified recombinant luciferase of Neonothopanus nambi, obtained according to Example 4, 1 mM of NADPH (Sigma-Aldrich, USA), 15 μl of purified recombinant hispidin hydroxylase of Neonothopanus nambi, obtained according to Example 4, 10 mM of ATP (ThermoFisher Scientific, USA), 1 mM of CoA (Sigma-Aldrich, USA), 1 mM of malonyl-CoA (Sigma-Aldrich, USA). The glass tube was placed into a luminometer GloMax 20/20 (Promega, USA). The reaction mixtures demonstrated bioluminescence at adding of 20 μM of caffeic acid into the solution (Sigma-Aldrich, USA). Maximum emission of the emitted luminescence was 520-535 nm.

For determination of caffeylpyruvate hydrolase functionality 10 μl of isolated recombinant protein solution were put into a glass tube, comprising 100 μl of the buffer (0.2 M of Na-phosphate buffer, 0.5 M of Na₂SO₄, 0.1% dodecylmaltoside (DDM) pH 8.0, 0.5 μl of luciferase of Neonothopanus nambi, 1 mM of NADPH (Sigma-Aldrich, USA), 15 μl of hispidin hydroxylase 10 mM of ATP (ThermoFisher Scientific, USA), 1 mM of CoA (Sigma-Aldrich, USA), 1 mM of malonyl-CoA (Sigma-Aldrich, USA), 30 μl of purified recombinant hispidin synthase. The glass tube was placed into a luminometer GloMax 20/20 (Promega, USA). Bioluminescence of the reaction mixture was detected at adding of 25 μM of caffeoyl pyruvate into the solution, being indicative of the test enzyme capability to decompose caffeoyl pyruvate to caffeic acid. Maximum emission of the emitted luminescence was 520-535 nm.

The obtained enzymes were used for obtaining luminescence (bioluminescence) in reaction with Neonothopanus nambi luciferase and hispidin hydroxylase, obtained according to Example 4. 5 μl of each isolated recombinant protein solution were put into a glass tube, comprising 100 μl of the buffer (0.2 M of Na-phosphate buffer, 0.5 M of Na₂SO₄, 0.1% dodecylmaltoside (DDM) pH 8.0), 1 mM of NADPH (Sigma-Aldrich, USA), 10 mM of ATP (ThermoFisher Scientific, USA), 1 mM of CoA (Sigma-Aldrich, USA), 1 mM of malonyl-CoA (Sigma-Aldrich, USA) and 0.2 μM of one of 3-arylacrylic acids: paracoumaric acid (Sigma-Aldrich, USA), cinnamic acid (Sigma-Aldrich, USA) or ferulic acid (Abcam, USA). In the other experiment instead of substituted 3-arylacrylic acid the analogues of fungal oxyluciferin—(2E,5E)-2-hydroxy-6-(4-hydroxyphenyl)-4-oxohexa-2,5-diene, (2E,5E)-2-hydroxy-4-oxo-6-phenylhexa-2,5-diene, or (2E,5E)-2-hydroxy-6-(4-hydroxy-3-methoxyphenyl)-4-oxohexa-2,5-diene acids—also at concentration of 0.2 μM were put into a glass tube. The glass tubes were placed into a luminometer. Activity of the isolated recombinant proteins resulted in luminescence in each of the described reactions.

### Example 13. Obtaining Hispidin from Caffeic Acid

Expression cassette, comprising nucleic acid coding Neonothopanus nambi hispidin-synthase (SEQ ID NOs 34, 35), under control of J23100 promoter, and expression cassette, comprising NpgA gene of 4′-phosphopantetheinyl transferase from Aspergillus nidulans (SEQ ID NOs 104, 105) under control of araBAD promoter, floxed by homology regions to SS9 site, were obtained synthetically and cloned into bacterial expression vector comprising Zeocin resistance cassette. The obtained structure was used for transformation and integration into E. coli BW25113 genome by means of lambda bacteriophage protein-mediated recombination, as described in Bassalo et al. [ACS Synth Biol. 2016 Jul. 15; 5(7):561-8], using selection for Zeocin resistance. Integration of full-length structure was confirmed by PCR from primers specific to SS9 homology regions, and then the correctness of the integrated structure was verified by sequencing of genomic DNA PCR product by Sanger method.

The obtained E. coli strain was used for producing hispidin. At the first step the bacteria were incubated in five 50 ml plastic tubes in LB medium within 10 hours at 200 rpm fluctuation at 37° C. 250 ml of the obtained culture were added to 3.3 litres of fermentation medium into a fermenter Biostat B5 (Braun, Germany) so that initial culture optical density at 600 nm was about 0.35. Fermentation medium comprised 10 g/l of peptone, 5 g/l of caffeic acid, 5 g/l of yeast extract, 10 g/l of NaCl, 25 g/l of glucose, 15 g/l of (NH₄)₂SO₄, 2 g/l of KH₂PO₄, 2 g/l of MgSO₄.7H₂O, 14.7 mg/l of CaCl₂, 0.1 mg/l of thiamine, 1.8 mg/l and 0.1% of the solution composed of: EDTA 8 mg/l, CaCl₂.6H₂O 2.5 mg/l, MnCl₂.4H₂O 15 mg/l, CuCl₂.2H₂O 1.5 mg/l, HsBO₃.3 mg/l, Na₂MoO₄.2H₂O 2.5 mg/l, Zn(CH₃COO)₂.2H₂O 13 mg/l, iron (III) citrate 100 mg/l, thiamine hydrochloride 4.5 mg/l. Fermentation was carried out at 37° C., with 31/min aeration and 200 rpm mixing. After 25 hours of cultivation arabinose was added to the culture up to final concentration of 0.1 mM. pH was automatically controlled by adding of NH₄OH, reducing pH to 7.0. The solution comprising 500 g/l of glucose, 5 g/l of caffeic acid, 2 g/l of arabinose, 25 g/l of trypton, 50 g/l of yeast extract, 17.2 g/l of MgSO₄.7H₂O, 7.5 g/l of (NH₄)SO₄, 18 g/l of ascorbic acid, was added to a fermenter to maintain glucose level every time when pH increased to 7.1. After 56 hours of cultivation the hispidin concentration in the medium was 1.23 g/l. Fermenter medium and also hispidin purified from it by HPLC were active in bioluminescence reaction with Neonothopanus nambi hispidin hydroxylase and luciferase.

### Example 13. Obtaining 3-Hydroxyhispidin from Caffeic Acid

Expression cassette, comprising nucleic acid coding Neonothopanus nambi hispidin hydroxylase (SEQ ID NOs 1, 2) under control of J23100 promoter, was obtained synthetically and cloned into bacterial expression vector comprising spectinomycin resistance gene. The obtained vector was transformed into E. coli cells expressing Neonothopanus nambi hispidin-synthase, Zeocin resistance gene and NpgA gene, obtained according to Example 12. The obtained bacteria were used for producing 3-hydroxyhispidin by fermentation according to the protocol described in Example 12, however with adding of spectinomycin at concentration of 50 mg/ml in all media used for cultivation. After 48 hours of cultivation the 3-hydroxyhispidin concentration in the medium was 2.3 g/l. Fermenter medium and also 3-hydroxyhispidin purified from it by HPLC were active in bioluminescence reaction with Neonothopanus nambi luciferase.

### Example 14 Obtaining Hispidin from Cell Metabolites and Tyrosine

E. coli strain, effectively producing tyrosine and caffeic acid, was obtained for producing biosynthetic hispidin from tyrosine. E. coli strain was obtained as described in [Lin and Yan. Microb Cell Fact. 2012 Apr. 4; 11:42]. E. coli BW25113 line with integrated mutant gene of acY (lacY A177C) permease at attB site providing uniform consumption of arabinose by bacteria cells was taken as a basis for strain development. Expression cassettes comprising coding sequences of Rhodobacter capsulatus tyrosine-ammonia-lyase genes (SEQ ID NOs: 106, 107), and the components HpaB and HpaC of E. coli 4-hydroxyphenyl acetate 3-monooxygenase-reductase (SEQ ID NOs: 108-111), each under control of constitutive J23100 promoter, were obtained synthetically and integrated into genome of E. coli strain as described in Example 12. At the next step the plasmid obtained according to Example 12 and comprising coding sequence of Neonothopanus nambi hispidin-synthase under control of constitutive J23100 promoter, Zeocin resistance cassette from pGAP-Z vector, and also NpgA gene were integrated into E. coli genome. Integration into E. coli genome was performed by means of lambda bacteriophage protein-mediated recombination according to the technique from [Bassalo et al., ACS Synth Biol. 2016; 5(7):561-568]. Integration of full-length structure was confirmed by PCR from primers specific to SS9 homology regions (5′-CGGAGCATTTTGCATG-3′ and 5′-TGTAGGATCAAGCTCAG-3′), and then the correctness of the integrated structure was verified by sequencing of genomic DNA PCR product by Sanger method. The obtained bacteria strain was used for producing biosynthetic hispidin in a fermenter.

Bacteria were cultivated in a fermenter, as described in Example 12, with the only difference—caffeic acid was not added to bacteria culture media. Biosynthetic hispidin was isolate from the medium by HPLC. The obtained strain was able to produce 1.20 mg/l of hispidin per 50 hours of fermentation. The obtained product purity was 97.3%. Adding of tyrosine to culture media at concentration of 10 g/ml enabled to increase hispidin output to 108.3 mg/ml.

### Example 15. Development of Autonomously Bioluminescent Yeast Pichia pastoris

For the purpose of autonomously bioluminescent yeast Pichia pastoris development there were synthesized expression cassettes comprising, under control of GAP promoter and tAOX1 terminator, coding sequences of Neonothopanus nambi luciferase (SEQ ID NOs: 79, 80), Neonothopanus nambi hispidin hydroxylase (SEQ ID NOs: 1, 2), Neonothopanus nambi hispidin-synthase (SEQ ID NOs: 34, 35), Neonothopanus nambi caffeylpyruvate hydrolase (SEQ ID NOs: 64, 65), Aspergillus nidulans NpgA protein (SEQ ID NOs: 104, 105), Rhodobacter capsulatus tyrosine-ammonia-lyase (SEQ ID NOs: 106, 107), and the components HpaB and HpaC of E. coli 4-hydroxyphenyl acetate 3-monooxygenase-reductase (SEQ ID NOs: 108-111). Each expression cassette was floxed by BsmBI restriction enzyme recognition sequences. Homology regions to MET6 Pichia pastoris gene (Uniprot F2QTU9), floxed by BsmBI restriction enzyme sites, were also obtained syntheticaly. Synthetic DNA was treated by BsmBI restriction enzymes and combined into one plasmid according to Golden Gate cloning protocol, described in [Iverson et al., ACS Synth Biol. 2016 Jan. 15; 5(1):99-103]. 10 fmol of each DNA fragment were mixed in reaction comprising normal strength buffer for DNA lygase (Promega, USA), units of DNA lygase activity (Promega, USA), 10 units of DNA restriction endonuclease activity in a total volume of 10 μl. The obtained reaction mixture was put into an amplifier and incubated at 16° C. and 37° C. according to the following protocol: 25 cycles of incubation at 37° C. within 1.5 min and at 16° C.—3 min, then single incubation at 50° C. within 5 min, and then single incubation at 80° C. within 10 min. 5 μl of reaction mixture were transformed into E. coli chemically competent cells. Correctness of plasmid DNA assembly was confirmed by Sanger method, and purified plasmid DNA product was used for transformation of Pichia pastoris GS11 cells by electroporation. Electroporation was carried out according to the method, using lithium acetate and dithiothreitol, described in [Wu and Letchworth, Biotechniques, 2004, 36:152-4]. Electroporated cells were dispersed in Petri dishes with RDB medium, comprising 1 M of sorbitol, 2% (w/v) of glucose, 1.34% (w/v) of yeast nitrogen base (YNB), 0.005% (w/v) of amino acids mixture, 0.00004% (w/v) of biotin and 2% (w/v) of agar. Integration of gene cassette into genome was confirmed by PCR from primers annealed at a homology region. The obtained yeast strain comprising correct genome insert was able to illuminate autonomously in contrast to wild yeast strain (FIG. 7, 8).

### Example 16. Development of Autonomously Bioluminescent Flowering Plants

For the purpose of autonomously bioluminescent flowering plants development based on pBI121 vector (Clontech, USA) there was created a binary vector for agrobacterium transformation comprising coding sequences of Neonothopanus nambi luciferase optimized for expression in plants (SEQ ID NO: 112), Neonothopanus nambi hispidin hydroxylase (SEQ ID NO: 103), Neonothopanus nambi hispidin-synthase (SEQ ID NO: 113), Neonothopanus nambi caffeylpyruvate hydrolase (SEQ ID NO: 114) and kanamycin resistance gene, each gene is under control of 35S promoter from cauliflower mosaic virus. Sequences for expression cassettes assembly were obtained synthetically, the vector was assembled according to Golden Gate cloning protocol, described in [Iverson et al., ACS Synth Biol. 2016 Jan. 15; 5(1):99-103].

Arabidopsis thaliana was transformed by co-cultivation of plant tissue with Agrobacterium tumefaciens bacteria of AGL0 strain [Lazo et al., Biotechnology, 1991 October; 9(10):963-7], comprising the created binary vector. Transformation was performed using co-cultivation of Arabidopsis thaliana root segments (C24 ecotype), as described in [Valvekens et al., 1988, Proc. Nat. Acad. Sci. USA 85, 5536-5540]. Arabidopsis thaliana roots were cultivated in agarized Gamborg medium B-5 with 20 g/l of glucose, 0.5 g/l of 2,4-dichlorophenoxyacetic acid and 0.05 g/l of kinetin within 3 days. Then, roots were cut into pieces 0.5 cm long and transferred into 10 ml of liquid Gamborg medium B-5 with 20 g/l of glucose, 0.5 g/l of 2,4-dichlorophenoxyacetic acid and 0.05 g/l of kinetin, and 1.0 ml of agrobacteria overnight culture medium was added. Explants with agrobacteria were co-cultivated within 2-3 minutes. Then, the explants were put on sterile filters in Petri dishes with agarized medium of the same composition. After 48 hours of incubation in a thermostat at 25° C. the explants were transferred to fresh medium with 500 mg/l of cefotaxime and 50 mg/l of kanamycin. In three weeks, regeneration of plants on selective medium, comprising 50 mg/l of kanamycin, was started. Transgenic plants took roots and were transferred to germination medium or soil. Bioluminescence was visualized by means of IVIS Spectrum In Vivo Imaging System (Perkin Elmer). More than 90% of transgenic plants emitted luminescence minimum by two orders of magnitude exceeding the signal from wild-type plants.

Nicotiana benthamiana was transformed by co-cultivation of plant tissue with Agrobacterium tumefaciens bacteria of AGL0 strain [Lazo et al., Biotechnology, 1991 October; 9(10):963-7], comprising the created binary vector. Transformation was performed using co-cultivation of Nicotiana benthamiana leaf segments. Then, leaves were cut into pieces 0.5 cm long and transferred into 10 ml of liquid Gamborg medium B-5 with 20 g/l of glucose, 0.5 g/l of 2,4-dichlorophenoxyacetic acid and 0.05 g/l of kinetin, and 1.0 ml of agrobacteria overnight culture medium was added. Explants with agrobacteria were co-cultivated within 2-3 minutes. Then, the explants were put on sterile filters in Petri dishes with agarized medium of the same composition. After 48 hours of incubation in a thermostat at 25° C. the explants were transferred to fresh medium with 500 mg/l of cefotaxime and 50 mg/l of kanamycin. In three weeks, regeneration of plants on selective medium, comprising 50 mg/l of kanamycin, was started. Transgenic plants took roots and were transferred to germination medium or soil. Bioluminescence was visualized by means of IVIS Spectrum In Vivo Imaging System (Perkin Elmer). More than 90% of transgenic plants emitted luminescence minimum by two orders of magnitude exceeding the signal from wild-type plants. Photos of autonomously luminescent Nicotiana benthamiana are given in FIG. 9.

For the purpose of development of autonomously bioluminescent Agrostis stolonifera L. there were cloned into pBI121 vector (Clontech, USA) the coding sequences of fungal luciferin metabolic cascade genes, optimized for expression in plants and floxed by BsaI restriction endonuclease sites:Neonothopanus nambi luciferase (SEQ ID NO: 126), Neonothopanus nambi hispidin hydroxylase (SEQ ID NO: 117), Neonothopanus nambi hispidin-synthase (SEQ ID NO: 127), Neonothopanus nambi caffeylpyruvate hydrolase (SEQ ID NO: 128) and herbicide glyphosate resistance gene (bar gene). Each sequence was under control of CmYLCV promoter [Stavolone et al., Plant Mol Biol. 2003 November; 53(5):663-73]. The sequences were synthesized according to standard technique. The vector was assembled according to Golden Gate cloning protocol. Transformation was performed by the method of embryogenic callus agrobacterium transformation. Overnight culture of Agrobacterium tumefaciens bacteria of AGL0 strain [Lazo et al., Biotechnology, 1991 October; 9(10):963-7], comprising the created binary vector, was added to liquid medium. After two days of co-cultivation in agarized Murashige and Skoog medium the plants were transferred to fresh medium with 500 mg/l of cefotaxime and 10 mg/l of phosphinothricin. Plant regeneration started in three weeks. Transgenic plants were replanted into the medium with half Murashige and Skoog salt content and 8 mg/l of phosphinothricin for rootage. Rooted plants were set out in a greenhouse. About 25% of the obtained plants with correct and complete integration into metabolic cascade genome had bioluminescence exceeding bioluminescence of control wild-type plants.

Organisms able to emit luminescence in certain tissues or at certain times of the day are of special interest. Such organisms consume resources required for luminescence more efficiently. For the purpose of development of autonomously bioluminescent roses emitting luminescence only in petals, there were selected several rose varieties with white petals. On the basis of pBI121 vector (Clontech, USA) there were created two binary vectors for agrobacterium transformation comprising metabolic cascade from the coding sequences of Neonothopanus nambi luciferase, Neonothopanus nambi hispidin hydroxylase, Neonothopanus nambi hispidin synthase, Neonothopanus nambi caffeylpyruvate hydrolase and neomycin resistance gene, which were optimized for expression in plants. All genes, except for luciferase gene, were put under control of cauliflower mosaic virus 35S promoter. In one of the vectors luciferase gene was put under control of rose chalcone synthase promoter, and in the other—under control of chrysanthemum chalcone UEP1 promoter. There were used synthetic nucleic acids required for vector assembly, floxed by BsaI restriction enzyme recognition sites, and the vector was assembled according to Golden Gate cloning protocol. Rosa hybrida L. cv. Tinike transgenic plants were obtained by co-cultivation of embryogenic callus with Agrobacterium tumefaciens bacteria of AGL0 strain [Lazo et al., Biotechnology, 1991 October; 9(10):963-7], comprising on of the above binary vectors. Cultivation was performed in liquid medium comprising Murashige and Skoog macro- and micro-salts, with addition of 1-2 mg/l of kinetin, 3 mg/l of 2,4-dichlorophenoxyacetic acid and 1 mg/l of 6-benzylaminopurine within 40 minutes. Callus was transferred to agarized medium of the same composition. In two days, the explants were transferred to fresh Murashige and Skoog medium with 500 mg/l of cefotaxime and 50 mg/l of kanamycin. Shoot formation and regeneration took place in 5-8 weeks. Shoots were transferred to propagation or rooting medium. Rooted shoots were set out into peat mixture in a greenhouse. Flowering was observed in 8 weeks. Plants with mature flowers were visualized in IVIS Spectrum In Vivo Imaging System (Perkin Elmer). All tested plants of each tested structure autonomously emitted luminescence minimum by three orders of magnitude exceeding the signal from wild-type plants. Luminescence was emitted form petal tissues only, confirming tissue-specific functioning of promoters.

For the purpose of development of autonomously bioluminescent plants, where bioluminescence is controlled by circadian rhythms and activated at night time there was used earlier obtained binary vector for agrobacterium transformation comprising coding sequences of Neonothopanus nambi luciferase, Neonothopanus nambi, hispidin hydroxylase, Neonothopanus nambi hispidin synthase, Neonothopanus nambi caffeylpyruvate hydrolase and neomycin resistance gene, and each gene is under control of 35S promoter from cauliflower mosaic virus. Promoter for expression of Neonothopanus nambi luciferase was replaced by promoter of CAT3 gene from Arabidopsis thaliana. Transcription from CAT3 gene promoter is controlled by circadian rhythms and activated at nighttime. CAT3 promoter sequence is known in the art [Michael and McClung, Plant Physiol. 2002 October; 130(2):627-38]. Arabidopsis thaliana was transformed by co-cultivation of plant tissue with Agrobacterium tumefaciens bacteria of AGL0 strain [Lazo et al., Biotechnology, 1991 October; 9(10):963-7], comprising the created binary vector. Transformation was performed using co-cultivation of Arabidopsis thaliana root segments (C24 ecotype), as described in [Valvekens et al., 1988, Proc. Nat. Acad. Sci. USA 85, 5536-5540]. Arabidopsis thaliana roots were cultivated in agarized Gamborg medium B-5 with 20 g/l of glucose, 0.5 g/l of 2,4-dichlorophenoxyacetic acid and 0.05 g/l of kinetin within 3 days. Then, roots were cut into pieces 0.5 cm long and transferred into 10 ml of liquid Gamborg medium B-5 with 20 g/l of glucose, 0.5 g/l of 2,4-dichlorophenoxyacetic acid and 0.05 g/l of kinetin, and 1.0 ml of agrobacteria overnight culture medium was added. Explants with agrobacteria were co-cultivated within 2-3 minutes. Then, the explants were put on sterile filters in Petri dishes with agarized medium of the same composition. After 48 hours of incubation in a thermostat at 25° C. the explants were transferred to fresh medium with 500 mg/l of cefotaxime and 50 mg/l of kanamycin. In three weeks, regeneration of plants on selective medium, comprising 50 mg/l of kanamycin, was started. Transgenic plants took roots and were transferred to germination medium, they were grown in natural day-night cycle conditions. Bioluminescence was visualized by means of IVIS Spectrum In Vivo Imaging System (Perkin Elmer), placing the plants into the instrument for 24 hours and recording bioluminescence intensity every half hour. Plants emitted luminescence within 24 hours, however, bioluminescence intensity was significantly modulated by circadian rhythms: integral luminous intensity at nighttime exceeded integral luminosity at day time more than 1000 times for 85% of tested plants.

### Example 17. Development of Transgenic Autonomously Bioluminescent Lower Plants

Autonomously bioluminescent moss Physcomitrella patens was developed by protoplast co-transformation with plasmids using the method described in Example 8. There were synthetically obtained expression cassettes, including, optimized for expression in plants, coding sequences of Neonothopanus nambi luciferase (SEQ ID NO: 112), Neonothopanus nambi hispidin hydroxylase (SEQ ID NO: 103), Neonothopanus nambi hispidin-synthase (SEQ ID NO: 113), Neonothopanus nambi caffeylpyruvate hydrolase (SEQ ID NO: 114) and kanamycin resistance gene, each is under control of rice actin 2 promoter. Expression cassettes were operatively cross-linked in pBI121 vector (Clontech, CWA) in such a way, that the structure including full metabolic cascade and kanamycin resistance gene were floxed by sequences coincident with moss genome target locus sequence. The vector was assembled according to Golden Gate cloning protocol Golden Gate [Iverson et al., ACS Synth Biol. 2016 Jan. 15; 5(1):99-103]. Guide RNA gene, complementary to target region in the moss genome, was also cloned into the vector. Plasmid with specified genes was co-transformed with plasmid for constitutive expression of Cas9 nuclease according to the polyethylenglycol transformation protocol described in [Cove et al., Cold Spring Harb Protoc., 2009, 2]. The obtained transformed protoplasts were incubated in dark conditions within 24 hours in BG-11 medium, and then were transferred to Petri dishes with BG-11 medium and 8.5% agar. Visualization in IVIS Spectrum In Vivo Imaging System (Perkin Elmer) was performed a month after growing in Petri dishes at continuous lighting. 70% of tested plants emitted luminescence exceeding the signal from wild-type plants minimum by an order of magnitude.

### Example 18. Development of Transgenic Luminescent Animals

Transgenic fish Danio rerio, comprising gene of Neonothopanus nambi hispidin hydroxylase were created according to the technique described in [Hisano et al., Sci Rep., 2015, 5:8841]. The technique includes expression of guide RNA and Cas9 nuclease for making a breakpoint in the region homologous to the guide RNA sequence. For the purpose of development of transgenic animals there were ordered synthetic DNA fragments comprising guide RNA sequences from pX330 plasmid, Addgene #42230 and mRNA of Cas9 nuclease under control of bacteriophage polymerase T7 promoter. The obtained fragments were used for transcription in vitro by means of reagents from MAXIscript T7 kit (Life Technologies, USA), and synthesized RNA was purified by means of DNA isolation kit (Evrogen, Russia).

Coding sequence of Neonothopanus nambi hispidin hydroxylase floxed by 50-nucleotide sequences from krtt1c19e Danio rerio gene, described in [Hisano et al., Sci Rep., 2015, 5:8841], was obtained synthetically and cloned into pEGFP/C1 plasmid base comprising pUC origin and kanamycin resistance cassette. The obtained vector, Cas9 nuclease mRNA and guide RNA were dissolved in injection buffer (40 mM HEPES (pH 7.4), 240 mM KCl with addition of 0.5% of phenol red) and injected into 1-2 cell embryos of the earlier obtained Danio rerio line, stably expressing Neonothopanus nambi luciferase, in the volume of about 1-2 nl. About 12 from 48 embryos survived the injection and demonstrated normal development on the fourth day after fertilization.

Hispidin solution was intravenously injected into Danio rerio larvae for recording bioluminescent signal according to the technique described in [Cosentino et al., J Vis Exp. 2010; (42): 2079]. Bioluminescence was recorded by means of IVIS Spectrum In Vivo Imaging System (Perkin Elmer). After recording, genomic DNA was isolated from larvae to confirm integration of hispidin hydroxylase into genome. All larvae with correct integration of Neonothopanus nambi hispidin hydroxylase gene into genome demonstrated bioluminescence intensity minimum by two orders of magnitude exceeding the signal outgoing from wild-type fish after hispidin solution injection.

### Example 19. Study of Caffeylpyruvate Hydrolase Effect on Luminescence of Autonomously Bioluminescent Organisms

For the purpose of study of caffeylpyruvate hydrolase effect on luminescence of autonomously bioluminescent organisms there was used a binary vector for agrobacterium transformation comprising coding sequences of Neonothopanus nambi luciferase, Neonothopanus nambi hispidin hydroxylase, Neonothopanus nambi hispidin synthase, Neonothopanus nambi caffeylpyruvate hydrolase and kanamycin resistance gene, and each gene is under control of 35S promoter from cauliflower mosaic virus, obtained according to Example 16, and control vector characterized in that caffeylpyruvate hydrolase sequence was removed from it. The vectors were used for transformation of Arabidopsis thaliana in the same conditions according to the protocol described in Example 16. Bioluminescence was visualized by means of IVIS Spectrum In Vivo Imaging System (Perkin Elmer). Comparison of bioluminescence intensities of the plants expressing all four genes of Neonothopanus nambi bioluminescent system with the plants expressing only luciferase, hispidin hydroxylase and hispidin synthase, has revealed that the plants additionally expressing caffeylpyruvate hydrolase have on average 8.3 times more bright bioluminescence. The data provided indicate that expression of caffeylpyruvate hydrolase enables to increase bioluminescent cascade efficiency, that results in increasing the intensity of luminescence emitted by plants.

### Example 20. Effect of External Adding Caffeic Acid on Transgenic Organism Bioluminescence

Autonomously bioluminescent transgenic plants Nicotiana benthamiana, obtained according to Example 16, were transferred to soil and cultured within eight weeks. Then, plant stem was cut and placed in water for two hours, after that, bioluminescence intensity was measured by IVIS Spectrum In Vivo Imaging System (Perkin Elmer). Then, plants were transferred to one of five water solutions at caffeic acid concentration of 0.4 g/l, 0.8 g/l, 1.6 g/l, 3.2 g/l, or 6.4 g/l, and control plants were placed in water. After two more hours of incubation in the caffeic acid solution or in water bioluminescence intensity was measured again. In all cases the bioluminescence intensity of the plants incubated in caffeic acid solution increased as compared to the intensity before placing in caffeic acid solution, and the largest changes were observed in plants incubated in the solution at a concentration of 6.4 g/l. Control plants incubated in water did not demonstrated significant change in bioluminescence intensity within four hours after incubation start.

### Example 21. Use of Fungal Bioluminescent System Genes for Activity Assay of Promoters and Intracellular Logical Integration of External Signals

Coding sequences of Neonothopanus nambi hispidin hydroxylase, hispidin synthase and luciferase were used for monitoring simultaneous activation of several promoters. Synthetic expression cassettes comprising coding sequence of Neonothopanus nambi hispidin-synthase (SEQ ID NOs: 34, 35) under control of E. coli araBAD promoter induced by arabinose, coding sequence of hispidin hydroxylase (SEQ ID NOs 1, 2) under control of T7/lacO promoter induced by IPTG, and luciferase gene (SEQ ID NOs: 79, 80) under control of pRha promoter induced by rhamnose, and also NpgA gene (SEQ ID NOs: 104, 105) under control of constitutive J23100 promoter (Registry of Standard Biological parts, Part:BBa_J23100). The obtained synthetic nucleic acids were cloned into MoClo_Level2 vector [Weber et al., PLoS One. 2011 Feb. 18; 6(2):e16765] instead of the insert comprising LacZ gene, using BpiI restriction endonuclease. The obtained vector was transformed into E. coli BL21 (NEB, USA) strain competent cells comprising genomic copy of T7 bacteriophage polymerase.

For the purpose of determining a possibility of recording simultaneous activation of several promoters the cells obtained at the previous step were grown within a night in a flask with 100 ml LB medium with addition of ampicillin at a concentration of 100 mg/l. The next day the cell culture aliquots were placed for 120 minutes at 24° C. and 200 rpm into one of the media with following composition:

1. LB medium with addition of 1% arabinose,

2. LB medium with addition of 0.2% rhamnose,

3. LB medium with addition of 0.5% IPTG,

4. LB medium with addition of 1% arabinose and 0.2% rhamnose,

5. LB medium with addition of 1% arabinose and 0.5% IPTG,

6. LB medium with addition of 0.2% rhamnose and 0.5% IPTG,

7. LB medium with addition of 1% arabinose, 0.2% rhamnose and 0.5% IPTG,

8. LB medium (control).

After incubation the cells were pelleted, the medium was replaced with phosphate-buffered saline with pH 7.4 (Sigma-Aldrich, USA) with addition of caffeic acid (Sigma-Aldrich, USA) at concentration of 1 g/l, the cells were resuspended by pipetting. Cell bioluminescence was analyzed in half an hour by means of luminometer GloMax 20/20 (Promega, USA). The experiment was repeated in triplicate. Of eight tested samples bioluminescence intensity was significantly different from bioluminescence of test bacteria incubated in LB medium (medium No. 8) only for bacteria incubated in medium No. 7 (LB medium with addition of 1% arabinose, 0.2% rhamnose and 0.5% IPTG). Therefore, bacteria luminescence was indicative of placing the bacteria into the medium ensuring simultaneous activation of three different promoters. In this experiment the bacteria cells integrated information about presence of substances, inducing promotor activity. in external medium and signaled by luminescence only when all three substances were present in the medium simultaneously, performing logical operation “AND” intracellularly.

Synthetic expression cassettes comprising (1) coding sequence of hispidin hydroxylase (SEQ ID NOs: 1, 2) under control of Odf2 promoter according to [Pletz et al., Biochim Biophys Acta. 2013 June; 1833(6):1338-46]; (2) coding sequence of hispidin-synthase (SEQ ID NOs: 34, 35) under control of cycline-dependent kinase CDK7 promoter; (3) luciferases (SEQ ID NOs: 79, 80) under control of CCNH gene promoter were cloned into pmKate2-keratin vector (Evrogen, Russia) instead of sequences of cytomegaloviral promoter abd mKate2-keratin insert. Also, the coding sequence of NpgA gene (SEQ ID NOs: 104, 105) was cloned into pmKate2-keratin vector instead of mKate2-keratin insert sequence. All obtained vectors were co-transfected into HEK293T cells by transfection agent FuGENE HD (Promega, USA) according to the manufacturer's protocol. 24 hours after transfection caffeic acid at concentration of 5 mg/ml was added to the medium and cell luminescence was detected by means of Leica TCS SP8 microscope. Luminescence enabled to identify simultaneous activation of Odf2, CCNH and CDK7 promotors, with luminescence intensity being related to cell cycle stage.

The obtained data indicate that fungal bioluminescent system genes could be used for monitoring simultaneous activation of several promoters, for detecting presence of different substances and their combinations in medium, and also for intracellular logical integration of external signals.

### Example 22. Identification of Hispidin in Plant Extracts

Coding sequences of Neonothopanus nambi hispidin hydroxylase and luciferase, obtained according to Example 1, were cloned into pET23 vector under control of T7 promoter. Purified plasmid DNA products were used for protein transcription and translation in vitro by means of PURExpress In Vitro Protein Synthesis Kit (NEB, USA). The obtained reaction mixture was used for analysis of presence and concentration of hispidin and its functional analogues in lysates of about 19 different plants (Chrysanthemum sp., Ananas comosus, Petunia atkinsiana, Picea abies, Urtica dioica, Solanum lycopersicum, Nicotiana benthamiana, Nicotiana tobacum, Arabidopsis thaliana, Rosa glauca, Rosa rubiginosa, Equisetum arvense, Equisetum telmateia, Polygala sabulosa, Rosa rugosa, Clematis tashiroi, Kalanchoe sp., Triticum aestivum, Dianthus caryophyllus) by adding 2 μl of plant lysate to 100 μl of the reaction mixture and recording luminescence intensity by luminometer GloMax (Promega, USA). It was determined that maximum concentration of hispidin and its functional analogues is in Equisetum arvense and Equisetum telmateia lysates. Hispidin or its functional analogues were also identified in Polygala sabulosa, Rosa rugosa and Clematis tashiroi lysates.

### Example 23. Identification of PKS Able to Catalyze Hispidin Synthesis and their Use for Producing Hispidin In Vitro and In Vivo

Fungal luciferin precursors, such as hispidin, relate to a group of polyketide derivatives. It is known in the art that enzymes involved in polyketide synthesis in plants relate to polyketide synthase protein superfamily, and plant polyketide synthases, in contrast with fungal polyketide synthases, are comparatively compact proteins using CoA ethers of acids, including 3-arylacrylic acids. No polyketide synthase, able to catalyze transformation of caffeic acid CoA ether into hispidin, has been known in the art, however, hispidin is present in many plant organisms.

Using bioinformatic analysis there were selected 11 polyketide synthases potentially able to catalyze hispidin synthesis from the following sources:

Aquilaria sinensis (2 enzymes),
Hydrangea macrophylla,
Arabidopsis thaliana,
Physcomitrella patens,
Polygonum cuspidatum,
Rheum palmatum,
Rheum tataricum,
Wachendorfia thyrsiflora,
Piper methysticum (two enzymes).

The selected nucleotide sequences for expression in Pichia pastoris yeast cells and Nicotiana benthamiana plant cells were optimized. The resulting nucleic acids were obtained synthetically and cloned into pGAPZ vector and used for verifying ability of the expressed proteins to synthesize hispidin.

For this purpose, in genome of Pichia pastoris GS115 yeast line, constitutively expressing Neonothopanus nambi luciferase and hispidin hydroxylase, obtained according to Example 1, there was additionally introduced pGAPZ plasmid, comprising gene of Arabidopsis thaliana coumarate-CoA ligase 1 (which nucleotide and amino acid sequence are shown in SEQ ID NOs: 140, 141), also obtained by oligonucleotide synthesis. The plasmid was linearized at restriction site Avril and used for transformation into Pichia pastoris GS115 cells.

The obtained yeast cells, constitutively expressing Neonothopanus nambi luciferase and hispidin hydroxylase and Arabidopsis thaliana coumarate-CoA ligase 1, were linearized by plasmids comprising coding PKS sequences, and dispersed in Petri dishes with RDB medium, comprising 1 M of sorbitol, 2% (weight/volume) of glucose, 1.34% (weight/volume) of yeast nitrogen base (YNB), 0.005% (weight/volume) of amino acids mixture, 0.00004% (weight/volume) of biotin and 2% (weight/volume) of agar. To identify enzymes having hispidin-synthase activity, the obtained colonies were sprayed with caffeic acid solution, detecting hispidin-synthase presence in cells by occurrence of luminescence. Luminescence emitted by colonies was detected by means of IVIS Spectrum CT (PerkinElmer, USA). Yeast line constitutively expressing luciferase, hispidin hydroxylase and coumarate-CoA ligase 1, and also wild yeast cells were used as negative control. Of tested genes 11 enzymes had hispidin-synthase activity, and their sequence is shown in SEQ ID NOs: 118, 120, 122, 124, 126, 128, 130, 132, 134, 136, 138. Amino acid sequences coded by then are shown in SEQ ID NOs: 119, 121, 123, 125, 127, 129, 131, 133, 135, 137, 139 respectively. The highest activity was demonstrated by enzymes from PKS1 and PKS2 from Aquilaria sinensis (SEQ ID NOs:119, 121), PKS from Arabidopsis thaliana (SEQ ID NO:123) and PKS from Hydrangea macrophylla (SEQ ID NO:125).

Nucleic acid coding PKS from Hydrangea macrophylla (SEQ ID NOs: 124, 125) was used for producing recombinant protein according to the technique described in Example 4. Presence of the expected recombinant product was confirmed by electrophoresis as bands of the expected length were available. Aliquots of the isolated recombinant protein were used for verifying functionality of: 30 μl of isolated recombinant protein solution were put into a glass tube, comprising 100 μl of the buffer (0.2 M of Na-phosphate buffer, 0.5 M of Na₂SO₄, 0.1% dodecylmaltoside (DDM) pH 8.0, all components—Sigma-Aldrich, USA), 0.5 μl of purified recombinant luciferase of Neonothopanus nambi, obtained according to Example 4, 1 mM of NADPH (Sigma-Aldrich, USA), 15 μl of purified recombinant hispidin hydroxylase of Neonothopanus nambi, obtained according to Example 4, 10 mM of ATP (ThermoFisher Scientific, USA), 1 mM of CoA (Sigma-Aldrich, USA), 1 mM of malonyl-CoA (Sigma-Aldrich, USA). The glass tube was placed into a luminometer GloMax 20/20 (Promega, USA). The reaction mixtures demonstrated bioluminescence at adding of 20 μM of caffeyl-CoA into the solution. Maximum emission of the emitted luminescence was 520-535 nm.

Nucleic acid coding PKS2 from Aquilaria sinensis (SEQ ID NO:120, 121) was used for producing hispidin producer strain. For this purpose there were synthesized the expression cassette comprising the nucleic acid SEQ ID NO:120 under control of constitutive J23100 promoter, and expression cassette comprising the nucleic acid SEQ ID NO: 140, coding 4-coumarate-CoA ligase 1 from Arabidopsis thaliana under control of araBAD promoter; bothe expression cassettes were floxed by homology regions to SS9 site. The expression cassettes were cloned into bacterial expression vector, comprising Zeocin resistance cassette, and were used for transformation and integration into E. coli BW25113 genome by means of lambda bacteriophage protein-mediated recombination, as described in Bassalo et al. [ACS Synth Biol. 2016 Jul. 15; 5(7):561-8], using selection for Zeocin resistance. Integration of full-length structure was confirmed by PCR from primers specific to SS9 homology regions, and then the correctness of the integrated structure was verified by sequencing of genomic DNA PCR product by Sanger method.

The obtained E. coli strain was used for producing hispidin. At the first step the bacteria were incubated in five 50 ml plastic tubes in LB medium within 10 hours at 200 rpm fluctuation at 37° C. 250 ml of the obtained culture were added to 3.3 litres of fermentation medium into a fermenter Biostat B5 (Braun, Germany) so that initial culture optical density at 600 nm was about 0.35. Fermentation medium comprised 10 g/l of peptone, 5 g/l of caffeic acid, 5 g/l of yeast extract, 10 g/l of NaCl, 25 g/l of glucose, 15 g/l of (NH₄)₂SO₄, 2 g/l of KH₂PO₄, 2 g/l of MgSO₄.7H₂O, 14.7 mg/l of CaCl₂, 0.1 mg/l of thiamine, 1.8 mg/l and 0.1% of the solution composed of: EDTA 8 mg/l, CoCl₂.6H₂O 2.5 mg/l, MnCl₂.4H₂O 15 mg/l, CuCl₂.2H₂O 1.5 mg/l, HBOs 3 mg/l, Na₂Mo₄.2H₂O 2.5 mg/l, Zn(CH₃COO)₂.2H₂O 13 mg/l, iron (III) citrate 100 mg/l, thiamine hydrochloride 4.5 mg/l. Fermentation was carried out at 37° C., with 3V/min aeration and 200 rpm mixing. After 25 hours of cultivation arabinose was added to the culture up to final concentration of 0.1 mM. pH was automatically controlled by adding of NH₄OH, reducing pH to 7.0. The solution comprising 500 g/i of glucose, 5 g/l of caffeic acid, 2 g/l of arabinose, 25 g/l of trypton, 50 g/l of yeast extract, 17.2 g/l of MgSO₄.7H₂O, 7.5 g/l of (NH₄)SO₄, 18 g/l of ascorbic acid, was added to a fermenter to maintain glucose level every time when pH increased to 7.1. After 56 hours of cultivation the hispidin concentration in the medium was 3.48 g/l. Fermenter medium and also hispidin purified from it by HPLC were active in bioluminescence reaction with Neonothopanus nambi hispidin hydroxylase and luciferase, obtained according to Example 4.

For the purpose of autonomously bioluminescent yeast Pichia pastoris development there were used expression cassettes comprising, under control of GAP promoter and tAOX1 terminator, coding sequences of Neonothopanus nambi luciferase (SEQ ID NOs: 79, 80), Neonothopanus nambi hispidin hydroxylase (SEQ ID NOs: 1, 2), Neonothopanus nambi hispidin-synthase (SEQ ID NOs: 34, 35), Neonothopanus nambi caffeylpyruvate hydrolase (SEQ ID NOs: 64, 65), Rhodobacter capsulatus tyrosine-ammonia-lyase (SEQ ID NOs: 106, 107), and the components HpaB and HpaC of E. coli 4-hydroxyphenyl acetate 3-monooxygenase-reductase (SEQ ID NOs: 108-111), obtained according to Example 15, and also there were synthesized similar expression cassettes comprising coding sequences of 4-coumarate-CoA ligase 1 from Arabidopsis thaliana (SEQ ID NOs: 140, 141) and three PKS: from Aquilaria sinensis (SEQ ID NOs:120, 121), PKS from Arabidopsis thaliana (SEQ ID NOs: 122, 123) and PKS from Hydrangea macrophylla (SEQ ID NO: 124, 125). Each expression cassette was floxed by BsmBI restriction enzyme recognition sequences. Homology regions to MET6 Pichia pastoris gene (Uniprot F2QTU9), floxed by BsmBI restriction enzyme sites, were also obtained syntheticaly. Synthetic DNA was treated by BsmBI restriction enzymes and combined into one plasmid according to Golden Gate cloning protocol, described in [Iverson et al., ACS Synth Biol. 2016 Jan. 15; 5(1):99-103]. There were produced three plasmids different in PKS in their composition. The obtained plasmids were used for producing transgenic yeasts Pichia pastoris according to the technique described in Example 15. Integration of gene cassette into genome was confirmed by PCR from primers annealed at a homology region. All three obtained yeast strains comprising correct genome insert were able to illuminate autonomously in contrast to wild yeast strain.

For development of autonomously bioluminescent flowering plants based on pBI121 vector (Clontech, USA) there was created a set of binary vectors for agrobacterium transformation comprising coding sequences of Neonothopanus nambi luciferase optimized for expression in plants (SEQ ID NO: 112), Neonothopanus nambi hispidin hydroxylase (SEQ ID NO: 103), Neonothopanus nambi caffeylpyruvate hydrolase (SEQ ID NO: 114), kanamycin resistance gene, and PKS (SEQ ID NOs: 122, 123), each gene is under control of 35S promoter from cauliflower mosaic virus. Sequences for expression cassettes assembly were obtained synthetically, the vector was assembled according to Golden Gate cloning protocol, described in [Iverson et al., ACS Synth Biol. 2016 Jan. 15; 5(1):99-103]. Nicotiana tabacum was transformed by co-cultivation of plant tissue with Agrobacterium tumefaciens bacteria of AGL0 strain [Lazo et al., Biotechnology, 1991 October; 9(10):963-7], comprising the created binary vector. Transformation was performed using co-cultivation of Nicotiana tabacum leaf segments. Then, leaves were cut into pieces 0.5 cm long and transferred into 10 ml of liquid Gamborg medium B-5 with 20 g/l of glucose, 0.5 g/l of 2,4-dichlorophenoxyacetic acid and 0.05 g/l of kinetin, and 1.0 ml of agrobacteria overnight culture medium was added. Explants with agrobacteria were co-cultivated within 2-3 minutes. Then, the explants were put on sterile filters in Petri dishes with agarized medium of the same composition. After 48 hours of incubation in a thermostat at 25° C. the explants were transferred to fresh medium with 500 mg/l of cefotaxime and 50 mg/l of kanamycin. In three weeks, regeneration of plants on selective medium, comprising 50 mg/l of kanamycin, was started. Transgenic plants took roots and were transferred to germination medium or soil. Bioluminescence was visualized by means of IVIS Spectrum In Vivo Imaging System (Perkin Elmer). More than % of transgenic plants emitted luminescence minimum by three orders of magnitude exceeding the signal from wild-type plants.

### Example 24. Nucleic Acid Combinations

Combination 1:

Composition: (a) Nucleic acid coding hispidin hydroxylase, which amino acid sequence is selected from the group of SEQ ID NOs: 2, 4, 6, 8 10, 12, 14, 16, 18, 20, 22, 24, 26, 28; and (b) Nucleic acid coding luciferase, which amino acid sequence is selected from the group: 80, 82, 84, 86, 88, 90, 92, 94, 96, 98.

The combination could be used for obtaining bioluminescence in expression systems in vitro or in vivo in the presence of a substance selected from the group of 6-(2-arylvinyl)-4-hydroxy-2H-pyran-2-ones, having the structural formula

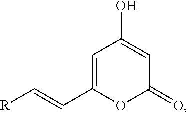

where 6 position substituent is 2-arylvinyl or 2-heteroarylvinyl substituent (R—CH═CH—), including 2-(3,4-dihydroxystyryl), 2-(4-hydroxystyryl), 2-(4-(diethylamino)styryl), 2-(2-(1H-indol-3-yl)vinyl), 2-(2-(1,2,3,5,6,7-hexahydropyrido[3,2,1-ij]quinolin-9-yl)vinyl), 2-(6-hydroxynaphthalene-2-yl)vinyl.

The said combination could also be used in study of two promoters dependency in heterologous expression systems.

The said combination could also be used for identifying hispidin and its analogues in biological objects.

The said combination could also be used for cell labeling by bioluminescence occurring in the presence of hispidin and its functional analogues.

Combination 2:

Composition: (a) Nucleic acid coding hispidin hydroxylase, which amino acid sequence is selected from the group of SEQ ID NOs: 2, 4, 6, 8 10, 12, 14, 16, 18, 20, 22, 24, 26, 28; and (b) Nucleic acid coding hispidin-synthase, which amino acid sequence is selected from the group of SEQ ID NOs: 35, 37, 39, 41, 43, 45, 47, 49, 51, 53, 55.

The combination could be used for producing fungal luciferin in expression systems in vitro or in vivo from a substance selected from substituted acrylic acid with the structural formula

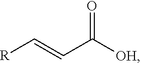

where R is aryl or heteroaryl (e.g. from caffeic acid).

Combination 3:

Includes all the components specified in Combination 2, and also nucleic acid coding luciferase, which amino acid sequence is selected from the group: 80, 82, 84, 86, 88, 90, 92, 94, 96, 98.

The combination could be used for obtaining bioluminescence in expression systems in vitro or in vivo in the presence of a substance selected from substituted acrylic acid with the structural formula

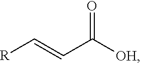

where R is aryl or heteroaryl.

The combination could be used for producing bioluminescent cells and transgenic organisms. The said combination could also be used in study of three promoters dependency in heterologous expression systems.

Combination 4:

Includes all the components specified in Combination 3, and also nucleic acid coding caffeylpyruvate hydrolase, which amino acid sequence is selected from the group of SEQ ID NOs: 65, 67, 69, 71, 73, 75.

The combination could be used for producing bioluminescent cells and transgenic organisms.

Combination 5:

Composition: (a) Nucleic acid coding hispidin-synthase, which amino acid sequence is selected from the group of SEQ ID NOs: 35, 37, 39, 41, 43, 45, 47, 49, 51, 53, 55; and (b) Nucleic acid coding gene of 4′-phosphopantetheinyl transferase, which amino acid sequence is shown in SEQ ID NO: 105

The combination could be used for producing hispidin from caffeic acid in expression systems in vitro and in vivo.

Combination 6:

Composition: (a) Nucleic acid coding hispidin-synthase, which amino acid sequence is selected from the group of SEQ ID NOs: 35, 37, 39, 41, 43, 45, 47, 49, 51, 53, 55; (b) Nucleic acid coding gene of 4′-phosphopantetheinyl transferase, which amino acid sequence is shown in SEQ ID NO: 105; and (c) nucleic acids coding enzymes of 3-arylacrylic acid biosynthesis with the structural formula

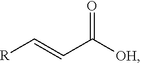

where R is aryl or heteroaryl from cell metabolites (e.g. nucleic acids coding tyrosine-ammonia-lyase and the components HpaB and HpaC of 4-hydroxyphenyl acetate 3-monooxygenase-reductase).

The combination could be used for producing hispidin from tyrosine in expression systems in vitro and in vivo.

Combinations 2-4 could also include the coding sequence of 4′-phosphopantetheinyl transferase NpgA gene (SEQ ID NOs: 104, 105) or other enzyme demonstrating the same activity.

Combination 7:

Composition: (a) Nucleic acid coding hispidin hydroxylase, which amino acid sequence is selected from the group of SEQ ID NOs: 2, 4, 6, 8 10, 12, 14, 16, 18, 20, 22, 24, 26, 28; and (b) Nucleic acid coding PKS, which amino acid sequence is selected from the group of SEQ ID NOs: 119, 121, 123, 125, 127, 129, 131, 133, 135, 137, 139.

The combination could be used for producing 3-hydroxyhispidin from caffeyl-CoA in expression systems in vitro or in vivo.

Combination 8:

Includes all the components specified in Combination 7, and also nucleic acid coding luciferase, which amino acid sequence is selected from the group: 80, 82, 84, 86, 88, 90, 92, 94, 96, 98. The combination could be used for obtaining bioluminescence in vitro or in vivo in the presence of caffeyl-CoA.

Combination 9:

Includes all the components specified in Combination 8, and also nucleic acid coding caffeylpyruvate hydrolase, which amino acid sequence is selected from the group of SEQ ID NOs: 65, 67, 69, 71, 73, 75.

The combination could be used for producing bioluminescent cells and transgenic organisms.

Combination 10:

Composition: (a) Nucleic acid coding PKS, which amino acid sequence is selected from the group of SEQ ID NOs: 119, 121, 123, 125, 127, 129, 131, 133, 135, 137, 139; and (b) Nucleic acid coding 4-coumarate-CoA ligase 1 from Arabidopsis thaliana, which amino acid sequence is shown in SEQ ID NO: 141.

The combination could be used for producing hispidin from caffeic acid in expression systems in vitro and in vivo.

Combination 11:

Includes all the components specified in Combination 10, and also nucleic acids coding enzymes of caffeic acid biosynthesis (e.g. nucleic acids coding tyrosine-ammonia-lyase and the components HpaB and HpaC of 4-hydroxyphenyl acetate 3-monooxygenase-reductase).

The combination could be used for producing hispidin from tyrosine in expression systems in vitro and in vivo.

### Example 25. Combinations of Recombinant Proteins

Combination 1:

Composition: (a) hispidin hydroxylase, which amino acid sequence is selected from the group of SEQ ID NOs: 2, 4, 6, 8 10, 12, 14, 16, 18, 20, 22, 24, 26, 28; and (b) hispidin-synthase, which amino acid sequence is selected from the group of SEQ ID NOs: 35, 37, 39, 41, 43, 45, 47, 49, 51, 53, 55.

The combination could be used for producing fungal luciferin from a substance selected from 3-arylacrylic acid with the structural formula

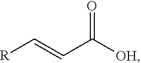

where R is aryl or heteroaryl (e.g. from caffeic acid).

Combination 2:

Includes the components specified in Combination 1, and also luciferase, which amino acid sequence is selected from the group: 80, 82, 84, 86, 88, 90, 92, 94, 96, 98.

The combination could be used for detecting in a sample presence of 3-arylacrylic acid with the structural formula

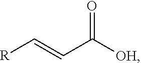

where R is aryl or heteroaryl (e.g. from caffeic acid).

Combination 3:

Composition: (a) hispidin hydroxylase, which amino acid sequence is selected from the group of SEQ ID NOs: 2, 4, 6, 8 10, 12, 14, 16, 18, 20, 22, 24, 26, 28; and (b) PKS, which amino acid sequence is selected from the group of SEQ ID NOs: 119, 121, 123, 125, 127, 129, 131, 133, 135, 137, 139; and (c) 4-coumarate-CoA ligase 1 from Arabidopsis thaliana, which amino acid sequence is shown in SEQ ID NO: 141. The combination could be used for producing fungal luciferin from caffeic acid.

### Example 25. Kits

In the examples below the nucleic acids could be included in the expression cassettes or vectors and operatively cross-linked to regulatory elements for their expression in a host cell. Alternatively, nucleic acids could comprise flanking sequences for its incorporation into the target vector. Nucleic acids could be included in promoter-free vectors intended for easy cloning of target regulatory elements.

Reagent Kit No. 1 includes a purified product of hispidin-synthase of the invention, and it could be used for producing hispidin from caffeic acid. The kit could also be used for producing the other of 6-(2-arylvinyl)-4-hydroxy-2H-pyran-2-one, having the structural formula

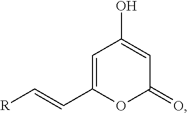

from the corresponding 3-arylacrylic acid with the structural formula

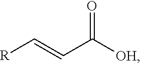

where R is aryl or heteroaryl.

Reagent kit could also include a reaction buffer. For example, 0.2 M sodium phosphate buffer (pH 8.0) laced with 0.5 M of Na₂SO₄, 0.1% of dodecylmaltoside (DDM), 1 mM of NADPH, 10 mM of ATP, 1 mM of CoA, 1 mM of malonyl-CoA, or components for reaction buffer preparation.

Reagent kit could also include deionized water.

Reagent kit could also include directions for use.

Reagent Kit No. 2 includes a purified product of hispidin synthase of the invention and purified product of hispidin hydroxylase of the invention, and it could be used for producing fungal luciferin from a substance selected from 3-arylacrylic acid with the structural formula

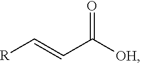

where R is aryl or heteroaryl (e.g. from caffeic acid).

Reagent kit could also include a reaction buffer: 0.2 M sodium phosphate buffer (pH 8.0) laced with 0.5 M of Na₂SO₄, 0.1% of dodecylmaltoside (DDM), 1 mM of NADPH, 10 mM of ATP, 1 mM of CoA, 1 mM of malonyl-CoA, or components for reaction buffer preparation.

Reagent kit could also include deionized water.

Reagent kit could also include directions for use.

Reagent Kit No. 3 includes a purified product of hispidin hydroxylase of the invention, and it could be used for producing fun al luciferin from 6-(2-arylvinyl)-4-hydroxy-2H-pyran-2-one, having the structural formula

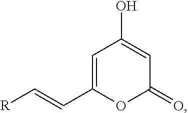

where R is aryl or heteroaryl. For example, the kit could be used for producing 3-hydroxyhispidin from hispidin.

Reagent kit could also include a reaction buffer. For example, 0.2 M sodium phosphate buffer (pH 8.0) laced with 0.5 M of Na₂SO₄, 0.1% of dodecylmaltoside (DDM), 1 mM of NADPH.

Reagent kit could also include deionized water.

Reagent kit could also include directions for use.

Reagent Kit No. 4 and No. 5 differ from the kits No. 2 and No. 3 in that they comprise purified luciferase, which substrate is 6-(2-arylvinyl)-3,4-dihydroxy-2H-pyran-2-one, having the structural formula

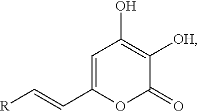

where R is aryl or heteroaryl.

The kits could be used for identifying 3-arylacrylic acid with the structural formula

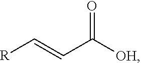

where R is aryl or heteroaryl (e.g. from caffeic acid), and/or 6-(2-arylvinyl)-4-hydroxy-2H-pyran-2-one, having the structural formula

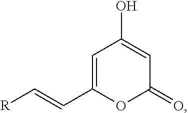

where R is aryl or heteroaryl, (e.g. hispidin) in biological specimens, e.g. in plant extracts, fungal extracts and in microorganisms.

Reagent kits could also include a reaction buffer (see description of kits 2 and 3) for reacting, or components for reaction buffer preparation.

Reagent kit could also include deionized water.

Reagent kit could also include directions for use.

Reagent kit could also include caffeic acid. For example, water solution of caffeic acid or residue for dissolving in water.

Reagent kit could also include hispidin.

Kit Applications

For identification of caffeic acid presence in test specimen it is required to add 5 μl of enzyme mixture to 95 μl of ice-cold reaction buffer in a cuvette, mix carefully, add 5 μl of the test specimen, mix carefully again, and place into a luminometer. Integrate bioluminescent signal within two minutes at maximum 30° C. make control measurements under the same conditions with addition of 5 μl of caffeic acid solution or 5 μl of water instead of the test specimen aliquot. It could be said that caffeic acid is present in the specimen in the detected amounts, if luminescence emitted by the test specimen exceeds a background signal recored from a specimen with water.

Sensitivity: the kit enables to determine presence of caffeic acid in a medium at concentration exceeding 1 nM.

Storage conditions: all kit components should be stored at temperature not exceeding −20° C.

For identification of hispidin presence in test specimen it is required to add 5 μl of enzyme mixture to 95 μl of ice-cold reaction buffer in a cuvette, mix carefully, add 5 μl of the test specimen, mix carefully again, and place into a luminometer. Integrate bioluminescent signal within two minutes at maximum 30° C. make control measurements under the same conditions with addition of 5 μl of hispidin or 5 μl of water instead of the test specimen aliquot. It could be said that hispidin is present in the specimen in the detected amounts, if luminescence emitted by the test specimen exceeds a background signal recored from a specimen with water.

Sensitivity: the kit enables to determine presence of hispidin in a medium at concentration exceeding 100 μM.

Storage conditions: all kit components should be stored at temperature not exceeding −20° C.

Reagent Kit No. 6 includes nucleic acid coding hispidin hydroxylase of the invention. For example, hispidin hydroxylase, which amino acid sequence is selected from the group of SEQ ID NOs: 2, 4, 6, 8 10, 12, 14, 16, 18, 20, 22, 24, 26, 28.

Reagent kit could also comprise directions for use of nucleic acid.

Reagent kit could also comprise deionized water or buffer for dissolving lyophilized nucleic acid and/or diluting nucleic acid solution.

Reagent kit could also comprise primers, complementary to regions of the said nucleic acid, for amplification of nucleic acid or its fragment.

Reagent kit could be used for producing recombinant hispidin hydroxylase of the invention or for hispidin hydroxylase expression in cells and/or cell lines, and/or organisms. After nucleic acid expression in cells, cell lines and/or organisms these cells, cell lines and/or organisms acquire the ability to catalyze transformation of exogenous or endogenous 6-(2-arylvinyl)-4-hydroxy-2H-pyran-2-one, having the structural formula

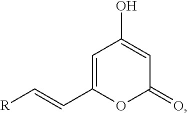

into 6-(2-arylvinyl)-3,4-dihydroxy-2H-pyran-2-one, having the structural formula

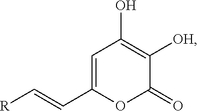

where R is aryl or heteroaryl. For example, they acquire the ability to catalyze transformation of hispidin into 3-hydroxyhispidin.

Reagent Kit No. 7 includes nucleic acid coding hispidin-synthase of the invention. For example, hispidin-synthase, which amino acid sequence is selected from the group of SEQ ID NOs: 35, 37, 39, 41, 43, 45, 47, 49, 51, 53, 55.

Reagent kit could also comprise directions for use of nucleic acid.

Reagent kit could also comprise deionized water or buffer for dissolving lyophilized nucleic acid and/or diluting nucleic acid solution.

Reagent kit could also comprise primers, complementary to regions of the said nucleic acid, for amplification of nucleic acid or its fragment.

Reagent kit could also comprise nucleic acid coding 4′-phosphopantetheinyl transferase, e.g. 4′-phosphopantetheinyl transferase, having amino acid sequence shown in SEQ ID NO 105.

Reagent kit could be used for producing recombinant hispidin synthase of the invention or for hispidin hydroxylase expression in cells and/or cell lines, and/or organisms.

After nucleic acid expression in cells, cell lines and/or organisms these cells, cell lines and/or organisms acquire the ability to catalyze transformation of 3-arylacrylic acid with the structural formula

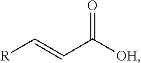

where R is aryl or heteroaryl, into 6-2-arylvinyl)-4-hydroxy-2H-pyran-2-one, having the structural formula

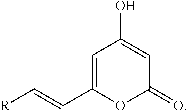

For example, they acquire the ability to catalyze transformation of caffeic acid into hispidin and/or cinnamic acid into (E)-4-hydroxy-6-styryl-2H-pyran-2-one and/or paracoumaric acid into bisnoryangonin and/or (E)-3-(6-hydroxynaphthalen-2-yl) of propenoic acid into (E)-4-hydroxy-6-(2-(6-hydroxynaphthalen-2-yl)vinyl)-2H-pyran-2-one and/or (E)-3-(1H-indol-3-yl) of propenoic acid into (E)-6-(2-(1H-indol-3-yl)vinyl)-4-hydroxy-2H-pyran-2-one.

Reagent kit could also comprise nucleic acids coding tyrosine-ammonia-lyase and the components HpaB and HpaC of 4-hydroxyphenyl acetate 3-monooxygenase-reductase. The kit with such composition could be used for producing hispidin from tyrosine in expression systems in vitro and in vivo.

Reagent Kit No. 8 includes nucleic acid coding hispidin synthase of the invention and nucleic acid coding hispidin hydroxylase of the invention. For example, hispidin-synthase, which amino acid sequence is selected from the group of SEQ ID NOs: 35, 37, 39, 41, 43, 45, 47, 49, 51, 53, 55; and hispidin hydroxylase, which amino acid sequence is selected from the group of SEQ ID NOs: 2, 4, 6, 8 10, 12, 14, 16, 18, 20, 22, 24, 26, 28.

Reagent kit could also include directions for use of nucleic acids.

Reagent kit could also comprise deionized water or buffer for dissolving lyophilized nucleic acid and/or diluting nucleic acid solution.

Reagent kit could also comprise primers, complementary to regions of the nucleic acids included into the kit, for amplification of these nucleic acids or their fragments.

Reagent kit could also comprise nucleic acid coding 4′-phosphopantetheinyl transferase, e.g. 4′-phosphopantetheinyl transferase, having amino acid sequence shown in SEQ ID NO 105.

Reagent kit could also comprise nucleic acids coding enzymes of 3-arylacrylic acid biosynthesis from cell metabolites, e.g. nucleic acids coding tyrosine-ammonia-lyase and the components HpaB and HpaC of 4-hydroxyphenyl acetate 3-monooxygenase-reductase.

The kit could be used for any purposes described for kits 6 and 7. The kit could be used for expression of hispidin hydroxylase and hispidin synthase in cells and/or cell lines, and/or organisms. After nucleic acid expression in cells, cell lines and/or organisms these cells, cell lines and/or organisms acquire the ability to produce 6-(2-arylvinyl)-3.4-dihydroxy-2H-pyran-2-one, having the structural formula

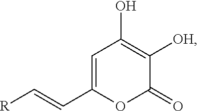

where R is aryl or heteroaryl, from the corresponding 3-arylacrylic acid with the structural formula

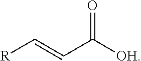

The kit could be used for expression of hispidin hydroxylase and hispidin synthase together with tyrosine-ammonia-lyase and the components HpaB and HpaC of 4-hydroxyphenyl acetate 3-monooxygenase-reductase in cells and/or cell lines, and/or organisms. After nucleic acid expression in cells, cell lines and/or organisms these cells, cell lines and/or organisms acquire the ability to produce hispidin from tyrosine and cell metabolites.

Reagent Kit No. 9 includes nucleic acid coding hispidin hydroxylase of the invention. For example, hispidin hydroxylase, which amino acid sequence is selected from the group of SEQ ID NOs: 2, 4, 6, 8 10, 12, 14, 16, 18, 20, 22, 24, 26, 28 and nucleic acid coding luciferase able to oxidize at least one of fungal luciferins with luminescence emission. For example, there could be selected luciferase, which amino acid sequence is selected from the group of SEQ ID NOs: 80, 82, 84, 86, 88, 90, 92, 94, 96, 98.

Reagent kit could also include directions for use of nucleic acids.

Reagent kit could also comprise deionized water or buffer for dissolving lyophilized nucleic acid and/or diluting nucleic acid solution.

Reagent kit could also comprise primers, complementary to regions of the nucleic acids included into the kit, for amplification of these nucleic acids or their fragments.

The kit could be used for labeling of cells and/or cell lines, and/or organisms, where the said cells, cell lines and/or organisms acquire bioluminescence ability in the presence of exogenous or endogenous fungal preluciferin as a result of expression of the said nucleic acids. For example, they acquire bioluminescence ability in the presence of hispidin.

The kit could be also used for study of target gene promoters co-activation.

The kit could also include a nucleic acid coding hispidin-synthase of the invention, e.g. hispidin-synthase, which amino acid sequence is selected from the group of SEQ ID NOs: 35, 37, 39, 41, 43, 45, 47, 49, 51, 53, 55. In this case the kit could be used for producing cells, cell lines and transgenic organisms capable of bioluminescence in the presence of exogenous or endogenous 3-arylacrylic acid with the structural formula

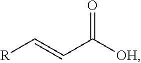

where R is aryl or heteroaryl. For example, in the presence of 3-arylacrylic acid selected from the following group: caffeic acid or cinnamic acid, or paracoumaric acid, or coumaric acid, or umbellic acid, or sinapic acid, or ferulic acid. In particular, the kit could be used for producing autonomously bioluminescent transgenic organisms, e.g. plants or fungi).

The kit could also include a nucleic acid coding 4′-phosphopantetheinyl transferase, e.g. 4′-phosphopantetheinyl transferase, having amino acid sequence shown in SEQ ID NO 105, or similar.

The kit could also comprise nucleic acids coding enzymes of 3-arylacrylic acid biosynthesis from cell metabolites.

The kit could also comprise a nucleic acid coding caffeylpyruvate hydrolase of the invention, e.g. caffeylpyruvate hydrolase, which amino acid sequence is selected from the group of SEQ ID NOs: 65, 67, 69, 71, 73, 75.

The kit could be also used for any purposes described for kits No. 6 and No. 8.

The kit could be also used for producing cell lines enabling to identify caffeic acid in test specimen.

Reagent Kit No. 10 includes Agrobacterium tumefaciens cells of AGL0 strain, carrying plasmid comprising coding sequences of hispidin hydroxylase, hispidin synthase, luciferase, phosphopantetheinyl transferase NpgA gene and antibiotic resistance gene (e.g. kanamycin) under control of suitable promoter, e.g. 35S promoter from cauliflower mosaic virus.

Reagent kit could also include primers for determining correctness of expression cassette integration into dicotyledon flowering plant cells.

Reagent kit could be used for growing autonomously bioluminescent dicotyledon plants.

Reagent kit could also include directions for use.

Method of application: Make transformation of a dicotyledon plant using agrobacteria cells from the kit according to the protocol perfectly suitable for this plant species. Make plant selection in antibiotic medium (e.g. kanamycin). Make correction of expression cassette full-length integration using PCR with kit primers.

Storage conditions: competent agrobacteria cells should be stored at a temperature not exceeding −70° C., it is allowed to store caffeic acid solution at temperatures not exceeding −20° C.

Reagent Kit No. 11

The kit includes a purified product of PKS and a purified product of hispidin hydroxylase of the invention, and it could be used for producing fungal luciferin from caffeyl-CoA. Reagent kit could also include a reaction buffer: 0.2 M sodium phosphate buffer (pH 8.0) laced with 0.5 M of Na₂SO₄, 0.1% of dodecylmaltoside (DDM), 1 mM of NADPH, 10 mM of ATP, 1 mM of malonyl-CoA, or components for reaction buffer preparation. Reagent kit could also include deionized water. Reagent kit could also include directions for use.

Reagent Kit No. 12

The kit includes a nucleic acid coding PKS and a nucleic acid coding hispidin hydroxylase of the invention. For example, PKS which amino acid sequence is selected from the group of SEQ ID NOs: 119, 121, 123, 125, 127, 129, 131, 133, 135, 137, 139 and hispidin hydroxylase, which amino acid sequence is selected from the group of SEQ ID NOs: 2, 4, 6, 8 10, 12, 14, 16, 18, 20, 22, 24, 26, 28.

Reagent kit could also include directions for use of nucleic acids. Reagent kit could also comprise deionized water or buffer for dissolving lyophilized nucleic acid and/or diluting nucleic acid solution. Reagent kit could also comprise primers, complementary to regions of the nucleic acids included into the kit, for amplification of these nucleic acids or their fragments.

Reagent kit could also comprise a nucleic acid coding coumarate-CoA ligase, e.g. coumarate-CoA ligase, having amino acid sequence shown in SEQ ID NO 141.

Reagent kit could also comprise nucleic acids coding enzymes of caffeic acid biosynthesis from cell metabolites, e.g. nucleic acids coding tyrosine-ammonia-lyase and the components HpaB and HpaC of 4-hydroxyphenyl acetate 3-monooxygenase-reductase. Reagent kit could also comprise a nucleic acid coding caffeylpyruvate hydrolase of the invention.

Reagent kit could be used for expression of hispidin hydroxylase and PKS in cells and/or cell lines, and/or organisms. After nucleic acid expression in cells, cell lines and/or organisms these cells, cell lines and/or organisms acquire the ability to produce 3-hydroxyhispidin from caffeic acid. The kit could also be used for expression of hispidin hydroxylase and PKS together with coumarate-CoA ligase, caffeoyl pyruvate hydrolase and/or combination of tyrosine-ammonia-lyase and the components HpaB and HpaC of 4-hydroxyphenyl acetate 3-monooxygenase-reductase in cells and/or cell lines, and/or organisms. After nucleic acid expression in cells, cell lines and/or organisms these cells, cell lines and/or organisms acquire the ability to produce 3-hydroxyhispidin from tyrosine and cell metabolites.

The kit could also comprise a nucleic acid coding luciferase able to oxidize 3-hydroxyhispidin with luminescence emission. In this case the kit could be used for labeling of cells and/or cell lines, and/or organisms, where the said cells, cell lines and/or organisms acquire bioluminescence ability in the presence of exogenous or endogenous hispidin and/or caffeyl-CoA, and/or caffeic acid as a result of expression of the said nucleic acids. For example, cells, cell lines and/or organisms acquire autonomous bioluminescence ability.

1. A fungal luciferin biosynthesis protein selected from the group:
(a) hispidin hydroxylases having the amino acid sequence that within at least 350 amino acids has at least 60% identity with the amino acid sequence selected from the following SEQ ID NOs group: 2, 4, 6, 8, 10, 12, 14, 16, 18, 20, 22, 24, 26, 28, or contains consensus sequences with the SEQ ID NOs 29-33 separated by non-conservative amino acid insertion segments, the hispidin hydroxylase catalyzing conversion of 6-(2-arylvinyl)-4-hydroxy-2H-pyran-2-one with the structural formula 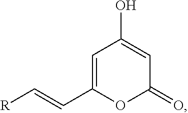
into 6-(2-arylvinyl)-3,4-dihydroxy-2H-pyran-2-one with the structural formula 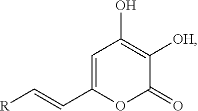
where R is aryl or heteroaryl;
(b) hispidin synthases having the amino acid sequence that has at least 45% identity with the amino acid sequence selected from the following SEQ ID NOs group: 35, 37, 39, 41, 43, 45, 47, 49, 51, 53, 55, or contains consensus sequences with the SEQ ID NOs 56-63 separated by non-conservative amino acid insertion segments, the hispidin synthase catalyzing conversion of 3-aryl acrylic acid with the structural formula 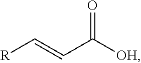
where R is aryl or heteroaryl, into 6-(2-arylvinyl)-4-hydroxy-2H-pyran-2-one with the structural formula 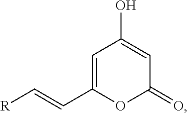
here R is aryl or heteroaryl;
(c) caffeylpyruvate hydroxylases having the amino acid sequence that has at least 60% identity with the amino acid sequence selected from the following SEQ ID NOs group: 65, 67, 69, 71, 73, 75, or contains consensus sequences with the SEQ ID NOs 76-78 separated by non-conservative amino acid insertion segments, the caffeylpyruvate hydroxylase catalyzing conversion of 6-aryl-2-hydroxy-4-oxohexa-2,5-dienoic acid with the structural formula 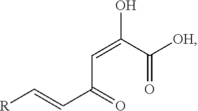
where R is aryl or heteroaryl, conversion into 3-arylacrylic acid with the structural formula

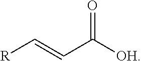

2. The protein according to claim 1, wherein the amino acid sequence of hispidin hydroxylase has at least 65% identity, or at least 70% identity, or at least 75% identity, or at least 80% identity, or at least 85% identity, or at least 90% identity, or at least 95% identity with an amino acid sequence selected from the following group of SEQ ID NOs: 2, 4, 6, 8 10, 12, 14, 16, 18, 20, 22, 24, 26, 28.

3. The protein according to claim 2, wherein the amino acid sequence of hispidin hydroxylase is selected from the following group of SEQ ID NOs: 2, 4, 6, 8 10, 12, 14, 16, 18, 20, 22, 24, 26, 28, or has at least 90%, 91%, 92%, 93%, 94%, 95%, 96%, 97%, 98%, 98%, or 99% identity with it.

4. The protein according to claim 1, wherein the amino acid sequence of hispidin synthase has at least 50% identity, or at least 55% identity, or at least 60% identity, or at least 65% identity, or at least 70% identity, or at least 75% identity, or at least 80% identity, or at least 85% identity, or at least 90% identity, or at least 95% identity with an amino acid sequence selected from the following group of SEQ ID NOs: 35, 37, 39, 41, 43, 45, 47, 49, 51, 53, 55.

5. The protein according to claim 4, wherein the amino acid sequence of hispidin synthase is selected from the following group of SEQ ID NOs: 35, 37, 39, 41, 43, 45, 47, 49, 51, 53, 55, or has at least 90%, 91%, 92%, 93%, 94%, 95%, 96%, 97%, 98%, 98%, or 99% identity with it.

6. The protein according to claim 1, wherein the amino acid sequence of caffeylpyruvate hydrolase has at least 65% identity, or at least 70% identity, or at least 75% identity, or at least 80% identity, or at least 85% identity, or at least 90% identity, or at least 95% identity with an amino acid sequence selected from the following group of SEQ ID NOs: 65, 67, 69, 71, 73, 75.

7. The protein according to claim 6, wherein the amino acid sequence of caffeylpyruvate synthase is selected from the following group of SEQ ID NOs: 65, 67, 69, 71, 73, 75, or has at least 90%, 91%, 92%, 93%, 94%, 95%, 96%, 97%, 98%, 98%, or 99% identity with it.

8. A fusion protein, which comprises operatively cross-linked hispidin hydroxylase, and/or hispidin synthase, and/or caffeylpyruvate hydrolase according to claim 1, and luciferase capable to oxidize fungal luciferin with light emission, and/or intracellular localization signal, and/or signal peptide.

9. The fusion protein according to claim 8, wherein amino acid sequence of luciferase is at least 40% identical, for example, at least 45% identical, or at least 50% identical, or at least 55% identical, or at least 60% identical, or at least 70% identical, or at least 75% identical, or at least 80% identical, or at least 85% identical to an amino acid sequence selected from the following SEQ ID NOs group: 80, 82, 84, 86, 88, 90, 92, 94, 96, 98.

10. The fusion protein according to claim 9, wherein the amino acid sequence has the SEQ ID No. 101.

11. The protein according to claim 1, wherein 6-(2-arylvinyl)-4-hydroxy-2H-pyran-2-one is selected from the group:
(E)-6-(3,4-dihydroxystyryl)-4-hydroxy-2H-pyran-2-one,
(E)-4-dihydroxy-6-styryl-2H-pyran-2-one,
(E)-4-hydroxy-6-(4-hydroxystyryl)-2H-pyran-2-one,
(E)-4-hydroxy-6-(2-hydroxystyryl)-2H-pyran-2-one,
(E)-4-hydroxy-6-(2,4-dihydroxystyryl)-2H-pyran-2-one,
(E)-4-hydroxy-6-(4-hydroxy-3,5-dimethoxystyryl)-2H-pyran-2-one,
(E)-4-hydroxy-6-(4-hydroxy-3-methoxystyryl)-2H-pyran-2-one,
(E)-4-hydroxy-6-(2-(6-hydroxynaphthalen-2-yl)vinyl)-2H-pyran-2-one,
(E)-6-(4-aminostyryl)-4-hydroxy-2H-pyran-2-one,
(E)-6-(4-(diethylamino)styryl)-4-hydroxy-2H-pyran-2-one,
(E)-6-(2-(1H-indol-3-yl)vinyl)-4-hydroxy-2H-pyran-2-one,
(E)-4-hydroxy-6-(2,3,6,7-tetrahydro-1H,5H-pyrido[3,2,1-ij]quinolin-9-yl)vinyl)-2H-pyran-2-one.

12. The protein according to claim 1, wherein 3-aryl acrylic acid is selected from the group comprising: caffeic acid, cinnamic acid, paracoumaric acid, coumaric acid, umbellic acid, sinapic acid, and ferulic acid.

13. A use of fungal luciferin biosynthesis protein selected from the group:
(a) the amino acid sequence that within at least 350 amino acids has at least 60% identity with the amino acid sequence selected from the following SEQ ID NOs group: 2, 4, 6, 8, 10, 12, 14, 16, 18, 20, 22, 24, 26, 28, or contains consensus sequences with the SEQ ID NOs 29-33 separated by non-conservative amino acid insertion segments, as hispidin hydroxylases catalyzing conversion of 6-(2-arylvinyl)-4-hydroxy-2H-pyran-2-one with the structural formula 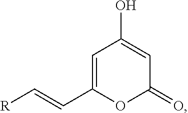
into 6-(2-arylvinyl)-3,4-dihydroxy-2H-pyran-2-one with the structural formula 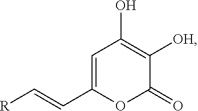
where R is aryl or heteroaryl;
(b) the amino acid sequence that has at least 45% identity with the amino acid sequence selected from the following SEQ ID NOs group: 35, 37, 39, 41, 43, 45, 47, 49, 51, 53, 55, or contains consensus sequences with the SEQ ID NOs 56-63 separated by non-conservative amino acid insertion segments, as hispidin synthases catalyzing conversion of 3-aryl acrylic acid with the structural formula 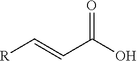
into 6-(2-arylvinyl)-4-hydroxy-2H-pyran-2-one with the structural formula 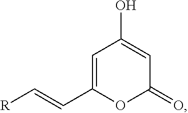
where R is aryl or heteroaryl;
(c) the amino acid sequence that has at least 60% identity with the amino acid sequence selected from the following SEQ ID NOs group: 65, 67, 69, 71, 73, 75, or contains consensus sequences with the SEQ ID NOs 76-78 separated by non-conservative amino acid insertion segments, as caffeylpyruvate hydroxylase catalyzing conversion of 6-aryl-2-hydroxy-4-oxohexa-2,5-dienoic acid with the structural formula 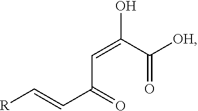
where R is aryl or heteroaryl, conversion into 3-arylacrylic acid with the structural formula

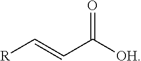

14. The use according to claim 13, wherein the amino acid sequence of hispidin hydroxylase has at least 65% identity, or at least 70% identity, or at least 75% identity, or at least 80% identity, or at least 85% identity, or at least 90% identity, or at least 95% identity with an amino acid sequence selected from the following group of SEQ ID NOs: 2, 4, 6, 8 10, 12, 14, 16, 18, 20, 22, 24, 26, 28.

15. The use according to claim 13, wherein the amino acid sequence of hispidin synthase has at least 50% identity, or at least 55% identity, or at least 60% identity, or at least 65% identity, or at least 70% identity, or at least 75% identity, or at least 80% identity, or at least 85% identity, or at least 90% identity, or at least 95% identity with an amino acid sequence selected from the following group of SEQ ID NOs: 35, 37, 39, 41, 43, 45, 47, 49, 51, 53, 55.

16. The use according to claim 13, wherein the amino acid sequence of caffeylpyruvate hydrolase has at least 65% identity, or at least 70% identity, or at least 75% identity, or at least 80% identity, or at least 85% identity, or at least 90% identity, or at least 95% identity with an amino acid sequence selected from the following group of SEQ ID NOs: 65, 67, 69, 71, 73, 75.

17. The use according to claim 13, wherein 6-(2-arylvinyl)-4-hydroxy-2H-pyran-2-one is selected from the group:
(E)-6-(3,4-dihydroxystyryl)-4-hydroxy-2H-pyran-2-one (hispidin),
(E)-4-dihydroxy-6-styryl-2H-pyran-2-one,
(E)-4-hydroxy-6-(4-hydroxystyryl)-2H-pyran-2-one (bisnoryangonin),
(E)-4-hydroxy-6-(2-hydroxystyryl)-2H-pyran-2-one,
(E)-4-hydroxy-6-(2,4-dihydroxystyryl)-2H-pyran-2-one,
(E)-4-hydroxy-6-(4-hydroxy-3,5-dimethoxystyryl)-2H-pyran-2-one,
(E)-4-hydroxy-6-(4-hydroxy-3-methoxystyryl)-2H-pyran-2-one,
(E)-4-hydroxy-6-(2-(6-hydroxynaphthalen-2-yl)vinyl)-2H-pyran-2-one,
(E)-6-(4-aminostyryl)-4-hydroxy-2H-pyran-2-one,
(E)-6-(4-(diethylamino)styryl)-4-hydroxy-2H-pyran-2-one,
(E)-6-(2-(1H-indol-3-yl)vinyl)-4-hydroxy-2H-pyran-2-one,
(E)-4-hydroxy-6-(2,3,6,7-tetrahydro-1H,5H-pyrido[3,2,1-ij]quinolin-9-yl)vinyl)-2H-pyran-2-one.

18. The use according to claim 13, wherein 3-aryl acrylic acid is selected from the group comprising: caffeic acid, cinnamic acid, paracoumaric acid, coumaric acid, umbellic acid, sinapic acid, and ferulic acid.

19. A nucleic acid encoding the fungal luciferin biosynthesis protein according to claim 1, selected from the group:
(a) hispidin hydroxylases having the amino acid sequence that within at least 350 amino acids has at least 60% identity with the amino acid sequence selected from the following SEQ ID NOs group: 2, 4, 6, 8, 10, 12, 14, 16, 18, 20, 22, 24, 26, 28, or contains consensus sequences with the SEQ ID NOs 29-33 separated by non-conservative amino acid insertion segments, the hispidin hydroxylase catalyzing conversion of 6-(2-arylvinyl)-4-hydroxy-2H-pyran-2-one with the structural formula 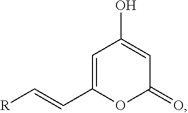
into 6-(2-arylvinyl)-3,4-dihydroxy-2H-pyran-2-one with the structural formula 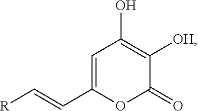
where R is aryl or heteroaryl;
(b) hispidin synthases having the amino acid sequence that has at least 45% identity with the amino acid sequence selected from the following SEQ ID NOs group: 35, 37, 39, 41, 43, 45, 47, 49, 51, 53, 55, or contains consensus sequences with the SEQ ID NOs 56-63 separated by non-conservative amino acid insertion segments, the hispidin synthase catalyzing conversion of 3-aryl acrylic acid with the structural formula 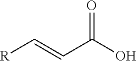
into 6-(2-arylvinyl)-4-hydroxy-2H-pyran-2-one with the structural formula 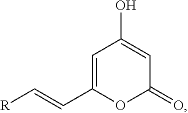
where R is aryl or heteroaryl;
(c) caffeylpyruvate hydroxylases having the amino acid sequence that has at least 60% identity with the amino acid sequence selected from the following SEQ ID NOs group: 65, 67, 69, 71, 73, 75, or contains consensus sequences with the SEQ ID NOs 76-78 separated by non-conservative amino acid insertion segments, the caffeylpyruvate hydroxylase catalyzing conversion of 6-aryl-2-hydroxy-4-oxohexa-2,5-dienoic acid with the structural formula 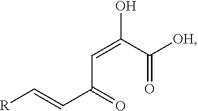
where R is aryl or heteroaryl, conversion into 3-arylacrylic acid with the structural formula

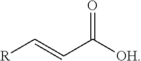

20. The nucleic acid according to claim 19, wherein the amino acid sequence of hispidin hydroxylase has at least 65% identity, or at least 70% identity, or at least 75% identity, or at least 80% identity, or at least 85% identity, or at least 90% identity, or at least 95% identity with an amino acid sequence selected from the following group of SEQ ID NOs: 2, 4, 6, 8 10, 12, 14, 16, 18, 20, 22, 24, 26, 28.

21. The nucleic acid according to claim 20, wherein the amino acid sequence is selected from the following group of SEQ ID NOs: 2, 4, 6, 8 10, 12, 14, 16, 18, 20, 22, 24, 26, 28, or has at least 96%, 97%, 98%, 98%, or 99% identity with it.

22. The nucleic acid according to claim 19, wherein the amino acid sequence of hispidin synthase has at least 50% identity, or at least 55% identity, or at least 60% identity, or at least 65% identity, or at least 70% identity, or at least 75% identity, or at least 80% identity, or at least 85% identity, or at least 90% identity, or at least 95% identity with an amino acid sequence selected from the following group of SEQ ID NOs: 35, 37, 39, 41, 43, 45, 47, 49, 51, 53, 55.

23. The nucleic acid according to claim 22, wherein the amino acid sequence of hispidin synthase is selected from the following group of SEQ ID NOs: 35, 37, 39, 41, 43, 45, 20, 49, 51, 53, 55, or has at least 96%, 97%, 98%, 98%, or 99% identity with it.

24. The nucleic acid according to claim 19, wherein the amino acid sequence of caffeylpyruvate hydrolase has at least 65% identity, or at least 70% identity, or at least 75% identity, or at least 80% identity, or at least 85% identity, or at least 90% identity, or at least 95% identity with an amino acid sequence selected from the following group of SEQ ID NOs: 65, 67, 69, 71, 73, 75.

25. The nucleic acid according to claim 24, wherein the amino acid sequence of caffeylpyruvate synthase is selected from the following group of SEQ ID NOs: 65, 67, 69, 71, 73, 75, or has at least 96%, 97%, 98%, 98%, or 99% identity with it.

26. The nucleic acid encoding the fusion protein according to claim 8.

27. The nucleic acid according to claim 19, wherein 6-(2-arylvinyl)-4-hydroxy-2H-pyran-2-one is selected from the group:
(E)-6-(3,4-dihydroxystyryl)-4-hydroxy-2H-pyran-2-one (hispidin),
(E)-4-dihydroxy-6-styryl-2H-pyran-2-one,
(E)-4-hydroxy-6-(4-hydroxystyryl)-2H-pyran-2-one (bisnoryangonin),
(E)-4-hydroxy-6-(2-hydroxystyryl)-2H-pyran-2-one,
(E)-4-hydroxy-6-(2,4-dihydroxystyryl)-2H-pyran-2-one,
(E)-4-hydroxy-6-(4-hydroxy-3,5-dimethoxystyryl)-2H-pyran-2-one,
(E)-4-hydroxy-6-(4-hydroxy-3-methoxystyryl)-2H-pyran-2-one,
(E)-4-hydroxy-6-(2-(6-hydroxynaphthalen-2-yl)vinyl)-2H-pyran-2-one,
(E)-6-(4-aminostyryl)-4-hydroxy-2H-pyran-2-one,
(E)-6-(4-(diethylamino)styryl)-4-hydroxy-2H-pyran-2-one,
(E)-6-(2-(1H-indol-3-yl)vinyl)-4-hydroxy-2H-pyran-2-one,
(E)-4-hydroxy-6-(2,3,6,7-tetrahydro-1H,5H-pyrido[3,2,1-ij]quinolin-9-yl)vinyl)-2H-pyran-2-one.

28. The nucleic acid according to claim 19, wherein 3-aryl acrylic acid is selected from the group comprising: caffeic acid, cinnamic acid, paracoumaric acid, coumaric acid, umbellic acid, sinapic acid, and ferulic acid.

29. An expression cassette comprising: (a) a domain of transcription initiation, which is functional in a host cell; (b) the nucleic acid according to claim 19, and (c) a domain of transcription termination, which is functional in the host cell.

30. A host cell containing at least one expression cassette according to claim 29 as a part of an extrachromosomal element or integrated into the cell genome as a result of introducing said cassette into said cell, wherein said cell expresses at least one of the functional proteins for fungal luciferin biosynthesis.

31. An antibody that binds to at least one protein according to claim 1.

32. A use of a nucleic acid encoding a fungal luciferin biosynthesis protein selected from the group:
(a) the amino acid sequence that within at least 350 amino acids has at least 60% identity with the amino acid sequence selected from the following SEQ ID NOs group: 2, 4, 6, 8, 10, 12, 14, 16, 18, 20, 22, 24, 26, 28, or contains consensus sequences with the SEQ ID NOs 29-33 separated by non-conservative amino acid insertion segments, to produce in in vitro or in vivo systems the hispidin hydroxylase catalyzing the reaction of 6-(2-arylvinyl)-4-hydroxy-2H-pyran-2-one with the structural formula 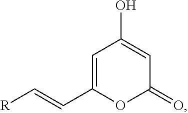
conversion into 6-(2-arylvinyl)-3,4-dihydroxy-2H-pyran-2-one with the structural formula 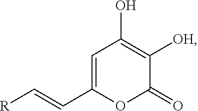
where R is aryl or heteroaryl;
(b) the amino acid sequence that has at least 45% identity with the amino acid sequence selected from the following SEQ ID NOs group: 35, 37, 39, 41, 43, 45, 47, 49, 51, 53, 55, or contains consensus sequences with the SEQ ID NOs 56-63 separated by non-conservative amino acid insertion segments, to produce in in vitro or in vivo systems the hispidin synthase catalyzing the reaction of conversion 3-aryl acrylic acid with the structural formula 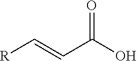
into 6-(2-arylvinyl)-4-hydroxy-2H-pyran-2-one with the structural formula 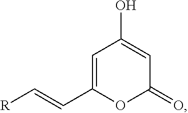
where R is aryl or heteroaryl;
(c) the amino acid sequence that has at least 60% identity with the amino acid sequence selected from the following SEQ ID NOs group: 65, 67, 69, 71, 73, 75, or contains consensus sequences with the SEQ ID NOs 76-78 separated by non-conservative amino acid insertion segments, to produce in in vitro or in vivo systems the caffeylpyruvate hydrolase catalyzing the reaction of an 6-aryl-2-hydroxy-4-oxohexa-2,5-dienoic acid with the structural formula 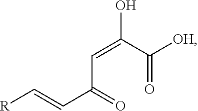
where R is aryl or heteroaryl, conversion into 3-arylacrylic acid with the structural formula

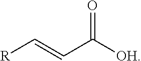

33. The use according to claim 32, wherein the nucleic acid is used to express a fungal luciferin biosynthesis protein contained in an expression cassette that also comprises a domain of transcription initiation, which is functional in a host cell and a domain of transcription termination, which is functional in the host cell.

34. The use according to claim 33, wherein the expression cassette is used in a host cell.

35. A method of biosynthesis a fungal luciferin with the chemical formula 6-(2-arylvinyl)-3,4-dihydroxy-2H-pyran-2-one and the structural formula 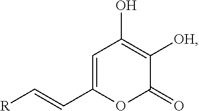
where R is aryl or heteroaryl, in either in vitro or in vivo system, which comprises combining at least one moiety of hispidin hydroxylase according to claim 1 with at least one moiety of 6-(2-arylvinyl)-4-hydroxy-2H-pyran-2-one having the structural formula 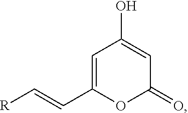
at least one NAD(P)H moiety, and at least one molecular oxygen moiety under physiological conditions.

36. The method according to claim 35, wherein the reaction is performed in a cell or organism, the method comprising introducing into the cell of the expression cassette that contains a hispidin hydroxylase encoding nucleic acid.

37. The method according to claim 36, comprising introducing into a cell or organism the expression cassette further containing: (a) a domain of transcription initiation, which is functional in a host cell; (b) a nucleic acid, which encodes the luciferase capable to oxidize fungal luciferin with light emission, and (c) a domain of transcription termination, which is functional in the host cell, wherein the said cell or organism acquires the ability to bioluminescence in the presence of said fungal luciferin.

38. The method according to claim 37, wherein the nucleic acid, which encodes the luciferase, is operatively fused with the nucleic acid, which encodes the hispidin hydroxylase to form the nucleic acid.

39. A method of biosynthesis a fungal luciferin with the chemical formula 6-(2-arylvinyl)-4-hydroxy-2H-pyran-2-one and the structural formula 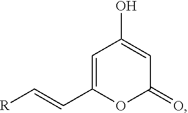
where R is aryl or heteroaryl, in either in vitro or in vivo system, which comprises combining at least one moiety of 3-aryl acrylic acid with the structural formula 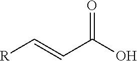
with at least one moiety of hispidin synthase according to claim 1, at least one moiety of coenzyme A, at least one ATP moiety, and at least two malonyl-CoA moieties under physiological conditions.

40. The method according to claim 39, wherein the reaction is performed in a cell or organism, the method comprising introducing into the cell of the expression cassette that contains a hispidin synthase encoding nucleic acid.

41. The method according to claim 40, further comprising introducing into the cell or organism a nucleic acid encoding a 4′-phosphopantotheinyl transferase and capable to transfer the 4-phosphopantotheinyl from coenzyme A to serine in the acyl transfer domain of polyketide synthases.

42. The method according to claim 41, wherein the 4′-phosphopantotheinyl transferase has an amino acid sequence at least 40% identical to amino acid sequence with SEQ ID No. 105.

43. The method according to claim 40, further comprising introducing into the cell or organism nucleic acids, which encode enzymes for biosynthesis of 3-aryl acrylic acid from cell metabolites with the structural formula 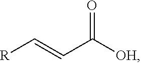
where R is aryl or heteroaryl.

44. The method according to claim 43, wherein the enzymes for biosynthesis of 3-aryl acrylic acid are selected from the group of:
(a) tyrosine ammonia-lyase with an amino acid sequence at least 40% identical to the amino acid sequence having SEQ ID No. 107; HpaB and HpaC components of 4-hydroxyphenylacetate 3-monooxygenase reductase at least 40% identical to the amino acid sequences of HpaB and HpaC components of 4-hydroxyphenylacetate 3-monooxygenase reductase of E. coli having SEQ ID NOs 109 and 111;
(b) phenylalanine ammonia-lyase with an amino acid sequence at least 40% identical to the amino acid sequence having SEQ ID No.117.

45. A method of biosynthesis a fungal luciferin with the chemical formula 6-(2-arylvinyl)-3,4-dihydroxy-2H-pyran-2-one and the structural formula 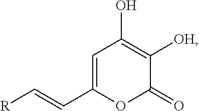
where R is aryl or heteroaryl, in either in vitro or in vivo system, which comprises combining at least one moiety of 3-aryl acrylic acid having the structural formula 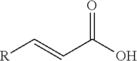
with at least one moiety of hispidin synthase according to claim 1; at least one moiety of coenzyme A; at least one ATP moiety; at least two malonyl-CoA moieties; at least one moiety of hispidin hydroxylase; at least one NAD(P)H moiety, and at least one molecular oxygen moiety under physiological conditions.

46. The method according to claim 45, wherein the reaction is performed in a cell or organism, the method comprising introducing into the cell of the expression cassette that contains a hispidin synthase encoding nucleic acid, and the expression cassette that contains a hispidin hydroxylase encoding nucleic acid.

47. The method according to claim 46, further comprising introducing into the cell or organism a nucleic acid encoding a 4′-phosphopantotheinyl transferase and capable to transfer the 4-phosphopantotheinyl from coenzyme A to serine in the acyl transfer domain of polyketide synthases.

48. The method according to claim 41, wherein the 4′-phosphopantotheinyl transferase has an amino acid sequence at least 40% identical to amino acid sequence with SEQ ID No. 105.

49. The method according to claim 46, further comprising introducing into the cell or organism nucleic acids, which encode enzymes for biosynthesis of 3-aryl acrylic acid from cell metabolites with the structural formula 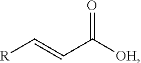
where R is aryl or heteroaryl.

50. The method according to claim 49, wherein the enzymes for biosynthesis of 3-aryl acrylic acid are selected from the group of:
(a) tyrosine ammonia-lyase with an amino acid sequence at least 40% identical to the amino acid SEQ ID No. 107; HpaB and HpaC components of 4-hydroxyphenylacetate 3-monooxygenase reductase at least 40% identical to the amino acid sequences of HpaB and HpaC components of 4-hydroxyphenylacetate 3-monooxygenase reductase of E. coli having SEQ ID NOs 109 and 111;
(b) phenylalanine ammonia-lyase with an amino acid sequence at least 40% identical to the amino acid sequence having SEQ ID No.117.

51. A method of producing transgenic bioluminescent cell or organism comprising introducing an expression cassette according to claim 29 into the cell or organism, said expression cassette comprising a hispidin hydroxylase encoding nucleic acid and containing (a) a domain of transcription initiation, which is functional in a host cell; (b) a nucleic acid, which encodes the luciferase capable to oxidize fungal luciferin with light emission, and (c) a domain of transcription termination, which is functional in the host cell, wherein said cell acquires the ability to bioluminescence in the presence of fungal preluciferin with the chemical formula 6-(2-arylvinyl)-4-hydroxy-2H-pyran-2-one and structural formula 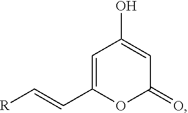
where R is aryl or heteroaryl.

52. The method according to claim 51, further comprising introducing into the cell or organism a nucleic acid encoding the hispidin synthase, as a part of an expression cassette, wherein said cell acquires the ability to bioluminescence in the presence of an exogenous or endogenous precursor of fungal preluciferin, which is 3-aryl acrylic acid with the structural formula 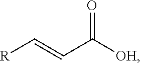
where R is aryl or heteroaryl.

53. The method according to claim 52, further comprising introducing into the cell or organism a nucleic acid encoding the caffeylpyruvate hydrolase.

54. The method according to claim 52, further comprising introducing into the cell or organism a nucleic acid encoding the 4′-phosphopantotheinyl transferase and capable to transfer the 4-phosphopantotheinyl from coenzyme A to serine in the acyl transfer domain of polyketide synthases.

55. The method according to claim 52, further comprising introducing into the cell or organism nucleic acids, which encode enzymes for biosynthesis of 3-aryl acrylic acid from cell metabolites.

56. The method according to claim 55, wherein the enzymes for biosynthesis of 3-aryl acrylic acid are selected from the group of:
(a) tyrosine ammonia-lyase with an amino acid sequence at least 40% identical to the amino acid SEQ ID No. 107; HpaB and HpaC components of 4-hydroxyphenylacetate 3-monooxygenase reductase at least 40% identical to the amino acid sequences of HpaB and HpaC components of 4-hydroxyphenylacetate 3-monooxygenase reductase of E. coli having SEQ ID NOs 109 and 111;
(b) phenylalanine ammonia-lyase with an amino acid sequence at least 40% identical to the amino acid sequence having SEQ ID No.117.

57. A method of producing transgenic bioluminescent cell or organism comprising introducing a nucleic acid according to claim 26 in the form of an expression cassette into the cell or organism, wherein said cell acquires the ability to bioluminescence in the presence of fungal preluciferin with the chemical formula 6-(2-arylvinyl)-4-hydroxy-2H-pyran-2-one and structural formula 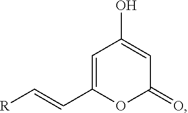
where R is aryl or heteroaryl.

58. The method according to claim 57, further comprising introducing into the cell or organism a nucleic acid encoding the hispidin synthase, as a part of an expression cassette, wherein said cell acquires the ability to bioluminescence in the presence of an exogenous or endogenous precursor of fungal preluciferin, which is 3-aryl acrylic acid with the structural formula 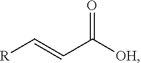
where R is aryl or heteroaryl.

59. The method according to claim 58, further comprising introducing into the cell or organism a nucleic acid encoding the caffeylpyruvate hydrolase.

60. The method according to claim 58, further comprising introducing into the cell or organism a nucleic acid encoding the 4′-phosphopantotheinyl transferase and capable to transfer the 4-phosphopantotheinyl from coenzyme A to serine in the acyl transfer domain of polyketide synthases.

61. The method according to claim 58, further comprising introducing into the cell or organism nucleic acids, which encode enzymes for biosynthesis of 3-aryl acrylic acid from cell metabolites.

62. The method according to claim 61, wherein the enzymes for biosynthesis of 3-aryl acrylic acid are selected from the group of:
(a) tyrosine ammonia-lyase with an amino acid sequence at least 40% identical to the amino acid SEQ ID No. 107; HpaB and HpaC components of 4-hydroxyphenylacetate 3-monooxygenase reductase at least 40% identical to the amino acid sequences of HpaB and HpaC components of 4-hydroxyphenylacetate 3-monooxygenase reductase of E. coli having SEQ ID NOs 109 and 111;
(b) phenylalanine ammonia-lyase with an amino acid sequence at least 40% identical to the amino acid sequence having SEQ ID No.117.

63. A transgenic organism capable of bioluminescence in the presence of fungal luciferin and/or fungal preluciferin, containing at least a nucleic acid encoding the hispidin hydroxylase according to claim 19 as a part of an extrachromosomal element or integrated into genome of a cell as a result of introducing an expression cassette into said cell and a nucleic acid encoding the luciferase capable to oxidize fungal luciferin with light emission.

64. A transgenic organism capable of autonomous bioluminescence, wherein said organism contains at least a nucleic acid encoding the hispidin hydroxylase claim 19; a nucleic acid encoding the hispidin synthase as a part of an extrachromosomal element or integrated into genome of a cell as a result of introducing an expression cassette into said cell, and a nucleic acid encoding the luciferase capable to oxidize fungal luciferin with light emission.

65. The transgenic organism according to claim 64, which contains a nucleic acid encoding the caffeylpyruvate hydrolase.

66. A vector for transferring a nucleic acid into a host cell comprising at least one nucleic acid according to claim 19.

67. A kit for producing fungal luciferin and/or fungal preluciferin comprising hispidin hydroxylase and hispidin synthase according to claim 1.

68. The kit for producing fungal luciferin and/or fungal preluciferin in in vitro and/or in vivo systems, comprising a nucleic acid encoding the hispidin hydroxylase and a nucleic acid encoding the hispidin synthase according to claim 19.

69. A kit for producing a bioluminescent cell or organism, comprising a nucleic acid encoding the hispidin hydroxylase, a nucleic acid encoding the hispidin synthase according to claim 19, and a nucleic acid encoding the luciferase capable to oxidize fungal luciferin with light emission.

70. The kit according to claim 69, further containing a nucleic acid, encoding a caffeylpyruvate hydrolase.

71. The kit according to claim 68 further comprising a nucleic acid encoding the 4′-phosphopantotheinyl transferase and/or nucleic acids encoding enzymes for biosynthesizing the 3-aryl acrylic acid.

72. An use of polyketide synthase with amino acid sequence that is at least 40%, or at least 45%, or at least 50%, or at least 55%, or at least 60%, at least 65%, or at least 70%, or at least 80%, or at least 85%, or at least 90%, or at least 91%, or at least 92%, or at least 93%, or at least 94%, or at least 95%, or at least 96%, or at least 97%, or at least 98%, or at least 99%, or completely identical to a sequence selected from the following SEQ ID NOs group: 119, 121, 123, 125, 127, 129, 131, 133, 135, 137, 139, to produce hispidin in an in vitro or in vivo system.

73. A method of producing hispidin in an in vitro or in vivo system comprising combining at least one PKS moiety according to claim 72 with at least two malonyl-CoA moieties and at least one caffeyl-CoA moiety under physiological conditions.

74. The method according to claim 73, wherein at least one caffeic acid moiety, at least one coenzyme A moiety, at least one coumarate-CoA ligase moiety, and at least one ATP moiety are added to the reaction mixture instead of at least one moiety of caffeyl-CoA.

75. The method according to claim 73, wherein the reaction is performed in a cell or organism, the method comprising introducing the expression cassette that contains a nucleic acid encoding the type III polyketide synthase.

76. The method according to claim 75, further comprising introducing a nucleic acid encoding the coumarate-CoA ligase into the cell or organism.

77. The method according to claim 76, wherein the coumarate-CoA ligase has an amino acid sequence that is at least 40%, or at least 45%, or at least 50%, or at least 55%, or at least 60%, or at least 65%, or at least 70%, or at least 80%, or at least 85%, or at least 90%, or at least 91%, or at least 92%, or at least 93%, or at least 94%, or at least 95%, or at least 96%, or at least 97%, or at least 98%, or at least 99%, or completely identical to the sequence with SEQ ID No. 141.

78. The method according to claim 75, further comprising introducing into the cell or organism nucleic acids, which encode enzymes for biosynthesis of caffeic acid.

79. The method according to claim 78, wherein the enzymes for biosynthesis of 3-aryl acrylic acid are selected from the group of:
(a) tyrosine ammonia-lyase with an amino acid sequence at least 40% identical to the amino acid SEQ ID No. 107; HpaB and HpaC components of 4-hydroxyphenylacetate 3-monooxygenase reductase at least 40% identical to the amino acid sequences of HpaB and HpaC components of 4-hydroxyphenylacetate 3-monooxygenase reductase of E. coli having SEQ ID NOs 109 and 111;
(b) phenylalanine ammonia-lyase with an amino acid sequence at least 40% identical to the amino acid sequence having SEQ ID No.117.

80. The method according to claim 51, further comprising introducing into a cell or organism an expression cassette containing: (a) a domain of transcription initiation, which is functional in a host cell; (b) a nucleic acid, which encodes the type III polyketide synthase, and (c) a domain of transcription termination, which is functional in the host cell, wherein said cell acquires the ability to bioluminescence in the presence of exogenous or endogenous caffeyl-CoA.

81. The method according to claim 80, further comprising introducing into a cell or organism an expression cassette containing: (a) a domain of transcription initiation, which is functional in a host cell; (b) a nucleic acid, which encodes the coumarate-CoA ligase, and (c) a domain of transcription termination, which is functional in the host cell, wherein said cell acquires the ability to bioluminescence in the presence of caffeic acid.

82. The method according to claim 81, wherein the coumarate-CoA ligase has an amino acid sequence that is at least 40%, or at least 45%, or at least 50%, or at least 55%, or at least 60%, or at least 65%, or at least 70%, or at least 80%, or at least 85%, or at least 90%, or at least 91%, or at least 92%, or at least 93%, or at least 94%, or at least 95%, or at least 96%, or at least 97%, or at least 98%, or at least 99%, or completely identical to the sequence with SEQ ID No. 141.

83. The method according to claim 80, further comprising introducing into the cell or organism a nucleic acid encoding the caffeylpyruvate hydrolase.

84. The method according to claim 80, further comprising introducing into the cell or organism nucleic acids, which encode enzymes for biosynthesis of 3-aryl acrylic acid from cell metabolites.

85. The method according to claim 84, wherein the enzymes for biosynthesis of 3-aryl acrylic acid are selected from the group of:
(a) tyrosine ammonia-lyase with an amino acid sequence at least 40% identical to the amino acid SEQ ID No. 107; HpaB and HpaC components of 4-hydroxyphenylacetate 3-monooxygenase reductase at least 40% identical to the amino acid sequences of HpaB and HpaC components of 4-hydroxyphenylacetate 3-monooxygenase reductase of E. coli having SEQ ID Nos 109 and 111;
(b) phenylalanine ammonia-lyase with an amino acid sequence at least 40% identical to the amino acid sequence having SEQ ID No.117.

86. The method according to claim 57, further comprising introducing into a cell or organism the expression cassette containing: (a) a domain of transcription initiation, which is functional in a host cell; (b) a nucleic acid, which encodes the type III polyketide synthase, and (c) a domain of transcription termination, which is functional in the host cell, wherein the said cell acquires the ability to bioluminescence in the presence of exogenous or endogenous caffeyl-CoA.

87. The method according to claim 86, further comprising introducing into a cell or organism an expression cassette containing: (a) a domain of transcription initiation, which is functional in a host cell; (b) a nucleic acid, which encodes the coumarate-CoA ligase, and (c) a domain of transcription termination, which is functional in the host cell, wherein said cell acquires the ability to bioluminescence in the presence of caffeic acid.

88. The method according to claim 87, wherein the coumarate-CoA ligase has an amino acid sequence that is at least 40%, or at least 45%, or at least 50%, or at least 55%, or at least 60%, or at least 65%, or at least 70%, or at least 80%, or at least 85%, or at least 90%, or at least 91%, or at least 92%, or at least 93%, or at least 94%, or at least 95%, or at least 96%, or at least 97%, or at least 98%, or at least 99%, or completely identical to the sequence with SEQ ID No. 141.

89. The method according to claim 86, further comprising introducing into the cell or organism a nucleic acid encoding the caffeylpyruvate hydrolase.

90. The method according to claim 87, further comprising introducing into the cell or organism nucleic acids, which encode enzymes for biosynthesis of 3-aryl acrylic acid from cell metabolites.

91. The method according to claim 90, wherein the enzymes for biosynthesis of 3-aryl acrylic acid are selected from the group of:
(a) tyrosine ammonia-lyase with an amino acid sequence at least 40% identical to the amino acid SEQ ID No. 107; HpaB and HpaC components of 4-hydroxyphenylacetate 3-monooxygenase reductase at least 40% identical to the amino acid sequences of HpaB and HpaC components of 4-hydroxyphenylacetate 3-monooxygenase reductase of E. coli having SEQ ID Nos 109 and 111;
(b) phenylalanine ammonia-lyase with an amino acid sequence at least 40% identical to the amino acid sequence having SEQ ID No.117.

92. A transgenic organism capable of bioluminescence in the presence of 3-hydroxyhispidine, and/or hispidin, and/or caffeic acid, produced using any of methods according to claim 80.

93. A kit for producing hispidin, comprising the polyketide synthase according to claim 72 and coumarate-CoA ligase or nucleic acids encoding them.

94. A kit for producing a bioluminescent cell or organism, comprising a nucleic acid encoding the hispidin hydroxylase, a nucleic acid encoding the polyketide synthase according to claim 72, and a nucleic acid encoding the luciferase capable to oxidize fungal luciferin with light emission.

95. The kit according to claim 94, further containing a nucleic acid encoding the caffeylpyruvate hydrolase.

96. The it according to claim 93, further comprising a nucleic acid encoding the coumarate-CoA ligase and/or nucleic acids encoding enzymes for biosynthesis the 3-aryl acrylic acid.


SEQUENCE LISTING (141 sequences)

SEQ ID NO: 1 - DNA, 1266 bp, Neonothopanus nambi
atggcatcgtttgagaattctctaagcgttttgattgtcggggccggacttggtgggcttgctgctgccatcgcgctgcgtcgccaagggcatgtcgtgaaaatatacgactcctctagcttcaaagccgaacttggtgcgggactcgctgtgccgcctaacaccttgcgcagtctacagcaacttggttgcaataccgagaacctcaatggtgtggataatctttgcttcactgcgatggggtatgacgggagtgtagggatgatgaacaacatgactgactatcgagaggcatacggtacttcttggatcatggtccaccgcgttgacttgcataacgagctgatgcgcgtagcacttgatccaggtgggctcggacctcctgcgacactccatcttaatcatcgtgtcacattctgcgatgtcgacgcttgcaccgtgacattcaccaacgggaccactcaatcagctgatctcatcgttggtgcagacggtatacgctctaccattcggcggtttgtcttagaagaagacgtgactgtgcctgcgtcaggaatcgtcgggtttcgatggcttgtacaagctgacgcgctggacccatatcctgaactcgactggattgttaaaaagcctcctctaggcgcgcgactgatctccactcctcagaatccacagtctggtgttggcttggctgacaggcgcactatcatcatctacgcatgtcgtggcggcaccatggtcaatgtccttgcagtgcatgatgacgaacgtgaccagaacaccgcagattggagtgtaccggcttccaaagacgatctatttcgtgttttccacgattaccatccacgctttcggcggcttttagagcttgcgcaggatattaatctctggcaaatgcgtgttgtacctgttttgaaaaaatgggttaacaagcgggtttgcttgttaggagatgctgcgcacgcttctttaccgacgttgggtcaaggttttggtatgggtctggaagatgccgtagcacttggtacactccttccaaagggtaccactgcatctcagatcgagactcgacttgcggtgtacgaacagctacgtaaggatcgtgcggaatttgttgcggctgaatcatatgaagagcaatatgttcctgaaatgcggggactttatctgaggtcaaaggaactgcgtgatagagtcatgggttatgatatcaaagtggagagcgagaaggttctcgagacgctcctaagaagttctaattctgcc

SEQ ID NO: 2 - protein, 422 aa, Neonothopanus nambi
MASFENSLSVLIVGAGLGGLAAAIALRRQGHVVKIYDSSSFKAELGAGLAVPPNTLRSLQQLGCNTENLNGVDNLCFTAMGYDGSVGMMNNMTDYREAYGTSWIMVHRVDLHNELMRVALDPGGLGPPATLHLNHRVTFCDVDACTVTFTNGTTQSADLIVGADGIRSTIRRFVLEEDVTVPASGIVGFRWLVQADALDPYPELDWIVKKPPLGARLISTPQNPQSGVGLADRRTIIIYACRGGTMVNVLAVHDDERDQNTADWSVPASKDDLFRVFHDYHPRFRRLLELAQDINLWQMRVVPVLKKWVNKRVCLLGDAAHASLPTLGQGFGMGLEDAVALGTLLPKGTTASQIETRLAVYEQLRKDRAEFVAAESYEEQYVPEMRGLYLRSKELRDRVMGYDIKVESEKVLETLLRSSNSA

SEQ ID NO: 3 - DNA, 1620 bp, Omphalotus olearius
atgacgccctccgagagtcctttgaatatctcgattgttggtgctgggctcggggggcttgctgcagctattgcgctgcgtcgtcaaggtcatatcatcagaatcttcgactcgtcaagttttaaaacggaactgggtgctggacttgctgttccacccaatacattacgcagtcttcaggaacttggctgtgatattcagaacttcaatgccgtggacaatctttgtttcaccgcgatgggctacgacgggagtgtagggatgatgaacaatatgactgactatcgtgaggcgtatggtgttccctgggtcatggtccaccgcgttgacctacataatgaactgcgacgtgtggcactcgatccagatggccttggacctcctgcagcattgcacctcaatcatcgtgtgacatcctgcgatgtcgattcctgcaccgtcacattcgctaacggaaccgctcatacagcggatctcatcgttggcgcggatggtatacgctcttccatccgacccttcgtgttgggagaagacgtaatcgtacctgcaacaggaatcgcaggatttcgatggctcatacaagccgaccggctagatgcgtatcctgaactcgactggattgtcaagaaccctcctctcggcgcgcgattgatttctgctccggctcggaaggaacgttctgtaatcagcgaagcccggcctgatcgacgtacgattatattatatgcgtgtcgtggtggtactattgtcaatgtccttgcggtgcacgacgacgaacgtgatcaggacaccgtagaatggagcgtgccagctaccaaagacgacctatttcgcgttttcaacgattatcacccaagatttcggcgacttctggacctggcggaggatgttaatctctggcagatgcgtgttgtgcctgttttgagacgatgggttaataaacgggtttgcttgctgggagatgcagcacatgcttctttaccgacattgggtcaaggttttggtatgggtctcgaggatgcggtggcacttggtacgcttcttccgagcgggactactgtgtcacagattgaaatccgactttgggtgtatgaaaaactgcgcaaggagcgtgctgaatttgtttcggctgaatcgtatgaagaacactgctctgtggattgctataaatctcataaagcccagtcgacatccaatacagtgacagaagcagacgacatcttggaagaaccaaagcctctgaagcctttatcgtctctgaaatggccgtacgttcccgaagaaccttcgtatcctgatcccctccaaagaaacgaccccaaacccctacaactcagacactacgaagcaatagctacatctcctgcagtacgggaggtcctatcgagtcatccgaatctccctgctttattgacgtctatcgacaaactgagaggttttgatcgcgaacgagctttagaaaaggcgttggaggttactgcgcctgcgcttgttgatgattcaagggctgtagcgctggaggacgatgtactcgcaatgagagcattggtagaagcgattgaaggtgctgttaggggcaataaagaagacgcattaggtctggattggactggtagtact

SEQ ID NO: 4 - protein, 540 aa, Omphalotus olearius
MTPSESPLNISIVGAGLGGLAAAIALRRQGHIIRIFDSSSFKTELGAGLAVPPNTLRSLQELGCDIQNFNAVDNLCFTAMGYDGSVGMMNNMTDYREAYGVPWVMVHRVDLHNELRRVALDPDGLGPPAALHLNHRVTSCDVDSCTVTFANGTAHTADLIVGADGIRSSIRPFVLGEDVIVPATGIAGFRWLIQADRLDAYPELDWIVKNPPLGARLISAPARKERSVISEARPDRRTIILYACRGGTIVNVLAVHDDERDQDTVEWSVPATKDDLFRVFNDYHPRFRRLLDLAEDVNLWQMRVVPVLRRWVNKRVCLLGDAAHASLPTLGQGFGMGLEDAVALGTLLPSGTTVSQIEIRLWVYEKLRKERAEFVSAESYEEHCSVDCYKSHKAQSTSNTVTEADDILEEPKPLKPLSSLKWPYVPEEPSYPDPLQRNDPKPLQLRHYEAIATSPAVREVLSSHPNLPALLTSIDKLRGFDRERALEKALEVTAPALVDDSRAVALEDDVLAMRALVEAIEGAVRGNKEDALGLDWTGST

SEQ ID NO: 5 - DNA, 1329 bp, Guyanagaster necrorhiza
atgcaacaaatcgacgaagtgtgcccattgaaagtgatcgttgtaggtgctggacttgggggcctttctgctgccattgcccttcgtaggcaaggccattgtgtccatatactggaatcgtcaagtttcaagagcgaacttggcgcaggtctcgcagtaccgcccaatactgtacgctcacttcgaggcctaggctgtaacatcgacaatctcaagcctgtggataatctttgtttcactgccatggcgcatgacggaagtcctggtatgatgaataacatgacggactatggccaggcgtatggagatccttgggtaatggcgcatcgtgttgaccttcacaatgagctcatgcgagtggcccttgaacccgaaaaaacgggacctcctgcccagcttcgtctggacagccaggtggcatcttgcaatgtagatgcctgtaccgtttctcttgtcgacggaacaatttattccgctgatcttatcgttggtgcagacggaattaggtctaccatacgctcctatgttttggacgcagaaatagacatacctcctaccggtatcgctggataccgttggctcacacctgcagaagctttggagccatatcccgaactcgactggatcatcaagaacccacccctaggagcacgtttaatcacagctcctgtacgccgaaacgacagcattgagcagtcgggtcctgctcctatttctgagaaggctgacaagcgtacgatcatcatctacgcgtgccggaatggtactatgcttaacgttctcggtgtacacgatgaccctcgcaaccagaacgaagttggatggaacgtgccagttacccaagaaagtttgctggatttttttaaagactatcatccccgattcaagcgtctgcttgagctggctgacaatgttcatctgtggcaaatgcgcgtcgtcccgcggcttgagacttggatcaataaacgcgtgtgtttgttgggcgattctgcgcatgcatcattaccaacactcggtcaaggcttcgggatgggacttgaggatgccgtagcccttgcaaccctccttccgatgggaaccaaagtgtctgacatcgagaaccgcctcgtcgcctacgaaagcttgcgtaaggggcgcgctgagtatgtggccatggaatcgttcgaacaacagaatatcccggaaaagcgaggcttgtatctcaggtcttctatgatgcgtgacgaaatcatgggttatgatgtcaaagccgaggctgagaaggtttttaaagaattaatgacctcgactgataaggttacataccgtccccatgtggactgtctatgg

SEQ ID NO: 6 - protein, 443 aa, Guyanagaster necrorhiza
MQQIDEVCPLKVIVVGAGLGGLSAAIALRRQGHCVHILESSSFKSELGAGLAVPPNTVRSLRGLGCNIDNLKPVDNLCFTAMAHDGSPGMMNNMTDYGQAYGDPWVMAHRVDLHNELMRVALEPEKTGPPAQLRLDSQVASCNVDACTVSLVDGTIYSADLIVGADGIRSTIRSYVLDAEIDIPPTGIAGYRWLTPAEALEPYPELDWIIKNPPLGARLITAPVRRNDSIEQSGPAPISEKADKRTIIIYACRNGTMLNVLGVHDDPRNQNEVGWNVPVTQESLLDFFKDYHPRFKRLLELADNVHLWQMRVVPRLETWINKRVCLLGDSAHASLPTLGQGFGMGLEDAVALATLLPMGTKVSDIENRLVAYESLRKGRAEYVAMESFEQQNIPEKRGLYLRSSMMRDEIMGYDVKAEAEKVFKELMTSTDKVTYRPHVDCLW

SEQ ID NO: 7 - DNA, 1284 bp, Panellus stipticus
atgtcacaaggcactgaagactcgccctcactcttcgcaatagttgtcggtgctggtctggtcggctttgccgctgccgtcgcgctgcgccgtcaaggtcaccgtgtcacgatctacgaatcttcaagctttaagacagaacttggggcgggcctcgcgatcccttcaaacacattgcgatgcttggttggccttggatgtactgtcgccaacatggatcccgtcaataatctttgttttacatcgatggcatacgatggtaccgcggggatgaaaagcgacagctccgactacgaggcgcagtatggcactccctggatcatggcccaccgcgtcgatctgcacaaggagcttcgtcgcctggcagtggatcccgagggcaccggtccccccgcagaactgcaccttagccaccgggttgtctcctgcgacgtcgaatccggctccgtcacgctcctggacggctccgttcagtcagcagacttgataattggggctgatggaattcgttcaaccgttcgcaaatttgtcgtaggcgaagaaatagcaatccccccatctcgtacggcgggcttccgctggctcacacaagcaagcgctcttgacccctaccccgaactggattggatcgtgaaaacgccgccgttgggtgctcgggtaatctccgctccgattcaacctccagaagtcacccacatcgaccaccgtacgatcgtcatctatgcctgtcgcggaggtcgtctcgtaaatgttctaggaatacatgaggatctgcgcgaccaggattctgtcgactggaacgtccccataacgcgagaagcgctgctccactttttcggagactaccacccacggttcaagcggctcttggagctcgcggaggacgtacacgtctggcagatgcgcgtggtccccccactgccgacatgggtgaacggccgcgtatgcatcatgggcgacgcagcgcatgcatcgcttcccacactgggacaagggtttggcatgggcctcgaagacgcggtcgcgctcgggacgctgctttctagttcgacgccgtcaagcgacattccaagccgtctcgtcgcgtacgagaagcttcgcaaagcgcgcgctgagtatgtttccaaagagtcgtacgaacagcagcatgtccgagaaaagagagggctgtatctccggtcccgagaaatgcgggatgtgatcatgggatacgacgtgaagaaggaggcagaacggatcttgagcgagatcagcattgcacaagaacagtgtgctgttcatgat

SEQ ID NO: 8 - protein, 428 aa, Panellus stipticus
MSQGTEDSPSLFAIVVGAGLVGFAAAVALRRQGHRVTIYESSSFKTELGAGLAIPSNTLRCLVGLGCTVANMDPVNNLCFTSMAYDGTAGMKSDSSDYEAQYGTPWIMAHRVDLHKELRRLAVDPEGTGPPAELHLSHRVVSCDVESGSVTLLDGSVQSADLIIGADGIRSTVRKFVVGEEIAIPPSRTAGFRWLTQASALDPYPELDWIVKTPPLGARVISAPIQPPEVTHIDHRTIVIYACRGGRLVNVLGIHEDLRDQDSVDWNVPITREALLHFFGDYHPRFKRLLELAEDVHVWQMRVVPPLPTWVNGRVCIMGDAAHASLPTLGQGFGMGLEDAVALGTLLSSSTPSSDIPSRLVAYEKLRKARAEYVSKESYEQQHVREKRGLYLRSREMRDVIMGYDVKKEAERILSEISIAQEQCAVHD

SEQ ID NO: 9 - DNA, 1284 bp, Panellus stipticus
atgtcacaaggcactgaagactcgccctcactcttcgcaatagttgtcggtgctggtctggtcggctttgccgctgccgtcgcgctgcgccgtcaaggtcaccgtgtcacgatctacgaatcttcaagctttaagacagaacttggggcgggcctcgcgatcccttcaaacacattgcgatgcttggttggccttggatgtactgtcgccaacatggatcccgtcaataatctttgttttacatcgatggcatacgatggtaccgcggggatgaaaagcgacagctccgactacgaggcgcagtatggcactccctggatcatggcccaccgcgtcgatctgcacaaggagcttcgtcgcctggcagtggatcccgagggcaccggtccccccgcagaactgcaccttagccaccgggttgtctcctgcgacgtcgaatccggctccgtcacgctcctggacggctccgttcagtcagcagacttgataattggggctgatggaattcgttcaaccgttcgcaaatttgtcgtaggcgaagaaatagcaatccccccatctcgtacggcgggcttccgctggctcacacaagcaagcgctcttgacccctaccccgaactggattggatcgtgaaaacgccgccgttgggtgctcgggtaatctccgctccgattcaacctccagaagtcacccacatcgaccaccgtacgatcgtcatctatgcctgtcgcggaggtcgtctcgtaaatgttctaggaatacatgaggatctgcgcgaccaggattctgtcgactggaacgtccccataacgcgagaagcgctgctccactttttcggagactaccacccacggttcaagcggctcttggagctcgcggaggacgtacacgtctggcagatgcgcgtggtccccccactgccgacatgggtgaacggccgcgtatgcatcatgggcgacgcagcgcatgcatcgcttcccacactgggacaagggtttggcatgggcctcgaagacgcggtcgcgctcgggacgctgcttcctagttcgacgccgtcaagcgacattccaagccgtctcgtcgcgtacgagaagcttcgcaaagcgcgcgctgagtatgtttccaaagagtcgtacgaacagcagcatgtccgagaaaagagagggctgtatctccggtcccgagaaatgcgggatgtgatcatgggatacgacgtgaagaaggaggcagaacggatcttgagcgagatcagcattgcacaagaacagtgtgctgttcatgat

SEQ ID NO: 10 - protein, 428 aa, Panellus stipticus
MSQGTEDSPSLFAIVVGAGLVGFAAAVALRRQGHRVTIYESSSFKTELGAGLAIPSNTLRCLVGLGCTVANMDPVNNLCFTSMAYDGTAGMKSDSSDYEAQYGTPWIMAHRVDLHKELRRLAVDPEGTGPPAELHLSHRVVSCDVESGSVTLLDGSVQSADLIIGADGIRSTVRKFVVGEEIAIPPSRTAGFRWLTQASALDPYPELDWIVKTPPLGARVISAPIQPPEVTHIDHRTIVIYACRGGRLVNVLGIHEDLRDQDSVDWNVPITREALLHFFGDYHPRFKRLLELAEDVHVWQMRVVPPLPTWVNGRVCIMGDAAHASLPTLGQGFGMGLEDAVALGTLLPSSTPSSDIPSRLVAYEKLRKARAEYVSKESYEQQHVREKRGLYLRSREMRDVIMGYDVKKEAERILSEISIAQEQCAVHD

SEQ ID NO: 11 - DNA, 1284 bp, Panellus stipticus
atgtcacaaggcactgaagactcgccctcactcttcgcaatagttgtcggtgctggtctggtcggctttgccgctgccgtcgcgctgcgccgtcaaggtcaccgtgtcactatctacgaatcttcaagctttaagacagaacttggggcgggcctcgcgatcccttcaaacacattgcgatgcttggttggccttggatgtactgtcgccaatctggatcccgtcaataatctttgttttacatcgatggcttacgatggtaccgcggggatgaaaagcgacagctccgactacgaggcgcagtatggcactccctggatcatggcccaccgcgtcgatctgcacaaggagcttcgtcgcctggcagtggatcccgagggcaccggtccccccgcagaactgcaccttagccaccgggttgtctcctgcgacgtcgaatccggctccgtcacgctcctggacggctccattcagtcagcagacttgataattggggctgatggaattcgttcaaccgttcgcaaatttgtcgtaggcgaagaaatagcaatccccccatctcgtacggcgggcttccgttggctcacacaagcaagcgctcttgacccctaccccgaactggattggatcgtgaaaacgccgccgttgggtgctcgggtaatctccgctccgattcaacctccagaagtcacccacatcgaccaccgtacgatcgtcatctatgcctgtcgcggaggtcgtctcgtaaatgttctaggaatacatgaggatctgcgcgaccaggattctgtcgactggaacgtccccataacgcgagaagcgctgctccactttttcggagactaccacccacggttcaagcggctcttggagctcgcggaggacgtacacgtctggcagatgcgcgtggtccccccactgccgacatgggtgaacggccgcgtatgcatcatgggcgacgcagcgcatgcatcgcttcccacactgggacaagggtttggcatgggcctcgaagacgcggtcgcgctcgggacgctgcttcctagttcgacgccgtcaggcgacattccaagccgtctcgtcgcgtacgagaagcttcgcaaagcgcgcgctgagtatgtttccaaagagtcgtacgaacagcagcatgtccgagaaaagagagggctgtatctccggtcccgagaaatgcgggatgtgatcatgggatacgacgtgaagaaggaggcagaacggatctttagcgagatcagcattgcacaagaacagcgtgctgttcatgat

SEQ ID NO: 12 - protein, 428 aa, Panellus stipticus
MSQGTEDSPSLFAIVVGAGLVGFAAAVALRRQGHRVTIYESSSFKTELGAGLAIPSNTLRCLVGLGCTVANLDPVNNLCFTSMAYDGTAGMKSDSSDYEAQYGTPWIMAHRVDLHKELRRLAVDPEGTGPPAELHLSHRVVSCDVESGSVTLLDGSIQSADLIIGADGIRSTVRKFVVGEEIAIPPSRTAGFRWLTQASALDPYPELDWIVKTPPLGARVISAPIQPPEVTHIDHRTIVIYACRGGRLVNVLGIHEDLRDQDSVDWNVPITREALLHFFGDYHPRFKRLLELAEDVHVWQMRVVPPLPTWVNGRVCIMGDAAHASLPTLGQGFGMGLEDAVALGTLLPSSTPSGDIPSRLVAYEKLRKARAEYVSKESYEQQHVREKRGLYLRSREMRDVIMGYDVKKEAERIFSEISIAQEQRAVHD

SEQ ID NO: 13 - DNA, 1248 bp, Neonothopanus gardneri
atggcactatctgagagtcctttgaacgtcttgatagtaggagcggggctcggggggcttgctgctgccatagcactacgtcgtcaagggcatatcgtgaaaatattcgattcctccagtttcaaaaccgaacttggtgcaggacttgctgtcccgcctaataccctgcgtagtctgcaggaactcgggtgcagtgtcgagaacctcaatgctgtggataatctttgcttcactgcgatggggtatgacgggagtgtaggaatgatgaacaatatgaccgactatcgagaggcgtacggtcatccttgggtcatggttcaccgtgtcgacttgcataatgagctgaagcgcgtggcgcttgatccagacggcctcggacctcctgcaactttgcatctcaaccatcgtgtcacattctgcgacatcgactcttgcactgtcacgttcgctaatgggacttctaaatcggcagatcttatcgtaggcgcagacggtatacgctctaccattcgcaagttcattcttggagaagacgtcgttatacccgcgtcaggaatagcagggtttcgatggcttgtgcaagctgacgcgctggatccgtatcctgaactcgactggatcgtgaagaaccctcctctaggagcccgactgatttccgctcctaggaatcaacagtctactgataggcgcactatcatcatctatgcgtgtcgtagcggcaccatggtcaacgtactcgcagtacatgatgatgatcgtgaccagaacgccgtagattggagtgcaccagcttccaaagatgatttattccacatcttccacgactaccacccacgattccagcggcttctggagctggcgcaagatatcaatctctggcaaatgcgtgttgttcctgttctgaaacaatgggttaacaaacgtgtttgcttgttaggagatgcggcacacgcttctttacctacattagggcagggatttggtatgggtctagaagatgccgtagcactgggtacgcttcttccaaagggggccacagtatctcagatcgagagccgactcgcggtgtatgaaattctgcgcaaggagcgtgctgaatttgtttcggctgagtcatatgaagagcagtacgttccagaaaaacgcgggctttacttaagatcgaaggaattgcgcgacagtgtcatgggttacgatattaaaatggagagcgagaaggttctcgtggcattacttagcggttcttca

SEQ ID NO: 14 - protein, 416 aa, Neonothopanus gardneri
MALSESPLNVLIVGAGLGGLAAAIALRRQGHIVKIFDSSSFKTELGAGLAVPPNTLRSLQELGCSVENLNAVDNLCFTAMGYDGSVGMMNNMTDYREAYGHPWVMVHRVDLHNELKRVALDPDGLGPPATLHLNHRVTFCDIDSCTVTFANGTSKSADLIVGADGIRSTIRKFILGEDVVIPASGIAGFRWLVQADALDPYPELDWIVKNPPLGARLISAPRNQQSTDRRTIIIYACRSGTMVNVLAVHDDDRDQNAVDWSAPASKDDLFHIFHDYHPRFQRLLELAQDINLWQMRVVPVLKQWVNKRVCLLGDAAHASLPTLGQGFGMGLEDAVALGTLLPKGATVSQIESRLAVYEILRKERAEFVSAESYEEQYVPEKRGLYLRSKELRDSVMGYDIKMESEKVLVALLSGSS

SEQ ID NO: 15 - DNA, 1257 bp, Mycena citricolor
atgaacacccccaataacgctctcgatgttattgttgtcggtgctggcctggttggtttcgcggccgctgctgctctacgccgacaaggtcatcgcgtgactatctacgagacttccagcttcaagaacgagctaggagctggccttgctattccacccaacactgtccgtggcctaattggcttgggatgtgtgattgagaacttggacccggtggagaatctatgcgtttccgtcgcttttgacggaagtgctggtatgcgcagtgaccagaccaactacgaagcaagctacggccttccctggatcatggtgcatcgcgtcgatttgcacaatgagcttcgtcgggttgctctcagtgccgaggggaacattggtcccccagccgagctacgcctggaccaccgagtcagctcgtgtgatgtcgagaaatgcactgtgacgctgagcaatggcgatacccaccacgcggatctgatcattggagcagacgggatccattctacaatccgatccttcgtcgtgggcgaggaaatcgtcattccgccctccaagacagccggtttccgctggctcacagagagtactgcgttggagccctatccggaattggactggattgtgaagatcccaccacttggcgcccggctgatctctgcgccaatgaaccctgcgccaccgcaggtcgaccaccggacgatcatcatctacgcctgtcgtggcagtacactgataaatgtactcggagtccatgaggatctccgcgatcaagatacagttccctggaatgcacccgtaacccaatcggagctgctccagttctttggcgattaccatccgcgattcaaacgattgttggagcttgcaaatgatgttcatgtgtggcagatgcgagtagtgccccgcttggagacctgggtcaatcgtcgggtttgcattatgggcgatgctgcgcatgcatcactccccacgctgggtcaaggtttcggaatggggctcgaggatgcagtcgctcttggaacactccttccgcttgggacaactcccgaagagatcccggaccgtctcaccctctggcaggatctcgtcaaacctcgggctgagtttgtcgcgactgaatcctacgaacagcagcatattcctgcgaaacggggactctatcttcgctcgcaggagatgcgcgactgggtcatgggatacgatgtccaggctgaggcacagaaggtcttggcgggagctgtgaatagatccaaggga

SEQ ID NO: 16 - protein, 419 aa, Mycena citricolor
MNTPNNALDVIVVGAGLVGFAAAAALRRQGHRVTIYETSSFKNELGAGLAIPPNTVRGLIGLGCVIENLDPVENLCVSVAFDGSAGMRSDQTNYEASYGLPWIMVHRVDLHNELRRVALSAEGNIGPPAELRLDHRVSSCDVEKCTVTLSNGDTHHADLIIGADGIHSTIRSFVVGEEIVIPPSKTAGFRWLTESTALEPYPELDWIVKIPPLGARLISAPMNPAPPQVDHRTIIIYACRGSTLINVLGVHEDLRDQDTVPWNAPVTQSELLQFFGDYHPRFKRLLELANDVHVWQMRVVPRLETWVNRRVCIMGDAAHASLPTLGQGFGMGLEDAVALGTLLPLGTTPEEIPDRLTLWQDLVKPRAEFVATESYEQQHIPAKRGLYLRSQEMRDWVMGYDVQAEAQKVLAGAVNRSKG

SEQ ID NO: 17 - DNA, 1260 bp, Mycena citricolor
atgaacacccccaataacgctctcgatgttattgttgtcggtgctggcctggttggtttcgcggccgctgctgctctacgccgacaaggtcatcgcgtgactatctatgagacttccagcttcaagaacgagttaggagctggcctggctattccacccaacactgtccgtggtctaattggcttgggatgtgtgattgagaacttggacccggtggagaatctatgcttcactgccgtcgcgtttgacggaagtgctggtatgcgcagcgaccaaaccaactacgaagcaagttacggccttccctggatcatggtgcatcgtgtcgatttgcacaacgagcttcgtcgggttgctctcagcgccgaggggaacactggtcccccagcggagctacgcctggaccaccgagtcagctcgtgtgatgtcgagaaatgcactgtgacgctgagcaatggggatacccaccacgcagatctgatcattggagcagacgggatccattctacaatccgatccttcgttgtgggcgaggaaatcgtcattccgccctccaagacagccggtttccgctggctcacagagagtactgcgttggagccctatccggaattggactggattgtgaagatcccaccacttggcgcccggctgatctctgcgccaatgaaccctgcgccaccgcaggtcgaccaccggacgatcatcatctacgcctgtcgtggcagtacactgataaatgtactcggagtccatgaggatctccgcgatcaagatacagtcccctggaatgcacccgtaacccaatcggagctgctccagttctttggcgattaccatccgcggttcaaacgattgttggagcttgcaaatgatgttcatgtgtggcagatgcgagtagtgccccgcttggagacctgggtcaatcgtcgggtttgcattatgggcgatgctgcgcatgcatcactccccacgctgggtcaaggtttcggaatggggctcgaggatgcagtcgctcttggaacactccttccgcttgggacaactcccgaagagatcccggaccgtctcaccctctggcaggatctcgtcaaacctcgggctgagtttgtcgcgactgaatcctacgaacagcagcatattcctgcgaaacggggactctatcttcgctcgcaggagatgcgcgactgggtcatgggatacgatgtccaggctgaggcacagaaggtcttggcgggagctgtgaatagatccaaggga

SEQ ID NO: 18 - protein, 420 aa, Mycena citricolor
MNTPNNALDVIVVGAGLVGFAAAAALRRQGHRVTIYETSSFKNELGAGLAIPPNTVRGLIGLGCVIENLDPVENLCFTAVAFDGSAGMRSDQTNYEASYGLPWIMVHRVDLHNELRRVALSAEGNTGPPAELRLDHRVSSCDVEKCTVTLSNGDTHHADLIIGADGIHSTIRSFVVGEEIVIPPSKTAGFRWLTESTALEPYPELDWIVKIPPLGARLISAPMNPAPPQVDHRTIIIYACRGSTLINVLGVHEDLRDQDTVPWNAPVTQSELLQFFGDYHPRFKRLLELANDVHVWQMRVVPRLETWVNRRVCIMGDAAHASLPTLGQGFGMGLEDAVALGTLLPLGTTPEEIPDRLTLWQDLVKPRAEFVATESYEQQHIPAKRGLYLRSQEMRDWVMGYDVQAEAQKVLAGAVNRSKG

SEQ ID NO: 19 - DNA, 1287 bp, Armillaria mellea
atgcaacaaatcgacgaagcgcgcccattgaaagtgatagtagtgggtgctggactttgtgggctttccgccgccattgcacttcgtaggcaagggcatcatgttcatatacttgaatcttcaagttttaagagcgagcttggcgcaggtctcgccgtcccacccaatactgtacgctctcttcgaggcctaggttgtaacatcgacaatctcaagcccgtggataatttgtgtttctctgccatggcgcatgacggaagcccaggcatgatgaataacatgacagactatcacaaggcgtacggtgatccttgggtaatggcacatcgtgtcgacctccataacgagctcttgcgagtggctttcgaccccgaaggaacagggcctcctgctcaacttcgtttgggcgtccaggtagtgacttgcgatatggaagcttgtacaatttcccttgtcgatggaacagtctgttccgccgatcttatcgtaggagctgacggtattaagtcgaccatacgctcctgtgttctaggcaaagaaatagacatacctcctaccggtatcgccggataccgctggctcataccggcagaagctttggagccctatcccgagctcgactggattatcaagaacccacccctaggagcacgtttaatcacggatcccgtacgccgaactgaacaaacggatgacggtaagaaggctgacaagcgcacgatcataatctatgcgtgccgcagtggcacgatgatcaacgttcttggtgtgcacgatgacctgcgcaaccagaatgaagtcggatggaacgtaccagtcacacgggaaaacttgctggagtttttcggggactaccacccacggtttaagcgtttactccagctagccgatagtattcatttgtggcaaatgcgtgttgtcccacggcttgacacatggattaatagatgcgtgtgtttgctgggcgattctgcacatgcgtcattaccaactctcgggcaaggcttcggaatgggtcttgaggatgccgtagctctcgcagccctccttccgatgggaaccaatgcgtctgacgttgagaaccgccttatcgcctacgaaagcttgcgtaaggagcgtgcagagtatgtagccacggaatcattagaacagcaggatattgcaggaaagcgaggcttgtatctcaggtctcctatgatgcgcgataaaataatgggttatgatattaaagcggaagctgagaaggttttaatcgaattaaaaaattcgacagctcagcaggtcact

SEQ ID NO: 20 - protein, 429 aa, Armillaria mellea
MQQIDEARPLKVIVVGAGLCGLSAAIALRRQGHHVHILESSSFKSELGAGLAVPPNTVRSLRGLGCNIDNLKPVDNLCFSAMAHDGSPGMMNNMTDYHKAYGDPWVMAHRVDLHNELLRVAFDPEGTGPPAQLRLGVQVVTCDMEACTISLVDGTVCSADLIVGADGIKSTIRSCVLGKEIDIPPTGIAGYRWLIPAEALEPYPELDWIIKNPPLGARLITDPVRRTEQTDDGKKADKRTIIIYACRSGTMINVLGVHDDLRNQNEVGWNVPVTRENLLEFFGDYHPRFKRLLQLADSIHLWQMRVVPRLDTWINRCVCLLGDSAHASLPTLGQGFGMGLEDAVALAALLPMGTNASDVENRLIAYESLRKERAEYVATESLEQQDIAGKRGLYLRSPMMRDKIMGYDIKAEAEKVLIELKNSTAQQVT

SEQ ID NO: 21 - DNA, 1278 bp, Armillaria fuscipes
atgcaacaaatcgacgaagcgcgtccattgaaggtgctagtcgtgggtgctggactctgtgggctttccgccgccatcgcacttcgtagacaagggcatcacgtccatatacttgaatcttcaagtttcaagagcgaacttggggcaggtctcgccgtgccacctaatactgtgcgctctcttcgaggtctcggctgtgacatagacaatctcaagcccgtggataatctttgtttcactgctatgtcgcatgacggaagcccaggcatgatgaataacatgacggactatcgcaaggcgtatggtgatccttgggtgatggcacatcgtgtcgaccttcataacgagcttatgcgagtggctctcgaccctgatgggacaggtcctcctgcccaacttcgtttgggcgtccaggttgtgtcttgcgatgttgaagcttgtactgtttcccttgtcgatggagaggtctgttccgccgatcttatcgttggagctgatggtatcaggtcaaccatacgctcctatgttctaggcaaagaaatagatatacctcctaccggcattgccggataccgctggcttacaccatcagaggctttggagccttttcccgaacttgattggattatcaagaacccacctctaggagcacgtctaatcaccgctcccatacgccggaacgaacaaatgaatgacggtgagatggctgacaagcgtacgatcatcatctacgcgtgccgcaacggcacaatgattaacgttctcggtgtgcacgatgacccgcgcaaccagaatgaagtcggatggaacgtgccagtaacccaagaaaaattgctcgaatttttcggagactaccacccacggttcaaaagtttacttcagctatctgatagtattcatttgtggcaaatgcgtgttgttccacggcttgacacatggatcaatcaacgtgtgtgtttgctgggcgattctgcacatgcgtcattaccaacgctcgggcaaggcttcgggatgggtcttgaggacgccatagctcttgcaaccctccttccgatgggcgccaaagtgtcggacattgagaatcgccttatcgcctacgaaagcctgcgtaaggagcgtgcagagtttgtagccacggaatcattagaacagcaagatattcccgaaaagcgaggcttgtatctcagatcccctatgatgcgcgataaaataatgggttacgatatcaaagccgaagctgaaaaggttttaatggagttattgagctcgaaagctcaa

SEQ ID NO: 22 - protein, 426 aa, Armillaria fuscipes
MQQIDEARPLKVLVVGAGLCGLSAAIALRRQGHHVHILESSSFKSELGAGLAVPPNTVRSLRGLGCDIDNLKPVDNLCFTAMSHDGSPGMMNNMTDYRKAYGDPWVMAHRVDLHNELMRVALDPDGTGPPAQLRLGVQVVSCDVEACTVSLVDGEVCSADLIVGADGIRSTIRSYVLGKEIDIPPTGIAGYRWLTPSEALEPFPELDWIIKNPPLGARLITAPIRRNEQMNDGEMADKRTIIIYACRNGTMINVLGVHDDPRNQNEVGWNVPVTQEKLLEFFGDYHPRFKSLLQLSDSIHLWQMRVVPRLDTWINQRVCLLGDSAHASLPTLGQGFGMGLEDAIALATLLPMGAKVSDIENRLIAYESLRKERAEFVATESLEQQDIPEKRGLYLRSPMMRDKIMGYDIKAEAEKVLMELLSSKAQ

SEQ ID NO: 23 - DNA, 1266 bp, Armillaria gallica
atgcaacaaatcgacgaagcgcgcgcattgaaagtgatagtcgtgggtgctggactttgcgggctttccgccgccattgtacttcgtaggcaagggcatcacgtccatatacttgaatcttcaagtttcaagagcgaacttggtgcaggtcttgccgtgccgcccaacactgtacgctctcttcgaggtctaggttgtaacatcgacaatctcaagcccgtggataatctttgtttcactgccatggctcatgacggaagcccaggcatgatgaataacatgacagactatcagaaggcgtacggcgatccttgggtaatggcgcatcgtgtcgacctccataacgagctcatgcgagtggctctcgaccctgaaggaacaggccctgctgcccagcttcgtttgggcgtccaggtggtgtcttgtgatgtggaagcttgtaccatttctcttgtcgatggatcaatctgtaccgccgatcttatcgtcggagctgatggtattaggtcaaccatacggtcctatgttttgggcaaagaaatagacatacctcctaccggtattgctggataccgctggctcacaccggcagaagctttggacccatatcccgaactcgactggattatcaagaacccacccctgggagcacgtttaatcacagctcccgttcgccgaaacgataaggcggatgacggtgagaaagctgacaagcgcacgatcataatctacgcgtgccgcagtggcactatgatcaacgttctcggtgtgcacgatgacccgcgcaaccagaatgaagtcggatggaacgtaccagttacccaggaaaatttgttggagtttttcgaagactaccacccacggtttaagcgtttacttcagctgaccgataacattcatttgtggcaaatgcgtgttgtcccgcggcttgacacatggattaataaacgcgtgtgtttgctgggcgattctgcacatgcgtcattaccaacactcgggcaaggcttcgggatgggtcttgaggacgccgtagctctcgcaaccctccttccgatgggaaccaaattgtctgacattgaaaaccgtcttgtcgcctacgaaagcttgcgtaaggagcgtgctgagtatgtagccacggaatcattagaacagcaggatattccgggaaagcgaggcttgtatctcaggtctcctgtgatgcgcgataaaataatgggttatgatatcaaagccgaagctgagaaggttttaatggaattgataacc

SEQ ID NO: 24 - protein, 422 aa, Armillaria gallica
MQQIDEARALKVIVVGAGLCGLSAAIVLRRQGHHVHILESSSFKSELGAGLAVPPNTVRSLRGLGCNIDNLKPVDNLCFTAMAHDGSPGMMNNMTDYQKAYGDPWVMAHRVDLHNELMRVALDPEGTGPAAQLRLGVQVVSCDVEACTISLVDGSICTADLIVGADGIRSTIRSYVLGKEIDIPPTGIAGYRWLTPAEALDPYPELDWIIKNPPLGARLITAPVRRNDKADDGEKADKRTIIIYACRSGTMINVLGVHDDPRNQNEVGWNVPVTQENLLEFFEDYHPRFKRLLQLTDNIHLWQMRVVPRLDTWINKRVCLLGDSAHASLPTLGQGFGMGLEDAVALATLLPMGTKLSDIENRLVAYESLRKERAEYVATESLEQQDIPGKRGLYLRSPVMRDKIMGYDIKAEAEKVLMELIT

SEQ ID NO: 25 - DNA, 1278 bp, Armillaria ostoyae
atgcaacaaatcgacgaagcgcacccattgacagtgatagtcgtgggtgctggactttgcgggctttccgccgccattgcacttcgtaggcaagggcatcacgtccatatacttgaatcttcaagtttcaaaagcgaacttggcgcaggtctcgccgtgccgcccaacactgtacgctctcttcgaggtctaggttgtaacatcgacaatctcaagcccgtggataatctttgtttcactgccatggcgcatgacggaagcccaggcatgatgaataacatgacagactatcacaaggcgtacggcgagccttgggtaatggcgcatcgtgtcgacctccataacgagctcatgcgagtggctctcgaccctgaaggaacaggtcctcctgctcagcttcgtttgggtgtccaggtggtgtcttgcgatgtggaagcttgtaccatttctcttgtcgatggatcaatctgttccgccgatcttatcgtcggagctgatggtattaggtcaaccatacgctcctatgttttgggcaaagaaatagacatacctcctaccggtattgctggataccgttggctcacaccggcagaagctttggagccatatcccgaactcgactggattatcaagaacccacccctgggagcacgtttaatcacggctcccgtacgccgaaacgataagacggatgacggtgagaagactgacaagcgcacgatcataatctacgcgtgccgcaatggcactatgatcaacgttctcggtgtgcacgatgacccgcgcaaccagaatgaagtcggatggaacgtaccagttacccaggaaaatttgttggagtttttcgaagactaccacccacggtttaagcgtttacttcagttggccgatagtattcatttgtggcaaatgcgtgttgtcccacggcttgacacatggattaataaacgcgtgtgtttgctgggcgattctgcacatgcgtcattaccaacactcgggcaaggcttcgggatgggtcttgaggacgccgtagctctcgcagccctccttccgatgggaaccaaagtgtctgacgttgagagtcgtcttatcgcctacgaaagcttgcgtaaggagcgtgctgagtatgtagccacggaatcattagaacagcagaatattccgggaaagcgaggcttgtatctcaggtctcctatgatgcgcgataaaataatgggttatgatatcaaagccgaagctgagaaggttttaatggaattaataacctcgacagctcag

SEQ ID NO: 26 - protein, 426 aa, Armillaria ostoyae
MQQIDEAHPLTVIVVGAGLCGLSAAIALRRQGHHVHILESSSFKSELGAGLAVPPNTVRSLRGLGCNIDNLKPVDNLCFTAMAHDGSPGMMNNMTDYHKAYGEPWVMAHRVDLHNELMRVALDPEGTGPPAQLRLGVQVVSCDVEACTISLVDGSICSADLIVGADGIRSTIRSYVLGKEIDIPPTGIAGYRWLTPAEALEPYPELDWIIKNPPLGARLITAPVRRNDKTDDGEKTDKRTIIIYACRNGTMINVLGVHDDPRNQNEVGWNVPVTQENLLEFFEDYHPRFKRLLQLADSIHLWQMRVVPRLDTWINKRVCLLGDSAHASLPTLGQGFGMGLEDAVALAALLPMGTKVSDVESRLIAYESLRKERAEYVATESLEQQNIPGKRGLYLRSPMMRDKIMGYDIKAEAEKVLMELITSTAQ

SEQ ID NO: 27 - DNA, 1266 bp, Mycena chlorophos
atgccctccaccgccgaatctccgcagccgctcaagatcgtcatcgtcggtgctgggctcgttggtctcgctgctgccattgcgcttcgtcgcgagggtcatcatgtagagatatacgaatcgtcgacgttcaagaccgaactcggcgccggtctcgcgataccgtgcaataccctccgtagcctcattgagctggggtgcattgtggctaacttgaacccggtggagaacctctgtttcacgtctatggcgcacgacggaagcgagtcaggcatgcgaagcgaccacaccgactacgaggcgcgttatgggaccccttgggtcatggcgcatcgcgtcgacatacacgcagagctgctccgaatggccaccacctccgatattcccggcccaccggcgacactgcatctcggccaacgcgtccttgcctgcaatgtgtccgactgctccattgcactggccaccggcaaaacgatctcagcggatctcgtcgttggtgccgatgggattcgctcgaccattcgagctgctgttcttggcgaagatatccacattccggcatcgggcactgccggcttccgatggctcgtagattccgccgcgctggatccctatcccgagctggactggattgtgaaagcccgtccgcttggcgctcgcgttatttctgccccgatgggcctcgcactcgaagatcatcgtaccattgtgatctatgcgtgtcgcggcggtaacttgatcaacgttcttgcggtccacgaagacaagcgcgaccaggaggctgtcccttggaatgtccctttgacgcgcgaagccctcttggacttctttagcgactaccacccgcgtttccgccgtctcttcgagctcgcgccggtcgacggaattcacgtctggcagatgcgggtcgtaccacctttggccaactggatccgtgaccgcgtttgcattctcggcgacgcggcgcatgcgtctcttcctactatgggccagggctttggccaaggtctcgaagacgccgttgcgctagcgactttgctcccgctaggaacgcgtagaacggatatccccgctcgtctagtggcgtatgaggggatgcgcaagcctcggaccgagtggattgcacgcgaatcgtttgagcagcaggccgtcgcggaaaagcgcggcatttacttgcgctctatcgaaatgcgcgatgcggttatggggtataatgttcgcgaggaggctaagcgcgtcttgtccgagctcactaaatctgattgt

SEQ ID NO: 28 - protein, 422 aa, Mycena chlorophos
MPSTAESPQPLKIVIVGAGLVGLAAAIALRREGHHVEIYESSTFKTELGAGLAIPCNTLRSLIELGCIVANLNPVENLCFTSMAHDGSESGMRSDHTDYEARYGTPWVMAHRVDIHAELLRMATTSDIPGPPATLHLGQRVLACNVSDCSIALATGKTISADLVVGADGIRSTIRAAVLGEDIHIPASGTAGFRWLVDSAALDPYPELDWIVKARPLGARVISAPMGLALEDHRTIVIYACRGGNLINVLAVHEDKRDQEAVPWNVPLTREALLDFFSDYHPRFRRLFELAPVDGIHVWQMRVVPPLANWIRDRVCILGDAAHASLPTMGQGFGQGLEDAVALATLLPLGTRRTDIPARLVAYEGMRKPRTEWIARESFEQQAVAEKRGIYLRSIEMRDAVMGYNVREEAKRVLSELTKSDC

SEQ ID NO: 29 - protein, 72 aa, Artificial sequence
  Consensus amino acid sequence 1 for hispidin hydroxylase
  MISC_FEATURE (1)..(72): X - any amino acid
VGAGLXGXXAAXXLRRXGHXXXXXXXSXFKXEXGAGXAXPXNTXXXLXXLGCXXXNXXXVXNLCXXXXXXDG

SEQ ID NO: 30 - protein, 33 aa, Artificial sequence
  Consensus amino acid sequence 2 for hispidin hydroxylase
  MISC_FEATURE (1)..(33): X - any amino acid
GMXXXXXXYXXXYGXXWXMXHRVDXHXELXRXA

SEQ ID NO: 31 - protein, 96 aa, Artificial sequence
  Consensus amino acid sequence 3 for hispidin hydroxylase
  MISC_FEATURE (1)..(96): X - any amino acid
GPXAXLXLXXXVXXCXXXXXXXXXXXGXXXXADLXXGADGIXSXXRXXXXXXXXXXPXXXXXGXRWXXXXXXLXXXPELDWXXKXXPLGARXXXXP

SEQ ID NO: 32 - protein, 57 aa, Artificial sequence
  Amino acid consensus sequence 4 for hispidin hydroxylase
  MISC_FEATURE (1)..(57): X - any amino acid
RTIXXYACRXXXXXNVLXXHXDXRXQXXXXWXXPXXXXXXXXXXXDYHPRFXXLXXL

SEQ ID NO: 33 - protein, 83 aa, Artificial sequence
  Consensus amino acid sequence 5 for hispidin hydroxylase
  MISC_FEATURE (1)..(82): X - any amino acid
WQXRVXPXLXXWXXXXXCXXGDXAHASLPTXGQGFGXGLEDAXALXXLLXXXXXXXXXXXRLXXXXXXXKXRXEXXXXESXEQ

SEQ ID NO: 34 - DNA, 4832 bp, Neonothopanus nambi
aaaaccatccttatattctcgctttgatgctggctgtatggaaacttggaggcaccttcgctcctattgatgtccattctcctgccgaattggtagctggcatgctgaacatagtctctccttcttgcttggttattccgagctcagatgtaactaatcaaactcttgcgtgcgatcttaatatccccgtcgttgcatttcacccacatcaatccactattcctgagctgaacaagaagtacctcaccgattctcaaatttctccggatcttcctttttcagatccaaaccggcctgctctgtacctcttcacttcgtccgccacttctcgaagtaatctcaaatgcgtgcctctcactcacacctttatcttacgcaacagcctctcgaagcgtgcatggtgcaagcgtatgcgtccagagacagactttgacggcatacgcgttcttggatgggccccgtggtctcacgtcctagcacacatgcaagacatcggaccactcaccttacttaatgccggatgctacgtttttgcgactactccatccacgtaccctacggaattgaaggacgacagggacctgatatcttgcgcggcaaatgctatcatgtacaagggcgtcaagtcatttgcttgtcttccctttgtactcggagggctgaaggcattatgcgagtctgagccatccgtgaaggcgcatctacaggtcgaggagagagctcaactcctgaagtctctgcaacacatggaaattcttgagtgtggaggtgccatgctcgaagcaagtgttgcgtcttgggctattgagaactgcattcccatttcgatcggtattggtatgacggagactggtggagcgctctttgcaggccccgttcaggccatcaaaaccgggttttcttcagaggataaattcattgaagatgctacttacttgctcgttaaggatgatcatgagagtcatgctgaggaggatattaacgagggtgaactagttgtgaaaagtaaaatgctcccacgaggctaccttggctatagtgatccttccttctcagtcgacgatgctggctgggttacatttagaacaggagacagatacagcgttacacctgacggaaagttttcctggctgggccggaacactgatttcattcagatgaccagtggtgagacgctggatccccgaccaattgagagctcgctctgcgaaagttctcttatttctagagcatgcgttatcggagataaatttctcaacgggcctgctgctgctgtttgtgcgatcattgagcttgagcccacagcggtggaaaaaggacaagctcactcgcgtgagatagcaagagttttcgcacctattaatcgagacctaccgcctcctcttaggattgcatggtcgcacgttttggttctccagccctcggagaagataccgatgacgaagaagggtaccatcttccgcaagaaaattgagcaggtgtttggctctgcgttgggtggcagctctggagataactctcaagccactgcggatgctggcgttgttcgacgagacgagttatcgaacactgtcaagcacataattagccgtgttttaggagtttccgatgacgaattactttggacgctatcatttgcggagttaggaatgacgtcagcactagccactcgcatcgccaacgagttgaacgaagttttagttggagttaatctccctatcaacgcttgctatatacatgtcgaccttccttctctaagcaatgccgtctatgcgaaacttgcacacctcaagttaccagatcgtactcccgaacccaggcaagcccctgtcgaaaactctggtgggaaggagatcgttgtcgttggccaggcctttcgtcttcctggctcaataaacgatgtcgcctctcttcgagacgcattcctggcgagacaagcatcatccattatcactgaaataccatccgatcgctgggaccacgccagcttctatcccaaggatatacgtttcaacaaggctggccttgtggatatagccaattatgatcatagctttttcggactgacggcaaccgaagcgctctatctgtcgccaactatgcgtctagcattagaagtttcgtttgaagcgctagagaatgctaatatcccggtgtcacaactcaagggttcgcaaacagcggtttatgttgctactacagatgacggatttgagacccttttgaatgccgaggccggctatgatgcttatacaagattctatggcactggtcgagcagcaagtacagcgagcgggcgcataagctgtcttcttgatgtccatggaccctctattactgttgatacggcatgcagtggaggggctgtttgtattgaccaagcaatcgactatctacaatcatcgagtgcagcagacaccgctatcatatgtgctagtaacacgcactgctggccaggctcgttcaggtttctttccgcacaagggatggtatccccaggaggacgatgcgcgacatttacaactgatgctgatggctacgtgccctctgagggcgcggtcgccttcatattgaaaacccgagaagcagctatgcgtgacaaggacactatcctcgcgacaatcaaagcgacacagatatcgcacaatggccgatctcaaggtcttgtggcaccgaatgtcaactcgcaagctgaccttcatcgctcgttgcttcaaaaagctggccttagcccggctgatatccgtttcattgaagctcatgggacaggaacgtcactgggagacctctcagaaattcaagctataaatgatgcttatacctcctctcagccgcgcacgaccggcccactcatagtcagcgcttccaaaacggtcattggtcataccgaaccagctggccccttggtcggtatgctgtcggtcttgaactctttcaaagaaggcgccgtccctggtctcgcccatcttaccgcagacaatttgaatccctcgctggactgttcttctgtgccacttctcattccctatcaacctgttcacctggctgcacccaagcctcaccgagctgctgtaaggtcatacggcttttcaggtaccctgggcggcatcgttctagaggctcctgacgaagaaagattagaagaagagctgccaaatgacaagcccatgttgttcgtcgtcagcgcaaagacacatacagcactaatcgaatacctggggcggtatctcgagttcctcttgcaggcgaacccccaagatttttgtgacatttgttatacaagctgcgttgggcgggagcactatagatatcgctatgcttgtgtagcaaatgatatggaggacctcataggccaactccagaaacgtttgggcagcaaggtgccgccaaagccgtcatacaaacgcggtgctttggcctttgccttttctggtcagggtacacaattccgagggatggcgacagagcttgcaaaagcgtactccggcttccgaaagatcgtgtcggatctcgcaaagagagctagcgagttgtcaggtcatgccattgaccgttttcttcttgcatatgacataggcgctgaaaatgtagctcctgatagtgaggcagaccagatttgcatctttgtgtatcagtgttctgtccttcgctggctgcagactatggggattagacccagtgcagtgataggccatagcctcggggagatctcagcttctgtggcggcaggagcactttctcttgactccgctttggatcttgtcatctcacgagctcgccttttgcgctcttcggcaagtgctcctgcaggaatggcagctatgtctgcctcgcaagacgaggttgtggagttgattgggaaactagacctcgacaaggctaattcgctcagcgtttcggtcataaatggtccccaaaatactgtcgtgtccggctcttcagcggctattgaaagcatagtggctttagcgaaagggagaaagatcaaagcgtctgccctgaatatcaatcaagcttttcatagtccatacgtcgacagtgccgtccctggtctccgtgcttggtcagaaaagcatatctcctcagctcggccattgcaaattccgctgtattcaacgttgttgggagcacaaatctctgagggagagatgttgaatccagatcactgggtcgaccatgcacggaagcctgtacagttcgcacaagcagccacaaccatgaaagaatccttcaccggagtcatcatagatatcggccctcaagtagtggcttggtcacttctgctctcgaacgggctcacgtccgtgactgcgctcgctgcgaaaagagggagaagtcaacaggtggctttcttaagcgccttggcggatttgtatcaagattacggtgttgttcctgattttgtcgggctttatgctcagcaggaagatgcttcgaggttgaagaagacggatatcttgacgtatccgttccagcggggcgaagagactctttctagtggttctagcactccgacattggaaaacacggatttggattccggtaaggaattacttatgggaccgactcgggggttgttacgcgcggacgacttgcgtgacagtatcgtttcttctgtgaaggatgttctggaactcaagtcaaatgaagacctcgatttgtctgaaagtctgaatgcgcttggtatggactcgatcatgttcgctcagttacggaagcgtattggggaaggactcggattgaatgttccgatggtttttctgtcggacgcgttttctattggtgagatggttagtaatcttgtggaacaggcggaggcgtctgaggacaat

SEQ ID NO: 35 - protein, 1678 aa, Neonothopanus nambi
MNSSKNPPSTLLDVFLDTARNLDTALRNVLECGEHRWSYRELDTVSSALAQHLRYTVGLSPTVAVISENHPYILALMLAVWKLGGTFAPIDVHSPAELVAGMLNIVSPSCLVIPSSDVTNQTLACDLNIPVVAFHPHQSTIPELNKKYLTDSQISPDLPFSDPNRPALYLFTSSATSRSNLKCVPLTHTFILRNSLSKRAWCKRMRPETDFDGIRVLGWAPWSHVLAHMQDIGPLTLLNAGCYVFATTPSTYPTELKDDRDLISCAANAIMYKGVKSFACLPFVLGGLKALCESEPSVKAHLQVEERAQLLKSLQHMEILECGGAMLEASVASWAIENCIPISIGIGMTETGGALFAGPVQAIKTGFSSEDKFIEDATYLLVKDDHESHAEEDINEGELVVKSKMLPRGYLGYSDPSFSVDDAGWVTFRTGDRYSVTPDGKFSWLGRNTDFIQMTSGETLDPRPIESSLCESSLISRACVIGDKFLNGPAAAVCAIIELEPTAVEKGQAHSREIARVFAPINRDLPPPLRIAWSHVLVLQPSEKIPMTKKGTIFRKKIEQVFGSALGGSSGDNSQATADAGVVRRDELSNTVKHIISRVLGVSDDELLWTLSFAELGMTSALATRIANELNEVLVGVNLPINACYIHVDLPSLSNAVYAKLAHLKLPDRTPEPRQAPVENSGGKEIVVVGQAFRLPGSINDVASLRDAFLARQASSIITEIPSDRWDHASFYPKDIRFNKAGLVDIANYDHSFFGLTATEALYLSPTMRLALEVSFEALENANIPVSQLKGSQTAVYVATTDDGFETLLNAEAGYDAYTRFYGTGRAASTASGRISCLLDVHGPSITVDTACSGGAVCIDQAIDYLQSSSAADTAIICASNTHCWPGSFRFLSAQGMVSPGGRCATFTTDADGYVPSEGAVAFILKTREAAMRDKDTILATIKATQISHNGRSQGLVAPNVNSQADLHRSLLQKAGLSPADIRFIEAHGTGTSLGDLSEIQAINDAYTSSQPRTTGPLIVSASKTVIGHTEPAGPLVGMLSVLNSFKEGAVPGLAHLTADNLNPSLDCSSVPLLIPYQPVHLAAPKPHRAAVRSYGFSGTLGGIVLEAPDEERLEEELPNDKPMLFVVSAKTHTALIEYLGRYLEFLLQANPQDFCDICYTSCVGREHYRYRYACVANDMEDLIGQLQKRLGSKVPPKPSYKRGALAFAFSGQGTQFRGMATELAKAYSGFRKIVSDLAKRASELSGHAIDRFLLAYDIGAENVAPDSEADQICIFVYQCSVLRWLQTMGIRPSAVIGHSLGEISASVAAGALSLDSALDLVISRARLLRSSASAPAGMAAMSASQDEVVELIGKLDLDKANSLSVSVINGPQNTVVSGSSAAIESIVALAKGRKIKASALNINQAFHSPYVDSAVPGLRAWSEKHISSARPLQIPLYSTLLGAQISEGEMLNPDHWVDHARKPVQFAQAATTMKESFTGVIIDIGPQVVAWSLLLSNGLTSVTALAAKRGRSQQVAFLSALADLYQDYGVVPDFVGLYAQQEDASRLKKTDILTYPFQRGEETLSSGSSTPTLENTDLDSGKELLMGPTRGLLRADDLRDSIVSSVKDVLELKSNEDLDLSESLNALGMDSIMFAQLRKRIGEGLGLNVPMVFLSDAFSIGEMVSNLVEQAEASEDN

SEQ ID NO: 36 - DNA, 4512 bp, Panellus stipticus
atgcctcctgctccttcctccatccttgatgtgttcaccgagactgcatacaatcctcgcacctccaaccgtcctgtagtagaatgtggcgagcacctctggacatactcacaacttgaagcagtttccaatgccatgtcgcaagacctggagaactcgatcggtctttacgcgaaggtcgcttttgtcggtgaaaaccatccatttgttttcgctctcatgcttgcggtctggaaaattgctggcacattcatccccattgatgcgcacattcctcccgccctcctagatggcatggtcgacattgtgaagccgatgcgcatctatgtatcatcggccgatacctctaattcctcctgggcctcagaactcgcggtcgagtccaaactcgcgtggtggcgacgcatgcagccaagcatcaacctagacaacacccgagtcctcggctgggccccgtggtcgcatgtattatcccacatgcaggacattggcaccgctaccattctcaccgcaggctgttacgtctttgcctctatcccttccacatatcagcttcagcaggcgccaacagatcttacgacccaagtcatcaacggtatcctcaataaaaacatctcggcgcttgctgcccttccatttgttttcggcggtatcaaagcagcgtgcgagtcgggcgatctggatgtggaggcacttctgggtgccctgcgccgcatgacgatgctcgaatgtggcggggctgcgctcgaccctgcaattgcaaattggacggatataaatggtgtatcgctcatggttgggattggaatgacggaaacgggtggtgcaatcttcgcggggagggcgaaagactcgctctccgggtttcttgctgagggcctgatctcagatgccagaattgagcttgacaagggtaaatctgatggctcggatgagggagagcttgttgtcacaagcaagctacttccacatggctacattggcttcgatgacgggtcattttccgtggatgcgcaagggtgggtgacgttcaagacaagggattgctatcgtgtcaaggactcgaagtttatttggctaggccgggccactgattacattcaggcgagcacatctctccttcctcagcgctttaaacatgaactgcgctatcagatgacaagcggcgaatcccttgacccgcgtcccctcgaaaatttcttacgctccgccgagttcatctccaatgtgtgcgtcataggtgacaacttcctccggggtgcctccacttctgtgtgggccgttatcgagctcacggacagcgcgcaccgtctggatgccgcttcagcgagaaagcaggtggcgcgcgtgctggcgccgctcaatggtggcctcccacccgctctccggatctcgatgtcctcggtgttgatattgagcggatcacagaaaattccgcgcacgaagaagggcgaaatcttccgtaagcagattgaggatctgtttggtgccgcgatgagcatgccgcctgaggctgagccggctctggaggatgaactaacaagtattgttggaaatgtcctcaacatatcggacagcgacatgttgacctcgatgacttttgccgaacttggaatgacctctgttctcgcagtaaaaatctcagcaaggctcaacgagcaccttgctgggcgggccgtcctcccggccaacatctgttacatctacatcgatactccatcactgattgccggcatccggaacttcctttcgccggcatcttctgatcaagagccatcgaactttgccaccacggcagacaagaaggacgaaatcgtcatcataggcaaagccttccggcttcccggcgggatcaacgacgactctgctctctgggctgctcttatgggcaagaacgactctgttattgcagacatcccgccagaccgttgggatcatgctagcttctatcccgcccacatctgcttgcgcaaggccgggcttatagatatgtccagctatgactatgggttctttggcctttcggcgactgaggcgtactatgtgtcgcccaccatgcgcgcagcgctcgaggtggcgttcgaagcgctggaggatgcgaacatcccggtgtcaagaatcaaggggacaaatacgagtgtttttgttgcaacgaaagatgatggatttgagacattgctgaatgcggcgcatggatttgatgatgctgacggatacgtcccttcagaaggcgccgtcgcttttatactgaagacacggacggcggcggagagggatggggatcgaattatggccatcatcaaagccacggaagtctcgcataatggaagatctcagggactcgccgcgccgaacgtcaaggcgcaagccgctcttcatagagcagtcttgcgcaaagctaagctcgaccctctggacatccatttcattgaagcccacggaactggaacaccgctgggcgatctttgtgaaatacaaggcataaatgaagcctttgtctccgcacgtccacgtgcggaggatccactcattgtcagcgcctcaaagtcttcccttgggcacaccgaaccttcggctggattggttgggatgctatctgtgttgatggccttgaagcacggcatcgtgcctgggttgctccatctccgagcggacaatgctaaccaccaactggacctgacacaagttccacttcgtatatcaccggaacctgtagtcatcgccgcatccaagccgcatcacgccatggtgctatcctatggattttcaggtacattggcagacattgttttggagagtccggaagaaccatcgtccccaaacccgggagccgcgggcccaatgatatttgtcctcagcgcgaagacttccgcggctctgtcggcatatatcaaggcttacattgcatttctacagaatgcagacgcgcacgagttttacaacatctgctacaccgcctgcgttggaagagaacattacaaacacagatttgcttgcgttgcaaatgaccttgctgatctgattcgccaattacaagactgtgcgagtgcgctggctccaaccaaaagtagtactggtgccttggcgttcgcgttccctggccaaggcgtgcaatttccaggcatggccgctgcacttgctaaaaggcattccatatttcgcgactatgtcatggaattcggtgatagagcacaagatctttgtggcttgcccatcgcaaagatgttgctggacgtggatgcagcagaggaagaagatatccacagtgacgtagaccagatttgcgtctttgtctatcagtattcgatgtgtcgatggcttagggagctcgggctcgaggcaagtgcggttatcgggcacagtttgggtgaaataacggccgcacttatcggggacgcatttacatttgaagtggctctcgatctcgttgtcactcgaggacggctactccgcccttctcaagggaatccaggcgggatggctgcgctggcatgccccgaggagaatgtcccggcgattttggaccaatgtcgtgtggacagtaccattagcgtctccgttatcaatggtccgaggagcctttgtgtatccggagcttccaacgacatcgacgagtttgtcaagatggcaaaacggcaaaacatcaaagcgactcgactgagggtggaccaaggatttcatagccctcgagttgatagtgccgcggttggactgcgtgcatggtcaggttctttttccaaatcatttcagccgttgcgcatcacattatactctacttctctgggtgctgcaatctcgaaaggagagattttgaatcagacgcattgggccgatcacgtccgccgtccggtcatattctcaaaagcagcagcagccatcctcgaggacaagtccattggcgcgatcctggatatcggaccacagacggtggcatggtctctccttctggcgaacggctgcaacgtcgcgtcagctgttgccctgtccggccgaagagtacaagatcaggaaacagcctttttatctgcactggcgaatctgtatcaaaatcacggggtgacgccgaattttcgcgtattttatgctcaccaggcagtccaggcgcgctatagaaccgtggacatcccgaagtatcccttccaacgccgacatcgatatccatcctacattccatcgcgcaatgccacgggtgccaacagactgaaagaaccattccgtagcgacctggatgaaccggctcaagacacggagcacaccgcggaactgagagtggacatgactccggagaagctccgggacgccctgatgcactgtgtgcgggacacattggaaggcgaagattttgatgaatcggaatccctcgtttcgcgtggaattgactccattacttttgcgggtttacggaagcgtgttcaagaacgacacggacttaatctttccatcattttctggtctgatgggttttctgtgaaagacatggtcgacagcctcatcgaacagcattttgtccac

SEQ ID NO: 37 - protein, 1504 aa, Panellus stipticus
MPPAPSSILDVFTETAYNPRTSNRPVVECGEHLWTYSQLEAVSNAMSQDLENSIGLYAKVAFVGENHPFVFALMLAVWKIAGTFIPIDAHIPPALLDGMVDIVKPMRIYVSSADTSNSSWASELAVESKLAWWRRMQPSINLDNTRVLGWAPWSHVLSHMQDIGTATILTAGCYVFASIPSTYQLQQAPTDLTTQVINGILNKNISALAALPFVFGGIKAACESGDLDVEALLGALRRMTMLECGGAALDPAIANWTDINGVSLMVGIGMTETGGAIFAGRAKDSLSGFLAEGLISDARIELDKGKSDGSDEGELVVTSKLLPHGYIGFDDGSFSVDAQGWVTFKTRDCYRVKDSKFIWLGRATDYIQASTSLLPQRFKHELRYQMTSGESLDPRPLENFLRSAEFISNVCVIGDNFLRGASTSVWAVIELTDSAHRLDAASARKQVARVLAPLNGGLPPALRISMSSVLILSGSQKIPRTKKGEIFRKQIEDLFGAAMSMPPEAEPALEDELTSIVGNVLNISDSDMLTSMTFAELGMTSVLAVKISARLNEHLAGRAVLPANICYIYIDTPSLIAGIRNFLSPASSDQEPSNFATTADKKDEIVIIGKAFRLPGGINDDSALWAALMGKNDSVIADIPPDRWDHASFYPAHICLRKAGLIDMSSYDYGFFGLSATEAYYVSPTMRAALEVAFEALEDANIPVSRIKGTNTSVFVATKDDGFETLLNAAHGFDDADGYVPSEGAVAFILKTRTAAERDGDRIMAIIKATEVSHNGRSQGLAAPNVKAQAALHRAVLRKAKLDPLDIHFIEAHGTGTPLGDLCEIQGINEAFVSARPRAEDPLIVSASKSSLGHTEPSAGLVGMLSVLMALKHGIVPGLLHLRADNANHQLDLTQVPLRISPEPVVIAASKPHHAMVLSYGFSGTLADIVLESPEEPSSPNPGAAGPMIFVLSAKTSAALSAYIKAYIAFLQNADAHEFYNICYTACVGREHYKHRFACVANDLADLIRQLQDCASALAPTKSSTGALAFAFPGQGVQFPGMAAALAKRHSIFRDYVMEFGDRAQDLCGLPIAKMLLDVDAAEEEDIHSDVDQICVFVYQYSMCRWLRELGLEASAVIGHSLGEITAALIGDAFTFEVALDLVVTRGRLLRPSQGNPGGMAALACPEENVPAILDQCRVDSTISVSVINGPRSLCVSGASNDIDEFVKMAKRQNIKATRLRVDQGFHSPRVDSAAVGLRAWSGSFSKSFQPLRITLYSTSLGAAISKGEILNQTHWADHVRRPVIFSKAAAAILEDKSIGAILDIGPQTVAWSLLLANGCNVASAVALSGRRVQDQETAFLSALANLYQNHGVTPNFRVFYAHQAVQARYRTVDIPKYPFQRRHRYPSYIPSRNATGANRLKEPFRSDLDEPAQDTEHTAELRVDMTPEKLRDALMHCVRDTLEGEDFDESESLVSRGIDSITFAGLRKRVQERHGLNLSIIFWSDGFSVKDMVDSLIEQHFVH

SEQ ID NO: 38 - DNA, 4803 bp, Panellus stipticus
atggcactggtctctcccacttgttcgtctcagatgcctcctgctccttcctccatccttgatgtgttcaccgagactgcatacaatcctcgcacctccaaccgtcctgtagtagaatgtggcgagcacgtctggacctactcacaacttgaagcagtttccaatgccatgtcgcaagacctggagaactcgatcggtctttacgcgaaggtcgcttttgtcggtgaaaaccatccatttgttttcgctctcatgcttgcagtctggaaaattgctggcacattcatccccattgatgcgcacattcctcccgccctcctagatggcatggtcgacattgtgaagccgatgtgcatctatgtatcatcggccgatacctctaattcctcctgggcctcagaactcgcggtcgaggttcgggtattccgtccggaagaatctacgattccggctttgaacgaacactatggaagatccagcatcactcccgctcagcatcggccaatactcaacgtggtccaggcagttcccctgacacacaaatttatcctcagcaactgtcagtccaaactcgcgtggtggcgacgcatgcagccaagcatcaacctagacaacacccgagtcctcggctgggccccgtggtcgcatgtattatcccacatgcaggacattggcaccgctaccattctcaccgcaggctgttacgtctttgcctctatcccttccacatatcagcttcagcaggcgccaacagatcttacgacccaagtcatcaacggtatcctcaataaaaacatctcggcgcttgctgcccttccatttgttttcggcggtatcaaagcagcgtgcgagtcgggcgatctggatgtggaggcacttctgggtgccctgcgccgcatgacgatgctcgaatgtggcggggctgcgctcgaccctgcaattgcaaattggacggatataaacggtgtatcgctcatggttgggattggaatgacagaaacgggcggtgcaatcttcgcggggagggcgaaagactcgctctccgggtttcttgctgagggtctgatctcagacgccagaattgagcttgacaagggtgaatctgatggctcggatggtacgacgttctccatcttctcgaccactgccggaattaaacctgattgtcagagggagagcttgttgtcacaagcaagctacttccacatggctacattggcttcgatgacgggtcattttccgtggatgcgcaagggtgacaacttcctccggggtgcctccacttctgtgtgggccattatcgagctcacggacagcgcgcaccgcctggatgccgcttcagcgagaaagcaggtggcgcgcgtactggcgccgctcaatggtggcctcccacccgctctccggatctcaatgtcctcggtgttgatattgagcggatcacagaaaattccgcgcacgaagaagggcgagatcttccgtaagcagattgaggatctgtttggtgccgcgatgagcatgccgcctgaggctgagccggctctggaggatgaactaacaagtattgttggaaatgtcctcaacatatcggacagcgacatgttgacctcgatgacttttgccgaacttggaatgacctctgttctggcagtaaaaatctcagcaaggctcaacgagcaccttgctgggcgggccgtcctcccggccaacatctgttacatctacatcgatactccatcactgattgccggcatccggaacttcctttcgccgacatcttctgatcaagagccatcgaactttgccaccacggcagacaagaaggacgagatcgtcatcataggcaaagcctttcggcttcccggcgggatcaacgacgactctgctctctgggctgctcttatgggcaagaacgactctgttattgcagacatcccgccagaccgttgggatcatgctagcttctatcccgcccacatctgcttgcgcaaggctgggcttatagatatgtccagctatgactatgggttctttggcctttcggcgactgaggcgtactatgtgtcgcccaccatgcgcgcagcgctcgaggtggcgttcgaagcgctggaggatgcgaacatcccggtgtcaagaattaaggggacaaatacgagtgtttttgttgcaacgaaagatgatggatttgagacattgctgaatgcggcgcatggatttgatgcgtatacccggttctacgggactgggcgggctccaagtactgccagtgggcgcataagctaccttcttgacatccatgggccctcactcacagtagatacggcctgtagcggaggaattgtctgcattgatcaagatataccagcgaatttatgtatcatagcgatcgcatacctccagtctggcgccggtgaatcagccatcgaacgacaatacaggtttctcacggcgcagaacatggcatcgcccactagccgctgttccaccttcactgcagatgctgacggatatgtcccttcagaaggcgccgtcgcttttatactgaagacacggactgcggcggagagggatggggatcgaattatggccatcatcaaagccacagaagtctcgcataatggaagatctcagggactcgccgcgccgaacgtcaaagcgcaagccgctcttcatagagcagtcttgcgcaaagctaagctcgaccctctggacatccatttcattgaagcccacggaactgttttaggaacaccgctgggtgatctttgtgaaatacaaggcataaatgaagcctttgtctccgcacgtccacgtgcggaggatccactcattgtcagcgcctccaagtcttcccttgggcacaccgaaccttcggctggattggttgggatgctatctgtgttgatggccttgaagcacggcatcgtgcctgggttgctccatctccgagcggacaatgctaaccaccaactggacctgacacgagtcccactgcgtatatcaccggaacctgtagtcatcgccgcatccaagccgcatcacgccatggtgctgctcacagtccgtacattggcagacattgttttggagagtccggaagaaccaccgtcccagaacccgggagccgcgggcccaatgatatttgtcctcagcgcgaagacttccgcggctctgtcggcatatatcaaggcttacattgcatttctacagaatgcagacgcgcacgagttttacaacatctgctacaccgcctgtgttggaagagaacattacaaacacagatttgcttgcgttgcaaatgaccttgctgatctgattcgccaattacaagactgtgcgagtgcgctggctccaaccaaaagtagtactggtgccttggcgttcgcgttccctggccaaggcgtgcaatttccaggcatggccgctgcacttgctaaaaggcattccatatttcgcgactatgtcatggaatttggtcatagagcacaagatctttgtggcttgcccatcgcaaagatgttgctggacgtggatgcagcagaggaagaagatatccacagtgacgtagaccagatttgcgtctttgtctatcagtattcgatgtgtcgatggcttagggagctcgggctcgaggcaagtgcggttatcgggcacagtttgggcgaaataacagccgcacttatcggggacgcatttacatttgaagtggctctcgatctcgttgtcactcgaggacggctactccgcccttctcaagggaatccaggcgggatggctgcgctggcatgccccgaggagaatgtcccagcgattttggaccaatgtcgtgtggacagtaccattagcgtctccgttatcaatggtccgaggagcctttgtgtatccggagcttccaacgacatcgacgagtttgtcaagatggcaaaacggcaaaacatcaaagcgactcgactgagggtggaccaaggatttcatagccctcgagttgatagtgccgcggttggactgcgtgaatggtcaggttctttttcgaaatcatttcagccgttgcgcatcacattatactctacgtctctgggtgctgcaatctcgaaaggagagattttgactcagacacattgggccgatcacgtccgccgtccggtcatattctcaaaagcagcagcagccatcctcgaggacaagtccattggcgcgatcctggatatcggaccacagacggtggcatggtctctccttctggcgaacggctgcaacgtcgcgtcagctgttgccctgtccggccgaagagtacaagatcaggaaacggcctttttatctgcactggcgaatctgtatcaaaatcacggggtgacgccgaattttcgcgtattttacgctcaccaggcagtccaggcgcgctatagaaccgtggacattccgaagtatcccttccaacgccgacatcgatatccatcctacattccatcgcgcaatgccacgggtgccaacagactgaaagaaccattccgtagcgacctggatgaaccggctcaagacacggagcacaccgcggaaatgagagtggacatgactccggagaagctccgggacgccctgatgcactgtgtgcgggacacactggaaggcgaagattttgatgaatcggaatccctcgtttcgcgtggaattgactccattacttttgcgggtctacggaagcgtgttcaagaacgacacggacttaatctttccatcattttctggtctgatgggttttctgtgaaagacatggtcgacagcctcatcgaacagcattttgtccac

SEQ ID NO: 39 - protein, 1601 aa, Panellus stipticus
MALVSPTCSSQMPPAPSSILDVFTETAYNPRTSNRPVVECGEHVWTYSQLEAVSNAMSQDLENSIGLYAKVAFVGENHPFVFALMLAVWKIAGTFIPIDAHIPPALLDGMVDIVKPMCIYVSSADTSNSSWASELAVEVRVFRPEESTIPALNEHYGRSSITPAQHRPILNVVQAVPLTHKFILSNCQSKLAWWRRMQPSINLDNTRVLGWAPWSHVLSHMQDIGTATILTAGCYVFASIPSTYQLQQAPTDLTTQVINGILNKNISALAALPFVFGGIKAACESGDLDVEALLGALRRMTMLECGGAALDPAIANWTDINGVSLMVGIGMTETGGAIFAGRAKDSLSGFLAEGLISDARIELDKGESDGSDGTTFSIFSTTAGIKPDCQRESLLSQASYFHMATLASMTGHFPWMRKGDNFLRGASTSVWAIIELTDSAHRLDAASARKQVARVLAPLNGGLPPALRISMSSVLILSGSQKIPRTKKGEIFRKQIEDLFGAAMSMPPEAEPALEDELTSIVGNVLNISDSDMLTSMTFAELGMTSVLAVKISARLNEHLAGRAVLPANICYIYIDTPSLIAGIRNFLSPTSSDQEPSNFATTADKKDEIVIIGKAFRLPGGINDDSALWAALMGKNDSVIADIPPDRWDHASFYPAHICLRKAGLIDMSSYDYGFFGLSATEAYYVSPTMRAALEVAFEALEDANIPVSRIKGTNTSVFVATKDDGFETLLNAAHGFDAYTRFYGTGRAPSTASGRISYLLDIHGPSLTVDTACSGGIVCIDQDIPANLCIIAIAYLQSGAGESAIERQYRFLTAQNMASPTSRCSTFTADADGYVPSEGAVAFILKTRTAAERDGDRIMAIIKATEVSHNGRSQGLAAPNVKAQAALHRAVLRKAKLDPLDIHFIEAHGTVLGTPLGDLCEIQGINEAFVSARPRAEDPLIVSASKSSLGHTEPSAGLVGMLSVLMALKHGIVPGLLHLRADNANHQLDLTRVPLRISPEPVVIAASKPHHAMVLLTVRTLADIVLESPEEPPSQNPGAAGPMIFVLSAKTSAALSAYIKAYIAFLQNADAHEFYNICYTACVGREHYKHRFACVANDLADLIRQLQDCASALAPTKSSTGALAFAFPGQGVQFPGMAAALAKRHSIFRDYVMEFGHRAQDLCGLPIAKMLLDVDAAEEEDIHSDVDQICVFVYQYSMCRWLRELGLEASAVIGHSLGEITAALIGDAFTFEVALDLVVTRGRLLRPSQGNPGGMAALACPEENVPAILDQCRVDSTISVSVINGPRSLCVSGASNDIDEFVKMAKRQNIKATRLRVDQGFHSPRVDSAAVGLREWSGSFSKSFQPLRITLYSTSLGAAISKGEILTQTHWADHVRRPVIFSKAAAAILEDKSIGAILDIGPQTVAWSLLLANGCNVASAVALSGRRVQDQETAFLSALANLYQNHGVTPNFRVFYAHQAVQARYRTVDIPKYPFQRRHRYPSYIPSRNATGANRLKEPFRSDLDEPAQDTEHTAEMRVDMTPEKLRDALMHCVRDTLEGEDFDESESLVSRGIDSITFAGLRKRVQERHGLNLSIIFWSDGFSVKDMVDSLIEQHFVH

SEQ ID NO: 40 - DNA, 5073 bp, Neonothopanus gardneri
atgaattccaaagtgaatcttccctccactttgcttgatgttttcctcgagatcgctggcgagccatctaccgcttcgcgtgatgttttggaatgtggcgagcacagatggacttaccagcagcttgacgttgtttcatctgctttagcccagcatctcaagtacactgtcggtctatctcctacagtcgcagtgatcagcgaaaatcatccttatgtttttgccttgatcctggctgtgtggaaagttgggggcattttcgcgcccctcgacgcacatgctcctgctgagttggtagctggcatgttaagcataatctctccttcgtgcttagtacttcagagcacagatgtagctaatcaaactcttgcgtgtgatctcgatattcctgttgaggtattccactcgcgtcaatccactattcctgaactaaacaagaaatatctcaccgattctgggttaccggcgggttttccgctctcagattcaaacaaaccggctctgtatctcttcacctcgtctgccacttctcggagcaatcttaaatgcgtgcctctcactcacgctttcatcctgagcaatagcctctcgaaacgcgcatggtgccaacgtatgcggccagagacagacttcgatggcatacgcgttcttggatgggctccatggtctcatgtcttggcgcacatgcaggacatcgggcccgtcactttactcaatgctggatgctacgtctttgctactatcccttcctcgtaccccacggatgtgcagagtgacagcaatttgatatctcatgtcgcaaatgctatcatacacaagggtgtaaaatcgtttgcttgtctcccttttgtactcggagggctgaaagcattatgcgagtccaagccatccgtcaaagcagatctacaggtcgaagagcaagctcagcttttgatctctctaaggcgcatggaaattcttgaatgcggaggtgcgatgctcgaagcgaatgttgcgtcttgggctatcgagaatcatattccggtctctattggtatcggtatgacagaaactggtggcgcgcttttcgctggacctgttcaggatatccggaccggtttttctgcggacaataaattcatcaaggacgctacctacttcctagttgcaaatggagatgaatctgggaacgatgtaactgaaggggagctagttgtgaaaagtaaaatgctcccgcgaggctatcttggctacaatgattcttccttttccgttgacgatgacggattggttacgttcaagacaggcgacagatacagtgttacacgcgacggaagattctcttggctaggcaggaatactgatttcattcagatgactagtggagagacattagatccccgacctgtcgagagttcgctctgccaaagtcctctcatctctcaagcatgcgttattggagacaagtttctcaacgggcctgccactgctgtttgcgcgatcgttgagcttgagccgacaatggtagaaacgggagaggctaactcgcgggacatagtccaagtctttgcacccatcaaccgagacctgcctcctcctttaaggatagcatggtcgcacattttaattctcaagcattccgagaagataccgatgacgaagaagggaacgattttccgtaagaagattgagcagatgtttggcgctgcattgggcattgcttccatccctacaaacagtgtcaatggatccgttggctccggagaagagcctcaagctactgcagatgagactgttctacaagatgaactatcaaataccgtgaagaaaataatcagccgtgttctaggagtaactgatgaggaattgctttggacactgtcattcgcagagctgggaatgacctcaacactggctattcgtatcgtaaacgagctgaacgaaactctcgttgggggcgatctccctatcaatgcttgctacatttatgtcgacctttcctctctgagcaaggctgtgtatgcgaaactggctcatctcgagctgtcagatcatgttcctgagctcaaacaagctcccttcaagtcttctggcgggaaagagattgtcattgtcggccaagcgtttcgtctacccggctcaatcaatgacgttgcctctcttcgggatgcattcctagcaagacagacatcgtccatcatcgctggaataccttccgatcgctgggaccatgccagcttctatcccaaggacatatgtttcgacaaggctggtcttgtggatatagctcattatgatcatagcttcttcggaatcacagcaacggaagcgctccatctgtctccaaccatgcgtcttgcactggaagtttcgtttgaggcgcttgagaatgctaatatcccaacgtcgcgattgaaaggctcgcagacggcagtttatgttgcaacaacagatgacggatttgagaccctgttgaatgctgaggctggctatgaagcttatacacggttttatggtactggtcgggcagcaagtacggcgagtgggcgcataagctatcttcttgatatacatggaccctccgtcactattgacacggcatgcagtggtggagttgtttgtatcgatcaagcaatcaactatttacaatcgtcgagttcagcggacaccgctatcgtgttcctttctgctcaagggatggtctccccgaggggacgatgtgcgacatttacggctgatgctgacggctatgtcccttctgagggtgcagttgctttcgtattgaaaactcgcgaagccgctatacgcgacaaagacaacattctcgctacaatcaaagcgacacagatttcgcacaacggccgctcacaaggacttgtagcaccgaacgtaaactcgcaagttgaccttcatcgttcgttgctcgagaaagctcgtcttagtcctgctgatgtccaattcattgaagctcatgggacaggaacgtcattgggagatctctcagagattcaagctataaacgctgcttacacttcctctcagccacgtacgaccggcccactcatagtcagcgcttccaagactgtcgttggccataccgaaccagctggtcctttggttggtatgttgtcggtcttgctgtctttccaggaaggcgcagtccctggtctcgctcatcttaccacacgtaacctcaatcctacgttggactattcttcagtgccgcttctcattccctctgaacctgttcgtctacaaacaccaaagccttatagagctgccgtaatgtcctacggcttttcgggtaccctggccggcctcgttctagagagccctgacgaacgtagctcagaagaagagccgcctgatgacaagccgatgctgttcgtcgttagcgcaaagacacacacggcactaattgaatacctgcaacggtatctcgagttcctcttacatgcgaatccccgcgatttctgtgatatctgctacacaagctgtgtcgggagggaacactatagatatcggtttgcttgtgttgcaaatgacatggaagacctcattggccaacttcaaagacgtttgtctagcaagttaccatcgaagccgctatacaaacgcggtgctctggcctttgcgttttctggacaggggacacagtttcaaggaatggcgacagatcttgcgaagaggtactctggcttccgaaaaatcgtttccggactcgcaaagagtgctggcgagctctcgggttacgctattgaccgttatcttctcgcgtatgacgttggtagcagtattgctactcctaatagtgaggtggaccagatctgcattttcgtataccaatgctctgtccttcgctggttggggagtattggaattaaaccaaacgtggtaatcggccatagccttggagagatttcggcttctgtggcggcaggggcactttctcttgacattgctctggatcttgtcatctcacgagctgggttgttgcgcccctcgacagatgttcctgcgggaatggctgctgtggccgcttcacaacaggaggtcattgagttgattgatgcgctggaccccgacaaggcaaattcgctcagtgtttcggttataaatggacctcaaaatatcgttgtgtcaggcgcttcagcagctattgagagaatggtcgcttctgcgaaggagaagaagatcaaagcttctgttctgaatattagccaagcttttcacagttcgtatgttgacagtgccattacgggtcttcgagcttggtcagaaaaacatatttcctcagcgataccactgcagattccgctgtattcgacattgctgggagctcggatatcaaagggccaaaaactgaacccagaccactgggtcgaccacgcacggaagcccgtacagttcgcacaagcagctacggcgatgaaagaaaccttcaccggagtcatcatggatatcggaccccaagcagtagcatggtcacttctgcttgcaaacggactcacatccgtaaccgcgcttgctgcgaagagagggaggagtcagcaggtggctttcttgagcgccttggcggagttgtatcaggattatggcattgttcctgattttgtggcgctttatgctcaggaggaggaaatagacaagttgaggaagacggatattttgacatatccgtttcagcgtgttaggaggtatccgagttttataccttcaaggcgtgtgagaggtggagaaattccttcgagccatcccagcgagtctgcgacgttggagaacacgaatgagggtacggctttgcgtgctgagtcgagggtgttgtgcagggaggatctgcatgatagcatcgttacctctgtgaaggatgttctagagctcaaaccaaatgaagatctagatttgtctgaaagcctgaacgcgcttggtgtggactctataatgtttgctcagttaaggaaacgtattggggagggactcgggttgagtatcccgacagtgttcctttcggatgccttttctattaatgagatggttaataatcttatggaacaggcggagacgcctggtgaagagggcgtaatgcaggagaat

SEQ ID NO: 41 - protein, 1691 aa, Neonothopanus gardneri
MNSKVNLPSTLLDVFLEIAGEPSTASRDVLECGEHRWTYQQLDVVSSALAQHLKYTVGLSPTVAVISENHPYVFALILAVWKVGGIFAPLDAHAPAELVAGMLSIISPSCLVLQSTDVANQTLACDLDIPVEVFHSRQSTIPELNKKYLTDSGLPAGFPLSDSNKPALYLFTSSATSRSNLKCVPLTHAFILSNSLSKRAWCQRMRPETDFDGIRVLGWAPWSHVLAHMQDIGPVTLLNAGCYVFATIPSSYPTDVQSDSNLISHVANAIIHKGVKSFACLPFVLGGLKALCESKPSVKADLQVEEQAQLLISLRRMEILECGGAMLEANVASWAIENHIPVSIGIGMTETGGALFAGPVQDIRTGFSADNKFIKDATYFLVANGDESGNDVTEGELVVKSKMLPRGYLGYNDSSFSVDDDGLVTFKTGDRYSVTRDGRFSWLGRNTDFIQMTSGETLDPRPVESSLCQSPLISQACVIGDKFLNGPATAVCAIVELEPTMVETGEANSRDIVQVFAPINRDLPPPLRIAWSHILILKHSEKIPMTKKGTIFRKKIEQMFGAALGIASIPTNSVNGSVGSGEEPQATADETVLQDELSNTVKKIISRVLGVTDEELLWTLSFAELGMTSTLAIRIVNELNETLVGGDLPINACYIYVDLSSLSKAVYAKLAHLELSDHVPELKQAPFKSSGGKEIVIVGQAFRLPGSINDVASLRDAFLARQTSSIIAGIPSDRWDHASFYPKDICFDKAGLVDIAHYDHSFFGITATEALHLSPTMRLALEVSFEALENANIPTSRLKGSQTAVYVATTDDGFETLLNAEAGYEAYTRFYGTGRAASTASGRISYLLDIHGPSVTIDTACSGGVVCIDQAINYLQSSSSADTAIVFLSAQGMVSPRGRCATFTADADGYVPSEGAVAFVLKTREAAIRDKDNILATIKATQISHNGRSQGLVAPNVNSQVDLHRSLLEKARLSPADVQFIEAHGTGTSLGDLSEIQAINAAYTSSQPRTTGPLIVSASKTVVGHTEPAGPLVGMLSVLLSFQEGAVPGLAHLTTRNLNPTLDYSSVPLLIPSEPVRLQTPKPYRAAVMSYGFSGTLAGLVLESPDERSSEEEPPDDKPMLFVVSAKTHTALIEYLQRYLEFLLHANPRDFCDICYTSCVGREHYRYRFACVANDMEDLIGQLQRRLSSKLPSKPLYKRGALAFAFSGQGTQFQGMATDLAKRYSGFRKIVSGLAKSAGELSGYAIDRYLLAYDVGSSIATPNSEVDQICIFVYQCSVLRWLGSIGIKPNVVIGHSLGEISASVAAGALSLDIALDLVISRAGLLRPSTDVPAGMAAVAASQQEVIELIDALDPDKANSLSVSVINGPQNIVVSGASAAIERMVASAKEKKIKASVLNISQAFHSSYVDSAITGLRAWSEKHISSAIPLQIPLYSTLLGARISKGQKLNPDHWVDHARKPVQFAQAATAMKETFTGVIMDIGPQAVAWSLLLANGLTSVTALAAKRGRSQQVAFLSALAELYQDYGIVPDFVALYAQEEEIDKLRKTDILTYPFQRVRRYPSFIPSRRVRGGEIPSSHPSESATLENTNEGTALRAESRVLCREDLHDSIVTSVKDVLELKPNEDLDLSESLNALGVDSIMFAQLRKRIGEGLGLSIPTVFLSDAFSINEMVNNLMEQAETPGEEGVMQEN

SEQ ID NO: 42 - DNA, 5298 bp, Guyanagaster necrorhiza
atggcggccgataggcacttttctcttctcgatgtctttctcgacgttgcccataatgctgagacgtcacaacgcaatattttggaatgcggcctggacacctggacatactcagatttggacatcatctcgtcggccctggcccaggatctcagtactaccttgggttgctctcccagggttgcagtcgtcagcgagaaccatccctatgtatttgctctcatgctggccgtatggaagcttggaggaatattcatccccatcgacgtccatgttcccgccgagcttttaacgggcatgctacgcatcgttgctccagactgcgtggtgattcctgagactgatctttctaatcagcgcgtcatctctgcactttacctccacgttattcccttcaatgtcaatgcgtcgacaatgtctacacttcgacagaaatatgtcctatctactcagaaacccttgctgtctgagttccctcttcctcatgttgatcgtgcatgcctctatctctttacgtcctccgcgtcttccaccgccaacttgaaatgcgtacctttgactcatgcacttatcctcagcaactgtcgttccaagcttgcatggtggcggcgtgttcgtccagaagaaaacatggatgggatgcgtgttttggggtgggcgccttggtcacatattctcgcgtacatgcaggatattgggacagcaacgctcctgaatgcaggctgctatgtctttgcatctgttccgtccacatatcctacgcagcacgtagccaatggcctgcaagaccccacttcaaatataatcaagtcgcttctcaaccgccgtatcacggcgttcgcatgtgtgccgttcattcttagtgaactgaaagctatgtgcgacccagcttccggtccggacgccaaggatcaaatgtgcttgggagctggggagaaagtgcgtcttataagcacactgcagaatctcatcatgttcgagtgtggaggcgctgcgctcgaggcagatatcacggattggaccgtcgaaaatggtatatcagtcatggtcggtattggaatgacggagaccgccggcacggttttggcagcgcgtgcacaagacgctcgttcgaatggctattctgccgaagaggctctcatcgctgatggcatactatcattggtcggacctgacgaggaggaaatggcctttgacggagaacttgtcgtgaaaagcaagctcatcccacatggatacatcaagtaccacgattcggcattttcagtggactcagatgactgggtgacgttcaaaactggggacaaatatcagcgtacaccagatggacatttcaagtggcttggaagaaagaccgattttattcagatgacaagcagcgaaaccttagatcccagacctatcgaaaaagccctctgcatgaaccccattatcgcaaatgcgtgtgtcattggtgacaggtttctaagggaacctgcgacgggtgtatgtgccattgtcgagatcaggtcagatgtggatatggcttccgccgaggttgacagggagattgcgaatgtcctcgctccaatcaatcgtgatcttcctccggctcttcgaatagcatggtctcgcgtactcataatccgacctcctcagaaaataccactcacaaagaaaggtgaggtgtttcgcaagaagattgaagatatatttggcaccttcttgggtgtcggtgttactaccaaggtggaagtggaccatgaaagtaaagaagatgatacggaacacatcgtgagacaggttgtgagcaatcttcttggagtccatgatcctgagctattgtctactttatctttcgctgagctcgggatgacctcatttatggccgttagcatcgttaacactttaaacaagcatattggcggcctcgcacttccacttaactcatgctacattcatattgatcttgattctcttgtagatgccatttcacttgaacgtggtcatcgaaagaaccctacgcccttttcttctaaccctttcctcgcctttgaatccagccagcaaaaggatatggagattgtgattgttggtaaagcattccgtttgcccggctcactcgacaatagcacctctctctgggaagctttgttgtcaaagagtgattcagtcatcggtgacatcccacccgatcgctgggatcatgcaagtttttacccccacgatatatgctttacgaaagcaggccttgtcgatgtttcccattatgactatagatttttcggtctcactgctacagaggcgttgtacgtatctccgacaatgcgcctcgccttggaagtgtcatttgaggctctggagaatgcaaatattcccttatccaagttgaaggggacacgaactgctgtttatgttgccactaaagacgatgggttcgagacactcttaaatgctgaacaaggctacgatgcctacacgcgattctacggcacgggacgtgctccgagtaccgcaagtggccgtataagctatcttcttgatattcatggaccttctgttaccgttgacacagcatgcagcggaggcattgtatgtatagaccaagccatcacttttctgcaatccggaggggcagatactgctattgtttgttcgagcaatacgcactgttggccgggatcatttatgttcctgacagcgcaaggcatggtctctccaaatggaagatgtgccacattcactacaaatgcagatggatatgtaccttcagaaggcgcagtggctttcgttctcaagacacacagcgcagcaacacgcgacaaagacaatatactggccgtgataaagtcaacagacgtgtctcataacggccgttctcaagggctagttgcaccaaacgtaaaggcacagacaaacctacaccagtcgctgttacgaaaagctgggctgcatcctgatctaattaactttatcgaagctcatggaacaggtacatctttaggagacctttcagaaatccagggtatcaataatgcctacacctcatctcgacctcgtccagccggtcctctgatcattagcgcgtcaaaaacggttttgggacatagtgaaccaactgcaggaatggccggcatcctcacaaccttgctcgcctttgagaaagagacggttcctggtctcaaccacttaacggaacataatttaaacccttcgcttgattgctctgcagtgcctctcctgattcctcacgagcctgttcatatcagtggtgcaaagccccatcgagctgcggttctatcatacggattcgcggggacactggctggtaccatcttagagagtccaccttgcgaccttacaaggcccttgtcaaacgacatacaagaacatcctatgattttcgtcgtcagtgggaaaacggtgccctctctggaagcatatctagagcggtatttggtatttttacgggtcgcaaaaccaagcgaattccatgacatctgctacaccacttgcatcgggagggagctgtataaataccggttctcctgcgttgcccgaaacatggccgacctcatttctcaaattgaacatcgactgacgacttgttccacttcgaaagagaagccccgtggctcgttaggattcgtgttctcgggtcaaggtacccagttccccggcatggcagcagcacttgccgaacgatattcagggttccgagcgctcgtctccaagtttgggcagatcgcccaagagcagtcgggctacccgatcgataggctgctcctcgaagttaccgatacattaccagaagcaaacagcgaggtcgaccaaatttgcatttttgtctaccaatattctgttctgcaatggctgcaacgtctaggcattcaaccgaaaacagtccttggtcacagtctaggagaaattaccgcctcagtcgcagttggcgccttctcttttaggtctgcattggaccttgtggtcacccgcgctcgtcttcttcgtcctcaaccaaaattctctgcgggaatggctgcagtagcagcgtccaaggaagaagttgaaggacttatagatatgctcaaacttgcggagtcgctgagcgttgcggttcataacagtccacggagtatcgttgtatcaggcgcatcagctgcggtcgatgccatggttgtcgctgctaaaaagcagggcttgaaggcctcccgcttaaaggttgaccaagcctttcacagcccttacgttgattctgctgtatcggggttgctcgactggtccaataagcatcgttcgaccttcctcccattgaacatccctttatactcaactttgactggcgcgcgtattccgaaaggagggaagttctgctgggatcactgggttaatcatgctcgaaagcccgtccagtttgcggcggcagctgcagcaatggacgaagatcaatccatcggtgttcttgttgatgttggaccccaacctgttgcgtggaccctccttcaagcgaataacctcctcaatacctcttcgattgctctatcagcaaaaattggaaaggatcaggagatggcgttgctctctgctttgagctacctcgtccaagagcacaacctttctctcagcttttatgagctttactctcagcgtcacggtactctgaagaagacagacgttcctacctacccgttccgtcgcgtccaccgctacccgactttcataccgtcacggaatagaagtcctgctgacatgagggtagctataccacctaccgacctctctgtccgaaagaatgtggatgcaacaccgcagtctcgtcgtgctggcctgatagcctgtcttaaagttatcctcgagttaacaccaggagaggaatttgacctttctgagactctcaatgctcgtggggtcgattcagttatgttcgcacagttgcggaagcgtgttggggaagaattcgacctagatataccaatgatctatttatcagacgtgttcacgatggaacagatgattgactacctcgtcgaacagtccagccccacatcaaagttggtagagatttcggttaatcaatcattagacggagaaggcctccggacagggcttgtatcatgccttagggacgtactggaaatctcctccgacgaagaacttgacttttccgaaactttgaacgctcgtggtacggactcaattatgttcgctcagctacgaaaacgtgtcggggaaggatttggtcttgaaattccgatgatatatctgtctgatgtgtttaccatggaagacatgatcaatttccttgtctcggagcgctcgtgc

SEQ ID NO: 43 - protein, 1766 aa, Guyanagaster necrorhiza
MAADRHFSLLDVFLDVAHNAETSQRNILECGLDTWTYSDLDIISSALAQDLSTTLGCSPRVAVVSENHPYVFALMLAVWKLGGIFIPIDVHVPAELLTGMLRIVAPDCVVIPETDLSNQRVISALYLHVIPFNVNASTMSTLRQKYVLSTQKPLLSEFPLPHVDRACLYLFTSSASSTANLKCVPLTHALILSNCRSKLAWWRRVRPEENMDGMRVLGWAPWSHILAYMQDIGTATLLNAGCYVFASVPSTYPTQHVANGLQDPTSNIIKSLLNRRITAFACVPFILSELKAMCDPASGPDAKDQMCLGAGEKVRLISTLQNLIMFECGGAALEADITDWTVENGISVMVGIGMTETAGTVLAARAQDARSNGYSAEEALIADGILSLVGPDEEEMAFDGELVVKSKLIPHGYIKYHDSAFSVDSDDWVTFKTGDKYQRTPDGHFKWLGRKTDFIQMTSSETLDPRPIEKALCMNPIIANACVIGDRFLREPATGVCAIVEIRSDVDMASAEVDREIANVLAPINRDLPPALRIAWSRVLIIRPPQKIPLTKKGEVFRKKIEDIFGTFLGVGVTTKVEVDHESKEDDTEHIVRQVVSNLLGVHDPELLSTLSFAELGMTSFMAVSIVNTLNKHIGGLALPLNSCYIHIDLDSLVDAISLERGHRKNPTPFSSNPFLAFESSQQKDMEIVIVGKAFRLPGSLDNSTSLWEALLSKSDSVIGDIPPDRWDHASFYPHDICFTKAGLVDVSHYDYRFFGLTATEALYVSPTMRLALEVSFEALENANIPLSKLKGTRTAVYVATKDDGFETLLNAEQGYDAYTRFYGTGRAPSTASGRISYLLDIHGPSVTVDTACSGGIVCIDQAITFLQSGGADTAIVCSSNTHCWPGSFMFLTAQGMVSPNGRCATFTTNADGYVPSEGAVAFVLKTHSAATRDKDNILAVIKSTDVSHNGRSQGLVAPNVKAQTNLHQSLLRKAGLHPDLINFIEAHGTGTSLGDLSEIQGINNAYTSSRPRPAGPLIISASKTVLGHSEPTAGMAGILTTLLAFEKETVPGLNHLTEHNLNPSLDCSAVPLLIPHEPVHISGAKPHRAAVLSYGFAGTLAGTILESPPCDLTRPLSNDIQEHPMIFVVSGKTVPSLEAYLERYLVFLRVAKPSEFHDICYTTCIGRELYKYRFSCVARNMADLISQIEHRLTTCSTSKEKPRGSLGFVFSGQGTQFPGMAAALAERYSGFRALVSKFGQIAQEQSGYPIDRLLLEVTDTLPEANSEVDQICIFVYQYSVLQWLQRLGIQPKTVLGHSLGEITASVAVGAFSFRSALDLVVTRARLLRPQPKFSAGMAAVAASKEEVEGLIDMLKLAESLSVAVHNSPRSIVVSGASAAVDAMVVAAKKQGLKASRLKVDQAFHSPYVDSAVSGLLDWSNKHRSTFLPLNIPLYSTLTGARIPKGGKFCWDHWVNHARKPVQFAAAAAAMDEDQSIGVLVDVGPQPVAWTLLQANNLLNTSSIALSAKIGKDQEMALLSALSYLVQEHNLSLSFYELYSQRHGTLKKTDVPTYPFRRVHRYPTFIPSRNRSPADMRVAIPPTDLSVRKNVDATPQSRRAGLIACLKVILELTPGEEFDLSETLNARGVDSVMFAQLRKRVGEEFDLDIPMIYLSDVFTMEQMIDYLVEQSSPTSKLVEISVNQSLDGEGLRTGLVSCLRDVLEISSDEELDFSETLNARGTDSIMFAQLRKRVGEGFGLEIPMIYLSDVFTMEDMINFLVSERSC

SEQ ID NO: 44 - DNA, 5034 bp, Mycena citricolor
atgtccgcctcttcctcctattctgaactggagtctccgacgtcgctgcttgacgtattcgtgcacgccgcgcgagacccccacaccgcgtcgcgtcgcgtgctggagtgcggctcggacacatggacatacgcggccttggatgcggtatccgatggcatagcgagggagctcgcgcccttcggtctggcgcccaaggtcgctgtggtcagcgagaaccatccatttgtgttcgcgctgctgttcgcagtctggaagctcgggggaacgttcatccccatcgacgcgcatgtcccctttgccatgctcacggggatggtgaacatcgtgaagccgacgtgtctctacctccccgcctccgccacatccaacatatctctcgtgaaggccttcgacatacggaccgtcgtatttgggcacaaggagaattccatgcaagctttgttcgacaagtactcattgcacgcggcgccgttgcacgcggcaccattgcattacgcgcctcccagtgccgaccatgcctgtctgtatctcttcacgtcgtcagcgtcctcgaccaaaaacctcaaggcggtcccgctgacgcatacactcgtcctgcgggggtgtcaatccaaactcgcttggtggcgccgagtccaacccggaaagaatctcgacgctatccggatcctcggctgggcaccctggtctcacgttctcgcctatatgcaggatatcgggacagcgacactgctcaacgcgggctgctacgtctttgcgaccatcccttcgtcctacccgcagctgcctaccactacccccgtcgatctgacgacttcgctcatcgacgcgctcgtcacgcggcgcgtcgcggcattcgcctgtgtccccttcgtcctggcgaacctcaaagccgcgtgccagagcaaccaccccgcgcgctcccagctgctcgaggcgctgcagaagacgatcatgctcgagtgcggtggggcggtgctggacgacgcgacggtggactgggccgagcgcaacggcatccgcatcttcacgggaatcgggatgaccgagactggcggggccgttttcgtcggcctcgcgagcgaatctagacgcgggtttctaccggaaggcctactcggtgatgcatccttctcgatctcgagtgacacggacgctcttgacgaaggggaacttgtcgtgaaaagtaaactcatcgcgcccggatatgtcggctacgacgacggagcgcactccgtggactgcgacggctgggtcacattcaggacaggagatcgatatcgccagacccagccagacgggcgctttacgtggctaggaaggatcacagacttcattcagatggtcagcggcgagtacctcgatccgcggcccctcgaggagagtctgcgtgcgtccccgctgatcgccaacgcctgcatcgtgggcgacgcgttcctcagcagcgcctcgacgagcatcctcgccatcatcgagctcgcgaccccggatctggcgcacacggcctccatccgggcgcagctcgcgcgcgtcctcgcgcccctcaaccgcgatctgcctccaccgcttcggatcgcagcgtcttcgatcctggttctggacgggatgcggaagatcccgaagacgaagaagggcgacatcttccgcaaaaagctggaagacacgttcggtgcggagtttgagcagatgctgcggaccgagaaagtcgggctgggtgacttggcggatgtggatgccggtatcactcgtatcatcggcaacttgctcggcatctccgacgacgagcttctgtcgaccatgtcgtttgctgagctcgggatgacctcgcttctcgccgtcaagatcgcgagcgaactaaacaagtttctggacggccgggctgtgctgcccacgaacatctgttacatacactttgacgtgccctcgctcacgagtagcgtccgagaaaggctatcctccgcgccctcctcgatcacatccgccgccagcgagcccgcggcctcgtcccccggcaccccccgcgccgacgaaatcgtgatcgtgggcaaggccttccggctgccggacggcgtgaacgacgacgccgcgctgtgggacgtcctgacgggcgagtccgcgtcgatcatcaaggacatccccgccgaccgctgggaccacgcgagcttctacccgaaggacatccactttggcagagctggtctggtggatgtcgcgcggttcgactacggctttttcggcatgacggcgagcgaggcgtattcgctgtcgccgacgatgcgcttggcgctcgaagtcgcgtatgaggcgttagaggacgcgaacattccgttccgggccgtcaagggctcgcgcatgggtgtgttcgtcgctgtgaaagacgatggattcgagaccctgttgcatgcggggcagggttatgacgcttacacgcggttctatggaactggaagggcgccgagcaccgccagcggccgcatcaactatctgctcgatctccacgggccgtcgatcaccgtcgacacggcctgcagcggcggcatcgtgtgcatcgaccaggctgtcacctatcttcaatcgggcgctgcagagacggcgatcgtgtgttcgagcaacacacactgctggcccggatccttcatgttcttgaccgcgcaaggcatggcctcccccaacggccggtgcgcatccttcacatcagacgccgacggatacgcgccgtcggagggcgcggtgggcttcgtgctgaagacgcggtctgccgctgtgcgcgacggcgatcggatactggctacgatcagggccacggagatcggacacaatggacggtctcagggactcgctgcgccgaatgtcaggtcccaggcggctgctcatcgggcggtgctgcggagagcgaggctggacccgtccgagattgacttcatcgaagcgcatgggactggcaccacattgggcgatctgtgcgaagtgcagggcatcaacgacagctttgtctcgcccaagaaacgcgccaaccctcttgtggtcagcgcgtccaagagcaccatcggccacaccgagccgtcggcgggtctcgtcgggatcctgtccgcgctgatgtcgttcgagaagcgcatcgtgccgagattggcatatctgactgagagcaatgtgaacccggcgctcgacgcgagtgtcgttccgctgcactttcccacaaagcacatcgagctgcgcgctgatgtgccgtacaaggccgtagtgatgtcgtacggattcgcaggtaccctagccgacatcgtcctcgagagcgaggtaccccaacccacgcccgcggtcgctcaggacacggccggccaacagccaatgctcttcgtgctcagcgccaaaaccccacgcgcactcgcagcctacatcgagctgtacctaggcttcctgcggcacgcggacccgggcctcttcgcgcgcatctgctacacggcgtgtgtcgcgcgcgagcactacaagcaccgcgtcgcgtgcgttgcgacggacctcgtcgacctcatcgcgcagctcgagacgcgtctggtgcagacggcgtatgcgggcggcggcggggcccgggccgcgcgcaccgggccgctggtgtttgcgttttcggggcagggcacgcagttcccggcgatggcggcgccgctggcgcggcggtacgcgcgcttcggggagatcgtggggggctgtgcgcgcatggcgcgcgagctgagcgggttccccgtggatggcattctcctcggagacgatgtgacgcccgtgaaggacaacagcgctgcggcggaggtgcacagcgaggtggaccagatctgcatatttgtgtaccagtatgcgatgtgtcggtggttgggcgagctgggtgtggagcccaaggctgccataggccacagcttgggagagatcacagcagcggtcatcgcaggagcactccctttcgaagccgccctcgatctggtcgtcacgcgcgcccggctgctcaagccgtgcgccgagcaaccgagcggcatggcggcgctcgcctgcaccccagacgtcgcatcgaagctcacactcggcgcgtcggtgtcggtgtcggtctacaacggcccgcagagcatctgcctgtccggcgcgtcggccgagctcgacgatgcggtgcgggccgcgaaggcgcggaacatcaaggcgacgcggctgcaggtcgaccagggcttccactcgccgtgcgtcgacgccgcggtgccggggctgcaggcgtggtgcgcggcgcaccgtgcgtcagctgcgccgctgaagatgccgctgtactcgaccgtgcgcggggacgtcgttccgaagggagcggcgctggatccggagcactgggtggcgcacgcgcggaacccggtgctgttcgcgcagacggcgcaggctctgaaagagtcccttccgcatgggatcactttggacgtcggcccgcaggcggtggcgtggtcgctgctgctgctgaacgggctcagcgcgacgcgcacggtcgctgccggggcgaagaagggcgcagaccaggagcgcgcgctgctgggggcgctgggggcgctgttcgagcagcacaaggtcacgccggactttgggcggctgtacgcgccgctcgagaagacgcggatcccgacgtacccctttgagcgcgcgcggtgctacccgacgttcataccctcgcgcttcgcgcacggggctgcggctgctgcgaagacagatggcgaggtcctgtcggccgaagaagaaaatgtggcacccgtgggattgctgacaaaggaggatctgcgtgcggctctcgtcgcgtgtctccgagcgacgctcgagctgcgccccgacgaagagctggacgaagcagagccgctgaccgtgcacggcgtggactcgatcgggtttgcgaagctgcgcaagcacgtcgaggaccgctgggggctggacatccccgtcgtgtactggtccgacgcgttcaccgtcggcgagatgctcggcaacttggtcggccagtatgacgtagtgtctactgctgcg

SEQ ID NO: 45 - protein, 1678 aa, Mycena citricolor
MSASSSYSELESPTSLLDVFVHAARDPHTASRRVLECGSDTWTYAALDAVSDGIARELAPFGLAPKVAVVSENHPFVFALLFAVWKLGGTFIPIDAHVPFAMLTGMVNIVKPTCLYLPASATSNISLVKAFDIRTVVFGHKENSMQALFDKYSLHAAPLHAAPLHYAPPSADHACLYLFTSSASSTKNLKAVPLTHTLVLRGCQSKLAWWRRVQPGKNLDAIRILGWAPWSHVLAYMQDIGTATLLNAGCYVFATIPSSYPQLPTTTPVDLTTSLIDALVTRRVAAFACVPFVLANLKAACQSNHPARSQLLEALQKTIMLECGGAVLDDATVDWAERNGIRIFTGIGMTETGGAVFVGLASESRRGFLPEGLLGDASFSISSDTDALDEGELVVKSKLIAPGYVGYDDGAHSVDCDGWVTFRTGDRYRQTQPDGRFTWLGRITDFIQMVSGEYLDPRPLEESLRASPLIANACIVGDAFLSSASTSILAIIELATPDLAHTASIRAQLARVLAPLNRDLPPPLRIAASSILVLDGMRKIPKTKKGDIFRKKLEDTFGAEFEQMLRTEKVGLGDLADVDAGITRIIGNLLGISDDELLSTMSFAELGMTSLLAVKIASELNKFLDGRAVLPTNICYIHFDVPSLTSSVRERLSSAPSSITSAASEPAASSPGTPRADEIVIVGKAFRLPDGVNDDAALWDVLTGESASIIKDIPADRWDHASFYPKDIHFGRAGLVDVARFDYGFFGMTASEAYSLSPTMRLALEVAYEALEDANIPFRAVKGSRMGVFVAVKDDGFETLLHAGQGYDAYTRFYGTGRAPSTASGRINYLLDLHGPSITVDTACSGGIVCIDQAVTYLQSGAAETAIVCSSNTHCWPGSFMFLTAQGMASPNGRCASFTSDADGYAPSEGAVGFVLKTRSAAVRDGDRILATIRATEIGHNGRSQGLAAPNVRSQAAAHRAVLRRARLDPSEIDFIEAHGTGTTLGDLCEVQGINDSFVSPKKRANPLVVSASKSTIGHTEPSAGLVGILSALMSFEKRIVPRLAYLTESNVNPALDASVVPLHFPTKHIELRADVPYKAVVMSYGFAGTLADIVLESEVPQPTPAVAQDTAGQQPMLFVLSAKTPRALAAYIELYLGFLRHADPGLFARICYTACVAREHYKHRVACVATDLVDLIAQLETRLVQTAYAGGGGARAARTGPLVFAFSGQGTQFPAMAAPLARRYARFGEIVGGCARMARELSGFPVDGILLGDDVTPVKDNSAAAEVHSEVDQICIFVYQYAMCRWLGELGVEPKAAIGHSLGEITAAVIAGALPFEAALDLVVTRARLLKPCAEQPSGMAALACTPDVASKLTLGASVSVSVYNGPQSICLSGASAELDDAVRAAKARNIKATRLQVDQGFHSPCVDAAVPGLQAWCAAHRASAAPLKMPLYSTVRGDVVPKGAALDPEHWVAHARNPVLFAQTAQALKESLPHGITLDVGPQAVAWSLLLLNGLSATRTVAAGAKKGADQERALLGALGALFEQHKVTPDFGRLYAPLEKTRIPTYPFERARCYPTFIPSRFAHGAAAAAKTDGEVLSAEEENVAPVGLLTKEDLRAALVACLRATLELRPDEELDEAEPLTVHGVDSIGFAKLRKHVEDRWGLDIPVVYWSDAFTVGEMLGNLVGQYDVVSTAA

SEQ ID NO: 46 - DNA, 4956 bp, Mycena chlorophos
atgaatccgccctcgtctatcctcgaagtcttccagcgtaccgccctcgacccctcgggcgccgaccggcgcgttctggaatgcgggcccgacttttggacctacgcgggtctcgacgcggtttccacaggccttgcggcggacttggctgctctcggagattctcccatcgtggctgtcgtcgctgagaaccatcccttcgtcttcgcactcatgtttgccgtctggaaattgcacgggacgtttgttcccatcgacgcgcatattccgtggaacctgctcgacggcatgttggacatcgtcaagccgacttgcatgtttctcgtcgagtcggataccaacaatatctcgaacaccaaggcccagggagtcgacttcgctgtgcgcctcttcggaggagaaggattcaccatcccggcgctctcggccaaatatgcggggaacgtctcgaatggcgcccccgagtcactcccttctccagacgcgactgctctgtatctgttcacgtcgtctgcctcgtcgcggcacaatttgaaggccgttccgctcacgcatcgattcattgccgctggctgcgaagcgaaactcgccttctggcaccgtcttcatcctcacaaccccaccgatgcgattcgcgtgctgggatgggctccgttgtcgcacgtcctcgcgcatatgcaggatatcggcaccgcggccctcctcaacgccggctgctatgtcttttcgacgatccccttgtcttacacctcagcagaaactcagcccgcgcaagatatcacctcggctctcattcactccgtgctccattacgaggtcaaagcatttgcgggcctgccttttgttattgcggcattcaaggctgcttgtgaaggcgggaacgaccgtctcctagcgcaactacgctccatgaccatgctcgagtgtggcggggcgcagttggacaaggacatcgtggattgggcagtcaagcaagcgatcccgctcgtggttggggtcgggatgacggaaacgggtggcgcgatactggcgggccccgtcggggatgcgtcggatgggtttcacccccaagggctgctgctagatgcacagttctcccttatcggcaatgacgatgaatcggaaggcgagctggtcatcaagagtcccaatcttccgcgcggatatctgaagtacgaggacggctcgttcgacatcgacgcgcagggtgtcgtcacgttcaagactggagacatctaccgcaaaagtgtcgagggtaaactcctttgggttgggcgtagtacggatttcatccagatggctaccggcgagacgctcgatccccgtcgtgttgaacgggcgctacgcttcgcatcggggatcaacgatgcctgcatcatcgggaatgcgttcctgaatggctcctctaccgcaatttacgccatcatcgagctcgctccgcgcaccgtcaacatcaataatgattcaaatgtctcccatctgcaggtggttgcccgcgccctgtctcctatcaaccgcgaccttcctcccgcattacgcattgtgttgtcttctgtattgattctggctgaaggcatgaagattccgaggacaaagaaaggcgaaatcttccggaagaagattgatgaagttttcggagctgctctccgggctttaggtcactcggcaactccaacagaggttgttcttgagcaggaaccagcggcagccagcaaacccatttttgacaagaacaagctgcagactgctattgcgcatagcttgggtctggatattctggagattgacctgctggacaagctgacctttgctgagcttggcatgacatctattcttgcaattcgggttgcagaagatctcaacaaattgctgcagggacaagttaccctgccagtcaatatctgctacctctatccagatgcccagctgttgtttgcagcagttcaggaacagctgctcaagcagcagcacccttcaaccccaactgctccctccgtgccggctttgctgtcagcaacttcctctgttccaattctcttgcaggaactagatgatgttgtcattgttggcaaatcattccggttgcctggcggaatctacgatgatcgagcactctgggcagctctcaccaatcaagctacccgaaaccccatctcatatatttcgggccagcgctgggaccatacaagcttttacccagctgatattgcattcttgcaggcagggttgcttgactccgaccactttacggattttgatgcagctttcttcgggatgaccgagaaagaggcatactatctgtccccgaccatgcggcttgctcttgaagtagcctttgaggctctagaagatgcaaatattcctgttggtcaggtgaaaggcactagcatgggagtatatgcagctgtcaaggatgatggattcgaaacccttttgaatgctgctcatgggtatgatgcctacacacgattctacggaactggacgggcaccaagtaccactagcgggcggatcagtcaaggagaatcagcgattgtctgctccagcaatacacactgttggcctggctccttcatgttcttgactgcccaaggaatggtgtctcctcatggacgttgtgcctccttcagtgctcaagcagatggatatgttccttcagagggtgctgttgcatttatcctgaagacccgcaaagcagcagttcgggatggaaaccaaatttttgccacaattcgggctgcggtggtatcacacaatggtcgatcacaaggtcttgcagcaccaaacattcaagcccaatccgagttgcatcaacaagcattgcagaaggcaaatatccaacccactgatattcattttgtggaaactcatggaacagggacttcgcttggggatgtctgtgagattcatgggataaatgctgcttttgcagcaggtcaccgtccctctggacctctcatcattagtgcaagcaaaggcactattggacatacagagccttctgcaggtcttgtgggcatcatggcggcactgctctccttcaagcatggccttgttcctgggctgatccatacatctcatgggcaactcaacccggcacttgatcaatccaaagttccgcttatcttcagcccacaaacaatttccctgggcggagaaaagccttacagatctgtggtcatgtcatatggctttgcaggcacactagcggatattgtgcttgaaggccctgctgaggaggctttttccgggccaggcaaaaacagcagtgctcctccgcctatgatctttgccctcagtgccaaatctgcatcagccctccaagaatacaagcagaagtacatcaccttcctgcagaatgttggctctggaggccaactgttcagcaagatctgtttgacttcttgcattgcccgagagcactacaagcatagattctcctgtgctgctcagaacacactggatcttcttctgcagctagagcactctgttgctgccagccacaaacctccaacaactcgtaccggaacagtcacctttgctttctctggacagggagcccaattccccagcatggatgcagctctggctcaaggctactctgccttcaaatccatcttgctggaacttggaaacaaggctgccaaactctctggattccccatcactgattgcctgttggcaacaacagcatcagctgatgaagaagccgtccatagtgaggtggaccaaatttgcatttttgttcatcaatatgcaatggctcttttcctcgagatgctaggaattgtccccggtgctgccataggccacagcttgggagagatcacagcagcggtcgttgctggtggactttcgtttgaacttggcctagagttggtcatcctccgtgcacatctgctccgtccagagcagaacaagcccgctggcatggctgccttggcatgctcagaagcggacttcctcaagtttccgtccaccgatgcaactatttctgttttcaactctcctcggagcattgcagtctctggagcagcaagctccattgagacagttcttactgctgccaaagagcagaatatcaaggccacgaagctcagggttgatcaaggattccatagcagctatgtggagcatgcgcttcccgggctcaagcactggtcagcaatgaattcaggctccttccaggcactcaggattccactctattcaactgcacttggccatgttgttcctgctggagagacccttcagccagatcactggatgaaccatacccgcaatgctgttcattttacgcaaactgcgcaggctctgaaagagtcccttccgcatgggatcactttggatcttggtcctcaggctgtagctcaaactctcctgctggccaatgaccatcctgttggccgcaccattggattgtgtggcaaacgcacaggagatcaaagacatgcattcctgctcgctcttgctgagctttaccagcagcatggtcttgtgcccaactttcatgcactttatggcgtagctgcccaggatctcaaggaccatctcaccagcctgccaacatatccattccaacgtgtccgctgctatcccagctacattccatcccgtcactccaacactcccgggaccaccgtggtgattgatgcaaagccgcgggatgaagtgaaacctgtggcagaggtctcaaagtcggacacggattcttccacatcattttcttcgaccattctcttccacattcgctccatccttgagcttcgcccccatgaggttctggatacgtctgaatccctcttgacgtacggggtcgactcgattgggtttgcagcactgcagaaggccctggagcagcagcatgggctaaacttgtcgattgtgttctggagcgacgtgttttccattgccgacattgtgaagaatcttgaggagcagaagagcttgaagatg

SEQ ID NO: 47 - protein, 1652 aa, Mycena chlorophos
MNPPSSILEVFQRTALDPSGADRRVLECGPDFWTYAGLDAVSTGLAADLAALGDSPIVAVVAENHPFVFALMFAVWKLHGTFVPIDAHIPWNLLDGMLDIVKPTCMFLVESDTNNISNTKAQGVDFAVRLFGGEGFTIPALSAKYAGNVSNGAPESLPSPDATALYLFTSSASSRHNLKAVPLTHRFIAAGCEAKLAFWHRLHPHNPTDAIRVLGWAPLSHVLAHMQDIGTAALLNAGCYVFSTIPLSYTSAETQPAQDITSALIHSVLHYEVKAFAGLPFVIAAFKAACEGGNDRLLAQLRSMTMLECGGAQLDKDIVDWAVKQAIPLVVGVGMTETGGAILAGPVGDASDGFHPQGLLLDAQFSLIGNDDESEGELVIKSPNLPRGYLKYEDGSFDIDAQGVVTFKTGDIYRKSVEGKLLWVGRSTDFIQMATGETLDPRRVERALRFASGINDACIIGNAFLNGSSTAIYAIIELAPRTVNINNDSNVSHLQVVARALSPINRDLPPALRIVLSSVLILAEGMKIPRTKKGEIFRKKIDEVFGAALRALGHSATPTEVVLEQEPAAASKPIFDKNKLQTAIAHSLGLDILEIDLLDKLTFAELGMTSILAIRVAEDLNKLLQGQVTLPVNICYLYPDAQLLFAAVQEQLLKQQHPSTPTAPSVPALLSATSSVPILLQELDDVVIVGKSFRLPGGIYDDRALWAALTNQATRNPISYISGQRWDHTSFYPADIAFLQAGLLDSDHFTDFDAAFFGMTEKEAYYLSPTMRLALEVAFEALEDANIPVGQVKGTSMGVYAAVKDDGFETLLNAAHGYDAYTRFYGTGRAPSTTSGRISQGESAIVCSSNTHCWPGSFMFLTAQGMVSPHGRCASFSAQADGYVPSEGAVAFILKTRKAAVRDGNQIFATIRAAVVSHNGRSQGLAAPNIQAQSELHQQALQKANIQPTDIHFVETHGTGTSLGDVCEIHGINAAFAAGHRPSGPLIISASKGTIGHTEPSAGLVGIMAALLSFKHGLVPGLIHTSHGQLNPALDQSKVPLIFSPQTISLGGEKPYRSVVMSYGFAGTLADIVLEGPAEEAFSGPGKNSSAPPPMIFALSAKSASALQEYKQKYITFLQNVGSGGQLFSKICLTSCIAREHYKHRFSCAAQNTLDLLLQLEHSVAASHKPPTTRTGTVTFAFSGQGAQFPSMDAALAQGYSAFKSILLELGNKAAKLSGFPITDCLLATTASADEEAVHSEVDQICIFVHQYAMALFLEMLGIVPGAAIGHSLGEITAAVVAGGLSFELGLELVILRAHLLRPEQNKPAGMAALACSEADFLKFPSTDATISVFNSPRSIAVSGAASSIETVLTAAKEQNIKATKLRVDQGFHSSYVEHALPGLKHWSAMNSGSFQALRIPLYSTALGHVVPAGETLQPDHWMNHTRNAVHFTQTAQALKESLPHGITLDLGPQAVAQTLLLANDHPVGRTIGLCGKRTGDQRHAFLLALAELYQQHGLVPNFHALYGVAAQDLKDHLTSLPTYPFQRVRCYPSYIPSRHSNTPGTTVVIDAKPRDEVKPVAEVSKSDTDSSTSFSSTILFHIRSILELRPHEVLDTSESLLTYGVDSIGFAALQKALEQQHGLNLSIVFWSDVFSIADIVKNLEEQKSLKM

SEQ ID NO: 48 - DNA, 5313 bp, Armillaria gallica
atggaggccgacggtcaccactctcttctcgatgtctttctcagcgttgcacatgattctgagaagtccaaacgtaatgtcttggaatgcggccaggatacctggacatactcagatttggacatcatctcgtcggccttggcacaggatctcaaagctaccttgggttgttttcccaaagttgcagtcgtcagcgagaaccatccctacgtgtttgctctcatgctggccgtttggaagcttgaagggatattcatccccatcgacgtccatattacagctgaccttctaaagggcatgctacgcattgttgcccccacttgtctggcgatcccagagaccgatatttccaaccagcgtgttgcctctgcgattggtatacatgttctccccttcaacgtgaatgcgtcgaccatgaatgcacttcgacagaaatacgacccatttactcagaatgcctcgctatctggatgcgcacttccttacgttgatcgcgcatgcctctatctctttacatcctccgcgtcctctactgccaacttgaaatgtgtacctttgacccatactcttatcctcagtaactgccgttccaagctcgcatggtggcggcgcgttcgtccggaaggcgaaatggatgggatacgtgttctagggtgggcaccttggtcgcatatccttgcctacatgcaagatattggcacggcaacgttcctgaatgccggctgctatgtctttgcgtccgttccatccacatatccttcacagctggcagcgaatggcctacaaggccccaccatgaatatcatcgattcacttcttgaacggcgagtcgccgcatttgcttgcgtaccgttcattttgagcgaactaaaagctatgtgcgagacggctgccagtccagatgacaagcatctcatgtgcttgagagctgaggagaaagttcgccttgtcagtgcgctgcagcggcttatgatgctcgagtgcggaggcgctgcgctcgagtcggatgtcacacgttgggccgtcgaaaatggcatatcggtcatggtcggcatcgggatgacggagacagtcggtacgctgtttgcagagcgcgcgcaagacgcctgttccaatggctattctgcgcaggacgccctcattgctgatggcatcatgtcactggtcgggtctgacaacgaggaagccaccttcgaaggggaactagtcgtgaagagcaagctcatcccacacggatacatcaactaccgtgattcgtcgttttcggtggactcggacggctgggtaacgttcaaaacgggagacaaatatcagcgcacgccagatggacgattcaaatggcttggaagaaagaccgattttattcagatgacaagcagcgaaacattggatcccagacccattgagcaagccctctgtgcgaatccaagtatcgcaaatgcatgcgtcattggtgacaggttcctgagagagcctgcgactagcgtatgcgccattgtcgagatcgggcctgaagtggacatgccttcgtccaagatcgacagagaaattgcgaatgccctcgctccaatcaatcgcgaccttcctcctgctcttcgcatatcatggtctcgcgtactcataattcgacctcctcagaaaataccagttacgaggaaaggcgatgtgttccgtaagaagattgaagatatgtttgggcctttccttggtgtcggcgtttctaccgaggtcgaagccggccatgaaactaaagaagatgacacggaacacatcgtgagacaggttgtgagcaatcttcttggcgtccatgatcctgagctattgtctgctttatctttcgctgagctgggcatgacctcatttatggctgttagcatcgtcaacgctctcaacaaacgcatcggcggcctcacccttccgtctaatgcatgctacatccatattgatcttgattctcttgtggacgcgatttcacttgaatatggtcatggaagtatccctgcagagttgccttctaaccctttccccgacatcgagtcccatcagcataatgataaggacattgtgatagtcggcaaggcattccgtttacccggctcactcaacagtactgcaactctctgggaagctttgttatcgaataacaattcggtcatcagtgatatcccatctgatcgctgggatcatgcaagcttttacccccacaatatatgtttcacgaaagcaggcctcgtcgatgttgcacattacgactacagattttttggcctcatggcgacagaggcgttgtacgtatctccgacgatgcgcctcgccttggaagtgtcatttgaagccctggagaacgcaaatattccgctatccaagctgaaggggacacaaaccgctgtctatgtcgccactaaagacgatggcttcgagacactcttaaatgccgagcaaggttacgatgcgtacacgcgattctacggcacgggtcgcgctccgagtaccgcaagcggtcgcataagctatcttcttgatattcatgggccatctattaccgttgatacagcatgcagcggaggcattgtatgtatggatcaagccatcactttcttgcaatccggaggggccgataccgcaattgtctgttcgaccaatacgcactgttggccgggatcattcatgttcctgacggcacaaggcatggtttctccaaatggaagatgcgctacattcactaccgatgcagacggatatgtgccttcggagggtgcagtggctttcattctcaagacgcgcagcgctgcaatacgcgacaatgacaatatactcgccgtgatcaaatcaacagatgtgtcccataacggccgttctcaagggctagttgcaccaaacgtaaaggcgcaggcaaacctacaccggtcgttgctacgaaaagctgggctgtttcctgatcaaattaactttatcgaagctcatggaacaggtacatctctaggagacctttcggaaatccagggcattaataacgcctacatctcatcacgacctcgtctggccggtccccttatcattagtgcatcgaaaacagttttaggacacagtgaaccaacagcagggatggccggcatcctcacagccttgcttgcccttgagaaagagacagttcctggtttaaatcacttaacggatcacaacctcaacccttcgcttgattgcagcgtagttcctctcctgattcctcacgagtctattcacattggtggtgcaaagccacatcgagctgcggttctgtcatacggcttcgcgggtacgctggccggtgctatcttagaaggaccaccttctggtgtaccatggccgtcgtcaaatgatatacaagaacaccctatgattttcgtcgtcagtgggaaaaccgtgcctacactggaagcgtacctgggacggtatttgacatttttgcgggtcgcaaaaacacacgacttccaggacatctgctacaccacttgcgtcgggagggagcactacaaataccggttctcctgcgttgcccgaaacatggcagaccttatttctcaaattgaacatcgactgacaactctttccacttcgaaacagaagcctcgcggctcgttagggttcatgttctcaggacaaggcacttgtttccctggtatggcttcagcacttgctgaacaatattcggggttccgaatgctcgtctctaagtttgggcaggctgcccaagagcggtccggttatccgatcgataggctgttgcttgaagtttctgatacattaccagaaacaaacagcgaggtcgaccaaatttgcatttttgtctaccaatattctgttctgcaatggttgcaatgtctaggcattcaaccgaaagcagtcctcggtcacagcctgggagaaattactgccgcagtcgcagctggcgccctttcgttcgaatctgcgttggatcttgtggtcacccgtgctcgtcttctccgtcccagaacaaaagactctgcaggaatggccgcagtagcagcgtccaaggaagaagttgaaggagttatagaaaccctccaacttgcaaactcgctaagcgttgcggttcacaacggtccgcggagtgttgttgtgtcaggcgcatcagcagaaatcgatgccctagttgtcgcagctaaagaacggggcttgaaagcctcccgcttaaaggttgaccaaggcttccacagcccttacgttgactctgcggttccgggtttactcgactggtcaaacaagcatcgttcgaccttctttcctttaaacattcctctatactcgactttgaccggcgagcttattccgaagggacggaggttcgtctcggatcactgggtaaaccatgctcgaaaacctgttcagtttgcggcggcagcggcagcggtggatgaagatcgatccattggtgtgctcgttgacgtcggaccccaacccgttgcgtggaccctccttcaagcaaacaaccttctcaatacctctgcagttgcgctatccgcaaaggccggaaaggaccaggagatggcgctgctcactgctttgagctacctcttccaagagcacaacctttctcccaacttccacgagctttactctcagcgtcatgggactctgcagaagacggacattcccacctacccattccaacgtgtccaccgctatccgaccttcataccgtcacgaaatcaaagtcctgctattgcaacggtagttataccgccacctcgcttctctgtccaaaaggctgcggatgtagcatcacagtcgaaggaatcagactgtcgagctggtttgatcagttgccttagagccatcctcgaattaacaccggaagaggagtttgacctttctgagactctcaacgctcgtggtatggattcgatcatgtttgcgcagctacggaagcgggttggggaagaattcaacctcgatatacccatgatctatctatcagacgtgttcacgatggaacagatggtcgactacctcgtcgaacagtccggatccacacccgcgtcaaagcacgtagaaacttcagctaatcaaccattagacgaagaagatctccggacggggctcttgtcatgcctgaggaacgtgctagaaattacccccgatgaagaacttgacctatctgaaactttgaatgctcgtggtgttgactcgatcatgttcgctcagctgcggaaacgcgttggggaaggttttggtgtggaaattccgatgatatatctgtctgacgtgtttaccatggaagacatgatcaatttcctcgtctccgagcggtcg

SEQ ID NO: 49 - protein, 1771 aa, Armillaria gallica
MEADGHHSLLDVFLSVAHDSEKSKRNVLECGQDTWTYSDLDIISSALAQDLKATLGCFPKVAVVSENHPYVFALMLAVWKLEGIFIPIDVHITADLLKGMLRIVAPTCLAIPETDISNQRVASAIGIHVLPFNVNASTMNALRQKYDPFTQNASLSGCALPYVDRACLYLFTSSASSTANLKCVPLTHTLILSNCRSKLAWWRRVRPEGEMDGIRVLGWAPWSHILAYMQDIGTATFLNAGCYVFASVPSTYPSQLAANGLQGPTMNIIDSLLERRVAAFACVPFILSELKAMCETAASPDDKHLMCLRAEEKVRLVSALQRLMMLECGGAALESDVTRWAVENGISVMVGIGMTETVGTLFAERAQDACSNGYSAQDALIADGIMSLVGSDNEEATFEGELVVKSKLIPHGYINYRDSSFSVDSDGWVTFKTGDKYQRTPDGRFKWLGRKTDFIQMTSSETLDPRPIEQALCANPSIANACVIGDRFLREPATSVCAIVEIGPEVDMPSSKIDREIANALAPINRDLPPALRISWSRVLIIRPPQKIPVTRKGDVFRKKIEDMFGPFLGVGVSTEVEAGHETKEDDTEHIVRQVVSNLLGVHDPELLSALSFAELGMTSFMAVSIVNALNKRIGGLTLPSNACYIHIDLDSLVDAISLEYGHGSIPAELPSNPFPDIESHQHNDKDIVIVGKAFRLPGSLNSTATLWEALLSNNNSVISDIPSDRWDHASFYPHNICFTKAGLVDVAHYDYRFFGLMATEALYVSPTMRLALEVSFEALENANIPLSKLKGTQTAVYVATKDDGFETLLNAEQGYDAYTRFYGTGRAPSTASGRISYLLDIHGPSITVDTACSGGIVCMDQAITFLQSGGADTAIVCSTNTHCWPGSFMFLTAQGMVSPNGRCATFTTDADGYVPSEGAVAFILKTRSAAIRDNDNILAVIKSTDVSHNGRSQGLVAPNVKAQANLHRSLLRKAGLFPDQINFIEAHGTGTSLGDLSEIQGINNAYISSRPRLAGPLIISASKTVLGHSEPTAGMAGILTALLALEKETVPGLNHLTDHNLNPSLDCSVVPLLIPHESIHIGGAKPHRAAVLSYGFAGTLAGAILEGPPSGVPWPSSNDIQEHPMIFVVSGKTVPTLEAYLGRYLTFLRVAKTHDFQDICYTTCVGREHYKYRFSCVARNMADLISQIEHRLTTLSTSKQKPRGSLGFMFSGQGTCFPGMASALAEQYSGFRMLVSKFGQAAQERSGYPIDRLLLEVSDTLPETNSEVDQICIFVYQYSVLQWLQCLGIQPKAVLGHSLGEITAAVAAGALSFESALDLVVTRARLLRPRTKDSAGMAAVAASKEEVEGVIETLQLANSLSVAVHNGPRSVVVSGASAEIDALVVAAKERGLKASRLKVDQGFHSPYVDSAVPGLLDWSNKHRSTFFPLNIPLYSTLTGELIPKGRRFVSDHWVNHARKPVQFAAAAAAVDEDRSIGVLVDVGPQPVAWTLLQANNLLNTSAVALSAKAGKDQEMALLTALSYLFQEHNLSPNFHELYSQRHGTLQKTDIPTYPFQRVHRYPTFIPSRNQSPAIATVVIPPPRFSVQKAADVASQSKESDCRAGLISCLRAILELTPEEEFDLSETLNARGMDSIMFAQLRKRVGEEFNLDIPMIYLSDVFTMEQMVDYLVEQSGSTPASKHVETSANQPLDEEDLRTGLLSCLRNVLEITPDEELDLSETLNARGVDSIMFAQLRKRVGEGFGVEIPMIYLSDVFTMEDMINFLVSERS

SEQ ID NO: 50 - DNA, 5310 bp, Armillaria ostoyae
atggaggccgacggtcactactctcttctcgatgtctttctcagcgttgcacatgattctgagaagtccaaacgtaatgtcttggaatgcggccaggatacttggacatactcggatttggacattatctcgtcggccctggcacaggatctcaaagctatcttgggttgttttcccaaagttgcagtcgtcagcgagaaccatccctacgtatttgctctcatgctggccgtatggaagcttgaagggatattcatccctatcgacgtccacgttacagctgaccttctaaagggcatgctacgcattgttgctcccacttgtctggtgatcccagagaccgatatttttaaccagcgtgttgcctctgcaattggtatacatgttctccccttcaacgtgaatgcgtcgaccatgactgcacttcgacagaaataccacccatttactcagaaagcctcgctatctgggtgcgcactgccttacgttgatcgcgcatgcctctatctctttacatcctccgcgtcctctactgccaatttgaaatgcgtacctttgacccatactcttatcctcagtaactgccgttccaagctcgcatggtggcggcgcgttcgtccggaaggcgaaatggatgagatacgtgttctagggtgggcaccttggtcgcatatccttgcctacatgcaagatattggcacggcaacgctcctgaacgccggttgctatgtctttgcgtccgttccatccacatatcctacacaactggcagcgaatggcttacaaggccctatcatgaatatcatcgattcacttcttgaacgacgagtcgccgcatttgcttgcgtaccgttcattttgagcgaactaaaagctatgtgcgagacggcttccagtccagacgacaagcatcaaatgtgcttgagagctgaggagaaagttcgccttgtcagtgcgctgcagcggcttataatgctcgagtgcggaggcgctgcgcttgagtcaggtgtcacacgttgggccgtcgagaatggcatatcagtcatggtcggcatcgggatgacggagacagtcggtacgctgtttgcagagcgcgcgcaagacgcccgttccaacggctattctgcgcaggacgccctcatttctgatgggatcatgtcactggtcgggtctgacaacgaggaagccaccctcgaaggggaactagtcgtcaagagcaagctcatccctcatggatacatcaagtaccgtgattcgtcgttttcggtggactcggacggctgggtaactttcaaaacgggagacaaatatcagcgcacgccagatggacgattcaaatggcttggaagaaagaccgattttattcagatgacaagcagcgaaacactggatcccagacccattgaggaagccctctgtgcgaatccaagtatcgcaaatgcatgcgtcattggtgacaggttcctgagggagcctgcgactagcgtatgcgccattgtcgagatcgggccggaagtggacatgccttcgtccaagatcgacagagaaattgcgaataccctcactccaatcaatcgcggccttcctcctgctcttcgcatatcatggtctcgcgtactcataattcgacctcctcagaaaataccagttacgaggaaaggtgatgtgttccgtaagaagattgaagatatgtttgggtctttccttggtgtcggcgtttctaccgaggtcgaagtcgaccatgaaactaaagaagatgatacgaaacacgtcgtgagacaggttgtaagcaatcttcttggagtccatgatcttgagttattgtctgctttatccttcgccgagctgggcatgacatcatttatggctgttagcatcgtcaacactctaaacaaacgcatcgacggcctcacccttccacctaatgcatgctacatccatattgatcttgattctcttgtggacgcgatttcacttgaacatggtcatgaaagtatccctgcagagttgccttctaaccctttccccgttatcgagtcccatcaacataacgataaggacattgtgatagtcggcaaggcattccgtttacccggctcactcaacaatactgcatctctctgggaagctttgttatcgaagaacagttcagtcatcagtgacatcccatccgatcgctgggatcacgcaagcttttacccccacgatatatgcttcacgaaagcaggcctcgtcgatgttgcacattacgactacagattttttggcctcacggcgacagaggcgttgtacgtatctccgacgatgcgcctcgccttggaagtgtcatttgaggccctggagaacgcaaatattccactatccaagctgaaggggacgcaaaccgccgtctatgtcgccactaaagacgatggcttcgagacactcttaaatgccgagcaaggctacgatgcatacacgcgattctacggtacgggtcgtgctccgagtaccgcaagcggccgtataagctatcttcttgatattcatgggccatctgtcaccgttgatacagcatgcagcggaggcattgtatgtatggatcaagccatcactttcttgcaatccggaggggccgataccgctattgtctgttcgagcaatacgcactgttggccgggatcattcatgttcctgacggcgcaaggcatggtttctcaaaatggaagatgcgctacatttactaccgatgcagacggatatgtaccttcggagggtgcagtggctttcattctcaagacgcgcagcgctgcgatacgcgacaacgacaatatactcgccgtgatcagatcaacagacgtgtcccataacggccgttctcaagggctagttgcaccaaacgtaaaggcgcagacaaacctacaccggtcgttgctacgaaaagctgggctgtttcctgatcaaattaactttatcgaagctcatgggacaggtacgtctctaggagacctttcggaaatccagggcattaataacgcctacacctcaacacgacctcgtctggccggtccccttatcattagcgcatcgaaaacagttttaggacacagtgaaccaacagcagggatggccggcatcctcacagccttgcttgcccttgagaaagagacagttcctggtctaaatcacttaacggagcacaaccttaacccttcgcttgattgcagcgtagttcctctcctgattcctcacgagtctattcacattggtggtgcaaagccacatcgagctgcggttctgtcatacggcttcgcgggtacgctggccggtgccatcttagagggaccaccttctgatgtaccaaggccgtcgtcaaataatatacaagaacaccctatgattttcgtcgtcagtgggaaaaccgtgcctgcactggaagcgtacctaggacggtatttggcatttttgcgggtcgcaaaaacacacgacttccatgacatctgctacactacttgcgtcgggagggagcactacaaataccggttctcctgcgttgcccgaaacatggcagaccttatttctcaaattgaacatcgactgacagctctttccacttcgaaacagaagcctcgcggctcgctagggttcatattctcaggacaaggcacttatttccctggtatggctgcagcacttgccgaacaatattcggggttccgagtgctcgtctctaagtttgggcaggctgcccaagagcggtcgggttatccgatcgataggctgttgcttgaagtttctgatacattgccagaaacaaacagcgaggtcgatcaaatttgcatttttgtctaccaatattctgttctgcaatggctgcagagtctaggcattcaaccgaaagcagtcctcggtcacagtctgggagaaattactgcagcagtcgcagctggtgccctttcgttcgaatctgcgttggaccttgtggtcacccgtgctcgtcttctccgtcctagagcaaaagattctgcaggaatggccgcagtagcagcatccaaggaagaagtcaaagggcttatagaaaccctccaacttgcggactcgctgagcgttgcggttcataacggtccgcggagtgttgttgtgtcaggcgcatcagccgaaatcgacgccctggttgtcgcagctaaagaacggggcttgaaggcctcccgcttaaaggttgaccaaggcttccacagcccttacgttgattctgcggttccaggtttactcgactggtcaaataagcatcgttcgaccttccttcctttgaacattcctttatactcgactttgactggcgagcttattccgaagggacggaggttcgtctcggatcactgggtaaaccatgctcgaaaacctgtccagtttgcggcggcagcggcagccgtggatgaagaccgatccattggtgtgctcgttgacgttggaccccaacccgtcgcgtggaccctccttcaagcaaacaaccttctcaatacctctgcagttgcactattcgcaaaggctggaaaggatcaggagatggcgctgcttactgctttgagctacctcgtccaagagcacaacctttctcccaacttccatgagctttactctcagcgtcatggtgctctgaagaagacagacgttcccacctacccattccgccgtgtccaccgctatccgaccttcataccgtcacgaaatcaaagtcctgctgctgcgacggtagctatgccgccaccccgcttctctgtccaaaagaatgcggatgtagcatcacagtcgaaggaatcagactgtcgagctggtttgatcagttgccttagagccatcctcgaattaacaccggaagaggagtttgacctttctgagactctcaacgctcgtggtatggattcgatcatgtttgcgcagctacggaagcgggttggggaagaattcgaccttgacatacccatgatctatttatcagatgtgttcacgatggaacagatggttgattacctcgtcaaacagtccggatccagacccgcattaaaacacgcagaaattccagttaatcaaccattagacgaagatctccggacgaggctcgtttcatgcctgaggaacgtgctagaaatcacccccgatgaagaacttgacctatctgaaactttgaacgctcgtggtgttgactcgatcatgttcgctcagctacgaaaacgcgttggggaaggatttggtgtggaaattccgatgatatatctgtccgacgtgtttaccatggaagacatgatcaatttcctcgtctctgagcgctcg

SEQ ID NO: 51 - protein, 1770 aa, Armillaria ostoyae
MEADGHYSLLDVFLSVAHDSEKSKRNVLECGQDTWTYSDLDIISSALAQDLKAILGCFPKVAVVSENHPYVFALMLAVWKLEGIFIPIDVHVTADLLKGMLRIVAPTCLVIPETDIFNQRVASAIGIHVLPFNVNASTMTALRQKYHPFTQKASLSGCALPYVDRACLYLFTSSASSTANLKCVPLTHTLILSNCRSKLAWWRRVRPEGEMDEIRVLGWAPWSHILAYMQDIGTATLLNAGCYVFASVPSTYPTQLAANGLQGPIMNIIDSLLERRVAAFACVPFILSELKAMCETASSPDDKHQMCLRAEEKVRLVSALQRLIMLECGGAALESGVTRWAVENGISVMVGIGMTETVGTLFAERAQDARSNGYSAQDALISDGIMSLVGSDNEEATLEGELVVKSKLIPHGYIKYRDSSFSVDSDGWVTFKTGDKYQRTPDGRFKWLGRKTDFIQMTSSETLDPRPIEEALCANPSIANACVIGDRFLREPATSVCAIVEIGPEVDMPSSKIDREIANTLTPINRGLPPALRISWSRVLIIRPPQKIPVTRKGDVFRKKIEDMFGSFLGVGVSTEVEVDHETKEDDTKHVVRQVVSNLLGVHDLELLSALSFAELGMTSFMAVSIVNTLNKRIDGLTLPPNACYIHIDLDSLVDAISLEHGHESIPAELPSNPFPVIESHQHNDKDIVIVGKAFRLPGSLNNTASLWEALLSKNSSVISDIPSDRWDHASFYPHDICFTKAGLVDVAHYDYRFFGLTATEALYVSPTMRLALEVSFEALENANIPLSKLKGTQTAVYVATKDDGFETLLNAEQGYDAYTRFYGTGRAPSTASGRISYLLDIHGPSVTVDTACSGGIVCMDQAITFLQSGGADTAIVCSSNTHCWPGSFMFLTAQGMVSQNGRCATFTTDADGYVPSEGAVAFILKTRSAAIRDNDNILAVIRSTDVSHNGRSQGLVAPNVKAQTNLHRSLLRKAGLFPDQINFIEAHGTGTSLGDLSEIQGINNAYTSTRPRLAGPLIISASKTVLGHSEPTAGMAGILTALLALEKETVPGLNHLTEHNLNPSLDCSVVPLLIPHESIHIGGAKPHRAAVLSYGFAGTLAGAILEGPPSDVPRPSSNNIQEHPMIFVVSGKTVPALEAYLGRYLAFLRVAKTHDFHDICYTTCVGREHYKYRFSCVARNMADLISQIEHRLTALSTSKQKPRGSLGFIFSGQGTYFPGMAAALAEQYSGFRVLVSKFGQAAQERSGYPIDRLLLEVSDTLPETNSEVDQICIFVYQYSVLQWLQSLGIQPKAVLGHSLGEITAAVAAGALSFESALDLVVTRARLLRPRAKDSAGMAAVAASKEEVKGLIETLQLADSLSVAVHNGPRSVVVSGASAEIDALVVAAKERGLKASRLKVDQGFHSPYVDSAVPGLLDWSNKHRSTFLPLNIPLYSTLTGELIPKGRRFVSDHWVNHARKPVQFAAAAAAVDEDRSIGVLVDVGPQPVAWTLLQANNLLNTSAVALFAKAGKDQEMALLTALSYLVQEHNLSPNFHELYSQRHGALKKTDVPTYPFRRVHRYPTFIPSRNQSPAAATVAMPPPRFSVQKNADVASQSKESDCRAGLISCLRAILELTPEEEFDLSETLNARGMDSIMFAQLRKRVGEEFDLDIPMIYLSDVFTMEQMVDYLVKQSGSRPALKHAEIPVNQPLDEDLRTRLVSCLRNVLEITPDEELDLSETLNARGVDSIMFAQLRKRVGEGFGVEIPMIYLSDVFTMEDMINFLVSERS

SEQ ID NO: 52 - DNA, 5322 bp, Armillaria fuscipes
atgaccatggaggtcgacagccactattctctgctcgatgtctttctcagcattgcacatgattctgacaagtccaaacgcaatgtcttggaatgcggcctggaggcctggacatactcggatttagacatcatctcgtcggctctggcacaggatctcaaagctaccttgggatgttttcccaaagttgcagtcgtcagcgagaaccatccctacgtgtttgctctcatgctggccgtctggaagctcgaagggatcttcatccccatagacgtccacgttacagctgaccttttaaagggcatgctacgcattgttgctcctacttgtctagtgatccctgagagcgatgtttccaaccggcgtgttgcctctgcgattggtatacgtgttctcccatttgatgcgaattcgtcaaccatgacggcacttcgacaaaagtacgaatcattcactcagaaagcctcgccatctgagtgcacacttgcccacgccgatcgtacatgcctctatctttttacatcctctgcatcctctaccgccaacttgaaatgtgtgcctttgacccatactcttatcctcaataactgccgtaccaagctcgcatggtggcagcgctttcgtccagaaagcgaaatggatgggatgcgtgttctagggtgggcaccttggtcgcatatccttgcctacatgcaagatatcggcacagcgacgctcctgaacgccggttgctatgtctttgcgtccattccatccacataccctacacaactagcagcaaatggcttacaaggccccactatgaatatcatcaacgcacttcttgaacgacaaattgccgcatttgcttgcgtgccgttcattttgagcgaactcaaagctatgtgcgagatgacttcctgtacaggaaaccaaatgtccctaagagccgaggagaaagttcgcctggtcagggtgctgcaggggcttgtaatgctcgagtgtggaggcgcggcacttgagtcagatgttacgcgttgggtcgttgagaatgacataccagtcatggttggcattgggatgacggaaacagtcggtacgctgttcgcagagcgcgcccaagacgtccgttccagcggatattccgcccaagacgctctcattgctgatggcattatgtcactggtcggttctgacaacgaggaagccactttcgaaggggaactagtcgtgaagagcaagctcatcccacagggatacatagggtaccgtgattcatcgttctcggtggactcagatggctgggtaacgttcaaaactggagataaatatcagcgcactccagatggacgattaaaatggcttgggagaaagaccgactttatccagatgacaagcagcgaaacactggatcccaggcccattgagcaaactctttgtgcgaatccatacgtcgcaaaagcatgcgtcattggtgacagattcctgagagatcccgcgaccagcgtatgtgccataattgagatcaggccggaagtggacatgccttcgtccaagatcgacagagaaattgcgaatgcccttgctccaatcaatcgcgacctccctcctgctcttcgcatatcatggtctcgcgtactcatgatcagaccccctcagaaaatcccagtaacgaggaaaggcgatgtgttccgtaagaagattgaagatatgtttgggtctttcctcggtgtcggtgtttctactgaggtcggagtcgaccatgaaactgaaaaagatgatacggaacacattgtgagacaggttgttactaatcttcttggagtccatgatcccgagctattgtctgctttatctttcgctgagcttggaatgacttcatttatggctgtcagcatcgtcaactctctaaacaagtacatcgatggcctcacccttccacctaatgcatgttacatccatattgatcttgattctcttgtggaggccatttcacgtgaacggggtcatggaagtaacgccacagagttgccttctcaacccgtccctgttgagttacatcaacctaacgataaggacgttgtgatagttggcaaggcattccgtttacctggctcactcgacagcactgcatctctatgggaagctttgttatcaaagaacaattcagtcgtcagtgaaatcccatccgatcgctgggatcacgcaagcttttacccccacgacatttgcttcacgaaagcaggcctcgtcgatgttgcccattacgattacagattctttggcctcacggctacagaggcattgtatgtatctccgacgatgcgtctcgccttggaagtgtcatttgaagccctggagaatgcgaatattccgttatccaatctgaagggaacacaaaccgctgtctatgtcgccaccaaagacgatggtttcgagacacttttaaatgccgagcagggctatgatgcctacacacgattctatggcacgggccgcgctccaagcaccgcaagtggccgtataagctatctacttgatattcatgggccatctgtcaccgttgatacagcatgcagcggaggcattgtgtgtatggatcaagccatcactttcttgcagtccggaggggcagatacagctattgtctgttcgagcaatacgcattgttggcctggatcatttatgttcctgacggcgcaaggcatggtttctccaaatggaagatgcgctacatttagtaccgatgcagacggatatgtgccttcagagggcgcagtagctttcgttctcaagacgcgtagcgcagcaatacgcgataatgacaatatcctcgccgtaatcaaatcaacagatgtgtctcataacggccgttctcaagggctggttgcacctaacgtgaaggcgcagacaaacctgcatcaatcgttgttacgaaaagctgggctgtttcctgatcaaatcaactttatcgaagcccatggaacaggtacatctctaggagacctctcagaaatccagggcatcaataacgcctacacctcaacacgacctcgtctagacggtccccttatcatcagcgcatcgaaaacagtgataggacacagcgaaccaactgcagggatggcgggcatcctcacagccttgcttgctcttgagaaagaaacagttcctggtctcaatcacttaacggagcacagccttaacccttcgcttgattgcagcatagtcccgctcctgattcctcacgagtctattcacattggtggggtaaagccacatcgagctgcggttctgtcatacggcttcgcgggtacactggcgggtgctatcttagagggaccgccttcagatgcaccaaggccgtcgtcaaataatgtgcaagatcaccctatgatttttgccctcagtgggaaaagcgcgtccgcactggaagcatacctaaggcggtatttggcattcctgcggattgcagatccacacgacttccataacatctgctacacttcctgtgtcgggagggagcactacaaatatcggttctcctgtgttgcccgaaacatggcagaccttatatctcaaattgaacatcgactgacaactgtttccattccgaaaccgaaacctcgtggctcaataggattcacgttctcaggacaaggcacttatttccctggcatggccgcagcactcactgaacaatattctggattccggacgctcgtctctaagcttgggcaggctgcgcaagagcggtcgggtcatccgattgacaggctgttacttgaagtttccggtacatcaccagaaacaaacagtgaggtcgagcaaatttgcacatttatctaccaatatgccgttctgcaatggttgcagagcctaggcgttcaaccgaaagcagtcctcggtcacagcctgggagaaattactgccgcagtcgcagctggtgccctgtcgttcgaatccgcgttggaccttgtggtgacccgtgctcgtcttctccgtcccgaaacaaaagattctgctgggatggtcgcggtagcaacgtccaaggatgaagttgaaggacttatagaaacactccaagttgcggacgcgctaagcgttgccgttcacaacggttcacggagtgttgtggtttcaggcacatcagcggaagttgatgccctggtcgtcgcagctaaagaacggggcttaaaggcttcccgcttaagagtcgaccaaggtttccacagcccttgcgttgattctgccgttcctggtttactcgactggtcaaatgagcatcgttccaccttccttcctttgaatatgcctttatactcgactttgaccggcgaggtcattcccaagggacggaaattcgtctgggatcactgggtaaaccatgctcgaaaacctgttcagtttgcaccggcagcaaaagcggtggacgaagaccgatccatcggtgtgctcgttgatgtaggacctcaacctgtcgcttggacccttttgcaagcaaacaacctttccaacacctctacggttgcgctattcgcgaaagccggaaaggatcaggagatggcactgctcactgctttgagctacctcttccaagagcacaacctttctcccaagtttcacgacctttatactgggtataatggtgctctgaagaagacggacattcccacgtacccattccaacgtgtccatcgctatcccaccttcataccatcacgaaatcagagtcctgctgtcgcgaaagcagtcgtgcagccgccccgcttctctatccaaaggaatcgagaagccacattacagtcgaaggaaccagatcaccgagcttgtttagtcacttgccttagagccatcctcgaattaacatcagaggaagaacttgacctctctgagaccctcaacgctcgtggcgtggactcgatcatgttttcacagctacggaagcgggttggagaagaattcaatctcgagatacccatgatctatttatcagacgtattcacgatggagcagatgattgactacctcgtcgaacagtccggatccaatcccgcatcaaagcaagtaggaactccggttaaccgactatcaggcgaagaagatcttcggacggggctcatctcatgcctgagggacgtgctagaaatcactcctgatgatgaacttgatcacccaaaagacctatctgaaactttaaatgctcgtggtgttgattcgataatgttcgctcagctacgaaaacgcgtcggggaaggatttggtgtggaaattccgatgatatatctgtctgatgtgtttaccatggaagacatgattaatttcctcgtttctgagcgctca

SEQ ID NO: 53 - protein, 1774 aa, Armillaria fuscipes
MTMEVDSHYSLLDVFLSIAHDSDKSKRNVLECGLEAWTYSDLDIISSALAQDLKATLGCFPKVAVVSENHPYVFALMLAVWKLEGIFIPIDVHVTADLLKGMLRIVAPTCLVIPESDVSNRRVASAIGIRVLPFDANSSTMTALRQKYESFTQKASPSECTLAHADRTCLYLFTSSASSTANLKCVPLTHTLILNNCRTKLAWWQRFRPESEMDGMRVLGWAPWSHILAYMQDIGTATLLNAGCYVFASIPSTYPTQLAANGLQGPTMNIINALLERQIAAFACVPFILSELKAMCEMTSCTGNQMSLRAEEKVRLVRVLQGLVMLECGGAALESDVTRWVVENDIPVMVGIGMTETVGTLFAERAQDVRSSGYSAQDALIADGIMSLVGSDNEEATFEGELVVKSKLIPQGYIGYRDSSFSVDSDGWVTFKTGDKYQRTPDGRLKWLGRKTDFIQMTSSETLDPRPIEQTLCANPYVAKACVIGDRFLRDPATSVCAIIEIRPEVDMPSSKIDREIANALAPINRDLPPALRISWSRVLMIRPPQKIPVTRKGDVFRKKIEDMFGSFLGVGVSTEVGVDHETEKDDTEHIVRQVVTNLLGVHDPELLSALSFAELGMTSFMAVSIVNSLNKYIDGLTLPPNACYIHIDLDSLVEAISRERGHGSNATELPSQPVPVELHQPNDKDVVIVGKAFRLPGSLDSTASLWEALLSKNNSVVSEIPSDRWDHASFYPHDICFTKAGLVDVAHYDYRFFGLTATEALYVSPTMRLALEVSFEALENANIPLSNLKGTQTAVYVATKDDGFETLLNAEQGYDAYTRFYGTGRAPSTASGRISYLLDIHGPSVTVDTACSGGIVCMDQAITFLQSGGADTAIVCSSNTHCWPGSFMFLTAQGMVSPNGRCATFSTDADGYVPSEGAVAFVLKTRSAAIRDNDNILAVIKSTDVSHNGRSQGLVAPNVKAQTNLHQSLLRKAGLFPDQINFIEAHGTGTSLGDLSEIQGINNAYTSTRPRLDGPLIISASKTVIGHSEPTAGMAGILTALLALEKETVPGLNHLTEHSLNPSLDCSIVPLLIPHESIHIGGVKPHRAAVLSYGFAGTLAGAILEGPPSDAPRPSSNNVQDHPMIFALSGKSASALEAYLRRYLAFLRIADPHDFHNICYTSCVGREHYKYRFSCVARNMADLISQIEHRLTTVSIPKPKPRGSIGFTFSGQGTYFPGMAAALTEQYSGFRTLVSKLGQAAQERSGHPIDRLLLEVSGTSPETNSEVEQICTFIYQYAVLQWLQSLGVQPKAVLGHSLGEITAAVAAGALSFESALDLVVTRARLLRPETKDSAGMVAVATSKDEVEGLIETLQVADALSVAVHNGSRSVVVSGTSAEVDALVVAAKERGLKASRLRVDQGFHSPCVDSAVPGLLDWSNEHRSTFLPLNMPLYSTLTGEVIPKGRKFVWDHWVNHARKPVQFAPAAKAVDEDRSIGVLVDVGPQPVAWTLLQANNLSNTSTVALFAKAGKDQEMALLTALSYLFQEHNLSPKFHDLYTGYNGALKKTDIPTYPFQRVHRYPTFIPSRNQSPAVAKAVVQPPRFSIQRNREATLQSKEPDHRACLVTCLRAILELTSEEELDLSETLNARGVDSIMFSQLRKRVGEEFNLEIPMIYLSDVFTMEQMIDYLVEQSGSNPASKQVGTPVNRLSGEEDLRTGLISCLRDVLEITPDDELDHPKDLSETLNARGVDSIMFAQLRKRVGEGFGVEIPMIYLSDVFTMEDMINFLVSERS

SEQ ID NO: 54 - DNA, 5304 bp, Armillaria mellea
atggaggccaacggtcactactctcttctcgatgtctttctcagcattgcacacgattctgacaagtccaaacgtaatgtcttggaatgcggtcaggatacctggacatactcagatttggacattatctcgtcggccctggcacaggatctccaagctacgctgggttgttttcccaaagttgcagtcgtcagcgagaaccatccctacatttttgctctcatgctggccgtttggaagctcgaagggatattcatccctgtcgacgtccatgttacagctgaccttctaaagggcatgttacatatcgtcgctcccacttgtctggtgatccctgagaccgatatttccaaccagcgtattgcttccgcgattggtatacatgttctccccttcagtgcgaatgcgtcgaccatgactgcacttcgacagaaatacgacctatgcattcagaaagcctcgctatctgagcgcgcacttcctcacgttgatcgcgcttgcctctatctctttacatcctctgcgtcctctactgccaacttgaaatgcgtacctttgacccatagtcttatcctcagtaactgccgttccaaactcgcatggtggcggcgcgttcgtccagaggaagaaatggatgggatacgtgttctagggtgggcaccttggtcccatatccttgcctacatgcaagatattggcacagcgacgctcctgaacgccggttgctacgtctttgcgtcaattccgtccacatatcctacacaactggtggtgaatggcttacaaggccccaccatgaatatcatcgactcacttcttaaacggcgaatcgtcgcatttgcttgtgtcccgttcattttgggcgaactaaaagctatgtgcgagacggcttcgggtccagatgtcaagcatcatatgggcttgagagctgaggagaaggttcgccttgttagggcactgcagcagcttatgatgctcgagtgcggaggcgctgcgctcgagtcagatgtcacgcgttgggttgtcgaaaatggcatatcggtcatggttggcatcgggatgacggagacagttggtacgctgtttgcagagcgcgcgcaagacgcccgttccaacggctactctgcgcagaacgccctcattactgatggcattatgtcactggtcgggcctgacaacgaggaagtcacctttgaaggggaactagtcgtgaagagcaagctcatcccacacgaatacatcaattaccgtgattcgtcgttctcggtggactcggatggctgggtaacgtttaaaacgggagataaatatcagcgcacaccagatggacgattcaaatggcttggaagaaagaccgactttattcagatgacaagcagtgaaacactggatcccagacccattgagaaaaccctctgtgcgaatccaagtattgtaaatgcatgcgtcattggtgacagattcctgagggagcctgcaactagcgtatgcgccattgtcgagatcaggccggaagtggacatcccttcgtccaagatcgacagggaaattgcgaatgcccttgctccaatcaatcgcgacctccctcctgctcttcgcatatcatggtctcgcgtactcataattcgacctcctcagaaaataccagttacaaggaaaggcgatgtgttccgtaagaagattgaagatgtatttgggtctttccttggtgtcggttctaccgagatcaaagtcgaccatgaatctaaagaagatgatacggaacacattgtgagacaggttgtcagcaatcttcttggagtccatgatcctgagctattgtctgctttatctttcgctgagctgggaatgacctcgtttatagctgttagtattgtcaacgctctaaacaagcgcatcagcggcctcacccttccacctaatgcgtgctacctccatattgatcttgattctcttgtgaacgccatttcacgtgaacatggtcatggaactaaccccgcagagttgccttctaaacttttccccgtcaccgagtcctatcaacgtaatgataaggacgttgtgataatcggcaaagcattccgtttaccaggctcactcgacaatactgcatctctctgggaagctttgttatcgaagaacaattcagtcgtcagtgacatcccatccgatcgctgggatcacgcaagcttttacccccacgatatatgcttcacgaaagcaggcctcgtcgatgttgcacattacgattacagattctttgggctcacagcgacagaggcattgtacgtatctccgacgatgcgcctcgccttggaagtgtcatttgaagcccttgagaacgcgaatattccgctatccaagctgaaggggacacaaacccctgtctatgtcgccactaaagacgatggctttgagacactcttaaatgctgagcaaggctacgatgcttacacgcgattctatggtacaggtcgcgctccgagcaccgcaagtggtcgtataagctatctactcgatattcatgggccatctgttaccgttgatacagcatgcagcggaggcattgtatgtatggatcaagccatcactttcttgcaatccggaagggccgataccgctattgtctgttcgagcaatacgcactgttggccgggatcatttatgttcctgacggcgcagggcatggtttctccacatggaagatgcgctacatttactaccgatgcagacggttatgtaccttcggagggtgctgtggctttcattctcaagacgcgcagtgctgcaatacgcgataatgacaatatactcgccgtgatcaaatcaacagatgtgtcccataacggccgttctcaagggctagttgcaccaaacgtaaaggcgcagacaagcctacaccgatcgttgctacgaaaagctggactatttcctgatcaaatcaattttatcgaagcccatggaacaggtacatctctaggagacctctcggaaatccagggcatcaacaacgcctacacctcaacacgacctcgtttggacggtccccttatcattagcgcgtcgaaaacagtgttgggacacagcgaaccaattgcggggatggccggcatcctcacagccttgctttccctcgagaaagaaacagtttttggtttaaatcacttaacagagcacaaccttaacccttcgcttgattgcagcctagttcctctcctgattcctcacgagtctattcacattggtggtgaaaaaccacatcgagctgcggttctgtcatacggtttcgcgggtacgctggccggtgccatcttagagggaccaccttcagatgtaccaaggccgtcgtcaagcgatatgcaagaacaccctatggttttcgtcctcagtgggaaaagcgtgcctgcactggaaacgtacctaagacggtatttggcatttttgcgcgccgcaaaaacaaacgacttccatagcatctgctacaccacttgcgtcgggagggagcattacaaataccggttctcctgcgttgcccaaaatatggcagaccttgtgtctcaaattgaacatcgactgacaactctttccaattcgaaacagaaacctcgtggctcgccagggtttatgttctcaggacaaggcacttatttccctggtatggctgcagcgcttgctgaacaatatttggggtttcgagtgctagtctctaggtttgggaaggctgctcaagagcggtcgggttatccgatcgataggctgttgcttgaagtttctgatacatcatcagaaacaaacagcgaggctgaccaaatttgcatttttgtctaccaatattccgttctgcaatggctgcagagtctaggcattcaaccgaaagcagtcctcggtcacagcctgggagaaattaccgccgcagtcgcagctggtgccctttcattcgaatctgcgttggaccttgtagtcacccgtgctcgtcttctccgtcctgaaacaaaagattcagcaggaatggccgcagtagcagcatccaaggaagaagttgaagaacttatagaaaacctccaacttgcgcatgcgttaagtgttgcggttcacaacggtccacggagtgttgttgtgtcaggcgcatcgaccgaaattgatgccctggtcgtcgcagctaaagaacggggcttgaaggcttcccgcttaagagttgaccaaggcttccatagcccttacgttgattctgccgttccgggtttactcgactggtcaagtgaacattgttcgaccttccttcctttaaatattcctttatactcgactttgaccggcgaagttattccaaagggacggaggttcgcctgggatcactgggtaaaccatgctcgaaaacctgttcagtttgcggcggcggcagcagcggtggacgaagaccgatccatcggcgtgctccttgatgttggaccccaacccgttgcgtggaccatccttcaagcaagcagccttttcaacacctctgcagttgcgctatttgcgaaggctggaaaggatcaggagatggcgctgcttactactttgagctacctcttccaagagcacaatctttgtcccaactttcacgagctttactctcagcgtcatggtgctcttaagaagacggacattcccacctacccattccaacgtgtccaccgctatccaaccttcataccatcacgaaatcaaagtcctgctgtcgcgaaggtagtagtcccatcgcgctttcctgtccaaaggaaaggggaagcaatatcacaatcgaacgaatcagattaccgagctggtttgatcacttgcctcagaaccatcctcgaattaacatcagaagaagagtttgacctttctgagaccctcaacgctcgtggtgtggattcgatcatgttttcacagctacggaagcgggttggggaagaattcgatctcgatatacccatgatctatttatcagacgtgttcacgatggaacagatgatcgactacctcgtcgaacagtccggatccagacccgcgccaaagcacgtagaaactccggttaacgaacctttaggcaaagatctccggacggggctcgtttcatgcctgaggaatgtactagaaatcacccccgatgaagaactcgacctatctgaaactttgaacgctcgtggtgtcgactcgatcatgttcgctcagctacgaaaacgcgtcggggaaggatttggcgtggaaattccgatgatatatctgtctgacgtgtttactatggaagacatgatcaatttcctcgtctctgagcacgcg

SEQ ID NO: 55 - protein, 1768 aa, Armillaria mellea
MEANGHYSLLDVFLSIAHDSDKSKRNVLECGQDTWTYSDLDIISSALAQDLQATLGCFPKVAVVSENHPYIFALMLAVWKLEGIFIPVDVHVTADLLKGMLHIVAPTCLVIPETDISNQRIASAIGIHVLPFSANASTMTALRQKYDLCIQKASLSERALPHVDRACLYLFTSSASSTANLKCVPLTHSLILSNCRSKLAWWRRVRPEEEMDGIRVLGWAPWSHILAYMQDIGTATLLNAGCYVFASIPSTYPTQLVVNGLQGPTMNIIDSLLKRRIVAFACVPFILGELKAMCETASGPDVKHHMGLRAEEKVRLVRALQQLMMLECGGAALESDVTRWVVENGISVMVGIGMTETVGTLFAERAQDARSNGYSAQNALITDGIMSLVGPDNEEVTFEGELVVKSKLIPHEYINYRDSSFSVDSDGWVTFKTGDKYQRTPDGRFKWLGRKTDFIQMTSSETLDPRPIEKTLCANPSIVNACVIGDRFLREPATSVCAIVEIRPEVDIPSSKIDREIANALAPINRDLPPALRISWSRVLIIRPPQKIPVTRKGDVFRKKIEDVFGSFLGVGSTEIKVDHESKEDDTEHIVRQVVSNLLGVHDPELLSALSFAELGMTSFIAVSIVNALNKRISGLTLPPNACYLHIDLDSLVNAISREHGHGTNPAELPSKLFPVTESYQRNDKDVVIIGKAFRLPGSLDNTASLWEALLSKNNSVVSDIPSDRWDHASFYPHDICFTKAGLVDVAHYDYRFFGLTATEALYVSPTMRLALEVSFEALENANIPLSKLKGTQTPVYVATKDDGFETLLNAEQGYDAYTRFYGTGRAPSTASGRISYLLDIHGPSVTVDTACSGGIVCMDQAITFLQSGRADTAIVCSSNTHCWPGSFMFLTAQGMVSPHGRCATFTTDADGYVPSEGAVAFILKTRSAAIRDNDNILAVIKSTDVSHNGRSQGLVAPNVKAQTSLHRSLLRKAGLFPDQINFIEAHGTGTSLGDLSEIQGINNAYTSTRPRLDGPLIISASKTVLGHSEPIAGMAGILTALLSLEKETVFGLNHLTEHNLNPSLDCSLVPLLIPHESIHIGGEKPHRAAVLSYGFAGTLAGAILEGPPSDVPRPSSSDMQEHPMVFVLSGKSVPALETYLRRYLAFLRAAKTNDFHSICYTTCVGREHYKYRFSCVAQNMADLVSQIEHRLTTLSNSKQKPRGSPGFMFSGQGTYFPGMAAALAEQYLGFRVLVSRFGKAAQERSGYPIDRLLLEVSDTSSETNSEADQICIFVYQYSVLQWLQSLGIQPKAVLGHSLGEITAAVAAGALSFESALDLVVTRARLLRPETKDSAGMAAVAASKEEVEELIENLQLAHALSVAVHNGPRSVVVSGASTEIDALVVAAKERGLKASRLRVDQGFHSPYVDSAVPGLLDWSSEHCSTFLPLNIPLYSTLTGEVIPKGRRFAWDHWVNHARKPVQFAAAAAAVDEDRSIGVLLDVGPQPVAWTILQASSLFNTSAVALFAKAGKDQEMALLTTLSYLFQEHNLCPNFHELYSQRHGALKKTDIPTYPFQRVHRYPTFIPSRNQSPAVAKVVVPSRFPVQRKGEAISQSNESDYRAGLITCLRTILELTSEEEFDLSETLNARGVDSIMFSQLRKRVGEEFDLDIPMIYLSDVFTMEQMIDYLVEQSGSRPAPKHVETPVNEPLGKDLRTGLVSCLRNVLEITPDEELDLSETLNARGVDSIMFAQLRKRVGEGFGVEIPMIYLSDVFTMEDMINFLVSEHA

SEQ ID NO: 56 - protein, 40 aa, Artificial sequence
  Consensus amino acid sequence of 1 hispidin synthases
  MISC_FEATURE: X - any amino acid
  MISC_FEATURE (1)..(40): X - any amino acid
VAXXXENHPXXXALXXAVWKXXGXFXPXDXHXXXXXXXGM

SEQ ID NO: 57 - protein, 36 aa, Artificial sequence
  Consensus amino acid sequence of 2 hispidin synthases
  MISC_FEATURE (1)..(36): X - any amino acid
LGWAPXSHXLXXMQDIGXXXXLXAGCYVFXXXPXXY

SEQ ID NO: 58 - protein, 33 aa, Artificial sequence
  Consensus amino acid sequence of 3 hispidin synthases
  MISC_FEATURE (6)..(33): X - any amino acid
ECGGAXLXXXXXXWXXXXXXXXXXGXGMTETXG

SEQ ID NO: 59 - protein, 8 aa, Artificial sequence
  Consensus amino acid sequence of 4 hispidin synthases
FAELGMTS

SEQ ID NO: 60 - protein, 25 aa, Artificial sequence
  Consensus amino acid sequence of 5 hispidin synthases
  MISC_FEATURE (2)..(24): X - any amino acid
IXXXRWDHXSFYPXXIXXXXAGLXD

SEQ ID NO: 61 - protein, 65 aa, Artificial sequence
  Consensus amino acid sequence of 6 hispidin synthases
  MISC_FEATURE (2)..(64): X - any amino acid
DXXFFGXXXXEAXXXSPTMRXALEVXXEALEXANIPXXXXKGXXXXVXXAXXDDGFETLLXAXXG

SEQ ID NO: 62 - protein, 50 aa, Artificial sequence
  Consensus amino acid sequence of 7 hispidin synthases
  MISC_FEATURE (5)..(47): X - any amino acid
ADGYXPSEGAVXFXLKTXXAAXRDXXXIXAXIXXXXXXHNGRSQGLXAPN

SEQ ID NO: 63 - protein, 9 aa, Artificial sequence
  Consensus amino acid sequence of 8 hispidin synthases
  MISC_FEATURE (8)..(8): X - any amino acid
GHSLGEIXA

SEQ ID NO: 64 - DNA, 867 bp, Neonothopanus nambi
atggcgccaatttcttcaacttggtctcgtctcattcgatttgtggctattgaaacgtccctcgtgcatatcggtgaaccgatagacgccaccatggacgtcggtctggcgagacgagaaggcaagacgatccaagcatacgagattattggatcaggctcggctctagacctctcagcccaagtatcgaagaatgtgctgactgtaagggaactcctgatgccgctttcaagagaggaaattaaaactgtacgatgcttggggttgaactaccctgttcatgccaccgaagcgaacgttgctgttccaaaattcccgaatttgttctacaaaccagtgacctcgctcattggccccgatggactcattaccatcccttccgttgtccaacccccgaaggagcatcagtccgattatgaagcggaacttgtcattgtcatcgggaaagcagcaaagaatgtatcggaggatgaggctttggattatgtattgggatacactgccgcgaacgatatttcgtttaggaaacaccagctagcagtctcacaatggtctttctcgaaaggatttggtagccttctactcactatccgtatggcacaaacccactcgggtaacattaatcgcttctccagagaccagattttcaatgtcaagaagacaatttccttcctgtcacaaggcactacactggaaccaggttctatcattttgactggtacacctgacggagtgggctttgtgcgcaatccaccactttaccttaaagatggagatgaagtaatgacctggattggaagtggaatcggaacattagccaatacagtgcaagaagagaagacttgcttcgctagtggcggacacgag

SEQ ID NO: 65 - protein, 289 aa, Neonothopanus nambi
MAPISSTWSRLIRFVAIETSLVHIGEPIDATMDVGLARREGKTIQAYEIIGSGSALDLSAQVSKNVLTVRELLMPLSREEIKTVRCLGLNYPVHATEANVAVPKFPNLFYKPVTSLIGPDGLITIPSVVQPPKEHQSDYEAELVIVIGKAAKNVSEDEALDYVLGYTAANDISFRKHQLAVSQWSFSKGFGSLLLTIRMAQTHSGNINRFSRDQIFNVKKTISFLSQGTTLEPGSIILTGTPDGVGFVRNPPLYLKDGDEVMTWIGSGIGTLANTVQEEKTCFASGGHE

SEQ ID NO: 66 - DNA, 963 bp, Neonothopanus gardneri
atggcgccgattttgacagtgagttccagttcaatggtgctgaatagctcaaaacaggctatattgaatatttctgaatatcaccagccctggactcgtctcattcgatttgtagccgttgagacgtcactcgtgcatattggtgaacccatcgaggtgactttggacgtcgggcaggcaaaatgtgaaggcaagacgatcaaagcgtacgagattattggatcagggtcggccttggacctctcagctcaggtatcgaagaatgtgctaaccgtaaaggaactcttgatgccgctttcgagagaagaggtcaagactgtgcggtgcttgggactgaactattttactcatgcttccaccgggcgcccgctgtcgactagattaccgactttgttctataagccagtgacttcactcatcggacctgaggcgttcattaatattccttccgctgttcaaccaccgaaggagcatcagtccgattatgaagcggagttggtaattattattgggagagcggcgaaggatgtaccggaagaggaggctttgaattatgttttgggatacaccgccgccaacgacatttcatttaggaaatatcaatttgcagtttcccagtggtgtttttcgaaaggtttcgataatacaaacccaatcggtccgtgcatcgtttccgcatcttccattccgaacccgcaagacatccagatccaatgcaaactgaacgggaatgtcgttcagaatggaaacaccagtgatcaaatttttaatatcaagaaaacagtcgcttttttgtcgcaaggaacaacacttgagtcaggatcaatcatcctgaccggtacgcctggcggagtgggatttgtgcgcgatccaccgctttaccttaaagatggagatgaagtagtgacttggattggaagtggggttggaagtttagtcaacgtagtaaaagaagagaagact

SEQ ID NO: 67 - protein, 321 aa, Neonothopanus gardneri
MAPILTVSSSSMVLNSSKQAILNISEYHQPWTRLIRFVAVETSLVHIGEPIEVTLDVGQAKCEGKTIKAYEIIGSGSALDLSAQVSKNVLTVKELLMPLSREEVKTVRCLGLNYFTHASTGRPLSTRLPTLFYKPVTSLIGPEAFINIPSAVQPPKEHQSDYEAELVIIIGRAAKDVPEEEALNYVLGYTAANDISFRKYQFAVSQWCFSKGFDNTNPIGPCIVSASSIPNPQDIQIQCKLNGNVVQNGNTSDQIFNIKKTVAFLSQGTTLESGSIILTGTPGGVGFVRDPPLYLKDGDEVVTWIGSGVGSLVNVVKEEKT

SEQ ID NO: 68 - DNA, 906 bp, Armillaria gallica
atggcgcctattatcactcaatggtccagacttatccgctttgtcgccgtcgaaacctctcgtgtacatattggacagcccgtagattctaacctggatgttggtctagcggcgtaccagggaatgctaatcaaggcttacgaaatacttggttctgctctcgatccatccgcccaaatgactagcaagatcctcactgtcaaacaactattaaccccgctgtctggcgaagatgtcaaggtcgttagatgcttgggtctcaactacccagcacatgcgaatgaagggaaagtagaagcacctaagtttcctaaccttttctataagccagtgacgtcgcttatcgggcccctcgcacctgtgatcattcctgcagtcgcacaaccttctgcaatacatcaatccgattatgaggctgaatttactgtcgtcataggcagggcagctaagaatatcactgaagcggaagctttagactatgttctcggctacaccggcggcaatgacgtgtcttttcgtcagcatcaatttgcggtctctcaatggtgtttctccaaaagttttgacaacacaaatccctttgggccatgcttagttgccgcatcgtctattcccgaccctcaaactgtggccattaagtttacactgaatggtcaaactgtccaagacggaactactgccgatcaacttttcagcgtcaaaaagaccatagcttatctttctcaaggcacgacgttacagccgggctccataattatgactggtactcccagtggcgttggattcgtccgaaacccacctctctacctcaaagatggagaccatatgttgacttggataagcggtggaatcggtacgcttgcaaacagcgtcgtggaggagaagactccgactcctggtttagat

SEQ ID NO: 69 - protein, 302 aa, Armillaria gallica
MAPIITQWSRLIRFVAVETSRVHIGQPVDSNLDVGLAAYQGMLIKAYEILGSALDPSAQMTSKILTVKQLLTPLSGEDVKVVRCLGLNYPAHANEGKVEAPKFPNLFYKPVTSLIGPLAPVIIPAVAQPSAIHQSDYEAEFTVVIGRAAKNITEAEALDYVLGYTGGNDVSFRQHQFAVSQWCFSKSFDNTNPFGPCLVAASSIPDPQTVAIKFTLNGQTVQDGTTADQLFSVKKTIAYLSQGTTLQPGSIIMTGTPSGVGFVRNPPLYLKDGDHMLTWISGGIGTLANSVVEEKTPTPGLD

SEQ ID NO: 70 - DNA, 906 bp, Armillaria ostoyae
atggcacctattattactcaatggtccagacttattcgctttgttgccgtcgagacctctcgtgtacatattggacagcccatagattctaccctggatattggtctagcggcgtaccagggaatgctaatcaaggcttatgaaatacttggttctgctctcgatccatccgcccaaatgaccagcaagatcctcaccgttaaacagctattaactccgctgtctggcgaagatgtcaaggtcgtccgatgcttgggtcttaactatccagctcatgcgaatgaagggaaagtagaagcgcctaagtttcctaaccttttctataagccagtgacatcgcttatcgggcccctcgcacctgtgatcattcctgcagtcgcacagccttctgcaatacatcaatccgattatgaggctgaatttactgtcgtcataggcagggcagctaagaatgtcactgaagcggaagctttagactatgttctcgggtacaccggcggcaatgatgtgtcttttcgtcagcatcaatttgcggtctctcaatggtgtttctctaaaagttttgacaatacaaatcccttcggtccatgcttagttgccgcatcgtctattcctgatcctcaaactgtggccattaagtttacattgaatggtgacaccgtccaagacggaactactgctgatcaacttttcagcgtcaaaaagaccatcgcttatctttctcagggcacgacgttacagccgggctccataattatgactggcactcccagtggtgttgggttcgtccaaaacccacctctctacctcaaagatggggatcaaatgttgacttggataagcggtggaatcggtacgcttgcaaacaacgtcgtagaggagaagactccgactcctcgtttagac

SEQ ID NO: 71 - protein, 302 aa, Armillaria ostoyae
MAPIITQWSRLIRFVAVETSRVHIGQPIDSTLDIGLAAYQGMLIKAYEILGSALDPSAQMTSKILTVKQLLTPLSGEDVKVVRCLGLNYPAHANEGKVEAPKFPNLFYKPVTSLIGPLAPVIIPAVAQPSAIHQSDYEAEFTVVIGRAAKNVTEAEALDYVLGYTGGNDVSFRQHQFAVSQWCFSKSFDNTNPFGPCLVAASSIPDPQTVAIKFTLNGDTVQDGTTADQLFSVKKTIAYLSQGTTLQPGSIIMTGTPSGVGFVQNPPLYLKDGDQMLTWISGGIGTLANNVVEEKTPTPRLD

SEQ ID NO: 72 - DNA, 891 bp, Armillaria mellea
atggcgcctattatcactcagtggtccagacttattcgctttgttgccgtcgagacttctcgtgtacattttggacagcccgtagattctaccctggatgttggtctagcggcgtaccagggtgtgttgatcaaggcttatgaaatacttggttctgctcttgatccatccgcccaaatgaccagcaagatcctcaccgtgaaacagctattaactccgctgtctggcgaggatgtcaaagtcgtccgatgcttgggtcttaactatccagcacatgcgaatgaagggaaagtagaagcacccaagtttcctaacctgttctataagccagtgacatcgcttatcgggcccctcgcgcctgtgatcattcctgcagtcgcacagccttctgcaatacatcaatctgattatgaggctgaatttactgttgtcctaggcagggcagctaagaatgtcactgaagctgaagccttggactatgttctcggttacaccggcggcaatgatgtgtcttttcggcagcatcaatttgctgtctctcaatggtgtttctccaaaagttttgacaatacaaatcccttcggtccatgcttagttgccgcgtcgtctattcctgatcctcaaactgtggccattaagtttacattgaatggcaacaccgtccaagatggaactactgctgatcaacttttcagcgtcagaaagaccatcgcttatctttctcaaggcacgacgttacagcctggctccataattatgaccggtactcccagtggcgttgggttcgtccgaaacccacctctctacctcaaagatggggatcaaatgttgacttggattagcggtggaatcggtacgcttgcaaacagcgtcatagaggagaagacccca

SEQ ID NO: 73 - protein, 297 aa, Armillaria mellea
MAPIITQWSRLIRFVAVETSRVHFGQPVDSTLDVGLAAYQGVLIKAYEILGSALDPSAQMTSKILTVKQLLTPLSGEDVKVVRCLGLNYPAHANEGKVEAPKFPNLFYKPVTSLIGPLAPVIIPAVAQPSAIHQSDYEAEFTVVLGRAAKNVTEAEALDYVLGYTGGNDVSFRQHQFAVSQWCFSKSFDNTNPFGPCLVAASSIPDPQTVAIKFTLNGNTVQDGTTADQLFSVRKTIAYLSQGTTLQPGSIIMTGTPSGVGFVRNPPLYLKDGDQMLTWISGGIGTLANSVIEEKTP

SEQ ID NO: 74 - DNA, 891 bp, Armillaria fuscipes
atggcacctattatcactcaatggtccagacttattcgctttgtctccattgagacttctggtgttcatattggacaacccgtagatcctacccttgacgtcggtctagcggcgtcccagggagtggcaatcaaggtttatgaaataattggttctgcgcttgatccatccgcccaagtgaccagcaaaatccttaccgtcaaacagctattaactccgctgtctggtgaggatgtcaaggtcgtccggtgcttgggtctgaactatccagcacatgccaatgaagggaaagtagaagcacccaagtttcctaaccttttctataagccagtgacatcgcttattgggcccctcgcgcctgtgatcattcctgcagtcgcacagccggccgcaatacatcaatctgattatgaggctgaatttactgttgtcattggcagggcagccaagaatgtcacggaagctgaagccctggactatgttcttggctacaccggcggtaatgatgtgtcttttcggaagcatcaatttgcagtctctcaatggtgtttctccaaaagttttgacaatacaaatccctttggaccatgcttagttgccgcttcgtctatccctgatcctcagaatgtggccattaagttcacgttgaatggtaacaccgttcaagatggaactactgctgaccaaattttcagcgttagaaagactatcgcttatctttctcaaggcacgacgttacagccaggctccatcattatgactggcactcccaatggcgttgggtttgtccgagacccacctctctacctcaaagacggggatcaaatgctgacttggattagcggtggaattggtacgcttgcgaatggcgtcgtagaagagaagacccgg

SEQ ID NO: 75 - protein, 297 aa, Armillaria fuscipes
MAPIITQWSRLIRFVSIETSGVHIGQPVDPTLDVGLAASQGVAIKVYEIIGSALDPSAQVTSKILTVKQLLTPLSGEDVKVVRCLGLNYPAHANEGKVEAPKFPNLFYKPVTSLIGPLAPVIIPAVAQPAAIHQSDYEAEFTVVIGRAAKNVTEAEALDYVLGYTGGNDVSFRKHQFAVSQWCFSKSFDNTNPFGPCLVAASSIPDPQNVAIKFTLNGNTVQDGTTADQIFSVRKTIAYLSQGTTLQPGSIIMTGTPNGVGFVRDPPLYLKDGDQMLTWISGGIGTLANGVVEEKTR

SEQ ID NO: 76 - protein, 42 aa, Artificial sequence
  Consensus amino acid sequence 1 caffeylpyruvate hydrolase
  MISC_FEATURE (2)..(39): X - any amino acid
WXRLIRFVXXETSXVHXGXPXXXXXDXGXAXXXGXXIXXYEI

SEQ ID NO: 77 - protein, 137 aa, Artificial sequence
  Consensus amino acid sequence 2 caffeylpyruvate hydrolase
  MISC_FEATURE (5)..(136): X - any amino acid
SALDXSAQXXXXXLTVXXLLXPLSXEXXKXVRCLGLNYXXHAXXXXXXXXXXPXLFYKPVTSLIGPXXXXXIPXXXQPXXXHQSDYEAEXXXXXGXAAKXXXEXEALXYVLGYTXXNDXSFRXXQXAVSQWXFSKXF

SEQ ID NO: 78 - protein, 69 aa, Artificial sequence
  Consensus amino acid sequence 3 caffeylpyruvate hydrolase
  MISC_FEATURE (3)..(65): X - any amino acid
DQXFXXXKTXXXLSQGTTLXXGSIIXTGTPXGVGFVXXPPLYLKDGDXXXTWIXXGXGXLXNXVXEEKT

SEQ ID NO: 79 - DNA, 801 bp, Neonothopanus nambi
atgcgcattaacattagcctctcgtctctcttcgaacgtctctccaaacttagcagtcgcagcatagcgattacatgtggagttgttctcgcctccgcaatcgcctttcccatcatccgcagagactaccagactttcctagaagtgggaccctcgtacgctccgcagaactttagaggatacatcatcgtctgtgtcctctcgctattccgccaagagcagaaagggctcgccatctatgatcgtcttcccgagaaacgcaggtggttggccgaccttccctttcgtgaaggaaccagacccagcattaccagccatatcattcagcgacagcgcactcaactggtcgatcaggagtttgccaccagggagctcatagacaaggtcatccctcgcgtgcaagcacgacacaccgacaaaacgttcctcagcacatcaaagttcgagtttcatgcgaaggccatatttctcttgccttctatcccaatcaacgaccctctgaatatccctagccacgacactgtccgccgaacgaagcgcgagattgcacatatgcatgattatcatgattgcacacttcatcttgctctcgctgcgcaggatggaaaggaggtgctgaagaaaggttggggacaacgacatcctttggctggtcctggagttcctggtccaccaacggaatggacttttctttatgcgcctcgcaacgaagaagaggctcgagtagtggagatgatcgttgaggcttccatagggtatatgacgaacgatcctgcaggaaagattgtagaaaacgccaag

SEQ ID NO: 80 - protein, 267 aa, Neonothopanus nambi
MRINISLSSLFERLSKLSSRSIAITCGVVLASAIAFPIIRRDYQTFLEVGPSYAPQNFRGYIIVCVLSLFRQEQKGLAIYDRLPEKRRWLADLPFREGTRPSITSHIIQRQRTQLVDQEFATRELIDKVIPRVQARHTDKTFLSTSKFEFHAKAIFLLPSIPINDPLNIPSHDTVRRTKREIAHMHDYHDCTLHLALAAQDGKEVLKKGWGQRHPLAGPGVPGPPTEWTFLYAPRNEEEARVVEMIVEASIGYMTNDPAGKIVENAK

SEQ ID NO: 81 - DNA, 783 bp, Omphalotus olearius
atgctcccagctttcatctacaaaccaaggctagtgatcacttgtgtattcgttctggcctccgcactcgcatttcccttcatacgcaaagattaccagactttcctggaggtgggaccctcgtacgccccgcagaacctccaaggatacatcatcgtctgtgtactctctctgttccggcaagaacagaaagacgtagcgatttatgatcgccttcctgagaaaaggaggtggttaggagacctcccgtttcgcgaggggccaagaccgagtatcactagccatatcatccagcgacagcgcacccaattggctgacgccgagttcgctaccaaagagctgataggcaaaatcatccctcgcgtccaagcccgacacaccaacacaacattcctcagcacatctaaattcgaattccacgcccaggccatcttccttttgccctctatcccaatcaacgaccctcaaaacattccaagccacgataccgttcgtcgcacgaaacgcgagatcgcgcatatgcatgattatcacgactgtacgttgcatctcgcacttgctgctcaagatgggaaggaggttttagagaaaggatggggtcagcgacatcctcttgctggacctggtgttcctggcccgccgacggagtggacgtttctttatgcaccgcgcagcgaagaggaggttcgggttgtggagatgattgttgaggcatcagttgtgtatatgacgaatgatcctgcggataaaatcgtagaagctactgtgcagggtactgaagaa

SEQ ID NO: 82 - protein, 261 aa, Omphalotus olearius
MLPAFIYKPRLVITCVFVLASALAFPFIRKDYQTFLEVGPSYAPQNLQGYIIVCVLSLFRQEQKDVAIYDRLPEKRRWLGDLPFREGPRPSITSHIIQRQRTQLADAEFATKELIGKIIPRVQARHTNTTFLSTSKFEFHAQAIFLLPSIPINDPQNIPSHDTVRRTKREIAHMHDYHDCTLHLALAAQDGKEVLEKGWGQRHPLAGPGVPGPPTEWTFLYAPRSEEEVRVVEMIVEASVVYMTNDPADKIVEATVQGTEE

SEQ ID NO: 83 - DNA, 798 bp, Armillaria gallica
atgtccttcatcgacagcatgaaacttgacctcgtcggacacctctttggcatcaggaatcgcggcttagccgccgcttgttgtgctctagcagtcgcctctactatcgccttcccttacattcgtagggactaccagacatttttatctggcggtccctcttacgctccccagaatatcagaggatatttcatcgtctgcgttctggccttgttccgtcaggagcaaaagggccttgcgatatatgatcgccttcccgagaagcgcaggtggctgcctgacttgcctcctcgcaatggcccgcggccgatcacgaccagccatataatccaaagacagcgcaaccaggcgccggaccccaagttcgccctcgaggaactcaaggccacggttattccacgggtgcaggctcgccatactgacctcacccatctcagcctatccaaattcgagttccatgctgaagcaattttcctgctcccctctgtacccatcgatgatccaaaaaatgttccaagtcacgacacggtgcgcaggacgaaaagggagatcgcgcatatgcacgactaccatgacttcacgctgcatcttgcactggccgcccaagacgggaaggaagtcgtgtcgaagggatgggggcagcgacaccccctagcaggccctggcgttcctggtccacctacggagtggacatttatttatgcgccacgtaacgaagaggaactggcagtggtggaaatgattatcgaggcatcaataggctatatgaccaatgaccctgctggagtagttatcgca

SEQ ID NO: 84 - protein, 266 aa, Armillaria gallica
MSFIDSMKLDLVGHLFGIRNRGLAAACCALAVASTIAFPYIRRDYQTFLSGGPSYAPQNIRGYFIVCVLALFRQEQKGLAIYDRLPEKRRWLPDLPPRNGPRPITTSHIIQRQRNQAPDPKFALEELKATVIPRVQARHTDLTHLSLSKFEFHAEAIFLLPSVPIDDPKNVPSHDTVRRTKREIAHMHDYHDFTLHLALAAQDGKEVVSKGWGQRHPLAGPGVPGPPTEWTFIYAPRNEEELAVVEMIIEASIGYMTNDPAGVVIA

SEQ ID NO: 85 - DNA, 798 bp, Armillaria ostoyae
atgtccttcatcgacagcatgaaacttgacttcgtcggacacctctttggcatcaggaatcgcggcttagccaccgcttgttgtgctgtggcagtcgcttctgccatcgccttcccttacattcgtagggactaccagacattcttatctggcggtccctcttacgctccccagaacatcaaaggatatctcatcgtctgcgtcctggccttgttccgtcaggagcaaaagggccttgcgatatatgaccgccttcccgagaagcgcaggtggctacctgacttgcctcctcgcaatggcccgcggcccatcacgaccagccatataatccaaagacagcgcaaccaggcgccagactccaagttcgccctcgaggaactcaaggctacggtcattccacgggtgcaggctcgccacactgacctcacccatctcagcctatccaagttcgagttccatgctgaagcaatcttcctgctcccctctgtacccatcgatgatccaaaaaatgttccaagtcatgacacggtgcgcaggacgaagagggagatcgcgcatatgcacgactaccatgactttacgttgcatcttgcactggccgcccaagacgggaaggaagtcgtggcgaagggatgggggcagcgacacccgctggcaggccctggcgttcctggtccacctacggagtggacgtttatttatgcgccacgtaacgaagaggaactggcagtggtggaaatgattatcgaggcatcaataggctatatgaccaatgaccctgctggaacagttatcgta

SEQ ID NO: 86 - protein, 266 aa, Armillaria ostoyae
MSFIDSMKLDFVGHLFGIRNRGLATACCAVAVASAIAFPYIRRDYQTFLSGGPSYAPQNIKGYLIVCVLALFRQEQKGLAIYDRLPEKRRWLPDLPPRNGPRPITTSHIIQRQRNQAPDSKFALEELKATVIPRVQARHTDLTHLSLSKFEFHAEAIFLLPSVPIDDPKNVPSHDTVRRTKREIAHMHDYHDFTLHLALAAQDGKEVVAKGWGQRHPLAGPGVPGPPTEWTFIYAPRNEEELAVVEMIIEASIGYMTNDPAGTVIV

SEQ ID NO: 87 - DNA, 798 bp, Armillaria mellea
atgtccttcttcgacagcgtgaaacttgacctcgtcggacgcctctttggcatcaggaatcgcggcttagctgttacttgttgtgctgtggcagtcgcctctatcatcgcgttcccttacattcgtagggactaccagacatttttatctgggggtccctcctacgctccccagaacatcagaggatacctcattgtctgcgtcctggccttgttccgtcaggagcaaaaaggccttgcgatatacgaccgccttcccgagaagcgcaggtggctacctgacttgcctcctcgcgatggcccacggcccatcacgaccagccatataatccaaagacagcgcaaccaggcgccggacctcaagttcgccctcgaggaactcaaggccacggtcattccacgggtgcaggctcgccacactgacctcacccatctcagcctatccaagttcgagttccatgctgaagcaatcttcctgctcccctctgtacccatcgatgatccaaagaatgtgccaagtcacgacacggtgcgcaggacgaagagggaaattgcgcatatgcacgactaccatgactacacgctgcatcttgcgttggccgcccaagacgggaaggaagtcgtatcaaagggatgggggcagcgacacccgctggcaggccctggcgttcctggtccaccgacggagtggacgtttatttatgcgccacgtaacgaagaggagctggcagtggtggaaatgattatcgaggcatcgataggctatatgaccaatgaccctgcaggaaaaactatcgca

SEQ ID NO: 88 - protein, 266 aa, Armillaria mellea
MSFFDSVKLDLVGRLFGIRNRGLAVTCCAVAVASIIAFPYIRRDYQTFLSGGPSYAPQNIRGYLIVCVLALFRQEQKGLAIYDRLPEKRRWLPDLPPRDGPRPITTSHIIQRQRNQAPDLKFALEELKATVIPRVQARHTDLTHLSLSKFEFHAEAIFLLPSVPIDDPKNVPSHDTVRRTKREIAHMHDYHDYTLHLALAAQDGKEVVSKGWGQRHPLAGPGVPGPPTEWTFIYAPRNEEELAVVEMIIEASIGYMTNDPAGKTIA

SEQ ID NO: 89 - DNA, 798 bp, Armillaria fuscipes
atgaccttcttggacagtatcaaacttgacctcgttgggcgcctctttggcatcaggaatcacggcgtagccgctgcctgttgtgctgcagcagttgcctctgccatcgtgttcccttatattcgtagggactaccagacatttctatctggcggcccttcctacgctccccagaacatcagaggatacatcattgtctgcgtcctagccttattccgccaggagcaaaaaggccttgcgatatatgaccgccttcccgagaagcgcaggtggttagctgacttgcctcctcgcaatggcccacggcccatcacaaccagtcatataattcaaagacagcgcaaccaggcgccagaccccaagttcgccctcgaagaactcaaggccacagtcattccacgggtacaggctcgccacactgacctcacccatctcagcctgtccaaattcgagtttcacgctgaagcaatcttcctgctcccctctgtacccatcgacgacccaaagaatatcccaagccatgacacagtgcgcaggacgaaaagggagatcgcgcatatgcacgactatcatgatttcacgctgcatcttgcactggctgcccaagacgggaaggaagtcgtatcaaagggatgggggcagcggcacccgctggcaggccctggtgtccctggtccaccaacggagtggacgtttatttacgcgccacggaacgaagaggagctggcagtagtggaaatgataattgaggcatcaataggctacatgaccaatgaccctgcaggatcagttattcca

SEQ ID NO: 90 - protein, 266 aa, Armillaria fuscipes
MTFLDSIKLDLVGRLFGIRNHGVAAACCAAAVASAIVFPYIRRDYQTFLSGGPSYAPQNIRGYIIVCVLALFRQEQKGLAIYDRLPEKRRWLADLPPRNGPRPITTSHIIQRQRNQAPDPKFALEELKATVIPRVQARHTDLTHLSLSKFEFHAEAIFLLPSVPIDDPKNIPSHDTVRRTKREIAHMHDYHDFTLHLALAAQDGKEVVSKGWGQRHPLAGPGVPGPPTEWTFIYAPRNEEELAVVEMIIEASIGYMTNDPAGSVIP

SEQ ID NO: 91 - DNA, 738 bp, Mycena citricolor
atggcttatcagctcacttggattcagactctcgtgctgggtgcccttgtggcaatggcagtagcgttccccttcatcaagaaagactacgagacgttcctgaagggcggcccctcctatgcgccccaaaacgttcgcggatacatcatcgtgctcgtgctcgcgctcttccgccaagagcagctcgggctggagatctacgaccgcatgcccgagaaacgtcgctggctcgcgaatctccctcagcgcgagggcccccgccccaagaccacaagtcacatcatccagcggcagctcagccagcacacggaccccgcattcggcgccgcgtacctcaaagacaccgtcattccgcgcgtccaggcgcggcacgcagccaacacgcacatcgcgcgctcgacgttcgagttccacgccgccgcgatcttcctgaacgcggacgtgccgctgcccgagggcctgcccgcaagcgagacggtgcggcggaccaagggcgagatcgcgcacatgcacgactaccacgacttcacgctgcacctcgcgctcgcggcggcggatgggaaggaggtggtcggcaagggctgggggcagcgccatccgctggcgggacccggtgtgccgggtccgccgaacgagtggacctttgtgtatgcgccgaggaatgaagaggagatgggcgtggtcgagcagatcgtagaggcggcgattgggtacatgtcgaacgtgcctgcgctggaa

SEQ ID NO: 92 - protein, 246 aa, Mycena citricolor
MAYQLTWIQTLVLGALVAMAVAFPFIKKDYETFLKGGPSYAPQNVRGYIIVLVLALFRQEQLGLEIYDRMPEKRRWLANLPQREGPRPKTTSHIIQRQLSQHTDPAFGAAYLKDTVIPRVQARHAANTHIARSTFEFHAAAIFLNADVPLPEGLPASETVRRTKGEIAHMHDYHDFTLHLALAAADGKEVVGKGWGQRHPLAGPGVPGPPNEWTFVYAPRNEEEMGVVEQIVEAAIGYMSNVPALE

SEQ ID NO: 93 - DNA, 747 bp, Panellus stipticus
atgaacatcaacctgaaagctctgatcggagtctgtgccgtgctcatcaccgctgcagtgttccccttcgttcgtaaagactatcacacctttcttgaaggtggaccatcctacgcgccgcagaatttgcaaggctatatcatcgtgttggtgctctcactctttcgaggggaggagacgggattggaaatatacgaccgcttgcccgaaaaacgccgctggctcgaggagctgcctgttcgcgaaggcccgcgcccaaagacaaccagccacatcattcagagacaattgaatcagcacgttgacccggacttcggaatgaactctttgaaaggctccgtcatccggcgccttcaatcccgccaccaggacataactcaactcgcactctcgaaattcgaattccacgccgaggccatatttctgcgccccgatatcgcgatcaacgatcccaaacacgtcccgagccacgacacggtgcgccgcacaaagcgcgagatagctcacatgcacgactaccatgattacacgtgtcatttggcgctcgcagcgcaggatgggaagcaagtgattgcaaaagggtggggccagagacatccgctcgcgggaccgggcatgccggggccgccgacggagtggacatttttgtatgcgccgaggaatgaggcggaggttcaagtgttggagacgattatcgaagcgtcaatcgggtacatgtcgaacgcaccagccttgggtgggagcgag

SEQ ID NO: 94 - protein, 249 aa, Panellus stipticus
MNINLKALIGVCAVLITAAVFPFVRKDYHTFLEGGPSYAPQNLQGYIIVLVLSLFRGEETGLEIYDRLPEKRRWLEELPVREGPRPKTTSHIIQRQLNQHVDPDFGMNSLKGSVIRRLQSRHQDITQLALSKFEFHAEAIFLRPDIAINDPKHVPSHDTVRRTKREIAHMHDYHDYTCHLALAAQDGKQVIAKGWGQRHPLAGPGMPGPPTEWTFLYAPRNEAEVQVLETIIEASIGYMSNAPALGGSE

SEQ ID NO: 95 - DNA, 792 bp, Neonothopanus gardneri
atgaatcttccgtctttcgtccaacgtctctccacagcaagcagtcgcagtatagcgattacttgcgtagttgtccttgcctctgcaatcgcctttcccttcatccgcagagactaccagaccttcctggaagtgggaccctcgtacgccccgcagaactttagaggatacatcatcgtctgtgtcctctcgttgttccgccaagaacaaaaaggactcgaaatctacgatcggctcccagagaaacgaaggtggttgtccgaccttccctttcgtgacgggcccagacccagcatcacaagccatatcattcaacgacagcgtacccaactagttgatccggacttcgctacccaggagctcataggcaaagtcatccctcgtgtgcaagcacgacacaccgacaaaacattcctcagcacctccaaattcgaatttcacgcaaaagccatattcctcctgccttccatcccaatcaacgaccctctgaacgttccaagccacgacactgtccgacgaacgaagcgcgagatcgcgcatatgcatgattatcatgattgcactcttcacatcgctctcgctgctcaggacggaaaggaggttttgaagaagggatgggggcaacgacacccactcgctggacctggagtgcccggcccaccgacggagtggacgtttctctatgcgcctcgaaacgaagaagaggttcgagttgtggagatgattattgaggctgccataggttacatgacgaatgatccggcaggaaaagttgtagaagccactggaaag

SEQ ID NO: 96 - protein, 264 aa, Neonothopanus gardneri
MNLPSFVQRLSTASSRSIAITCVVVLASAIAFPFIRRDYQTFLEVGPSYAPQNFRGYIIVCVLSLFRQEQKGLEIYDRLPEKRRWLSDLPFRDGPRPSITSHIIQRQRTQLVDPDFATQELIGKVIPRVQARHTDKTFLSTSKFEFHAKAIFLLPSIPINDPLNVPSHDTVRRTKREIAHMHDYHDCTLHIALAAQDGKEVLKKGWGQRHPLAGPGVPGPPTEWTFLYAPRNEEEVRVVEMIIEAAIGYMTNDPAGKVVEATGK

SEQ ID NO: 97 - DNA, 753 bp, Mycena chlorophos
atggtccaactcacccgaacctccggattcatcgccgctgcggccattgttgctgccatcgccttcccgttcattcgtcgagactaccagacgttccttcgtggtgggccgtcctatgccccacagaacatccgcggctatatcatcgtcctggttctgtccctcttccgcggcgaggagaagggtcttgcaatctacgagccccttcctgagaagcgcacatggctgccggagcttccgcggcgcgcgggagaccggcccaagacgacgagccacatcatccaacggcagctcgaccagtaccccgacccggactttgtcctcaaagccctgaaagcgacggtcatcccgcgtgtccaagcccggcacacagacaagactcacctcgcgctgtccaagttcgagttccatgctgaggccatcttcgtgcgcccggaaatcgccatcgacgacccgaagcatatccccagccacgacacggtgcgacggacgaagcgcgagattgcgcacatgcacgactatcacgactgcacgctgcatttggcgctagcggcgcaggacgcgaagcaggtgctgcagaagggctggggccagcgccatccgctggcagggcctgggatgcccgggccgcccacggagtggacgttcttgtatgccccgaggaccgaggaggaagtgaaggttgtggagaccattgtcgaggcctctatcgcgtacatgacgaacgcggagaagccggtcgagctggtgcag

SEQ ID NO: 98 - protein, 251 aa, Mycena chlorophos
MVQLTRTSGFIAAAAIVAAIAFPFIRRDYQTFLRGGPSYAPQNIRGYIIVLVLSLFRGEEKGLAIYEPLPEKRTWLPELPRRAGDRPKTTSHIIQRQLDQYPDPDFVLKALKATVIPRVQARHTDKTHLALSKFEFHAEAIFVRPEIAIDDPKHIPSHDTVRRTKREIAHMHDYHDCTLHLALAAQDAKQVLQKGWGQRHPLAGPGMPGPPTEWTFLYAPRTEEEVKVVETIVEASIAYMTNAEKPVELVQ

SEQ ID NO: 99 - DNA, 801 bp, Artificial sequence
  Optimized for mammalian expression (humanized) nucleic acid, encoding protein SEQ ID No80
atgcgcattaacatctccctttcatctcttttcgagcgattgagcaaactgagttccaggagtattgcaatcacttgtggggttgtcctcgcgagcgccatcgcattccccatcatccggagagattatcagacgtttcttgaggtgggccctagctatgcaccacagaacttccgaggatatatcatcgtgtgtgtactgtcactgtttaggcaagaacaaaagggattggctatctatgataggttgcctgagaaacggcggtggctcgctgatctcccatttagagaggggacacgaccgagcatcacttcacacatcatacaaagacagcgaacgcagctcgttgaccaagagttcgcaactagggaactgattgataaggtgatacccagagtacaggcgcgacacaccgataaaacttttctttccacctctaaattcgagttccatgccaaagctattttcttgttgccttccataccgattaatgatcctctgaatattccatcccacgacacagttcgacggacgaaacgcgaaattgcgcacatgcacgactatcacgattgcactttgcacctggcactggctgctcaagacggaaaagaagttctgaaaaagggttgggggcaaagacatccgctggcgggacccggtgtacctgggccgcctacggaatggacatttttgtacgcaccgaggaacgaagaggaggccagggtcgttgagatgattgttgaggctagtattgggtacatgacgaatgatccggctggtaaaattgttgaaaatgcaaag

SEQ ID NO: 100 - DNA, 1266 bp, Artificial sequence
  Optimized for mammalian expression (humanized) nucleic acid, encoding protein SEQ ID No2
atggcgtcattcgagaacagtcttagcgtgctgatcgtcggcgctgggctcggcggtcttgctgcggcaatcgccctcaggcgacaaggacacgttgttaaaatctatgacagctcatcatttaaggcagagttgggcgcaggcctcgcggtccccccaaacactttgagatcactgcaacaactgggttgtaatactgagaaccttaacggcgtggataacctctgcttcactgcaatgggttacgatggcagtgtgggtatgatgaacaatatgaccgattatagggaggcgtacggcactagctggataatggtccatcgggttgatctccacaatgagcttatgcgcgtagcgttggatccgggcggattgggacccccagctaccttgcacttgaatcaccgcgtgactttttgtgatgtcgacgcatgcacagtaaccttcaccaatgggacgactcagtcagcggatctcatcgtcggcgccgacggtatacgatccactatccgcagattcgtcctggaggaagatgtcacagttccggcatccggaatcgttggtttccgctggctcgtccaggctgatgctttggatccttaccctgaacttgactggattgttaaaaagccccctctcggcgctaggttgataagtacgcctcaaaacccgcagtctggggtaggtctcgcggatcgcagaacgatcattatatacgcgtgtcgaggaggtactatggtaaacgtacttgccgtccatgacgatgagagggatcagaatacggcagattggtccgtgccagctagcaaggatgatcttttcagagtttttcacgactatcatcctcgatttcggcggctgttggagttggcgcaagacatcaatctgtggcagatgcgggtggtccccgttctgaagaaatgggtgaacaaaagagtctgtctcttgggggatgcagcgcatgcgtccctccctaccttggggcagggtttcggcatggggttggaggacgccgtagcccttgggactttgcttccaaaggggacgacagcatcccaaatagaaacaagacttgccgtatatgagcagctccgaaaagatcgcgccgagttcgtcgcggctgagtcctacgaagaacaatatgtaccagaaatgaggggactttacctgcgatccaaagaattgcgcgaccgggtaatgggctatgacataaaggtggagtccgaaaaggtcttggagacactgttgcggtcaagcaattccgcc

SEQ ID NO: 101 - DNA, 2112 bp, Artificial sequence
  Nucleic acid encoding the fusion protein Hispidin-hydroxylase and luciferase
atggcatcgtttgagaattctctaagcgttttgattgtcggggccggacttggtgggcttgctgctgccatcgcgctgcgtcgccaagggcatgtcgtgaaaatatacgactcctctagcttcaaagccgaacttggtgcgggactcgctgtgccgcctaacaccttgcgcagtctacagcaacttggttgcaataccgagaacctcaatggtgtggataatctttgcttcactgcgatggggtatgacgggagtgtagggatgatgaacaacatgactgactatcgagaggcatacggtacttcttggatcatggtccaccgcgttgacttgcataacgagctgatgcgcgtagcacttgatccaggtgggctcggacctcctgcgacactccatcttaatcatcgtgtcacattctgcgatgtcgacgcttgcaccgtgacattcaccaacgggaccactcaatcagctgatctcatcgttggtgcagacggtatacgctctaccattcggcggtttgtcttagaagaagacgtgactgtgcctgcgtcaggaatcgtcgggtttcgatggcttgtacaagctgacgcgctggacccatatcctgaactcgactggattgttaaaaagcctcctctaggcgcgcgactgatctccactcctcagaatccacagtctggtgttggcttggctgacaggcgcactatcatcatctacgcatgtcgtggcggcaccatggtcaatgtccttgcagtgcatgatgacgaacgtgaccagaacaccgcagattggagtgtaccggcttccaaagacgatctatttcgtgttttccacgattaccatccacgctttcggcggcttttagagcttgcgcaggatattaatctctggcaaatgcgtgttgtacctgttttgaaaaaatgggttaacaagcgggtttgcttgttaggagatgctgcgcacgcttctttaccgacgttgggtcaaggttttggtatgggcctggaagatgccgtagcacttggtacactccttccaaagggtaccactgcatctcagatcgagactcgacttgcggtgtacgaacagctacgtaaggatcgtgcggaatttgttgcggctgaatcatatgaagagcaatatgttcctgaaatgcggggactttatctgaggtcaaaggaactgcgtgatagagtcatgggttatgatatcaaagtggagagcgagaaggttctcgagacgctcctaagaagttctaattctgcctccggaactgggggcaacgcctctgacggtggtgggtctggtggtatgcgcattaacattagcctctcgtctctcttcgaacgtctctccaaacttagcagtcgcagcatagcgattacatgtggagttgttctcgcctccgcaatcgcctttcccatcatccgcagagactaccagactttcctagaagtgggaccctcgtacgctccgcagaactttagaggatacatcatcgtctgtgtcctctcgctattccgccaagagcagaaagggctcgccatctatgatcgtcttcccgagaaacgcaggtggttggccgaccttccctttcgtgaaggaaccagacccagcattaccagccatatcattcagcgacagcgcactcaactggtcgatcaggagtttgccaccagggagctcatagacaaggtcatccctcgcgtgcaagcacgacacaccgacaaaacgttcctcagcacatcaaagttcgagtttcatgcgaaggccatatttctcttgccttctatcccaatcaacgaccctctgaatatccctagccacgacactgtccgccgaacgaagcgcgagattgcacatatgcatgattatcatgattgcacacttcatcttgctctcgctgcgcaggatggaaaggaggtgctgaagaaaggttggggacaacgacatcctttggctggtcctggagttcctggtccaccaacggaatggacttttctttatgcgcctcgcaacgaagaagaggctcgagtagtggagatgatcgttgaggcttccatagggtatatgacgaacgatcctgcaggaaagattgtagaaaacgccaag

SEQ ID NO: 102 - protein, 704 aa, Artificial sequence
  Fusion protein amino acid sequence hispidin-hydroxylase and luciferase
MASFENSLSVLIVGAGLGGLAAAIALRRQGHVVKIYDSSSFKAELGAGLAVPPNTLRSLQQLGCNTENLNGVDNLCFTAMGYDGSVGMMNNMTDYREAYGTSWIMVHRVDLHNELMRVALDPGGLGPPATLHLNHRVTFCDVDACTVTFTNGTTQSADLIVGADGIRSTIRRFVLEEDVTVPASGIVGFRWLVQADALDPYPELDWIVKKPPLGARLISTPQNPQSGVGLADRRTIIIYACRGGTMVNVLAVHDDERDQNTADWSVPASKDDLFRVFHDYHPRFRRLLELAQDINLWQMRVVPVLKKWVNKRVCLLGDAAHASLPTLGQGFGMGLEDAVALGTLLPKGTTASQIETRLAVYEQLRKDRAEFVAAESYEEQYVPEMRGLYLRSKELRDRVMGYDIKVESEKVLETLLRSSNSASGTGGNASDGGGSGGMRINISLSSLFERLSKLSSRSIAITCGVVLASAIAFPIIRRDYQTFLEVGPSYAPQNFRGYIIVCVLSLFRQEQKGLAIYDRLPEKRRWLADLPFREGTRPSITSHIIQRQRTQLVDQEFATRELIDKVIPRVQARHTDKTFLSTSKFEFHAKAIFLLPSIPINDPLNIPSHDTVRRTKREIAHMHDYHDCTLHLALAAQDGKEVLKKGWGQRHPLAGPGVPGPPTEWTFLYAPRNEEEARVVEMIVEASIGYMTNDPAGKIVENAK

SEQ ID NO: 103 - DNA, 1266 bp, Artificial sequence
  Nucleic acid optimized for expression in plants, encoding protein SEQ ID No2
atggcatcttttgagaattctctgagcgttttgattgtcggggccggacttggtgggcttgctgctgccatcgccctgcgtcgacaagggcatgtcgtgaaaatatacgactcctctagcttcaaagccgaacttggtgctggactcgctgtgccgcctaacaccttgcgtagtctccagcaacttggttgcaataccgagaacctcaatggtgtggataatctttgcttcactgctatggggtatgacgggagtgtagggatgatgaacaacatgactgactatcgagaggcatacggtacttcttggatcatggtccacagagttgacttgcataacgagctgatgagggtagcacttgatccaggtgggctcggacctcctgcaacactccatcttaatcatcgtgtcacattctgcgatgtcgacgcttgcaccgtgacattcaccaacgggaccactcaatcagctgatctcatcgttggtgcagacggtataagatctaccattcgaaggtttgtcctggaagaagacgtgactgtgcctgcatcaggaatcgtcgggtttcgatggcttgtacaagctgacgctctggacccatatcctgaactcgactggattgttaaaaagcctcctcttggcgcacgactgatctccactcctcagaatccacagtctggtgttggcttggctgacaggaggactatcatcatctacgcatgtcgtggcggcaccatggtcaatgtccttgcagtgcatgatgacgaacgtgaccagaacaccgcagattggagtgtaccggcttccaaagacgatctctttcgtgttttccacgattaccatccacgatttaggaggcttctcgagcttgctcaggatattaatctctggcaaatgcgtgttgtacctgttttgaaaaaatgggttaacaagagggtttgcttgctcggagatgctgcacacgcttctctgccgacgttgggtcaaggttttggtatgggtctggaagatgccgtagcacttggtacactccttccaaagggtaccactgcatctcagatcgagactcgacttgctgtgtacgaacagttgcgtaaggatcgtgccgaatttgttgccgctgaatcatatgaagagcaatatgttcctgaaatgcgaggactttatctgaggtcaaaggaactgcgtgatagagtcatgggttatgatatcaaagtggagagcgagaaggttctcgagacgctccttagaagttctaattctgcc

SEQ ID NO: 104 - DNA, 1032 bp, Aspergillus nidulans
atggtgcaggacacgtcatctgcttctacttcccccattttgacacgttggtacatcgacacaagacccttaacagcatccacggcagcacttcctcttctggaaacgttgcagcctgcagatcagattagtgtacagaagtattatcatttaaaggataaacatatgagtcttgcttctaacttattgaagtatctgtttgttcatcgtaattgtcgtattccctggagttctatcgtgatctcccgtacacctgatcctcaccgtcgtccatgctacattcctccctcaggctctcaggaagattccttcaaagacggatacactggtatcaatgttgagttcaatgtatcacaccaagccagtatggtcgccattgctggaaccgcatttactcccaattctggtggcgattctaaactgaagcctgaggtgggaatcgacatcacatgtgtaaacgagcgtcagggccgtaacggtgaggagcgttcattggaatctcttcgtcaatatatcgacatcttctctgaagtattctccacggctgagatggcaaacatccgtcgtcttgatggcgttagtagttcttctttaagtgctgatcgtcttgttgactatggttaccgtcttttttatacatactgggccctgaaggaagcctatatcaagatgactggtgaagcattgcttgcaccatggttacgtgagctggagttctccaatgttgttgcccctgccgctgttgctgaatccggtgactctgccggcgacttcggtgagccatacacaggagtccgtacgactttatataaaaatttagttgaggatgtgcgtatcgaggtagccgcccttggtggcgattatctttttgcaaccgcagctcgtggaggaggaattggagccagttcaaggcccggaggcggccctgatggtagtggtatccgttcacaggacccttggcgtccatttaagaaacttgatatcgagcgtgacattcagccatgtgcaacaggtgtatgcaactgccttagt

SEQ ID NO: 105 - protein, 344 aa, Aspergillus nidulans
MVQDTSSASTSPILTRWYIDTRPLTASTAALPLLETLQPADQISVQKYYHLKDKHMSLASNLLKYLFVHRNCRIPWSSIVISRTPDPHRRPCYIPPSGSQEDSFKDGYTGINVEFNVSHQASMVAIAGTAFTPNSGGDSKLKPEVGIDITCVNERQGRNGEERSLESLRQYIDIFSEVFSTAEMANIRRLDGVSSSSLSADRLVDYGYRLFYTYWALKEAYIKMTGEALLAPWLRELEFSNVVAPAAVAESGDSAGDFGEPYTGVRTTLYKNLVEDVRIEVAALGGDYLFATAARGGGIGASSRPGGGPDGSGIRSQDPWRPFKKLDIERDIQPCATGVCNCLS

SEQ ID NO: 106 - DNA, 1593 bp, Rhodobacter capsulatus
atgactcttcaatcccaaactgctaaggattgcttggcactggatggtgcactgactttggtgcagtgcgaggcaatcgccacgcatcgtagtcgtatttccgttacgccagccttgcgtgaacgttgtgctcgtgcccacgcacgtttggagcacgcaatcgcagagcagcgtcatatttacggtatcaccactggatttggacccctggctaaccgtcttattggagccgatcagggcgctgagctgcaacagaatctgatttaccatcttgcaacgggcgttggacccaagctgtcctgggcagaggcccgtgcacttatgttggcacgtcttaattccattttgcagggcgctagtggtgcttctcctgaaacgatcgaccgtatcgtcgcagtacttaacgctggctttgctcctgaagttcccgcacaaggtactgttggcgcaagtggcgacttgactccccttgcccacatggtactggccctgcaaggccgtggacgtatgatcgatcctagtggacgtgtgcaagaggctggagccgtcatggatcgtctttgcggaggccctcttacgttggccgcacgtgacggccttgctctggtcaacggtacaagtgctatgactgctatcgctgctcttactggagtcgaggccgctcgtgctatcgatgctgcacttcgtcactccgccgttttgatggaagtcctgtctggacatgcagaagcctggcaccccgcctttgcagagttgagaccccacccaggccagttgcgtgctactgaacgtttggcccaggccctggatggcgcaggccgtgtatgccgtactctgacggctgcccgtcgtcttactgcagccgacctgcgtccagaggaccaccccgcacaagacgcttattctctgcgtgtcgttcctcaactggttggtgccgtatgggatactcttgactggcacgaccgtgtcgtgacctgtgagcttaattcagtaaccgataaccccatttttccagagggttgcgccgttcctgcacttcacggaggaaacttcatgggagtccacgtcgcactggcttcagacgctttgaatgcagccctggttacccttgccggtcttgtagagcgtcaaattgcccgtcttaccgacgagaaacttaataaaggtctgcctgctttccttcacggtggacaggcaggccttcagagtggcttcatgggcgctcaggttacagcaactgccctgttggctgagatgcgtgcaaatgcaactccagtctcagtacaatccttgtccacaaacggtgcaaaccaagacgtcgtatctatgggaactatcgccgcacgtcgtgctcgtgcacaattgttgccactgtcacagattcaggctatcttggctttggcattggcccaggctatggatcttcttgacgatcccgagggtcaagccggatggtctttgacagcccgtgatttgcgtgaccgtatccgtgccgtttctcccggactgcgtgccgaccgtccactggcaggccatatcgaagctgttgctcagggacttcgtcacccatctgctgcagccgatccaccagcc

SEQ ID NO: 107 - protein, 531 aa, Rhodobacter capsulatus
MTLQSQTAKDCLALDGALTLVQCEAIATHRSRISVTPALRERCARAHARLEHAIAEQRHIYGITTGFGPLANRLIGADQGAELQQNLIYHLATGVGPKLSWAEARALMLARLNSILQGASGASPETIDRIVAVLNAGFAPEVPAQGTVGASGDLTPLAHMVLALQGRGRMIDPSGRVQEAGAVMDRLCGGPLTLAARDGLALVNGTSAMTAIAALTGVEAARAIDAALRHSAVLMEVLSGHAEAWHPAFAELRPHPGQLRATERLAQALDGAGRVCRTLTAARRLTAADLRPEDHPAQDAYSLRVVPQLVGAVWDTLDWHDRVVTCELNSVTDNPIFPEGCAVPALHGGNFMGVHVALASDALNAALVTLAGLVERQIARLTDEKLNKGLPAFLHGGQAGLQSGFMGAQVTATALLAEMRANATPVSVQSLSTNGANQDVVSMGTIAARRARAQLLPLSQIQAILALALAQAMDLLDDPEGQAGWSLTARDLRDRIRAVSPGLRADRPLAGHIEAVAQGLRHPSAAADPPA

SEQ ID NO: 108 - DNA, 1560 bp, Escherichia coli
atgaaacctgaagattttcgtgcttccacccagcggccctttactggagaagaatatctgaagtctcttcaagacggccgtgagatctatatttacggcgagcgtgtaaaggatgttacaacgcaccctgcttttcgtaatgctgctgcctcagttgcccaactttatgacgccctgcataaacccgaaatgcaggactcactttgttggaatacggacacggggtccggtggttacacacacaagttctttcgtgtcgccaaatctgccgatgacttgcgtcaacaacgtgatgctatcgctgaatggtcacgtctgagttacggctggatgggccgtacgcccgactataaggccgcctttggttgcgcattgggtgcaaatcccggcttttatggacagtttgaacaaaatgcacgtaactggtatactcgtatccaggaaaccggcttgtactttaatcatgctattgttaacccacctatcgatcgtcatttgccaaccgataaggtcaaggatgtatatattaaacttgaaaaggagacggacgctggtatcattgtgtctggcgctaaagtggtggctacgaacagtgcattgacgcattacaatatgattggttttggttccgcccaagtgatgggcgagaaccccgactttgccttgatgtttgttgcccctatggacgctgacggagtaaaacttatttcacgtgcctcatatgaaatggtagctggtgccactggttctccttatgattatcctctttcatcccgttttgacgaaaacgacgccatcctggtcatggacaatgttcttattccctgggagaacgtgctgatctaccgtgacttcgatcgttgccgtcgttggactatggaaggaggtttcgctcgtatgtatccactgcaggcctgtgttcgtttggctgtgaagttggactttatcacagcccttcttaaaaagtcacttgagtgtactggaacgttggagttccgtggtgtacaggcagatctgggcgaagtagtagcctggcgtaatactttctgggcactgtctgactctatgtgctccgaggcaacaccatgggtgaacggcgcatacctgcccgatcacgccgctcttcaaacatatcgtgtgcttgctcccatggcttatgctaaaatcaaaaatattatcgagcgtaatgtaacttctggtctgatttacctgccatccagtgctcgtgaccttaacaatcctcaaatcgatcagtatcttgcaaaatatgtgcgtggctcaaacggaatggaccatgtgcagcgtatcaaaatcctgaagttgatgtgggatgccatcggttctgaatttggcggacgtcatgaactgtatgagattaactactctggatctcaagacgaaatcagacttcagtgtttgcgtcaggcccaaaattctggaaatatggataaaatgatggctatggtagaccgttgtctgagtgagtacgatcaagatggttggacagttcctcatcttcataataatgacgatattaatatgcttgataagttgcttaaa

SEQ ID NO: 109 - protein, 520 aa, Escherichia coli
MKPEDFRASTQRPFTGEEYLKSLQDGREIYIYGERVKDVTTHPAFRNAAASVAQLYDALHKPEMQDSLCWNTDTGSGGYTHKFFRVAKSADDLRQQRDAIAEWSRLSYGWMGRTPDYKAAFGCALGANPGFYGQFEQNARNWYTRIQETGLYFNHAIVNPPIDRHLPTDKVKDVYIKLEKETDAGIIVSGAKVVATNSALTHYNMIGFGSAQVMGENPDFALMFVAPMDADGVKLISRASYEMVAGATGSPYDYPLSSRFDENDAILVMDNVLIPWENVLIYRDFDRCRRWTMEGGFARMYPLQACVRLAVKLDFITALLKKSLECTGTLEFRGVQADLGEVVAWRNTFWALSDSMCSEATPWVNGAYLPDHAALQTYRVLAPMAYAKIKNIIERNVTSGLIYLPSSARDLNNPQIDQYLAKYVRGSNGMDHVQRIKILKLMWDAIGSEFGGRHELYEINYSGSQDEIRLQCLRQAQNSGNMDKMMAMVDRCLSEYDQDGWTVPHLHNNDDINMLDKLLK

SEQ ID NO: 110 - DNA, 510 bp, Escherichia coli
atgcagcttgatgagcagcgtttgcgttttcgtgacgctatggcatcacttagtgccgctgtgaacatcatcacgactgagggagacgccggtcagtgcggaatcacagcaaccgcagtatgtagtgttacggacactcccccatctctgatggtctgtatcaatgccaatagtgctatgaatccagtctttcagggtaatggtaaactttgtgtcaacgtcctgaatcacgagcaagagttgatggctcgtcattttgcaggaatgactggtatggctatggaagagcgtttttccttgtcttgttggcaaaaaggaccactggcccagcctgtattgaaaggttctttggcttccctggagggtgagatccgtgacgttcaagccattggaactcatctggtatatttggtagaaatcaagaatattatcctgtcagccgagggccatggtctgatctactttaaacgtcgttttcaccccgtgatgctggagatggaggcagccatt

SEQ ID NO: 111 - protein, 170 aa, Escherichia coli
MQLDEQRLRFRDAMASLSAAVNIITTEGDAGQCGITATAVCSVTDTPPSLMVCINANSAMNPVFQGNGKLCVNVLNHEQELMARHFAGMTGMAMEERFSLSCWQKGPLAQPVLKGSLASLEGEIRDVQAIGTHLVYLVEIKNIILSAEGHGLIYFKRRFHPVMLEMEAAI

SEQ ID NO: 112 - DNA, 801 bp, Artificial sequence
  Nucleic acid optimized for expression in plants, encoding protein SEQ ID No80
atgaggataaatatctctttgtctagtctctttgagagactgagcaaattgtcatcccgatccatcgcaattacctgcggcgtagtcttggcaagtgcaatagcattcccaattatccgaagagactatcaaacgttccttgaagtcggtccgagctatgccccacagaacttccgaggctatatcatcgtttgtgtcttgtcactttttaggcaagagcagaaagggttggcaatctatgacaggcttccagaaaagaggcgttggcttgccgacttgccgtttcgtgaagggacgaggccatccataacctcccacattatacagcgtcagcgtactcagctggtagatcaagagtttgcaaccagagaacttatcgataaggtgatcccacgtgtgcaagcaagacacacagacaaaacttttctcagtacgtcaaaatttgagtttcatgcaaaggccatcttccttctcccctctatccctattaatgatcctcttaacattccctcacacgatacggtaagaagaaccaagcgtgagatcgctcacatgcatgattaccatgattgcactctgcacttggctcttgctgctcaggatggtaaggaagttttgaagaaggggtggggccagcgtcacccactggccggaccaggagtcccaggccctcctactgagtggaccttcctttacgcaccaaggaacgaagaggaggccagagttgtcgagatgatagtcgaagctagtattggctacatgactaacgatcctgctggtaagattgtcgaaaatgccaag

SEQ ID NO: 113 - DNA, 5034 bp, Artificial squence
  Optimized for expression in plants of a nucleic acid, encoding protein SEQ ID No35
atgaattccagcaagaatcctccttccactcttcttgatgtttttctggatactgccaggaaccttgataccgctttgaggaatgtcttggaatgcggcgaacacagatggtcctacagagagcttgatactgtttcatctgctctcgcccagcatcttaggtacactgtcggtcttagtcctactgtcgccgtcatcagtgaaaaccatccttatattctcgctttgatgctggctgtatggaaacttggaggcaccttcgctcctattgatgtccattctcctgccgaattggtagctggcatgctgaacatagtctctccttcttgcttggttattccgagctcagatgtaactaatcaaactcttgcctgcgatcttaatatccccgtcgttgcatttcacccacatcaatccactattcctgagctgaacaagaagtacctcaccgattctcaaatttctccggatcttcctttttcagatccaaacagacctgctctgtacctcttcacttcatccgccacttctcgaagtaatctcaaatgcgtgcctctcactcacacctttatcctccgaaacagcctctctaagcgtgcatggtgcaagcgtatgcgtccagagacagactttgacggcatacgtgttcttggatgggccccgtggtctcacgtcctcgcacacatgcaagacatcggaccactcaccctgcttaatgccggatgctacgtttttgcaactactccatccacgtaccctacggaattgaaggacgacagggacctgatatcttgcgccgcaaatgctatcatgtacaagggcgtcaagtcatttgcttgtcttccctttgtactcggagggctgaaggcactctgcgagtctgagccatccgtgaaggcccatcttcaggtcgaggagagagctcaactcctgaagtctctgcaacacatggaaattcttgagtgtggaggtgccatgctcgaagcaagtgttgcctcttgggctattgagaactgcattcccatttctatcggtattggtatgacggagactggtggagccctctttgcaggccccgttcaggccatcaaaaccgggttttcttcagaggataaattcattgaagatgctacttacttgctcgttaaggatgatcatgagagtcatgctgaggaggatattaacgagggtgaactcgttgtgaaaagtaaaatgctcccacgaggctaccttggctatagtgatccttccttctcagtcgacgatgctggctgggttacatttagaacaggagacagatacagcgttacacctgacggaaagttttcctggctgggccgaaacactgatttcattcagatgaccagtggtgagacgctggatccccgaccaattgagagcagtctctgcgaaagttctcttatttctagagcatgcgttatcggagataaatttctcaacgggcctgctgctgctgtttgtgctatcattgagcttgagcccacagctgtggaaaaaggacaagctcacagccgtgagatagcaagagttttcgcacctattaatcgagacttgccgcctcctcttaggattgcatggagtcacgttttggttctccagcccagtgagaagataccgatgacgaagaagggtaccatcttcaggaagaaaattgagcaggtgtttggctctgccttgggtggcagctctggagataactctcaagccactgctgatgctggcgttgttcgacgagacgagctttcaaacactgtcaagcacataattagccgtgttctcggagtttccgatgacgaactcctttggacgttgtcatttgccgagctcggaatgacgtcagcattggccactcgaatcgccaacgagttgaacgaagttttggttggagttaatctccctatcaacgcttgctatatacatgtcgaccttccttctctgagcaatgccgtctatgccaaacttgcacacctcaagctgccagatcgtactcccgaacccaggcaagcccctgtcgaaaactctggtgggaaggagatcgttgtcgttggccaggcctttcgtcttcctggctcaataaacgatgtcgcctctcttcgagacgcattcctggccagacaagcatcatccattatcactgaaataccatccgataggtgggaccacgccagcttctatcccaaggatatacgtttcaacaaggctggccttgtggatatagccaattatgatcatagctttttcggactgacggcaaccgaagcactctatctgtccccaactatgcgtttggcactcgaagtttcctttgaagccttggagaatgctaatatcccggtgtcacaactcaagggttctcaaacagctgtttatgttgctactacagatgacggatttgagacccttttgaatgccgaggccggctatgatgcttatacaagattctatggcactggtcgagcagcaagtacagccagcgggcgtataagctgtcttcttgatgtccatggaccctctattactgttgatacggcatgcagtggaggggctgtttgtattgaccaagcaatcgactatctgcaatcaagcagtgcagcagacaccgctatcatatgtgctagtaacacgcactgctggccaggcagcttcaggtttctttccgcacaagggatggtatccccaggaggacgatgcgcaacatttacaactgatgctgatggctacgtgccctctgagggcgctgtcgccttcatattgaaaacccgagaagcagctatgcgtgacaaggacactatcctcgccacaatcaaagctacacagatatcacacaatggccgatctcaaggtcttgtggcaccgaatgtcaacagccaagctgaccttcatagaagcttgcttcaaaaagctggccttagcccggctgatatccgtttcattgaagctcatgggacaggaacgtcactgggagacctctcagaaattcaagctataaatgatgcttatacctcctctcagccgcgtacgaccggcccactcatagtcagcgcttccaaaacggtcattggtcataccgaaccagctggccccttggtcggtatgctgagtgtcttgaactctttcaaagaaggcgccgtccctggtctcgcccatcttaccgcagacaatttgaatcccagtctggactgttcttctgtgccacttctcattccctatcaacctgttcacctggctgcacccaagcctcaccgagctgctgtaaggtcatacggcttttcaggtaccctgggcggcatcgttctcgaggctcctgacgaagaaagactggaagaagagctgccaaatgacaagcccatgttgttcgtcgtcagcgcaaagacacatacagcacttatcgaatacctggggcgttatctcgagttcctcttgcaggcaaacccccaagatttttgtgacatttgttatacaagctgcgttgggcgtgagcactatagatatcgatatgcttgtgtagcaaatgatatggaggacctcataggccaactccagaaacgtttgggcagcaaggtgccgccaaagccgtcatacaaacgtggtgctttggcctttgccttttctggtcagggtacacaattccgagggatggccacagagcttgcaaaagcatactccggcttccgaaagatcgtgtccgatctcgcaaagagagctagcgagttgtcaggtcatgccattgaccgttttcttcttgcatatgacataggcgctgaaaatgtagctcctgatagtgaggcagaccagatttgcatctttgtgtatcagtgttctgtccttcgttggctgcagactatggggattagacccagtgcagtgataggccatagcctcggggagatctcagcttctgtggcagcaggagcactttctcttgactccgctttggatcttgtcatctcacgagctcgtcttttgaggtcttcagcaagtgctcctgcaggaatggcagctatgtctgcctctcaagacgaggttgtggagttgattgggaaactcgacctcgacaaggctaattctctcagcgtttcagtcataaatggtccccaaaatactgtcgtgtccggctcttcagctgctattgaaagcatagtggctcttgccaaagggagaaagatcaaagcctctgccctgaatatcaatcaagcttttcatagtccatacgtcgacagtgccgtccctggtctccgtgcttggtcagaaaagcatatctcctcagctagaccattgcaaattccgctgtattcaacgttgttgggagcacaaatctctgagggagagatgttgaatccagatcactgggtcgaccatgcacgaaagcctgtacagttcgcacaagcagccacaaccatgaaagaatccttcaccggagtcatcatagatatcggccctcaagtagtggcttggtcacttctgctcagtaacgggctcacgtccgtgactgctctcgctgcaaaaagagggagaagtcaacaggtggctttcctcagcgccttggccgatttgtatcaagattacggtgttgttcctgattttgtcgggctttatgctcagcaggaagatgcttcaaggttgaagaagacggatatcttgacgtatccgttccagcgtggcgaagagactctttctagtggttctagcactccgacattggaaaacacggatttggattccggtaaggaactccttatgggaccgactagagggttgctccgtgctgacgacttgcgtgacagtatcgtttcttctgtgaaggatgttctggaactcaagtcaaatgaagacctcgatttgtctgaaagtctgaatgcacttggtatggacagcatcatgttcgctcagctccgaaagcgtattggggaaggactcggattgaatgttccgatggtttttctgagcgacgccttttctattggtgagatggttagtaatcttgtggaacaggcagaggcttctgaggacaat

SEQ ID NO: 114 - DNA, 867 bp, Artificial sequence
  Optimized for expression in plants of a nucleic acid, encoding protein SEQ ID No65
atggctccaatttcttcaacttggtctcgtctcattcgatttgtggctattgaaacgtccctcgtgcatatcggtgaaccgatagacgccaccatggacgtcggtctggccagacgagaaggcaagacgatccaagcatacgagattattggatcaggcagtgctcttgacctctcagcccaagtaagtaagaatgtgctgactgtaagggaactcctgatgccgctttcaagagaggaaattaaaactgtacgatgcttggggttgaactaccctgttcatgccaccgaagcaaacgttgctgttccaaaattcccgaatttgttctacaaaccagtgacctccctcattggccccgatggactcattaccatcccttccgttgtccaacccccgaaggagcatcagtccgattatgaagcagaacttgtcattgtcatcgggaaagcagcaaagaatgtaagtgaggatgaggctttggattatgtattgggatacactgccgctaacgatatttcctttaggaaacaccagcttgcagtctcacaatggtctttctctaaaggatttggtagccttttgctcactatccgtatggcacaaacccactctggtaacattaatagattctccagagaccagattttcaatgtcaagaagacaatttccttcctgtcacaaggcactacactggaaccaggttctatcattttgactggtacacctgacggagtgggctttgtgcgaaatccaccactttaccttaaagatggagatgaagtaatgacctggattggaagtggaatcggaacattggccaatacagtgcaagaagagaagacttgcttcgctagtggcggacacgag

SEQ ID NO: 115 - protein, 9 aa, Artificial sequence
  Amino acid linker sequence
GGSGGSGGS

SEQ ID NO: 116 - DNA, 1569 bp, Artificial sequence
  Optimized for expression in plants of a nucleic acid, encoding a phenylalanine-ammonium-lyase Streptomyces maritimus (SEQ ID No 117)
atgacattcgtgatagagctggacatgaatgtcactcttgatcagttagaggatgctgcaagacaaagaacacccgtggaattgtctgcacctgtacgttcaagggtccgtgcttcacgtgatgttttagttaaattcgtacaagatgagcgtgtgatttacggtgtaaatacttcaatgggcggattcgtggatcacctagtacctgtgagtcaggcccgtcaattgcaagagaatttaatcaatgcagttgccacaaatgttggagcatatttagatgatacaacggccaggacgattatgttgagtaggattgtatccctggcaaggggcaattctgccataactcccgccaaccttgacaaattagtggcagtgttgaacgcaggaatcgtcccatgcatccctgaaaaaggttcattaggtacttcaggtgacttaggccctctggcagctatcgccttagtatgcgctggccagtggaaggcacgttacaatggacaaataatgccaggccgtcaggctctatcagaggccggtgtcgaacctatggaactttcatataaggacggcctagcacttataaacggtacgtctggaatggtaggccttggaactatggttttacaagccgctaggaggttagtggacaggtatttgcaagtaagtgccttatctgtagaaggcttggcaggcatgactaaaccttttgaccctcgtgtacacggagttaagccccatagaggtcaaagacaagtagcttcccgtctatgggaaggattggccgattctcaccttgccgtgaacgagcttgatactgaacaaacccttgcaggtgaaatgggcacggtggcaaaggctggaagtttagccatagaggatgcctactccattcgttgcacccctcagatcctgggtccagtcgtagacgtattggataggatcggtgcaacattacaagacgagctgaattcttcaaacgataaccccattgtccttcctgaggaagcagaagtattccataatggacactttcatggacagtatgtcgccatggcaatggatcacctaaatatggctcttgctactgttacgaatcttgccaatagaagagtcgacagatttctagataaatcaaattccaatggactaccagcctttctttgccgtgaagacccaggccttaggcttggtttgatgggcggccagttcatgactgcatcaatcaccgctgaaacccgtacactgacaatcccaatgtctgtacaatcacttacttccacggcagattttcaagacatagtatcctttggcttcgtagccgcaaggagagctagagaggtgttgacaaacgctgcttatgtggtagcctttgaactactgtgtgcctgtcaggctgtggacatcagaggagcagacaagctatcttctttcacccgtcctctatatgaaagaaccaggaaaattgttccctttttcgacagagatgaaacaattactgactacgttgagaagttagctgctgatttaatagcaggcgagcctgtagatgccgctgtggctgcacat

SEQ ID NO: 117 - protein, 523 aa, Streptomyces maritimus
MTFVIELDMNVTLDQLEDAARQRTPVELSAPVRSRVRASRDVLVKFVQDERVIYGVNTSMGGFVDHLVPVSQARQLQENLINAVATNVGAYLDDTTARTIMLSRIVSLARGNSAITPANLDKLVAVLNAGIVPCIPEKGSLGTSGDLGPLAAIALVCAGQWKARYNGQIMPGRQALSEAGVEPMELSYKDGLALINGTSGMVGLGTMVLQAARRLVDRYLQVSALSVEGLAGMTKPFDPRVHGVKPHRGQRQVASRLWEGLADSHLAVNELDTEQTLAGEMGTVAKAGSLAIEDAYSIRCTPQILGPVVDVLDRIGATLQDELNSSNDNPIVLPEEAEVFHNGHFHGQYVAMAMDHLNMALATVTNLANRRVDRFLDKSNSNGLPAFLCREDPGLRLGLMGGQFMTASITAETRTLTIPMSVQSLTSTADFQDIVSFGFVAARRAREVLTNAAYVVAFELLCACQAVDIRGADKLSSFTRPLYERTRKIVPFFDRDETITDYVEKLAADLIAGEPVDAAVAAH

SEQ ID NO: 118 - DNA, 1173 bp, Artificial sequence
  Nucleotide sequence PKS from Aquilaria sinensis, optimized for expression in Nicotiana benthamiana
atgggatctcaagatgttgctggaggagctcttaagggagttaatccaggaaaggctactattcttgctcttggaaaggcttttccatatcaacttgttatgcaagaatttcttgttgatggatattttaagaatacttcttgtaaggatcaagaacttaagcaaaagcttgctagactttgtaagactactactgttaagactagatatgttgttatgtctgaagaaattcttaataagtatccagaacttgctgttgaaggaattccaactcttaagcaaagacttgatattggaaatgaagctcttactgaaatggctattgaagcttctcaagcttgtattaagaagtggggaagaccagcttctgaaattactcatcttgtttatgtttcttcttctgaagctagacttccaggaggagatctttatcttgctcaaggacttggactttctccaagaactaagagagttgttctttattttatgggatgttctggaggagttgctggacttagagttgctaaggatattgctgaaaataatccaggatctagagttcttcttgctacttctgaaactactattgttggatttaagccaccatctgctcatagaccatatgatcttgttggagttgctctttttggagatggagctggagctatggttattggatctgatccacttccaggaactgaatctccactttttgaacttcatactgctattcaaaattttcttccaaatactgaaaagactattgatggaagacttactgaagaaggaatttcttttaagcttgctagagaacttccacaaattgttgaagatcatattgaaggattttgtggacaacttactggagttattggactttctcataagcaatataataagatgttttgggctgttcatccaggaggaccagctattcttaatagagttgaaaagagacttgatcttcatccaaataagcttgatgcttctagaagagctcttgaagattatggaaatgcttcttctaattctattgtttatgttcttgattatatgattgaagaaactcttaagatgaagactgaatctcttgaaccatctgaatggggacttattcttgcttttggaccaggagttacttttgaaggaattcttgctagaaatcttgctgtt

SEQ ID NO: 119 - protein, 391 aa, Aquilaria sinensis
MGSQDVAGGALKGVNPGKATILALGKAFPYQLVMQEFLVDGYFKNTSCKDQELKQKLARLCKTTTVKTRYVVMSEEILNKYPELAVEGIPTLKQRLDIGNEALTEMAIEASQACIKKWGRPASEITHLVYVSSSEARLPGGDLYLAQGLGLSPRTKRVVLYFMGCSGGVAGLRVAKDIAENNPGSRVLLATSETTIVGFKPPSAHRPYDLVGVALFGDGAGAMVIGSDPLPGTESPLFELHTAIQNFLPNTEKTIDGRLTEEGISFKLARELPQIVEDHIEGFCGQLTGVIGLSHKQYNKMFWAVHPGGPAILNRVEKRLDLHPNKLDASRRALEDYGNASSNSIVYVLDYMIEETLKMKTESLEPSEWGLILAFGPGVTFEGILARNLAV

SEQ ID NO: 120 - DNA, 1164 bp, Artificial sequence
  Nucleotide sequence PKS2 from Aquilaria sinensis, optimized for expression in Nicotiana benthamiana
atgtctcaagctattgctgataatgcttatagacatcatcttaagagagctccaactccaggaaaggctactgttcttgctcttggaaaggcttttccaaagcaagttattccacaagaaaatcttgttgaaggatatattagagatactaagtgtgaagatgtttctattaaggaaaagcttgaaagactttgtaagactactactgttaagactagatatactgttatgtctaaggaaattcttgataattatccagaacttgttactgaaggatctccaactattagacaaagacttgaaattgctaatccagctgttgttgaaatggctaaggaagcttctcttgcttgtattaagcaatggggaagaccagctggagatattactcatattgtttatgtttcttcttctgaaattagacttccaggaggagatctttatcttgctaatgaacttggacttaagaatgatattaatagaattatgctttattttcttggatgttatggaggagttactggacttagagttgctaaggatattgctgaaaataatccaggatctagaattcttcttactacttctgaaactactattcttggatttagaccaccaaataagtctagaccatatgatcttgttggagctgctctttttggagatggagctgctgctgttattattggagctaatccagaaattggaagagaatctccatttatggaacttaattttgctcttcaacaatttcttccaggaactcatggagttattgatggaagactttctgaagaaggaattaattttaagcttggaagagatcttccacaaaagattgaagataatattgaagatttttgtagaaagcttatgattaaggctgatggagatcttaaggaatttaatgaacttttttgggctgttcatccaggaggaccagctattcttaatagacttgaatctattcttgatcttaagaatggaaagcttgaatgttctagaagagctcttatggattatggaaatgtttcttctaatactattttttatgttatggaatatatgagagaagaacttaagagagaaggatctgaagaatggggacttgctcttgcttttggaccaggaattacttttgaaggaattcttcttagatctctt

SEQ ID NO: 121 - protein, 388 aa, Aquilaria sinensis
MSQAIADNAYRHHLKRAPTPGKATVLALGKAFPKQVIPQENLVEGYIRDTKCEDVSIKEKLERLCKTTTVKTRYTVMSKEILDNYPELVTEGSPTIRQRLEIANPAVVEMAKEASLACIKQWGRPAGDITHIVYVSSSEIRLPGGDLYLANELGLKNDINRIMLYFLGCYGGVTGLRVAKDIAENNPGSRILLTTSETTILGFRPPNKSRPYDLVGAALFGDGAAAVIIGANPEIGRESPFMELNFALQQFLPGTHGVIDGRLSEEGINFKLGRDLPQKIEDNIEDFCRKLMIKADGDLKEFNELFWAVHPGGPAILNRLESILDLKNGKLECSRRALMDYGNVSSNTIFYVMEYMREELKREGSEEWGLALAFGPGITFEGILLRSL

SEQ ID NO: 122 - DNA, 1185 bp, Artificial sequence
  Nucleotide sequence PKS from Arabidopsis thaliana, optimized for expression in Nicotiana benthamiana
atgtctaattctagaatgaatggagttgaaaagctttcttctaagtctactagaagagttgctaatgctggaaaggctactcttcttgctcttggaaaggcttttccatctcaagttgttccacaagaaaatcttgttgaaggatttcttagagatactaagtgtgatgatgcttttattaaggaaaagcttgaacatctttgtaagactactactgttaagactagatatactgttcttactagagaaattcttgctaagtatccagaacttactactgaaggatctccaactattaagcaaagacttgaaattgctaatgaagctgttgttgaaatggctcttgaagcttctcttggatgtattaaggaatggggaagaccagttgaagatattactcatattgtttatgtttcttcttctgaaattagacttccaggaggagatctttatctttctgctaagcttggacttagaaatgatgttaatagagttatgctttattttcttggatgttatggaggagttactggacttagagttgctaaggatattgctgaaaataatccaggatctagagttcttcttactacttctgaaactactattcttggatttagaccaccaaataaggctagaccatatgatcttgttggagctgctctttttggagatggagctgctgctgttattattggagctgatccaagagaatgtgaagctccatttatggaacttcattatgctgttcaacaatttcttccaggaactcaaaatgttattgaaggaagacttactgaagaaggaattaattttaagcttggaagagatcttccacaaaagattgaagaaaatattgaagaattttgtaagaagcttatgggaaaggctggagatgaatctatggaatttaatgatatgttttgggctgttcatccaggaggaccagctattcttaatagacttgaaactaagcttaagcttgaaaaggaaaagcttgaatcttctagaagagctcttgttgattatggaaatgtttcttctaatactattctttatgttatggaatatatgagagatgaacttaagaagaagggagatgctgctcaagaatggggacttggacttgcttttggaccaggaattacttttgaaggacttcttattagatctcttacttcttct

SEQ ID NO: 123 - protein, 395 aa, Arabidopsis thaliana
MSNSRMNGVEKLSSKSTRRVANAGKATLLALGKAFPSQVVPQENLVEGFLRDTKCDDAFIKEKLEHLCKTTTVKTRYTVLTREILAKYPELTTEGSPTIKQRLEIANEAVVEMALEASLGCIKEWGRPVEDITHIVYVSSSEIRLPGGDLYLSAKLGLRNDVNRVMLYFLGCYGGVTGLRVAKDIAENNPGSRVLLTTSETTILGFRPPNKARPYDLVGAALFGDGAAAVIIGADPRECEAPFMELHYAVQQFLPGTQNVIEGRLTEEGINFKLGRDLPQKIEENIEEFCKKLMGKAGDESMEFNDMFWAVHPGGPAILNRLETKLKLEKEKLESSRRALVDYGNVSSNTILYVMEYMRDELKKKGDAAQEWGLGLAFGPGITFEGLLIRSLTSS

SEQ ID NO: 124 - DNA, 1197 bp, Artificial sequence
  Nucleotide sequence PKS from Hydrangea macrophylla, optimized for expression in Nicotiana benthamiana
atggctactaagtctgttgctgttgaagaaatgtgtaaggctcaaaaggctggaggaccagctactattcttgctattggaactgctgttccatctaattgttattatcaatctgaatatccagatttttattttagagttactaagtctgatcatcttactgatcttaagtctaagtttaagagaatgtgtgaaagatcttctattaagaagagatatatgcatcttactgaagaaattcttgaagaaaatccaaatatgtgtacttttgctgctccatctattgatggaagacaagatattgttgttaaggaaattccaaagcttgctaaggaagctgcttctaaggctattaaggaatggggacaaccaaagtctaatattactcatcttgttttttgtactacttctggagttgatatgccaggatgtgattatcaacttactagacttcttggacttagaccatctattaagagacttatgatgtatcaacaaggatgtcatgctggaggaactggacttagacttgctaaggatcttgctgaaaataataagggagctagagttcttgttgtttgttctgaaatgactgttattaattttagaggaccatctgaagctcatatggattctcttgttggacaatctctttttggagatggagcttctgctgttattgttggatctgatccagatctttctactgaacatccactttatcaaattatgtctgcttctcaaattattgttgctgattctgaaggagctattgatggacatcttagacaagaaggacttacttttcatcttagaaaggatgttccatctcttgtttctgataatattgaaaatactcttgttgaagcttttactccaattcttatggattctattgattctattattgattggaattctattttttggattgctcatccaggaggaccagctattcttaatcaagttcaagctaaggttggacttaaggaagaaaagcttagagtttctagacatattctttctgaatatggaaatatgtcttctgcttgtgttttttttattatggatgaaatgagaaagagatctatggaagaaggaaagggaactactggagaaggacttgaatggggagttctttttggatttggaccaggatttactgttgaaactattgttcttcattctgttccaatt

SEQ ID NO: 125 - protein, 399 aa, Hydrangea macrophylla
MATKSVAVEEMCKAQKAGGPATILAIGTAVPSNCYYQSEYPDFYFRVTKSDHLTDLKSKFKRMCERSSIKKRYMHLTEEILEENPNMCTFAAPSIDGRQDIVVKEIPKLAKEAASKAIKEWGQPKSNITHLVFCTTSGVDMPGCDYQLTRLLGLRPSIKRLMMYQQGCHAGGTGLRLAKDLAENNKGARVLVVCSEMTVINFRGPSEAHMDSLVGQSLFGDGASAVIVGSDPDLSTEHPLYQIMSASQIIVADSEGAIDGHLRQEGLTFHLRKDVPSLVSDNIENTLVEAFTPILMDSIDSIIDWNSIFWIAHPGGPAILNQVQAKVGLKEEKLRVSRHILSEYGNMSSACVFFIMDEMRKRSMEEGKGTTGEGLEWGVLFGFGPGFTVETIVLHSVPI

SEQ ID NO: 126 - DNA, 1254 bp, Artificial sequence
  Nucleotide sequence PKS from Physcomitrella patens, optimized for expression in Nicotiana benthamiana
atggcttctagaagagttgaagctgcttttgatggacaagctgttgaacttggagctactattccagctgctaatggaaatggaactcatcaatctattaaggttccaggacatagacaagttactccaggaaagactactattatggctattggaagagctgttccagctaatactacttttaatgatggacttgctgatcattatattcaagaatttaatcttcaagatccagttcttcaagctaagcttagaagactttgtgaaactactactgttaagactagatatcttgttgttaataaggaaattcttgatgaacatccagaatttcttgttgatggagctgctactgtttctcaaagacttgctattactggagaagctgttactcaacttggacatgaagctgctactgctgctattaaggaatggggaagaccagcttctgaaattactcatcttgtttatgtttcttcttctgaaattagacttccaggaggagatctttatcttgctcaacttcttggacttagatctgatgttaatagagttatgctttatatgcttggatgttatggaggagcttctggaattagagttgctaaggatcttgctgaaaataatccaggatctagagttcttcttattacttctgaatgtactcttattggatataagtctctttctccagatagaccatatgatcttgttggagctgctctttttggagatggagctgctgctatgattatgggaaaggatccaattccagttcttgaaagagctttttttgaacttgattgggctggacaatcttttattccaggaactaataagactattgatggaagactttctgaagaaggaatttcttttaagcttggaagagaacttccaaagcttattgaatctaatattcaaggattttgtgatccaattcttaagagagctggaggacttaagtataatgatattttttgggctgttcatccaggaggaccagctattcttaatgctgttcaaaagcaacttgatcttgctccagaaaagcttcaaactgctagacaagttcttagagattatggaaatatttcttcttctacttgtatttatgttcttgattatatgagacatcaatctcttaagcttaaggaagctaatgataatgttaatactgaaccagaatggggacttcttcttgcttttggaccaggagttactattgaaggagctcttcttagaaatctttgt

SEQ ID NO: 127 - protein, 397 aa, Physcomitrella patens
MSKTVEDRAAQRAKGPATVLAIGTATPANVVYQTDYPDYYFRVTKSEHMTKLKNKFQRMCDRSTIKKRYMVLTEELLEKNLSLCTYMEPSLDARQDILVPEVPKLGKEAADEAIAEWGRPKSEITHLIFCTTCGVDMPGADYQLTKLLGLRSSVRRTMLYQQGCFGGGTVLRLAKDLAENNAGARVLVVCSEITTAVNFRGPSDTHLDLLVGLALFGDGAAAVIVGADPDPTLERPLFQIVSGAQTILPDSEGAINGHLREVGLTIRLLKDVPGLVSMNIEKCLMEAFAPMGIHDWNSIFWIAHPGGPTILDQVEAKLGLKEEKLKSTRAVLREYGNMSSACVLFILDEVRKRSMEEGKTTTGEGFDWGVLFGFGPGFTVETVVLHSMPIPKADEGR

SEQ ID NO: 128 - DNA, 1179 bp, Artificial sequence
  Nucleotide sequence PKS from Polygonum cuspidatum, optimized for expression in Nicotiana benthamiana
atggctccagctgttgctgatattagaaaggctcaaagagctgaaggaccagctactgttcttgctattggaactgctactccaccaaattgtgtttatcaaaaggattatccagattattattttagagttactaattctgatcatatgactgatcttaaggaaaagtttagaagaatgtgtgaaaagtctaatattgaaaagagatatatgtatcttactgaagaaattcttaaggaaaatccaaatatgtgttcttatatgcaaacttcttctcttgatactagacaagatatggttgtttctgaagttccaagacttggaaaggaagctgctcaaaaggctattaaggaatggggacaaccaaagtctaagattactcatgttattatgtgtactacttctggagttgatatgccaggagctgattatcaacttactaagcttcttggacttcatccatctgttaagagatttatgatgtatcaacaaggatgttttgctggaggaactgttcttagacttgctaaggatcttgctgaaaataatagaggagctagagttcttgttgtttgttctgaaattactgctatttgttttagaggaccaactgatactcatccagattctatggttggacaagctctttttggagatggatctggagctgttattattggagctgatccagatctttctattgaaaagccaatttttgaacttgtttggactgctcaaactattcttccagattctgaaggagctattgatggacatcttagagaagttggacttacttttcatcttcttaaggatgttccaggacttatttctaagaatattgaaaagaatcttactgaagctttttctccacttaatgtttctgattggaattctcttttttggattgctcatccaggaggaccagctattcttgatcaagttgaaactaagcttggacttaaggaagaaaagcttaaggctactagacaagttcttaatgattatggaaatatgtcttctgcttgtgttctttttattatggatgaaatgagaaagaagtctgttgaaaatggacatgctactactggagaaggacttgaatggggagttctttttggatttggaccaggacttactgttgaaactgttgttcttcattctgttccagttgctaat

SEQ ID NO: 129 - protein, 393 aa, Polygonum cuspidatum
MAPAVADIRKAQRAEGPATVLAIGTATPPNCVYQKDYPDYYFRVTNSDHMTDLKEKFRRMCEKSNIEKRYMYLTEEILKENPNMCSYMQTSSLDTRQDMVVSEVPRLGKEAAQKAIKEWGQPKSKITHVIMCTTSGVDMPGADYQLTKLLGLHPSVKRFMMYQQGCFAGGTVLRLAKDLAENNRGARVLVVCSEITAICFRGPTDTHPDSMVGQALFGDGSGAVIIGADPDLSIEKPIFELVWTAQTILPDSEGAIDGHLREVGLTFHLLKDVPGLISKNIEKNLTEAFSPLNVSDWNSLFWIAHPGGPAILDQVETKLGLKEEKLKATRQVLNDYGNMSSACVLFIMDEMRKKSVENGHATTGEGLEWGVLFGFGPGLTVETVVLHSVPVAN

SEQ ID NO: 130 - DNA, 1152 bp, Artificial squence
  Nucleotide sequence of the mutant L132S PKS from Rheum palmatum, optimized for expression in Nicotiana benthamiana
atggctactgaagaaatgaagaagcttgctactgttatggctattggaactgctaatccaccaaattgttattatcaagctgattttccagatttttattttagagttactaattctgatcatcttattaatcttaagcaaaagtttaagagactttgtgaaaattctagaattgaaaagagatatcttcatgttactgaagaaattcttaaggaaaatccaaatattgctgcttatgaagctacttctcttaatgttagacataagatgcaagttaagggagttgctgaacttggaaaggaagctgctcttaaggctattaaggaatggggacaaccaaagtctaagattactcatcttattgtttgttgttctgctggagttgatatgccaggagctgattatcaacttactaagcttcttgatcttgatccatctgttaagagatttatgttttatcatcttggatgttatgctggaggaactgttcttagacttgctaaggatattgctgaaaataataagggagctagagttcttattgtttgttctgaaatgactactacttgttttagaggaccatctgaaactcatcttgattctatgattggacaagctattcttggagatggagctgctgctgttattgttggagctgatccagatcttactgttgaaagaccaatttttgaacttgtttctactgctcaaactattgttccagaatctcatggagctattgaaggacatcttcttgaatctggactttcttttcatctttataagactgttccaactcttatttctaataatattaagacttgtctttctgatgcttttactccacttaatatttctgattggaattctcttttttggattgctcatccaggaggaccagctattcttgatcaagttactgctaaggttggacttgaaaaggaaaagcttaaggttactagacaagttcttaaggattatggaaatatgtcttctgctactgttttttttattatggatgaaatgagaaagaagtctcttgaaaatggacaagctactactggagaaggacttgaatggggagttctttttggatttggaccaggaattactgttgaaactgttgttcttagatctgttccagttatttct

SEQ ID NO: 131 - protein, 384 aa, Artificial sequence
  Amino acid sequence of the mutant L132S PKS from Rheum palmatum, optimized for expression in Nicotiana benthamiana
MATEEMKKLATVMAIGTANPPNCYYQADFPDFYFRVTNSDHLINLKQKFKRLCENSRIEKRYLHVTEEILKENPNIAAYEATSLNVRHKMQVKGVAELGKEAALKAIKEWGQPKSKITHLIVCCSAGVDMPGADYQLTKLLDLDPSVKRFMFYHLGCYAGGTVLRLAKDIAENNKGARVLIVCSEMTTTCFRGPSETHLDSMIGQAILGDGAAAVIVGADPDLTVERPIFELVSTAQTIVPESHGAIEGHLLESGLSFHLYKTVPTLISNNIKTCLSDAFTPLNISDWNSLFWIAHPGGPAILDQVTAKVGLEKEKLKVTRQVLKDYGNMSSATVFFIMDEMRKKSLENGQATTGEGLEWGVLFGFGPGITVETVVLRSVPVIS

SEQ ID NO: 132 - protein, 1173 aa, Artificial sequence
  Nucleotide sequence of PKS from Rheum tataricum, optimized for expression in Nicotiana benthamiana
ATGGCTCCAGAAGAATCTAGACATGCTGAAACTGCTGTTAATAGAGCTGCTACTGTTCTTGCTATTGGAACTGCTAATCCACCAAATTGTTATTATCAAGCTGATTTTCCAGATTTTTATTTTAGAGCTACTAATTCTGATCATCTTACTCATCTTAAGCAAAAGTTTAAGAGAATTTGTGAAAAGTCTATGATTGAAAAGAGATATCTTCATCTTACTGAAGAAATTCTTAAGGAAAATCCAAATATTGCTTCTTTTGAAGCTCCATCTCTTGATGTTAGACATAATATTCAAGTTAAGGAAGTTGTTCTTCTTGGAAAGGAAGCTGCTCTTAAGGCTATTAATGAATGGGGACAACCAAAGTCTAAGATTACTAGACTTATTGTTTGTTGTATTGCTGGAGTTGATATGCCAGGAGCTGATTATCAACTTACTAAGCTTCTTGGACTTCAACTTTCTGTTAAGAGATTTATGTTTTATCATCTTGGATGTTATGCTGGAGGAACTGTTCTTAGACTTGCTAAGGATATTGCTGAAAATAATAAGGAAGCTAGAGTTCTTATTGTTAGATCTGAAATGACTCCAATTTGTTTTAGAGGACCATCTGAAACTCATATTGATTCTATGGTTGGACAAGCTATTTTTGGAGATGGAGCTGCTGCTGTTATTGTTGGAGCTAATCCAGATCTTTCTATTGAAAGACCAATTTTTGAACTTATTTCTACTTCTCAAACTATTATTCCAGAATCTGATGGAGCTATTGAAGGACATCTTCTTGAAGTTGGACTTTCTTTTCAACTTTATCAAACTGTTCCATCTCTTATTTCTAATTGTATTGAAACTTGTCTTTCTAAGGCTTTTACTCCACTTAATATTTCTGATTGGAATTCTCTTTTTTGGATTGCTCATCCAGGAGGAAGAGCTATTCTTGATGATATTGAAGCTACTGTTGGACTTAAGAAGGAAAAGCTTAAGGCTACTAGACAAGTTCTTAATGATTATGGAAATATGTCTTCTGCTTGTGTTTTTTTTATTATGGATGAAATGAGAAAGAAGTCTCTTGCTAATGGACAAGTTACTACTGGAGAAGGACTTAAGTGGGGAGTTCTTTTTGGATTTGGACCAGGAGTTACTGTTGAAACTGTTGTTCTTTCTTCTGTTCCACTTATTACT

SEQ ID NO: 133 - protein, 391 aa, Rheum tataricum
MAPEESRHAETAVNRAATVLAIGTANPPNCYYQADFPDFYFRATNSDHLTHLKQKFKRICEKSMIEKRYLHLTEEILKENPNIASFEAPSLDVRHNIQVKEVVLLGKEAALKAINEWGQPKSKITRLIVCCIAGVDMPGADYQLTKLLGLQLSVKRFMFYHLGCYAGGTVLRLAKDIAENNKEARVLIVRSEMTPICFRGPSETHIDSMVGQAIFGDGAAAVIVGANPDLSIERPIFELISTSQTIIPESDGAIEGHLLEVGLSFQLYQTVPSLISNCIETCLSKAFTPLNISDWNSLFWIAHPGGRAILDDIEATVGLKKEKLKATRQVLNDYGNMSSACVFFIMDEMRKKSLANGQVTTGEGLKWGVLFGFGPGVTVETVVLSSVPLIT

SEQ ID NO: 134 - DNA, 1182 bp, Artificial sequence
  Nucleotide sequence PKS from Wachendorfia thyrsiflora, optimized for expression in Nicotiana benthamiana
atggcttctactgaaggaattcaagcttatagaaataatatggctgaaggaccagctactattatggctattggaactgctaatccaccaaatgttgttgatgcttctacttttccagattattattggagagttactaattctgaacatctttctccagaatatagagttaagcttaagagaatttgtgaaagatcttctattagaaagagacatcttgttcttactgaacaacttcttaaggaaaatccaactcttactacttatgttgatgcttcttatgatgaaagacaatctattgttcttgatgctgttccaaagcttgcttgtgaagctgctgctaaggctattaaggaatggggaagaccaaagactgatattactcatatggttgtttgtactggagctggagttgatgttccaggagttgattataagatgatgaatcttcttggacttccaccaactgttaatagagttatgctttataatgttggatgtcatgcttctggaactgttcttagaattgctaaggatcttgctgaaaataataagggagctagagttcttgttgtttcttctgaagtttctgttatgttttttagaggaccagctgaaggagatgttgaaattcttcttggacaagctctttttggagatggatctgctgctattattgttggagctgatccaattgaaggagttgaaaagccaatttttcaaattttttctgcttctcaaatgactcttccagaaggagaacatcttgttgctggacatcttagagaacttggacttacttttcatcttaagccacaacttccaaatactgtttcttctaatattcataagccacttaagaaggcttttgaaccacttaatattactgattggaattctattttttggattgttcatccaggaggaagagctattcttgatcaagttcaagaaaagattggacttgaagaaaataagcttgatgtttctagatatgttcttgctgaaaatggaaatatgatgtctgcttctgttttttttattatggatgaaatgagaaagagatctgctgctcaaggatgttctactactggagaaggacatgaatggggagttctttttggatttggaccaggactttctattgaaactgttgttcttcattctgttccactttctatt

SEQ ID NO: 135 - protein, 394 aa, Wachendorfia thyrsiflora
MASTEGIQAYRNNMAEGPATIMAIGTANPPNVVDASTFPDYYWRVTNSEHLSPEYRVKLKRICERSSIRKRHLVLTEQLLKENPTLTTYVDASYDERQSIVLDAVPKLACEAAAKAIKEWGRPKTDITHMVVCTGAGVDVPGVDYKMMNLLGLPPTVNRVMLYNVGCHASGTVLRIAKDLAENNKGARVLVVSSEVSVMFFRGPAEGDVEILLGQALFGDGSAAIIVGADPIEGVEKPIFQIFSASQMTLPEGEHLVAGHLRELGLTFHLKPQLPNTVSSNIHKPLKKAFEPLNITDWNSIFWIVHPGGRAILDQVQEKIGLEENKLDVSRYVLAENGNMMSASVFFIMDEMRKRSAAQGCSTTGEGHEWGVLFGFGPGLSIETVVLHSVPLSI

SEQ ID NO: 136 - DNA, 1191 bp, Artificial sequence
  Nucleotide sequence PKS1 from Piper methysticum, optimized for expression in Nicotiana benthamiana
atgtctaagactgttgaagatagagctgctcaaagagctaagggaccagctactgttcttgctattggaactgctactccagctaatgttgtttatcaaactgattatccagattattattttagagttactaagtctgaacatatgactaagcttaagaataagtttcaaagaatgtgtgatagatctactattaagaagagatatatggttcttactgaagaacttcttgaaaagaatctttctctttgtacttatatggaaccatctcttgatgctagacaagatattcttgttccagaagttccaaagcttggaaaggaagctgctgatgaagctattgctgaatggggaagaccaaagtctgaaattactcatcttattttttgtactacttgtggagttgatatgccaggagctgattatcaacttactaagcttcttggacttagatcttctgttagaagaactatgctttatcaacaaggatgttttggaggaggaactgttcttagacttgctaaggatcttgctgaaaataatgctggagctagagttcttgttgtttgttctgaaattactactgctgttaattttagaggaccatctgatactcatcttgatcttcttgttggacttgctctttttggagatggagctgctgctgttattgttggagctgatccagatccaactcttgaaagaccactttttcaaattgtttctggagctcaaactattcttccagattctgaaggagctattaatggacatcttagagaagttggacttactattagacttcttaaggatgttccaggacttgtttctatgaatattgaaaagtgtcttatggaagcttttgctccaatgggaattcatgattggaattctattttttggattgctcatccaggaggaccaactattcttgatcaagttgaagctaagcttggacttaaggaagaaaagcttaagtctactagagctgttcttagagaatatggaaatatgtcttctgcttgtgttctttttattcttgatgaagttagaaagagatctatggaagaaggaaagactactactggagaaggatttgattggggagttctttttggatttggaccaggatttactgttgaaactgttgttcttcattctatgccaattccaaaggctgatgaaggaaga

SEQ ID NO: 137 - protein, 397 aa, Piper methysticum; identical to SEQ ID NO: 127

SEQ ID NO: 138 - DNA, 1182 bp, Artificial sequence
  Nucleotide sequence of PKS2 from Piper methysticum, optimized for expression in Nicotiana benthamiana
atgtctaagatggttgaagaacattgggctgctcaaagagctagaggaccagctactgttcttgctattggaactgctaatccaccaaatgttctttatcaagctgattatccagatttttattttagagttactaagtctgaacatatgactcaacttaaggaaaagtttaagagaatttgtgataagtctgctattagaaagagacatcttcatcttactgaagaacttcttgaaaagaatccaaatatttgtgctcatatggctccatctcttgatgctagacaagatattgctgttgttgaagttccaaagcttgctaaggaagctgctactaaggctattaaggaatggggaagaccaaagtctgatattactcatcttattttttgtactacttgtggagttgatatgccaggagctgattatcaacttactactcttcttggacttagaccaactgttagaagaactatgctttatcaacaaggatgttttgctggaggaactgttcttagacatgctaaggattttgctgaaaataatagaggagctagagttcttgctgtttgttctgaatttactgttatgaatttttctggaccatctgaagctcatcttgattctatggttggaatggctctttttggagatggagcttctgctgttattgttggagctgatccagattttgctattgaaagaccactttttcaacttgtttctactactcaaactattgttccagattctgatggagctattaagtgtcatcttaaggaagttggacttactcttcatcttgttaagaatgttccagatcttatttctaataatatggataagattcttgaagaagcttttgctccacttggaattagagattggaattctattttttggactgctcatccaggaggagctgctattcttgatcaacttgaagctaagcttggacttaataaggaaaagcttaagactactagaactgttcttagagaatatggaaatatgtcttctgcttgtgtttgttttgttcttgatgaaatgagaagatcttctcttgaagaaggaaagactacttctggagaaggacttgaatggggaattcttcttggatttggaccaggacttactgttgaaactgttgttcttagatctgttccaatttctactgctaat

SEQ ID NO: 139 - protein, 394 aa, Piper methysticum
MSKMVEEHWAAQRARGPATVLAIGTANPPNVLYQADYPDFYFRVTKSEHMTQLKEKFKRICDKSAIRKRHLHLTEELLEKNPNICAHMAPSLDARQDIAVVEVPKLAKEAATKAIKEWGRPKSDITHLIFCTTCGVDMPGADYQLTTLLGLRPTVRRTMLYQQGCFAGGTVLRHAKDFAENNRGARVLAVCSEFTVMNFSGPSEAHLDSMVGMALFGDGASAVIVGADPDFAIERPLFQLVSTTQTIVPDSDGAIKCHLKEVGLTLHLVKNVPDLISNNMDKILEEAFAPLGIRDWNSIFWTAHPGGAAILDQLEAKLGLNKEKLKTTRTVLREYGNMSSACVCFVLDEMRRSSLEEGKTTSGEGLEWGILLGFGPGLTVETVVLRSVPISTAN

SEQ ID NO: 140 - DNA, 1734 bp, Arabidopsis thaliana
atggaagaagattataagatggctccacaagaacaagctgtttctcaagttatggaaaagcaatctaataataataattctgatgttatttttagatctaagcttccagatatttatattccaaatcatctttctcttcatgattatatttttcaaaatatttctgaatttgctactaagccatgtcttattaatggaccaactggacatgtttatacttattctgatgttcatgttatttctagacaaattgctgctaattttcataagcttggagttaatcaaaatgatgttgttatgcttcttcttccaaattgtccagaatttgttctttcttttcttgctgcttcttttagaggagctactgctactgctgctaatccattttttactccagctgaaattgctaagcaagctaaggcttctaatactaagcttattattactgaagctagatatgttgataagattaagccacttcaaaatgatgatggagttgttattgtttgtattgatgataatgaatctgttccaattccagaaggatgtcttagatttactgaacttactcaatctactactgaagcttctgaagttattgattctgttgaaatttctccagatgatgttgttgctcttccatattcttctggaactactggacttccaaagggagttatgcttactcataagggacttgttacttctgttgctcaacaagttgatggagaaaatccaaatctttattttcattctgatgatgttattctttgtgttcttccaatgtttcatatttatgctcttaattctattatgctttgtggacttagagttggagctgctattcttattatgccaaagtttgaaattaatcttcttcttgaacttattcaaagatgtaaggttactgttgctccaatggttccaccaattgttcttgctattgctaagtcttctgaaactgaaaagtatgatctttcttctattagagttgttaagtctggagctgctccacttggaaaggaacttgaagatgctgttaatgctaagtttccaaatgctaagcttggacaaggatatggaatgactgaagctggaccagttcttgctatgtctcttggatttgctaaggaaccatttccagttaagtctggagcttgtggaactgttgttagaaatgctgaaatgaagattgttgatccagatactggagattctctttctagaaatcaaccaggagaaatttgtattagaggacatcaaattatgaagggatatcttaataatccagctgctactgctgaaactattgataaggatggatggcttcatactggagatattggacttattgatgatgatgatgaactttttattgttgatagacttaaggaacttattaagtataagggatttcaagttgctccagctgaacttgaagctcttcttattggacatccagatattactgatgttgctgttgttgctatgaaggaagaagctgctggagaagttccagttgcttttgttgttaagtctaaggattctgaactttctgaagatgatgttaagcaatttgtttctaagcaagttgttttttataagagaattaataaggttttttttactgaatctattccaaaggctccatctggaaagattcttagaaaggatcttagagctaagcttgctaatggactttctggatatccatatgatgttccagattatgct

SEQ ID NO: 141 - protein, 578 aa, Arabidopsis thaliana
MEEDYKMAPQEQAVSQVMEKQSNNNNSDVIFRSKLPDIYIPNHLSLHDYIFQNISEFATKPCLINGPTGHVYTYSDVHVISRQIAANFHKLGVNQNDVVMLLLPNCPEFVLSFLAASFRGATATAANPFFTPAEIAKQAKASNTKLIITEARYVDKIKPLQNDDGVVIVCIDDNESVPIPEGCLRFTELTQSTTEASEVIDSVEISPDDVVALPYSSGTTGLPKGVMLTHKGLVTSVAQQVDGENPNLYFHSDDVILCVLPMFHIYALNSIMLCGLRVGAAILIMPKFEINLLLELIQRCKVTVAPMVPPIVLAIAKSSETEKYDLSSIRVVKSGAAPLGKELEDAVNAKFPNAKLGQGYGMTEAGPVLAMSLGFAKEPFPVKSGACGTVVRNAEMKIVDPDTGDSLSRNQPGEICIRGHQIMKGYLNNPAATAETIDKDGWLHTGDIGLIDDDDELFIVDRLKELIKYKGFQVAPAELEALLIGHPDITDVAVVAMKEEAAGEVPVAFVVKSKDSELSEDDVKQFVSKQVVFYKRINKVFFTESIPKAPSGKILRKDLRAKLANGLSGYPYDVPDYA